(19) United States
(12) Patent Application Publication
(10) Pub. No.: US 2026/0139023 A1
(43) Pub. Date: May 21, 2026

(54) NOVEL IRISIN PEPTIDES AND METHODS OF USE THEREOF

(71) Applicant: Dana-Farber Cancer Institute, Inc., Boston, MA (US)

(72) Inventors: Bruce M. Spiegelman, Waban, MA (US); Mu A, Brookline, MA (US)

(21) Appl. No.: 19/119,300

(22) PCT Filed: Oct. 10, 2023
(86) PCT No.: PCT/US2023/034805

§ 371 Date: Apr. 8, 2025

Related U.S. Application Data
(60) Provisional application No. 63/415,058, filed on Oct. 11, 2022.

(51) Int. Cl. C07K 14/575; A61K 38/00

(52) U.S. Cl. CPC C07K 14/575; A61K 38/00

(57) ABSTRACT

The present invention provides novel irisin peptides (e.g, irisin glycosylation mutants and biologically active fragments thereof). Also provided are methods for preventing or reducing degeneration of dopaminergic neurons and/or preventing or ameliorating at least one motor deficit in a subject in need thereof, such as in a subject with α-synucleinopathy, using the novel irisin peptides to modulate irisin-induced integrin signaling. The novel irisin peptides can also be used in methods for increasing expression of brain-derived neurotrophic factor (BDNF), and/or treating or preventing neurological diseases or disorders that would benefit from decreased neuronal cell death and/or increased neuronal survival in a subject. In addition, the novel irisin peptides may be used in methods for preventing or treating muscular atrophy or muscular dystrophy.

### CROSS-REFERENCE TO RELATED APPLICATIONS

This application claims the benefit of priority to U.S. Provisional Application Ser. No. 63/415,058, filed on Oct. 11, 2022; the entire contents of said application is incorporated herein in its entirety by this reference.

### BACKGROUND OF THE INVENTION

Irisin, a PGC-1α-dependent myokine, is a hormone formed by the cleavage of FNDC5 in muscle cells, generally in response to exercise. Naturally occurring irisin is a 12 kDa (112 amino-acid-long) N-terminal polypeptide (Norrbom et al., PGC-1alpha mRNA expression is influenced by metabolic perturbation in exercising human skeletal muscle. J Appl Physiol (1985). 2004; 96(1):189-94; Bostrom et al., A PGC1-alpha-dependent myokine that drives brown-fat-like development of white fat and thermogenesis. Nature. 2012; 481(7382):463-8). Irisin exists in human plasma at 3-5 ng/mL and its level in human blood is elevated following endurance exercise (Jedrychowski et al., Detection and Quantitation of Circulating Human Irisin by Tandem Mass Spectrometry. Cell Metab. 2015; 22(4):734-40). In bone and fat, the effects of irisin are mediated via av integrins, which use αVβ5 as their major receptor (Kim et al., Cell. 2018; 175(7):1756-68 e17). Irisin is glycosylated, and contains a fibronectin type III domain, which is relatively common and is shared by fibronectin plus many other protein (Bork et al., Proposed acquisition of an animal protein domain by bacteria. Proc Natl Acad Sci USA. 1992; 89(19):8990-4).

Irisin has been functionally associated with thermogenic programs (Bostrom et al. (2012) Nature 481:463-468; Oguri et al. (2020) Cell 182:563-577), bone remodeling (Colaianni et al. (2015) Proc. Natl. Acad. Sci. U.S.A. 112:12157-12162; Kim et al. (2018) Cell 175:1756-1768; Estell et al. (2020) eLife 9:e58172), and cognition (Wrann et al. (2013) Cell Metab. 18:649-659; Wrann (2015) Brain Plast. 1:55-61). Thus, modulation of irisin has important implications in methods of preventing or reducing degeneration of dopaminergic (DA) neurons preventing or ameliorating at least one motor deficit and/or preventing or ameliorating at least one symptom of cognitive dysfunction or dementia, such as to prevent or treat an α-synucleinopathy, such as Parkinson's disease, Lewy body dementia, Alzheimer's disease multiple system atrophy (MSA), or a neuroaxonal dystrophy; acting on neurons of the central and/or peripheral nervous system to enhance BDNF expression/activity and increase neuronal survival and function to thereby prevent or treat undesired neurological disorders; and increasing muscle physiology, such as to ameliorate the effects of a muscular dystrophy and/or muscular atrophy.

However, naturally occurring irisin is not ideally suited as a direct treatment for gaining exercise-induced beneficial effects. Natural form of irisin does not contain the RGD motif that has been identified in most integrin ligands as the key integrin-binding motif (Van Agthoven et al., Structural basis for pure antagonism of integrin alphaVbeta3 by a high-affinity form of fibronectin. Nat Struct Mol Biol. 2014; 21(4):383-8). Furthermore, integrin ligands usually contain multiple synergetic sites that help increase ligand binding affinity by positioning the RGD motif to the ligand binding pocket, but irisin itself, as a 12 kDa small polypeptide, has really high affinity for integrin receptor, indicating a non-canonical way of ligand binding for integrins. In addition, some irisin from mammalian cells exhibits a short half-life in vivo, and thus posing a serious challenge to its therapeutic applications.

### SUMMARY OF THE INVENTION

The present invention is based in part on the discovery of novel and non-naturally occurring forms of irisin that comprise site-specific glycosylation mutations that exhibit increased stability and receptor binding affinity. In some embodiments, the novel and non-naturally occurring forms of irisin comprise site-specific glycosylation mutations that exhibit increased half-life. The present disclosure provides an agent comprising at least one irisin polypeptide (e.g., an irisin glycosylation mutant, or any other irisin mutant disclosed herein) and biologically active fragments thereof, nucleic acids encoding such polypeptides, cells comprising the irisin polypeptides disclosed herein, and methods of treatment using such an agent. The agent disclosed herein can be administered to modulate (e.g., decrease or reduce) a level or amount of α-synuclein in cells of a subject in need thereof. The agent disclosed herein may also be administered to prevent or reduce degeneration of dopaminergic (DA) neurons, prevent or ameliorate at least one motor deficit and/or at least one symptom of cognitive dysfunction or dementia in a subject in need thereof. Administration of the agent disclosed herein may be used to treat Parkinson's disease, Lewy body dementia, Alzheimer's disease, multiple system atrophy (MSA), a neuroaxonal dystrophy, or Lou Gehrig's disease (ALS) in a subject in need thereof. The agent disclosed herein may be brought into contact with cells (e.g., in vivo, ex vivo, or in vitro) to increase expression of brain-derived neurotrophic factor (BDNF) in a subject in need thereof. Also disclosed herein are methods for treating or preventing a neurological disease or disorder in a subject, comprising administering to the subject an agent disclosed herein that increases BDNF expression or activity in central or peripheral nervous system of the subject, such that the neurological disease or disorder is treated or prevented. The agent disclosed herein may be brought into contact (e.g., in vivo, ex vivo, or in vitro) with muscle tissue that is affected by muscular dystrophy or muscular atrophy to increase muscle physiology of the muscle tissue.

Administration of the agent disclosed herein may also be used to prevent or treat muscular dystrophy or muscular atrophy in a subject in need thereof. The muscular dystrophy may be associated with a mutation in a dystrophin gene, and may be Becker, congenital, Duchenne, distal, Emery-Dreifuss, facioscapulohumeral, limb-girdle, myotonic, or oculopharyngeal muscular dystrophy. The muscular atrophy may be caused by disuse, trauma, or disease.

In some aspects, provided herein are polypeptides comprising a glycosylated residue at position 36, a glycosylated residue at position 81, and a non-N-linked glycosylated residue at position 45. In some embodiments, the non-N-linked glycosylated residue at position 45 is a non-canonical N-linked glycosylated residue at position 45. In some embodiments, the polypeptide is a non-naturally occurring form of irisin. In some embodiments, the residue at position 45 is mutated such that it cannot be N-linked glycosylated. In some embodiments, the irisin polypeptide comprises a Q residue at position 45. In some embodiments, the irisin polypeptide comprises a non-hydrophobic residue at position 45. In some embodiments, the non-hydrophobic residue at position 45 is selected from the list consisting of glutamine, threonine, serine, cysteine, arginine, histidine, lysine, aspartic acid, and glutamic acid. In some embodiments, the non-hydrophobic residue at position 45 does not cause misfolding within the irisin polypeptide. The irisin polypeptide may comprise an amino acid sequence having at least 80%, 85%, 90%, 95%, or 99% sequence identity to the amino acid sequence set forth in SEQ ID NO: 1 or SEQ ID NO: 13. In some embodiments, the irisin polypeptide may comprise SEQ ID NO: 1 or SEQ ID NO: 13. In some embodiments, the irisin polypeptide comprises a mutation to at least one residue listed in Table 2. In some embodiments, the irisin polypeptide comprises SEQ ID NO: 14 and a mutation to at least one residue listed in Table 2.

In some embodiments, the irisin polypeptide does not comprise a signal polypeptide. The irisin polypeptide may not comprise a FNDC5 signal polypeptide. In some embodiments, the irisin polypeptide comprises a signal polypeptide. In some embodiments, the irisin glycosylation mutant polypeptide comprises a signal polypeptide. The FNDC5 signal polypeptide may comprise the amino acid sequence of SEQ ID NO: 12. The irisin polypeptide may further comprise one or more polyhistidine (His)-Tag(s), optionally wherein the irisin polypeptide comprises two or more, five or more, or ten or more His-Tags. The irisin polypeptide may further comprise a human rhinovirus 3C protease (HRV-3C) protease tag. In some embodiments, the irisin polypeptide further comprises a GFP sequence. The irisin polypeptide may further comprise a Strep-II tag, optionally wherein the irisin polypeptide comprises at least two Strep-II tags. In some embodiments, the irisin polypeptide comprises the amino acid sequence set forth in SEQ ID NO: 15.

In some embodiments, the irisin polypeptide binds to an irisin receptor with at least 1-fold, at least 5-fold, or at least 10-fold more binding affinity to an irisin receptor compared to wild-type irisin. In some embodiments, the irisin polypeptide binds to an irisin receptor with at least 1-fold, at least-5 fold, or at least-10 fold increased binding affinity for αVβ5 and/or integrin compared to wild-type irisin. In some embodiments, the irisin polypeptide comprises at least 1-fold, at least 5-fold, or at least 10-fold increased stability compared to wild-type irisin. In some embodiments, the irisin polypeptide comprises at least 1-fold, at least 5-fold, or at least 10-fold increased half-life compared to wild-type irisin.

In some aspects, the irisin polypeptide is fused to one or more heterologous polypeptides at its N-terminus and/or C-terminus, and/or within any interstrand loop regions.

In some embodiments, the irisin polypeptide comprises an amino acid modification, post-translational modification, and/or a heterologous amino acid sequence that stabilizes the irisin polypeptide and/or increases its half-life.

Also provided herein are nucleic acids encoding the irisin polypeptide. In some embodiments, the nucleic acid is within an expression vector. The expression vector may be a viral expression vector, optionally wherein the viral expression vector is an adeno-associated viral (AAV) vector.

Another aspect of the invention provides cells comprising any one of the irisin polypeptides disclosed herein or the nucleic acids disclosed herein.

In some embodiments, these irisin agents (e.g., an irisin polypeptide disclosed described herein or a biologically active fragment thereof) can act to prevent or reduce degeneration of dopaminergic (DA) neurons, preventing or ameliorating at least one motor deficit and/or one symptom of cognitive dysfunction or dementia.

In some aspects, provided herein are methods to prevent or treat an α-synucleinopathy, such as Parkinson's disease, Lewy body dementia, Alzheimer's disease multiple system atrophy (MSA), Lou Gehrig's disease (ALS), or a neuroaxonal dystrophy, and a wide variety of muscle conditions, such as a muscular dystrophy (e.g., DMD) and muscle atrophy (e.g., atrophy from disuse, such as post-surgery or with limb immobilization; atrophy caused by trauma, such as an injury; or atrophy resulting from a disease state, such as spinal muscular atrophy or Charcot-Marie-Tooth disease), comprising administering to the subject an agent comprising: i) an irisin polypeptide disclosed herein or biologically active fragments thereof, or ii) a nucleic acid disclosed herein or biologically active fragments thereof.

Another aspect of the invention provides methods of preventing or reducing degeneration of dopaminergic (DA) neurons, preventing or ameliorating at least one motor deficit and/or at least one symptom of cognitive dysfunction or dementia in a subject in need thereof, the method comprising administering to the subject an agent comprising: i) an irisin polypeptide disclosed herein or biologically active fragments thereof, or ii) the nucleic acid encoding an irisin polypeptide disclosed herein or biologically active fragments thereof. In some embodiments, the subject is afflicted with an α-synucleinopathy. The subject may be afflicted with Parkinson's disease, Lewy body dementia, Alzheimer's disease, multiple system atrophy (MSA), a neuroaxonal dystrophy, or Lou Gehrig's disease (ALS). The motor deficit may be selected from the group consisting of: tremor at rest, such as a slight tremor in the hands or feet; rigidity (stiffness) of limbs, neck, or shoulders; difficulty balancing (postural instability); slowness of movement or gradual loss of spontaneous movement (bradykinesia); trouble standing after sitting; stiffness in the limbs; and moving more slowly. In yet another embodiment, the symptom of cognitive dysfunction or dementia is selected from the group consisting of confusion; poor motor coordination; loss of short-term or long-term memory; identity confusion; and impaired judgment.

Another aspect of the invention provides methods of decreasing or reducing a level or amount of α-synuclein in cells of a subject in need thereof, the method comprising administering to the subject an agent comprising: i) an irisin polypeptide disclosed herein or biologically active fragments thereof, or ii) a nucleic acid encoding an irisin polypeptide disclosed herein or biologically active fragments thereof. In some embodiments, the subject is afflicted with an α-synucleinopathy. In some embodiments, the cells are neurons or glia. In some embodiments, the subject is afflicted with Parkinson's disease, Lewy body dementia, Alzheimer's disease, multiple system atrophy (MSA), a neuroaxonal dystrophy, or Lou Gehrig's disease (ALS). In some embodiments, the cells are cancer cells, optionally wherein the cells are melanoma cells. The subject may be afflicted with a cancer characterized by or caused by an increase of α-synuclein. In some embodiments, the α-synuclein is pathogenic α-synuclein.

Also provided herein are methods of treating or preventing Parkinson's disease, Lewy body dementia, Alzheimer's disease, multiple system atrophy (MSA), a neuroaxonal dystrophy, or Lou Gehrig's disease (ALS) in a subject, the method comprising administering to the subject an agent comprising: i) an irisin polypeptide disclosed herein or biologically active fragments thereof, or ii) an nucleic acid encoding an irisin polypeptide disclosed herein or biologically active fragments thereof. In some embodiments, the agent is administered in a therapeutically effective amount to treat Parkinson's disease, Lewy body dementia, Alzheimer's disease, multiple system atrophy (MSA), a neuroaxonal dystrophy, or Lou Gehrig's disease (ALS).

Another aspect of the invention provides methods of increasing expression of brain-derived neurotrophic factor (BDNF) by a cell, comprising contacting the cell with an agent, wherein the agent comprises i) an irisin polypeptide disclosed herein or biologically active fragments thereof, or ii) a nucleic acid encoding an irisin polypeptide disclosed herein or biologically active fragments thereof. In some embodiments, the step of contacting occurs in vivo, ex vivo, or in vitro. In some embodiments, the cells are neurons. In some embodiments, the neurons are selected from the group consisting of hippocampal neurons, cerebellar neurons, sciatic nerve neurons, dopaminergic neurons, and substantia nigra neurons. In some embodiments, the method further comprises contacting the cell with an additional agent that increases the expression of the BDNF.

In some embodiments, the invention provides methods for treating or preventing a neurological disease or disorder in a subject, comprising the step of administering to the subject an agent comprising: i) an irisin polypeptide disclosed herein or biologically active fragments thereof, or ii) a nucleic acid encoding an irisin polypeptide disclosed herein or a biologically active fragment thereof, that increases BDNF expression or activity in central or peripheral nervous system of the subject, such that the neurological disease or disorder is treated or prevented. In some embodiments, the neurological disease or disorder would benefit from decreased neuronal cell death and/or increased neuronal survival, optionally wherein the neurological disease or disorder is selected from the group consisting of Alzheimer's disease, Parkinson's disease, Huntington's disease, Pick's disease, Kuf's disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dytonia syndrome, Leigh syndrome, Leber hereditary optic neuropathy(LHON), parkinsonism, dystonia, motor neuron disease, neuropathy-ataxia and retinitis pimentosa (NARP), maternal inherited Leigh syndrome (MILS), Friedreich ataxia, hereditary spastic paraplegia, Mohr-Tranebjaerg syndrome, Wilson disease, sporatic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, korsakoff's psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, and hyperactivity and movement disorders.

Also provided herein are methods of increasing muscle physiology of a muscle tissue, the method comprising contacting the muscle tissue with an agent comprising: i) an irisin polypeptide disclosed herein or biologically active fragments thereof, or ii) a nucleic acid encoding an irisin polypeptide disclosed herein or biologically active fragments thereof. In some embodiments, the step of contacting occurs in vivo, ex vivo, or in vitro. In some embodiments, the muscle tissue is skeletal muscle tissue, cardiac muscle tissue, and/or smooth muscle tissue.

The muscle tissue may be affected by a muscular dystrophy and/or comprises a muscle cell having a mutation in a gene associated with a muscular dystrophy, optionally wherein the gene is dystrophin. The muscular dystrophy may be selected from the group consisting of Becker, congenital, Duchenne, distal, Emery-Dreifuss, facioscapulohumeral, limb-girdle, myotonic, and oculopharyngeal muscular dystrophy. In some embodiments, the muscular dystrophy is Duchenne muscular dystrophy (DMD).

The muscle tissue may be affected by muscular atrophy. The muscle tissue may be affected by muscular atrophy resulting from disuse, trauma or a disease other than muscular dystrophy. The disease may be Charcot-Marie-Tooth disease or spinal muscular atrophy. The muscle physiology may be selected from the group consisting of increasing the expression of at least one neuromuscular junction biomarker; decreasing a biomarker of muscle injury, optionally wherein the biomarker is creatine kinase; decreasing the proportion of injured muscle cells in a tissue, optionally wherein the injured muscle cells are detected using an Evans blue staining assay; increasing the time of muscle activity, optionally wherein the time of muscle activity is measured using a running assay; increasing muscle strength, optionally wherein the muscle strength is measured using a forelimb grip strength assay; increasing lean muscle mass; regeneration of muscle tissue; decreasing fat mass; and inhibition of muscle atrophy. The neuromuscular junction biomarker may be selected from the group consisting of PGC-1α, acetylcholine receptor cluster (Chrne), acetylcholinesterase (AchE), utrophin, and GA binding protein transcription factor subunit alpha (GABPA).

Another aspect of the invention provides methods for preventing or treating muscular atrophy in a subject, comprising administering to the subject an agent comprising: i) an irisin polypeptide disclosed herein or biologically active fragments thereof, or ii) a nucleic acid encoding an irisin polypeptide disclosed herein or biologically active fragments thereof. The muscular atrophy may be caused by disuse, trauma, or disease. In some embodiments, the disease is Charcot-Marie-Tooth disease or spinal muscular atrophy.

Also provided herein are methods for preventing or treating a muscular dystrophy in a subject, comprising the step of administering to the subject an agent comprising: i) an irisin polypeptide disclosed herein or biologically active fragments thereof, or ii) the nucleic acid encoding an irisin polypeptide disclosed herein or biologically active fragments thereof. In some embodiments, the muscle cells of the subject comprise a mutation in a gene associated with a muscular dystrophy, optionally wherein the gene is dystrophin. In yet another embodiment, the muscular dystrophy is selected from the group consisting of Becker, congenital, Duchenne, distal, Emery-Dreifuss, facioscapulohumeral, limb-girdle, myotonic, and oculopharyngeal muscular dystrophy. In yet another embodiment, the muscular dystrophy is Duchenne muscular dystrophy (DMD).

The methods may further comprise administering conjointly to the subject an additional agent that increases expression or activity of the irisin polypeptide.

The agent may be administered systemically, optionally wherein systemic administration is intravenous or subcutaneous. In some embodiments, the agent is administered in a pharmaceutically acceptable formulation. In some embodiments, the agent is administered at least once a day, at least one a week, or at least once a month. In another embodiment, the agent is administered to the subject for greater than a number of months equal to 3, 4, 5, 6, 7, 8, 9, 10, 11, 12, 13, 14, 15, 16, 17, 18, 19, 20, 21, 22, 23, or 24, optionally the agent is administered to the subject for the duration of the subject's life.

The subject may be a mammal, optionally wherein the mammal is a rodent, a primate, or a human.

### BRIEF DESCRIPTION OF THE FIGURES

The patent application file contains at least one drawing executed in color. Copies of this patent or patent application publication with color drawing(s) will be provided by the Office upon request and payment of the necessary fee.

FIGS. 1A-1H show that eHsp90α is required for irisin binding to integrin αVβ5. FIG. 1A shows the schematic of the construct for recombinant human αVβ5 ectodomain protein production from mammalian cells. The domain boundaries are as follows: αV, 32-991; β5, 24-717. FIGS. 1B and 1D show the biolayer inferometry (BLI) measurement of binding of irisin to αVβ5. Purified irisin-His at the indicated concentration was infused over a sensor chip with immobilized clasped αVβ5 in the presence of 1 mM MgCl₂ and 1 mM CaCl₂). FIG. 1C shows the silver-stained SDS-polyacrylamide gel electrophoresis (SDS-PAGE) of affinity-purified αVβ5 fractions 7-20 collected following ion exchange. FIG. 1E shows a protein-elution profile of the indicated protein complexes from Superdex 200 increase 30/100GL (optical density (OD)₂₈₀). FIG. 1F shows the Coomassie-stained SDS-PAGE of the deglycosylated recombinant human Hsp90α and peak fractions from FIG. 1E. FIG. 1G shows the fluorescence anisotropy measurement of binding of αVβ5 and the αVβ5/Hsp90α complex to 50 nM A488-irisin-His in the presence of 1 mM MgCl₂ and 1 mM CaCl₂). FIG. 1H shows the fluorescence anisotropy competition assay for the αVβ5/Hsp90α complex binding by irisin: 50 nM A488-irisin-His and 125 nM αVβ5 were mixed with varying concentrations of unlabeled irisin or irisin-His, and anisotropy was recorded. Probe alone: 50 nM A488-irisin-His.

FIGS. 2A-2I show that eHsp90α level is increased with exercise in muscle extracellular fluid and in plasma. FIG. 2A shows the schematic of acute exercise and interstitial fluids (IF) isolation procedure and processing. FIG. 2B shows the anti-Hsp90α western blot showing Hsp90α protein levels in IF samples taken from the mouse without acute exercise (basal), or from the mice rested for the indicated amounts of time after acute exercise. 10 μg of total IF protein was loaded for each sample as shown by Ponceau staining. FIG. 2C shows the anti-Hsp90α western blot showing Hsp90α protein levels in gastrocnemius muscle samples taken from the mouse without acute exercise (basal), or from the mice rested for the indicated amounts of time after acute exercise. 10 μg of total muscle protein was loaded for each sample as shown by Ponceau staining. FIG. 2D shows the anti-Hsp90α western blot showing Hsp90α protein levels in plasma samples taken from the same group of mice from FIG. 2C. 10 μg of total plasma protein was loaded for each sample as shown by Ponceau staining. FIG. 2E shows the anti-Hsp90α and anti-HspA14 (control) western blot showing Hsp90α and HspA14 protein levels in plasma samples taken from five mice pre-exercise and post-exercise (1 hr). 10 μg of total plasma protein was loaded for each sample as shown by Ponceau staining. FIG. 2F shows the quantitative mass spectrometry showing the fold of changes of the indicated chaperone proteins identified in the IF samples from the exercised mice (1 hr post acute exercise) compared to the sedentary group (significant if p-value<0.05, FDR q-value<0.05). Genes are indicated along the x-axis, with bars representative of fold expression for sedentary (left) and after exercise (right) for each corresponding gene. FDR values of the significantly upregulated genes are indicated in the bar graph. FIG. 2G shows the western blots illustrating the Hsp90α detected in immunodepleted plasma. FIG. 2H shows the western blots illustrating the Hsp90α detected in immunodepleted muscle extracellular fluid. FIG. 2I shows the diagram of the proposed model: irisin is induced by exercise and driven by PGC1α, while the level of circulating Hsp90α is increased with exercise independent of PGC1α, which indicates that irisin and Hsp90α function together to medicate adaptations to exercise.

FIGS. 3A-3H show that eHsp90α is required for optimal cellular actions of irisin. FIG. 3A shows the fluorescence confocal images showing A488-irisin binding in HEK293T cells. HEK293T cells were either transiently transfected with control vector or full-length αV and β5 plasmids. 2 nM Hsp90α was used for 1 hr pretreatment, and 2 nM A488-irisin-His was subsequently used for 5 min treatment. FIG. 3B shows the anti-phosphorylated FAK (Y397) and anti-FAK western blots showing the levels of integrin signaling upon irisin and/or Hsp90α treatments. HEK293T cells were transfected and treated in the same way as FIG. 3A, except for the addition of the shown amounts of unlabeled irisin-His (0.1 nM or 1 nM) and Hsp90α (1 nM). Anti-HA and anti-Myc antibodies were used to probe the levels of the ectopically expressed αV and β5. FIG. 3C shows the immunofluorescence confocal images showing cell surface Hsp90α in SK-Mel2 cells. Live cells were treated with either control IgG or anti-Hsp90α at 4° C. FIG. 3D shows the quantification of the percentage of SK-Mel2 cells expressing cell surface Hsp90α in FIG. 3C (significant if p-value<0.05 by unpaired t-test). FIG. 3E shows the fluorescence confocal images showing A647-irisin binding in SK-Mel2 cells. Live cells were pretreated with either control IgG or anti-Hsp90α at 4° C. for 1 hr followed by 2 nM A647-irisin-His treatment at room temperature for 5 min. FIG. 3F shows the quantification of the percentage of A647-positive cells in FIG. 3E (significant if p-value<0.05 by unpaired t-test). FIG. 3G shows the crystal violet assay showing does-dependent inhibition of the cell viability of SK-Mel2 upon irisin treatment. Concentrations of irisin-His used for the treatments were indicated (one-way ANOVA). Samples as shown along the x-axis from left to right: left-most grey bar: control treatment with PBS, followed by 10 ng/mL, then 30 ng/mL, 50 ng/mL and 100 ng/mL (right-most bar). FIG. 3H shows the crystal violet assay showing the inhibition of irisin-mediated effect in SK-Mel2 cells by anti-Hsp90α or control antibody. Samples as shown along the x-axis from left to right: left-most grey bar: control treatment with PBS, followed by irisin-His+control antibody, followed by irisin-His+Hsp90α antibody (right-most bar). 50 ng/mL of irisin-His was used (one-way ANOVA).

FIGS. 4A-4F show that Hsp90α activates αVβ5 for irisin binding. FIG. 4A shows the flow charts of the steps used in three different methods for analyzing αVβ5-Apo and αVβ5/Hsp90α cryo-EM samples. FIG. 4B shows the 2D classes (generated by method 1) of αVβ5 particles in each of the three conformational states and the numbers (quantified by all three methods) of particles in each state. FIG. 4C shows the quantification of the percentage of distinguished particles (“likely open” particles were not included) in each of the three conformational states. For each sample, the conformation states represented from left to right are closed (left) extended closed (middle) and open (right). FIG. 4D shows the fluorescence anisotropy assay for A488-irisin binding by αVβ5, the αVβ5/Hsp90α complex in the presence of 1 mM MgCl₂ and 1 mM CaCl₂), or αVβ5 in the presence of 1 mM MnCl₂. 50 nM A488-irisin-His was used in the assay. FIG. 4E shows the cartoon diagram showing a two-step process of the irisin action through αVβ5. Irisin alone has low affinity for the closed-state αVβ5. Hsp90α, Mn²⁺ ion, or other possible factors, “opens” αVβ5, allowing for high-affinity irisin binding and effective signaling transduction through its integrin receptor. FIG. 4F shows the TALON pull-downs performed using 1 μM bead-bound clasped and tagged αVβ5. These were mixed with 2 μM untagged Hsp90α without bound nucleotide (Hsp90α-Apo) or Hsp90α charged with the indicated nucleotides, and bound samples were analyzed by Coomassie staining and anti-Hsp90α western blot.

FIGS. 5A-5G show that biophysical characterization of the irisin/αVβ5 complex suggests an unconventional ligand-integrin interaction. FIG. 5A shows the MicroScale Thermophoresis (MST) measurement of the binding stoichiometry between A488-irisin-His and αVβ5 in the presence of 1 mM MnCl₂. MST responses were recorded at varying irisin:αVβ5 ratios with the total molar concentration of irisin plus αVβ5 constant (10 μM).

FIG. 5B shows the size-exclusion chromatography and multiangle light scattering (SEC-MALS) determination of the absolute protein and glycan molecular mass of irisin-mam. 100 μg irisin-His was used in the assay. LS: light scattering; Total: total molecular mass of glycosylated irisin; Protein: molecular mass of the irisin protein; Glycan: molecular mass of the glycan. FIG. 5C shows the Hydrogen/Deuterium exchange mass spectrometry (HDX-MS) mapping of the protected sites on αVβ5 in the irisin/αVβ5 complex. The measured relative deuterium level of peptides in αVβ5-Apo at each deuteration time point was subtracted from the deuterium level of the corresponding peptide in the irisin/αVβ5 complex (D_{cplx}−Dₐₚₒ), and the differences were colored according to the scale shown on the right. Peptides are shown from N- to C-terminus top to bottom, referring to the domain architecture on the left. The amount of time in deuterium is shown at the bottom. All deuterium uptake values used to generate these difference maps can be found FIG. 5E. FIG. 5D shows the regions of αVβ5 protected from HDX in the irisin/αVβ5 complex (purple) on αVβ5 space filling structural model based on FIG. 5C. αVβ5 structural model was generated from αVβ3 (PDB 1M1X) with 15 predicted by AlphaFold. αV subunit is in grey, and 15 subunit is in wheat. FIGS. 5E-5G show the hydrogen/deuterium exchange mass spectrometry (HDX-MS) experimental details used to generate FIGS. 5C-5D. FIG. 5E shows the HDX-MS data summary and list of experimental parameters. FIG. 5F show the digestive coverage map for αV subunit. FIG. 5G show the digestive coverage map for 15 subunit.

FIGS. 6A-6E show the atomic resolution model of the irisin/αVβ5 complex. FIG. 6A shows the schematic of the irisin/αVβ5 complex modeling. Experimental results (red) were incorporated into the procedure in the indicated steps. FIG. 6B shows the space filling structural model of the irisin/αVβ5 complex. αV subunit is in beige, 15 subunit is in grey, and irisin is in magenta. The N- and C-terminus of irisin are highlighted in yellow and green, respectively. FIG. 6C shows the space filling structural model of the irisin/FNO/αV5 complex. αV subunit is in beige, β5 subunit is in grey, irisin is in magenta, and FN10 is in yellow. FN10-αVβ3 complex structure (PDB 4MMX) was used for fibronectin alignment to dock FN10 onto αVβ5. FIG. 6D shows the electrostatic potential surfaces of irisin (top) and αVβ5 (bottom). The surface charge distribution is displayed as blue for basic/positive, red for acidic/negative, and white for neutral. One acidic amino acid-rich region (A) and one basic amino acid-rich region (B) were shown on both irisin and αVβ5 at the interface. FIG. 6E shows the irisin-αVβ5 interactions in the irisin/αVβ5 complex model. Electrostatic interactions are in dashed lines between the atoms involved. The hydrophobic interactions are represented by arcs with spokes radiating towards the ligand atoms they contact, and the contacted atoms are shown with spokes radiating back.

FIGS. 7A-7F show the validation of the irisin/αVβ5 complex model. FIG. 7A shows the fluorescence anisotropy assay for A488-FN10 binding by αVβ5 in the presence of 1 mM MnCl₂. 50 nM A488-FN10 was used in the assay. FIG. 7B shows the fluorescence anisotropy competition assay for the αVβ5 binding by irisin or FN10: 50 nM A488-FN10 and 500 nM αVβ5 were mixed with varying concentrations of unlabeled irisin-His or FN10, and anisotropy was recorded. Probe alone: 50 nM A488-FN10. FIG. 7C shows the fluorescence resonance energy transfer (FRET) assay using 50 nM A488-FN10 and 50 nM unlabeled or A555 labeled irisin-His in the presence or absence of 500 nM αVβ5. FRET efficiency was calculated as the ratio of F₅₅₅/(F₄₈₈+F₅₅₅) where F₅₅₅ is the acceptor emission and F₄₈₈ is the donor emission (one-way ANOVA). FIG. 7D shows the fluorescence anisotropy assay for A488-irisin-bac binding by αVβ5 in the presence of 1 mM MnCl₂. FIG. 7E shows the fluorescence anisotropy competition assay for the αVβ5 binding by irisin-mam or irisin-bac: 50 nM A488-irisin-bac and 1000 nM αVβ5 were mixed with varying concentrations of unlabeled irisin-mam (irisin-His) or irisin-bac, and anisotropy was recorded. Probe alone: 50 nM A488-irisin-bac. FIG. 7F shows the fluorescence anisotropy competition assay for the αVβ5 binding by irisin-WT or irisin-R75E: 50 nM A488-irisin-WT and 1000 nM αVβ5 were mixed with varying concentrations of unlabeled irisin-WT or irisin-R75E, and anisotropy was recorded. Probe alone: 50 nM A488-irisin-WT.

FIGS. 8A-8D show blot results. FIG. 8A shows the silver-stained SDS-PAGE of the affinity-purified recombinant human αVβ5 and irisin-His. Anti-αV and anti-β5 western blots were used to show αV and 15 in the purified sample. FIG. 8B shows the identification of proteins from 90 kDa gel band of fraction 20 in FIG. 1C by mass spectrometry. FIG. 8C shows the TALON pull-downs performed using 1 μM bead-bound control peptide dimer (HRV3C-acidic stretch-2×strepII/HRV3C-basic stretch-8×His) or clasped and tagged αVβ5. These were mixed with 2 μM untagged Hsp90α, and bound samples were analyzed by Coomassie staining. FIG. 8D shows that irisin has no detectable binding affinity to Hsp90α in any of its nucleotide states using fluorescence anisotropy binding assay.

FIGS. 9A-9B show blot results. FIG. 9A shows the anti-phosphorylated FAK (Y397) and anti-FAK western blots showing the levels of integrin signaling upon unlabeled irisin-His (1 nM), A488-irisin-His (1 nM) or A488 alone (1 nM) treatments. HEK293T cells were transfected and treated in the same way as FIG. 3A. Anti-HA and anti-Myc were used to probe the levels of the ectopically expressed αV and β5. FIG. 9B shows the co-immunoprecipitation assay of endogenous cell surface αV and β5 using SK-Mel2 cells. Endogenous cell surface Hsp90α was captured by anti-Hsp90α in live cells at 4° C.

FIGS. 10A-10C show electron micrograph results. FIG. 10A shows the electron micrographs of negative stain samples of recombinant Hsp90α with or without cross-linking. FIG. 10B shows the 2D classes of the cross-linked Hsp90a. The EM maps of the “extended” and the “fastened” Hsp90α dimers are taken from Southworth, D. R. and D. A. Agard, Species-dependent ensembles of conserved conformational states define the Hsp90 chaperone ATPase cycle. Mol Cell, 2008. 32(5): p. 631-40. FIG. 10C shows the quantification of the percentage of the distinguished particles (“likely open” particles were included) in each of the three conformational states. For each sample, the conformation states represented from left to right are closed (left) extended closed (middle) and open (right).

FIG. 11 shows the structure-based amino acid sequence alignment of irisin and fibronectin III domains (FNIII) containing RGD and RGD-like motifs using PROMALS3D. RGD and RGD-like motifs (highlighted in yellow) of FNIII-containing integrin ligands are aligned with a “QGQ” motif (red) of irisin.

FIGS. 12A-12B show the effects of Hsp90α on irisin-mediated responses in C2C12 cells. FIG. 12A shows the images taken from optical microscope of C2C12 cells with different treatments with Hsp90α and Hsp90a. C2C12 cells were seeded in confluence and differentiated in DMEM containing 2% horse serum for 8 days. PBS, 2 nM irisin, 2 nM Hsp90α, and 2 nM irisin plus Hsp90α were used to treat cells from day 1 of differentiation on a daily basis. Images were taken from the objectives of the regular optical microscope. Cells treated with irisin plus Hsp90α showed higher density and longer muscle fibers compared to other conditions. FIG. 12B shows the Q-PCR analysis of mRNA levels of the indicated genes involved in differentiation and hypertrophic growth of muscle cells. For each condition, bars showing the relative mRNA levels for MyoD (left bar), PGC1α (middle bar) and MyoG (right bar) are depicted.

FIGS. 13A-13C show the characterization of irisin glycosylation. FIG. 13A shows the detection of protein thermal stability of native glycosylated and deglycosylated irisin using Protein Thermal Shift assay. FIG. 13B is schematic of the construct for recombinant irisin (WT and mutant) production. FIG. 13C shows the western blot illustrating the detection of irisin expression and secretion in Epi293 cells that were transfected with the indicated irisin plasmids. Cell lysates and cell culture medium samples were prepared on the indicated date post transfection. Irisin level was probed with anti-irisin antibody using western blot.

FIGS. 14A-14B show the mammalian recombinant irisin production construct and sequence. FIG. 14A shows the mammalian recombinant irisin production construct. FIG. 14B shows the sequences and modifications of the recombinant irisin and the irisin N45Q mutant.

FIG. 15 shows the silver staining SDS-PAGE of recombinant irisin and irisin N45Q mutant purified from mammalian cells.

FIGS. 16A-16B show the fluorescence anisotropy assay measurement of binding affinity of irisin and irisin N45Q mutant for αVβ5 receptor. FIG. 16A shows the fluorescence anisotropy assay measurement of direct binding affinity of each of irisin and irisin N45Q mutant for αVβ5 receptor. FIG. 16B shows the fluorescence anisotropy assay measurement of binding affinity of irisin and irisin N45Q mutant for αVβ5 receptor in a competition binding assay.

FIG. 17 shows the western blot illustrating the irisin-induced integrin signaling in HEK293 cells with or without ectopically expressed αVβ5 receptor.

### DETAILED DESCRIPTION OF THE INVENTION

The present disclosure provides irisin polypeptides disclosed herein comprising a glycosylated residue at position 36, a glycosylated residue at position 81, and a non-N-linked glycosylated residue at position 45; and an agent comprising irisin derived polypeptides disclosed herein (e.g., irisin glycosylation mutants) and biologically active fragments thereof, or nucleic acids encoding such polypeptides, and methods of treatment using such agent.

Irisin is a small polypeptide that is secreted by muscle and other tissues into the blood of mice and humans (P. Bostrom et al., A PGC1-alpha-dependent myokine that drives brown-fat-like development of white fat and thermogenesis. Nature 481, 463-468 (2012); M. P. Jedrychowski et al., Detection and Quantitation of Circulating Human Irisin by Tandem Mass Spectrometry. Cell Metab 22, 734-740 (2015)). The amino acid sequence is conserved 100% between mice and humans, suggesting a critical, conserved function. Importantly, the expression of irisin and its precursor protein FNDC5, are increased in muscle with many forms of exercise in rodents and humans. Irisin levels increase in the blood of humans with exercise training by Tandem Mass Spectrometry (M. P. Jedrychowski et al., Detection and Quantitation of Circulating Human Irisin by Tandem Mass Spectrometry. Cell Metab 22, 734-740 (2015)). In adipose cells, osteocytes and osteoclasts, integrin αV/P5 is the major functioning receptor for irisin (H. Kim et al., Irisin Mediates Effects on Bone and Fat via alphaV Integrin Receptors. Cell 175, 1756-1768 e1717 (2018)).

Physical activity can possibly prevent and ameliorate the symptoms of multiple forms of neurodegeneration, including Alzheimer's Disease (AD) and PD (K. S. Bhalsing, M. M. Abbas, L. C. S. Tan, Role of Physical Activity in Parkinson's Disease. Ann Indian Acad Neurol 21, 242-249 (2018); B. M. Brown, J. J. Peiffer, R. N. Martins, Multiple effects of physical activity on molecular and cognitive signs of brain aging: can exercise slow neurodegeneration and delay Alzheimer's disease?Mol Psychiatry 18, 864-874 (2013); S. H. Choi et al., Combined adult neurogenesis and BDNF mimic exercise effects on cognition in an Alzheimer's mouse model. Science 361 (2018); I. Marques-Aleixo et al., Preventive and Therapeutic Potential of Physical Exercise in Neurodegenerative Diseases. Antioxid Redox Signal 34, 674-693 (2021)). U.S. Application No. 63/310,873, hereby incorporated by reference in its entirety, shows that modulators of FNDC5 or biologically active fragments thereof (e.g., irisin) can act to prevent or reduce degeneration of dopaminergic (DA) neurons preventing or ameliorating at least one motor deficit and/or preventing or ameliorating at least one symptom of cognitive dysfunction or dementia, such as to prevent or treat an α-synucleinopathy, such as Parkinson's disease, Lewy body dementia, Alzheimer's disease multiple system atrophy (MSA), or a neuroaxonal dystrophy.

Elevated expression of FNDC5 in the liver via the use of adenoviral vectors, and elevations of irisin in the blood, stimulated an “exercise-like” program of gene expression in the hippocampus (C. D. Wrann et al., Exercise induces hippocampal BDNF through a PGC-1alpha/FNDC5 pathway. Cell Metab 18, 649-659 (2013)). Moreover, the expression of FNDC5 with these same viral vectors rescued memory deficits in a mouse model of AD (M. V. Lourenco et al., Exercise-linked FNDC5/irisin rescues synaptic plasticity and memory defects in Alzheimer's models. Nat Med 25, 165-175 (2019)). Most recently, irisin was shown to be the active moiety regulating cognitive function in four separate mouse models. Importantly, elevation of the blood levels of the mature, cleaved irisin was sufficient to improve cognitive function and reduce neuroinflammation in two distinct models of AD (M. R. Islam et al., Exercise hormone irisin is a critical regulator of cognitive function. Nat Metab 3, 1058-1070 (2021)). Furthermore, irisin itself crossed the blood-brain barrier (BBB), at least when the protein was produced from the liver with these AAV vectors. WO2015/051007 hereby incorporated by reference in its entirety, discloses that FNDC5 or, irisin polypeptides or fragments thereof, can act on neurons of the central and/or peripheral nervous system to enhance BDNF expression/activity and increase neuronal survival and function to thereby prevent or treat undesired neurological disorders.

PGC-1α is also induced in skeletal muscle with exercise and is a major mediator of the beneficial effects of exercise in this tissue (Finck and Kelly (2006) J. Clin. Invest. 116, 615-622). PGC-1α was initially discovered as a transcriptional co-activator of mitochondrial biogenesis and oxidative metabolism in brown fat (Puigserver et al. (1998) Cell 92, 829-839 and Spiegelman (2007) Novartis Foundation Sympos. 287, 60-69). PGC-1α is induced in muscle by endurance exercise (Russell et al. (2003) Diabetes 52(12):2874-81, Pilegaard et al. (2003) J Physiol. 546(Pt 3): 851-858, Safdar et al. (2011) J Biol. Chem. 286(12):10605-17), and ectopic expression of PGC-1α recapitulates endurance training (Handschin and Spiegelman (2008) Nature, 454(7203): 463-469). PGC-1α expressed in skeletal muscle can affect other organs (Handschin et al. (2007) J Clin. Investigation, 117(11):3463-3474). PGC1α regulates neuromuscular junction gene expression and acetylcholine receptor (AChR) clusters in muscle. Transgenic expression of PGC-1α ameliorates muscular dystrophy in the mdx mouse, a model of Duchenne muscular dystrophy (DMD), as demonstrated by decreases in markers of muscle injury such as Evans blue staining of muscle cells and serum concentrations of creatine kinase (Handschin et al. (2007) Genes Dev. 21:770-783). Upregulation of FNDC5 or biologically active fragments thereof have been reported by U.S. Patent Application No. 63/073,255, hereby incorporated by reference in its entirety, to be useful to increase muscle physiology, such as to ameliorate the effects of a muscular dystrophy and muscular atrophy.

Accordingly, the compositions and methods described herein would be useful in methods of treating and preventing diseases that involve modulating the expression level of α-synuclein and/or brain-derived neurotrophic factor (BDNF), and improving muscular physiology. Data have shown that although the recombinant irisin is bioactive, its in vivo half-life is very short (˜40 min), making it challenging to be employed as a therapeutic agent. Herein, new, non-naturally occurring, forms of irisin glycosylation mutants (e.g., N45Q) are described. In some embodiments, these irisin polypeptides exhibit intermediate thermal stability, bioactivity and in vivo half-life, compared to the WT irisin (low stability, high bioactivity and short half-life). Taken together, these data suggest the potential therapeutic value of the irisin glycosylation mutants (e.g., N45Q) in the treatment of Parkinson's disease and other neurodegenerative diseases and disorders that involve α-synuclein, neurological diseases or disorders that would benefit from increased expression of brain-derived neurotrophic factor (BDNF), muscular dystrophy or muscular atrophy that requires increased muscle physiology, or any other disease or disorder disclosed herein.

In some aspects, the irisin polypeptides disclosed herein can act to prevent or reduce degeneration of dopaminergic (DA) neurons preventing or ameliorating at least one motor deficit and/or preventing or ameliorating at least one symptom of cognitive dysfunction or dementia. The irisin polypeptides disclosed herein may also be used to prevent or treat an α-synucleinopathy, such as Parkinson's disease, Lewy body dementia, Alzheimer's disease multiple system atrophy (MSA), Lou Gehrig's disease (ALS), or a neuroaxonal dystrophy. Administration of the irisin polypeptides may be useful to increase expression of brain-derived neurotrophic factor (BDNF) in neuron cells. By increasing BDNF expression or activity in central or peripheral nervous system of a subject, the irisin polypeptides disclosed herein can also be used to treat or prevent neurological diseases or disorders that would benefit from decreased neuronal cell death and/or increased neuronal survival. The polypeptides generated herein can also be used to address a wide variety of muscle conditions by increasing muscle physiology of a muscle tissue. Exemplary applications include preventing or treating muscular atrophy (e.g., atrophy from disuse, such as post-surgery or with limb immobilization; atrophy caused by trauma, such as an injury; or atrophy resulting from a disease state, such as spinal muscular atrophy or Charcot-Marie-Tooth disease). Other exemplary applications include preventing or treating muscular dystrophy (e.g., Becker, congenital, Duchenne, distal, Emery-Dreifuss, facioscapulohumeral, limb-girdle, myotonic, and oculopharyngeal muscular dystrophy), optionally wherein muscle cells of the subject comprise a mutation in a gene associated with a muscular dystrophy (e.g., dystrophin).

### Definitions

In order that the present invention may be more readily understood, certain terms are first defined. Additional definitions are set forth throughout the detailed description.

The articles “a” and “an” are used herein to refer to one or to more than one (i.e., to at least one) of the grammatical object of the article. By way of example, “an element” means one element or more than one element.

The term “binding” or “interacting” refers to an association, which may be a stable association, between two molecules, e.g., between a polypeptide encompassed by the present invention and a binding partner, due to, for example, electrostatic, hydrophobic and/or hydrogen-bond interactions under physiological conditions. Exemplary interactions include protein-protein, protein-nucleic acid, protein-small molecule, and small molecule-nucleic acid interactions.

The term “half-life” refers to the turnover rate or the time it takes to replace half of a molecule (e.g., irisin and its glycosylation mutants) as it is aged and broken down within a cell. Half-life is an important characteristic of proteostasis which is a cellular process that includes control of concentrations, conformations, binding interactions, and locations of individual proteins, enabling cells to change their physiology for successful organismal development and aging while under constant challenges from intrinsic and environmental stimuli.

The term “stability” of a protein (e.g., irisin and its glycosylation mutants) refers to patterns indicating the survival of the protein over time. These patterns may be determined with respect to the integrity of the primary and conformational structure of the correctly folded protein, by studying its transformations such as protein unfolding, denaturation, degradation, conformational change, enzymatic modification and proteolytic cleavage. The half-life of a protein's activity may also be primarily considered as a measure of its stability from a pharmacological and biotechnological perspective.

The term “glycosylation” refers to the covalent modification to a protein (e.g., irisin) that introduces attachment of glycans (e.g., oligosaccharides and polysaccharides) to various sites on the protein via an N-linkage, an O-linkage, or both. In living cells, such modification is common to newly synthesized proteins, is highly regulated, and is species- and cell-specific. Depending on the type of glycans and location of the attachment site, glycosylation of a polypeptide may lead to different physical and biochemical properties. Thus, mutations that lead to the glycosylation discrepancies can alter the protein's properties such as binding affinity, half-life, and stability.

As used herein, the term “integrin” refers to the plasma membrane receptors that are expressed in a wide variety of cells and bind to specific ligands in the extracellular matrix. The specific ligands bound by integrins can contain an arginine-glycine-aspartic acid tripeptide (Arg-Gly-Asp; RGD) or a leucine-aspartic acid-valine tripeptide, and include, for example, fibronectin, vitronectin, osteopontin, tenascin, and von Willebrand's factor. The integrins comprise a superfamily of heterodimers composed of an α subunit and a β subunit. Numerous a subunits, designated, for example, αv, α5 and the like, and numerous β subunits, designated, for example, β1, β2, β3, β5 and the like, have been identified, and various combinations of these subunits are represented in the integrin superfamily, including α5β1, αvβ3 and αVβ5. There are at least 18 α and eight β subunits are in humans, generating 24 known heterodimers (Takada et al. (2007) Gen. Biol. 8:215). The superfamily of integrins can be subdivided into families, for example, as αv-containing integrins, including αvβ3 and αVβ5, or the β1-containing integrins, including α5β1 and αvβ1. Integrins are expressed in a wide range of organisms, including C. elegans, Drosophila sp., amphibians, reptiles, birds, and mammals, including humans.

As used herein, the term “administering” a substance, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to an animal by any suitable route for delivery of the therapeutic agent to the desired location in the animal, including delivery by either the parenteral or oral route, intramuscular injection, subcutaneous/intradermal injection, intravenous injection, buccal administration, transdermal delivery and administration by the intranasal or respiratory tract route.

The term “α-synucleinopathy” includes any neurodegenerative disease or disorder characterized by the accumulation of α-synuclein in cells or tissue (e.g., in neurons, nerve fibers, glial cells, cancer cells, and the like). In some embodiments, accumulation of α-synuclein is associated with multisystem neurodegeneration, and underlies a wide spectrum of clinical syndromes, movement disorders/parkinsonism (Parkinson's disease, pantothenate kinase-associated neurodegeneration), dementia (Parkinson's disease dementia, dementia with Lewy body), and autonomic dysfunction (pure autonomic failure, multiple system atrophy). Pathogenetically, they can arise from disturbances in the metabolism of α-synuclein (for example, increased synthesis or oligomer formation due to insufficient degradation). Therefore, as used herein, “α-synuclein” and “pathogenic α-synuclein” are interchangeable, and can refer to an increase in the amount of misfolded and/or mutated α-synuclein, or to an increase in the overall levels of any form, including wild type, of α-synuclein in cells or tissues to predispose or cause a pathogenic condition (e.g., a α-synucleinopathy or a cancer disclosed herein) in a subject. Although trace levels of phosphorylated α-synuclein are detectable in healthy brains, much of the α-synuclein accumulated within Lewy bodies in Parkinson's disease brains is phosphorylated on serine 129 (Ser-129). Therefore, increased levels of α-synuclein may refer not only to total levels of wild-type α-synuclein, but any mutated form of α-synuclein or phosphorylated form of α-synuclein, such as α-synuclein phosphorylated on serine 129 (Ser-129). Duplication, triplication and of the α-synuclein locus can cause an α-synucleinopathy (Giobbie-Hurder, A., et al., (2017). An immunogenic personal neoantigen vaccine for patients with melanoma. Nature, 547(7662), 217-221, Singleton, A. B., et al., (2003). α-synuclein locus triplication causes Parkinson's disease. Science (New York, N.Y.), 302(5646), 841). Polymorphisms in the α-synuclein gene can increase or decrease one's risk of developing α-synucleinopathy, based on the expression of α-synuclein (Pedersen, C. C., et al., (2021). A systematic review of associations between common SNCA variants and clinical heterogeneity in Parkinson's disease. NPJ Parkinson's disease, 7(1), 54). A subject with increased levels of α-synuclein includes patients whose measured levels of a pathogenic form of α-synuclein (such as a phosphorylated form) are increased, even when wild type levels are constant or decreasing. Increased levels of α-synuclein phosphorylated on serine 129 (Ser-129). Similarly, decreasing or reducing levels of α-synuclein include decreasing or reducing the amount of misfolded α-synuclein, mutated α-synuclein, wild type α-synuclein, or overall levels of α-synuclein. A subject afflicted with an α-synucleinopathy may show degeneration of dopaminergic neurons, at least one motor deficit (e.g., tremor at rest, such as a slight tremor in the hands or feet; rigidity (stiffness) of limbs, neck, or shoulders; difficulty balancing (postural instability); slowness of movement or gradual loss of spontaneous movement (bradykinesia); trouble standing after sitting; stiffness in the limbs, or moving more slowly) and/or at least one symptom of cognitive dysfunction or dementia (e.g., confusion, poor motor coordination, loss of short-term or long-term memory, identity confusion, or impaired judgment).

α-synuclein is a highly conserved protein belonging to a multigene family that includes β-synuclein and γ-synuclein. α-synuclein is strongly expressed in neurons, highly enriched in presynaptic terminals, and transported predominantly in the slow component. Axonal transport abnormalities of α-synuclein have may cause or be associated with synucleinopathies. This is based on the observation that axonal α-synuclein pathology is pronounced in the disease and also on experimental evidence suggesting that α-synuclein may play a role in transport of presynaptic vesicles. As used herein, α-synucleinopathies also include age-related retardation in the normal transport of α-synuclein (K. A. Jellinger, Synucleinopathies, Encyclopedia of Movement Disorders, Academic Press, 2010, Pages 203-207). Non-limiting, representative examples of α-synucleinopathies include Parkinson's disease, Lewy body dementia, multiple system atrophy (MSA), Alzheimer's disease and a neuroaxonal dystrophy.

Parkinson's disease is a progressive neurodegenerative disease characterized by tremor and bradykinesia. A portion of patients with Parkinson's disease have a family history of the condition, and family-linked cases can result from genetic mutations in a group of genes—LRRK2, PARK2, PARK7, PINK1 or the SNCA gene.

Dementia with Lewy bodies (i.e., Lewy Body dementia) is characterized by the accumulation of aggregated α-synuclein protein in Lewy bodies, similar to Parkinson's disease and Parkinson's disease dementia. However, it is can also be accompanied by aggregation of amyloid-beta and tau proteins.

Alzheimer's disease is a progressive neurodegenerative disease most often associated with memory deficits and cognitive decline. The cardinal pathological features of the disease include the presence of amyloid plaques and neurofibrillary tangles. Dominantly inherited familial AD (FAD) can be caused by mutations in amyloid precursor protein (APP), presenilin 1 (PSEN1) or PSEN2 genes. Early onset Alzheimer's disease (EOAD) is defined by those affected before age 65; and though they are slightly more common than FAD cases.

More common late onset AD (LOAD) is considered sporadic, although genetic risk factors have been identified, most notably apolipoprotein E gene (APOE). Pathology indicative, although not exhaustive, symptoms of Alzheimer's disease include moderate cortical atrophy that is most marked in multimodal association cortices and limbic lobe structures, extracellular amyloid plaques, Hirano bodies, granulovacuolar degeneration (GRVD) cerebral amyloid angiopathy (CAMA) and/or intracellular neurofibrillary tangles. Greater than 50% of AD patients have alpha-synuclein pathology in addition to tau and amyloid beta. See Twohig, D., & Nielsen, H. M. (2019). α-synuclein in the pathophysiology of Alzheimer's disease. Molecular neurodegeneration, 14(1), 23.

Multiple system atrophy is a progressive brain disorder that affects movement and balance and disrupts the function of the autonomic nervous system. The autonomic nervous system controls body functions that are mostly involuntary, such as regulation of blood pressure. The most frequent autonomic symptoms associated with multiple system atrophy are a sudden drop in blood pressure upon standing (orthostatic hypotension), urinary difficulties, and erectile dysfunction in men. Two major types of multiple system atrophy have been described, which are distinguished by their major signs and symptoms at the time of diagnosis. In one type, known as MSA-P, a group of movement abnormalities called parkinsonism are predominant. These abnormalities include unusually slow movement (bradykinesia), muscle rigidity, tremors, and an inability to hold the body upright and balanced (postural instability). The other type of multiple system atrophy, known as MSA-C, is characterized by cerebellar ataxia, which causes problems with coordination and balance. This form of the condition can also include speech difficulties (dysarthria) and problems controlling eye movement.

Infantile neuroaxonal dystrophy (INAD) is a rare neurodegenerative disease characterized by regression of acquired motor skills, delayed motor coordination and eventual loss of voluntary muscle control. Biallelic mutations in the PLA2G6 gene have been identified as the most frequent cause of INAD.

The term “muscular atrophy” refers to the wasting or thinning of muscle tissue. Muscular atrophy can occur due to aging, improper nutrition, lack of use, such as from a sedentary lifestyle (e.g., being bedridden) or confinement (e.g., long periods in a seated position or in a low-gravity environment) otherwise known as common atrophies of immobilization. Muscular atrophy can also result from trauma, such as a sports injury or injuries sustained in an accident. Muscular atrophy can also have a pathological cause such as disease (e.g., muscular dystrophy and spinal muscular atrophy or Charcot-Marie-Tooth disease).

The terms “muscular dystrophy” and “muscular dystrophy disease or disorder” as used herein, refer to one or more of the genetic diseases belonging to the nine major groups of muscular dystrophy. The nine types of muscular dystrophy include Becker, congenital, Duchenne, distal, Emery-Dreifuss, facioscapulohumeral, limb-girdle, myotonic, and oculopharyngeal muscular dystrophy. These diseases are characterized by progressive degeneration and weakness of skeletal and/or cardiac muscles. Muscular dystrophies are peripheral conditions that directly affect muscle cells and tissues and are not considered to be neuronal conditions that affect the central and/or peripheral nervousor blood/brain barrier disorders (e.g., multiple sclerosis).

Becker muscular dystrophy primarily affects voluntary muscles such as the muscles in the hips, pelvic area, thighs, shoulders, and heart. Mutations in the dystrophin gene that result in partially functional dystrophin are associated with Becker muscular dystrophy.

Duchenne muscular dystrophy (DMD) is an X-linked genetic disorder characterized by progressive muscle degeneration. Mutations in the dystrophin gene that result in no or minimally functional dystrophin are associated with DMD.

Emery-Dreifuss muscular dystrophy is characterized by wasting and weakness of the muscles in the shoulders, upper arms, and calf muscles.

Facioscapulohumeral muscular dystrophy is characterized by degeneration or weakness of muscles of the face, shoulder blades, and upper arms.

Oculopharyngeal muscular dystrophy is characterized by weakness of the eyelids (ocular) and throat (pharyngeal) muscles.

Several of the muscular dystrophy groups include multiple types or subtypes. For example, congenital muscular dystrophy refers to one of the following muscular dystrophy diseases: CMD with adducted (drawn inward) thumbs, ophthalmoplegia (paralyzed eye muscles) and intellectual disability, CMD with cardiomyopathy, CMD with central nervous system atrophy and absence of large myelinated fibers in peripheral nervous system, CMD with cerebellar atrophy (diminished size of the cerebellum, a part of the brain involved in motor control), CMD with desmin inclusions (abnormal accumulations of the muscle protein desmin in some muscle fibers), CMD with integrin alpha 7 mutations, CMD with joint hyperlaxity (abnormally flexible joints), CMD with familial junctional epidermolysis bullosa, CMD with muscle hypertrophy (enlargement of muscles); also called MDC1C, CMD with muscle hypertrophy and respiratory failure; also called MDC1B, CMD with muscle hypertrophy and severe intellectual disability; also called MDC1D, CMD with myasthenic syndrome, CMD with (early) spinal rigidity, CMD with spinal rigidity and lamin A/C abnormality, CMD with spinal rigidity and selenoprotein deficiency, CMD with structural abnormalities of mitochondria (energy-producing subunits of cells), Fukuyama CMD; also called MDDGA4, Merosin-deficient CMD; also called MDC1A, Merosin-positive CMD; this is an old term referring to a variety of CMD types in which merosin is normal, Santavuori muscle-eye-brain disease, Ullrich CMD, Walker-Warburg syndrome: MDDGA type, Walker-Warburg syndrome: MDDGA1 type, Walker-Warburg syndrome: MDDGA2 type, Walker-Warburg syndrome: MDDGA3 type; same as Santavuori muscle-eye-brain disease, Walker-Warburg syndrome: MDDGA4 type; same as Fukuyama CMD, Walker-Warburg syndrome: MDDGB5 type; same as CMD with muscle hypertrophy (MDC1C), Walker-Warburg syndrome: MDDGA6 type; same as CMD with muscle hypertrophy and severe intellectual disability (MDC1D), Walker-Warburg syndrome: MDDGA7 type, Walker-Warburg syndrome: MDDGA8 type, Walker-Warburg syndrome: MDDGA10 type, Walker-Warburg syndrome: MDDGA11 type, and Walker-Warburg syndrome: MDDGA12 type.

Limb-girdle muscular dystrophy is a group of diseases that manifest in the proximal muscles around the hips and shoulders. Limb-girdle muscular dystrophies are subdivided into two types (Type 1 (dominantly inherited) and Type 2 (recessively inherited)) and further classified based on what gene is mutated. Type 1 limb-girdle muscular dystrophies include LGMD1A, LGMD1B, LGMD1C, LGMD1D, LGMD1E, LGMD1F, LGMD1G, and LGMD1H. Type 2 limb-girdle muscular dystrophies include LGMD2A, LGMD2B, LGMD2C, LGMD2D, LGMD2E, LGMD2F, LGMD2G, LGMD2H, LGMD2I, LGMD2J, LGMD2K, LGMD2L, LGMD2M, LGMD2N, LGMD20, LGMD2P, LGMD2Q, LGMD2R, LGMD2S, LGMD2T, LGMD2U, LGMD2V, LGMD2W, LGMD2X, and LGMD2Y.

Distal muscular dystrophy is class of diseases that generally affect the distal muscles in the arms, hands, legs, and feet. Distal muscular dystrophies include distal myopathy with vocal cord and pharyngeal weakness; Finnish (tibial) distal myopathy; Gowers-Laing distal myopathy; hereditary inclusion-body myositis type 1; Miyoshi distal myopathy; Nonaka distal myopathy; VCP Myopathy/IBMPFD; Welander's distal myopathy; and ZASP-related myopathy.

Myotonic muscular dystrophy is characterized by weakness and wasting or shrinking of voluntary muscles in the face, neck, and lower arms and legs. Muscles involved in breathing (e.g., rib and diaphragm muscles) may also be affected. Myotonic muscular dystrophy is divided into two types, with Type 2 being the milder form of the disease.

A “dystrophic phenotype” as used herein refers to muscular degeneration and/or weakness in a subject possessing a genetic determinant for a muscular dystrophy disease or disorder. The muscular degeneration and weakness that characterizes muscular dystrophy can manifest in a variety of signs or symptoms of the disease including, but not limited to, progressive muscular wasting, poor balance, scoliosis, progressive difficulty walking, a waddling gait, calf deformation, limited range of movement, respiratory difficulty, cardiomyopathy, and muscle spasms.

As used herein, the term “dystrophin gene or polynucleotide” refers to the gene associated with certain muscular dystrophies, such as Duchenne and Becker muscular dystrophies. The dystrophin gene encodes the dystrophin polypeptide, which is a structural protein in muscle cells and mutations in the dystrophin gene causing muscular dystrophies are both well-known and annotated in the art (see, for example, the mutation entry database at umd.be/DMD/W_DMD/index.html; dmd.nl/database.html; Takeshima et al. (2010) J Hum. Genet. 55:79-388; Li et al. (2015) Orphanet J Rar Dis. 10:5; Bladen et al. (2015) Hum. Mutat. 36:395-402, and the like)). As contemplated herein, dystrophin polypeptide and polynucleotide are intended to include fragments, variants (e.g., allelic variants) and derivatives thereof. The nucleotide and amino acid sequences of human dystrophin, which correspond to GenBank Accession number NM_004006.3 and NP_003997.2 respectively. At least 17 isoforms are known for human dystrophin (NM_000109.4 and NP_000100.3; NM_004006.3 and NP 003997.2; NM_004009.3 and NP 004000.1; NM_004010.3 and NP_004001.1; NM 004011.4 and NP_004002.3; NM_004012.4 and NP_004003.2; NM_004013.2 and NP_004004.1; NM_004014.2 and NP 004005.1; NM_004015.3 and NP 004006.1; NM_004016.3 and NP_004007.1; NM_004017.3 and NP_004008.1; NM_004018.3 and NP 004009.1; NM_004019.3 and NP 004010.1; NM_004020.3 and NP_004011.2; NM_004021.3 and NP_004012.2; NM_004022.2 and NP_004013.1; NM_004023.3 and NP_004014.2). Nucleic acid and polypeptide sequences of dystrophin orthologs in organisms other than human are well-known and include, for example, chimpanzee dystrophin (XM_016943540.1 and XP_016799029.1; XM_016943544.1 and XP_016799033.1; XM_016943545.1 and XP_016799034.1; XM_016943537.1 and XP_016799026.1; XM_016943547.2 and XP_016799036.1; XM_016943548.1 and XP_016799037.1; XM_016943550.1 and XP_016799039.1; XM_016943549.1 and XP_016799038.1; XM_016943551.2 and XP_016799040.1; XM_016943539.2 and XP_016799028.1; XM_016943538.1 and XP_016799027.1; XM_016943542.1 and XP_016799031.1; XM_016943541.1 and XP_016799030.1; XM_016943543.1 and XP_016799032.1; XM_016943536.2 and XP_016799025.1; XM_016943546.2 and XP_016799035.1; and XM_016943552.1 and XP_016799041.1), dog dystrophin (NM_001003343.1 and NP_001003343.1; XM_014111344.2 and XP_013966819.1; XM_014111347.2 and XP_013966822.1; XM_014111342.2 and XP_013966817.1; XM_014111345.2 and XP_013966820.1; XM_005641030.3 and XP_005641087.1; XM_014111346.2 and XP_013966821.1; XM_005641029.3 and XP_005641086.1; XM 014111343.2 and XP 013966818.1; XM 022415519.1 and XP 022271227.1; XM_014111349.2 and XP_013966824.1; XM_022415520.1 and XP_022271228.1; XM_022415521.1 and XP_022271229.1; XM_005641038.2 and XP_005641095.1; XM_005641037.2 and XP_005641094.1; and XM_014111350.2 and XP_013966825.1), mouse dystrophin (NM_001314034.1 and NP_001300963.1; NM_001314035.1 and NP_001300964.1; NM_001314036.1 and NP_001300965.1; NM_001314037.1 and NP_001300966.1; NM_001314038.1 and NP_001300967.1; NM_007868.6 and NP 031894.1; XM_006527768.3 and XP_006527831.1; XM_017318379.2 and XP_017173868.1; XM_017318378.1 and XP_017173867.1; XM_017318377.1 and XP_017173866.1; XM_017318376.1 and XP_017173865.1; XM_017318374.1 and XP_017173863.1; XM_017318373.1 and XP_017173862.1; XM_006527767.3 and XP_006527830.1; XM_017318375.1 and XP_017173864.1; XM_030251215.1 and XP_030107075.1; and XM_006527773.2 and XP_006527836.1), chicken dystrophin (NM_205299.2 and NP_990630.2), and Drosophila dystrophin (NM_001043263.2 and NP_001036728.1; NM_001275801.1 and NP_001262730.1; NM_001275803.1 and NP_001262732.1; NM_001043259.2 and NP_001036724.1; NM_001043257.2 and NP_001036722.1; NM_001043258.2 and NP_001036723.1; NM_001043261.2 and NP_001036726.1; NM_001275804.1 and NP_001262733.1; NM_001043256.2 and NP_001036721.1; NM_001043260.2 and NP_001036725.1; NM_001043262.2 and NP_001036727.1; NM_001275802.1 and NP_001262731.1). In addition, numerous anti-dystrophin antibodies having a variety of characterized specificities and suitabilities for various immunochemical assays are commercially available and well-known in the art, including antibody LSB8434from Lifespan Biosciences, antibody TA318925 from Origene, antibodies ab218198, ab15277, and ab7164 from Abcam, and antibody sc-33697 from Santa Cruz Biotechnology, and the like.

As used herein, the terms “neurological diseases” or “neurological disorders” refers to a host of undesirable conditions affecting neurons in the brain of a subject. Representative examples of such conditions include, without limitation, Alzheimer's disease, Parkinson's disease, Huntington's disease, Pick's disease, Kuf's disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dytonia syndrome, Leigh syndrome, Leber hereditary optic neuropathy(LHON), parkinsonism, dystonia, motor neuron disease, neuropathy-ataxia and retinitis pimentosa (NARP), maternal inherited Leigh syndrome (MILS), Friedreich ataxia, hereditary spastic paraplegia, Mohr-Tranebjaerg syndrome, Wilson disease, sporatic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, korsakoff's psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity and movement disorders. As used herein, the term “movement disorder” includes neurological diseases or disorders that involve the motor and movement systems, resulting in a range of abnormalities that affect the speed, quality and ease of movement. Movement disorders are often caused by or related to abnormalities in brain structure and/or function. Movement disorders include, but are not limited to (i) tremors: including, but not limited to, the tremor associated with Parkinson's Disease, physiologic tremor, benign familial tremor, cerebellar tremor, rubral tremor, toxic tremor, metabolic tremor, and senile tremor; (ii) chorea, including, but not limited to, chorea associated with Huntington's Disease, Wilson's Disease, ataxia telangiectasia, infection, drug ingestion, or metabolic, vascular or endocrine etiology (e.g., chorea gravidarum or thyrotoxicosis); (iii) ballism (defined herein as abruptly beginning, repetitive, wide, flinging movements affecting predominantly the proximal limb and girdle muscles); (iv) athetosis (defined herein as relatively slow, twisting, writhing, snake-like movements and postures involving the trunk, neck, face and extremities); (v) dystonia (defined herein as a movement disorder consisting of twisting, turning tonic skeletal muscle contractions, most, but not all of which are initiated distally); (vi) paroxysmal choreoathetosis and tonic spasm; (vii) tics (defined herein as sudden, behaviorally related, irregular, stereotyped, repetitive movements of variable complexity); (viii) tardive dyskinesia; (ix) akathesia, (x) muscle rigidity, defined herein as resistance of a muscle to stretch; (xi) postural instability; (xii) bradykinesia; (xiii) difficulty in initiating movements; (xiv) muscle cramps; (xv) dyskinesias and (xvi) myoclonus.

The term “BDNF” refers to brain-derived neurotrophic factor and is a neurotrophin. The term, “neurotrophins” refers to a class of structurally related growth factors that promote neural survival and differentiation. They stimulate neurite outgrowth, suggesting that they can promote regeneration of injured neurons, and act as target-derived neurotrophic factors to stimulate collateral sprouting in target tissues that produce the neurotrophin (Korsching (1993) J Neurosci. 13: 2739). Brain-derived neurotrophic factor (BDNF) was initially characterized as a basic protein present in brain extracts and capable of increasing the survival of dorsal root ganglia (Leibrock et al. (1989) Nature 341:149). When axonal communication with the cell body is interrupted by injury, Schwann cells produce neurotrophic factors such as nerve growth factor (NGF) and BDNF. Neurotrophins are released from the Schwann cells and dispersed diffusely in gradient fashion around regenerating axons, which then extend distally along the neurotrophins' density gradient (Ide (1996) Neurosci. Res. 25:101). Local application of BDNF to transected nerves in neonatal rats has been shown to prevent massive death of motor neurons that follows axotomy (DiStefano et al. (1992) Neuron, 8:983; Oppenheim et al. (1992) Nature 360:755; and Yan et al. (1992) Nature 360:753). The mRNA titer of BDNF increases to several times the normal level four days after axotomy and reaches its maximum at 4 weeks (Meyer et al. (1992) J. Cell Biol. 119:45). Moreover, BDNF has been reported to enhance the survival of cholinergic neurons in culture (Nonomura et al. (1995) Brain Res. 683:129). In addition, nucleic acid and polypeptides sequences of BDNF orthologs in numerous species are well known in the art and include human BDNF (NM_001143805.1, NP_001137277.1, NM_001143806.1, NP_001137278.1, NM 001143807.1, NP_001137279.1, NM_001143808.1, NP_001137280.1, NM 001143809.1, NP_001137281.1, NM_001143810.1, NP_001137282.1, NM_001143811.1, NP_001137283.1, NM_001143812.1, NP_001137284.1, NM 001143813.1, NP_001137285.1, NM_001143814.1, NP_001137286.1, NM 001143815.1, NP_001137287.1, NM_001143816.1, NP_001137288.1, NM 001709.4, NP 001700.2, NM_170731.4, NP 733927.1, NM_170732.4, NP_733928.1, NM_170733.3, NP_733929.1, NM_170734.3, NP_733930.1, NM_170735.5, and NP_733931.1), chimpanzee BDNF (NM_001012441.1 and NP_001012443.1), monkey BDNF (XM_001089568.2 and XP_001089568.2), dog BDNF (NM_001002975.1 and NP_001002975.1), cow BDNF (NM_001046607.2 and NP_001040072.1), mouse BDNF (NM_001048139.1, NP_001041604.1, NM_001048141.1, NP_001041606.1, NM_001048142.1, NP_001041607.1, NM_007540.4, and NP_031566.4), rat BDNF (NM_001270630.1, NP_001257559.1, NM_001270631.1, NP_001257560.1, NM_001270632.1, NP_001257561.1, NM 001270633.1, NP_001257562.1, NM_001270634.1, NP_001257563.1, NM 001270635.1, NP_001257564.1, NM_001270636.1, NP_001257565.1, NM 001270637.1, NP_001257566.1, NM_001270638.1, NP_001257567.1, NM 012513.4, and NP_036645.2), chicken BDNF (NM_001031616.1 and NP_001026787.1), and zebrafish BDNF (NM_131595.2 and NP_571670.2). In addition, numerous anti-BDNF antibodies having a variety of characterized specificities and suitabilities for various immunochemical assays are commercially available and well known in the art, including antibody pa1014 from Boster Immunoleader, antibody BDNF-#9 from DSHB Iowa, antibody 209-401-C27 from Rockland, antibody BML-SA665 from Enzo Life Sciences, antibody EB08117 from Everest Biotech, antibody AHP1831 from AbD Serotec, antibody ANT-010 from Alomone, and the like.

As used herein, the term “neurodegenerative disease” or “neurodegenerative disorder” encompass a subset of neurological diseases characterized by involving a progressive loss of neurons or loss of neuronal function. Accordingly, the term “neurodegeneration” refers to the progressive loss or function of at least one neuron or neuronal cell. The ordinarily skilled artisan will appreciate that the term “progressive loss” can refer to cell death or cell apoptosis. The ordinarily skilled artisan would further appreciate that “neuronal cell loss” refers to the loss of neuronal cells. The loss of neuronal cells may be a result of a genetic predisposition, congenital dysfunction, apoptosis, ischemic event, immune-mediated, free-radical induced, mitochondrial dysfunction, lesion formation, misregulation or modulation of a central nervous system-specific pathway or activity, chemical induced, or any injury that results in a loss of neuronal cells, as well as a progressive loss of neuronal cells. Thus, a neurodegenerative disorder or neurodegenerative disease, as used in the current context, includes any abnormal physical or mental behavior or experience where the death of neuronal cells is involved in the etiology of the disorder, or is affected by the disorder. As used herein, neurodegenerative diseases encompass disorders affecting the central and peripheral nervous systems, and include such afflictions as memory loss, stroke, dementia, personality disorders, gradual, permanent or episodic loss of muscle control. Examples of neurodegenerative disorders or diseases for which the current invention can be used preferably include, but are not limited to, Alzheimer's Disease, Parkinson's Disease, Huntington's Disease, Lou Gehrig's disease or amyotrophic lateral sclerosis (ALS), Pick's disease, prion diseases, dystonia, dementia with Lewy bodies, multiple system atrophy, progressive supranuclear palsy, Friedreich's Ataxia, temporal lobe epilepsy, stroke, traumatic brain injury, mitochondrial encephalopathies, Guillain-Barre syndrome, multiple sclerosis, epilepsy, myasthenia gravis, chronic idiopathic demyelinating disease (CID), neuropathy, ataxia, dementia, chronic axonal neuropathy and stroke.

As used herein, the term “neuronal” or “neuron” refers to one or more cells that are a morphologic and functional unit of the brain, spinal column, and peripheral nerves consisting of nerve cell bodies, dendrites, and axons. Neuronal cell types can include, but are not limited to, typical nerve cell body showing internal structure, horizontal cell from cerebral cortex, Martinotti cell, bipolar cell, unipolar cell, Purkinje cell, and pyramidal cell of motor area of cerebral cortex. Exemplary neuronal cells can include, but are not limited to, cholinergic, adrenergic, noradrenergic, dopaminergic, serotonergic, glutaminergic, GABAergic, and glycinergic.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. The names of the natural amino acids are abbreviated herein in accordance with the recommendations of IUPAC-IUB.

The term “biological sample” when used in reference to a diagnostic assay is intended to include tissues, cells and biological fluids isolated from a subject, as well as tissues, cells and fluids present within a subject pleural fluid, pus, saliva, sebum, semen, serum, sweat, synovial fluid, tears, urine, vaginal lubrication, vitreous humor, vomit).

The terms “cancer” or “tumor” refer to the presence of cells possessing characteristics typical of cancer-causing cells, such as uncontrolled proliferation, immortality, metastatic potential, rapid growth and proliferation rate, and certain characteristic morphological features.

In some embodiments, the term “muscle tissue” refers a collection of myocytes and neuromuscular junctions. The myocytes may be individual or organized as myofibrils and/or muscle fibers. In some embodiments, the term “muscle tissue” means a muscle cell (e.g., a myocyte either alone or in connection with a motor neuron).

Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., myelomas like multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), myeloma, multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma, or breast carcinoma. The epithelial cancers may be characterized in various other ways including, but not limited to, serous, endometrioid, mucinous, clear cell, Brenner, or undifferentiated. In some embodiments, the cancer comprises a solid tumor. In some embodiments, the tumor is an adenocarcinoma, an adrenal tumor, an anal tumor, a bile duct tumor, a bladder tumor, a bone tumor, a blood born tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngreal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

The term “isolated polypeptide” refers to a polypeptide, in certain embodiments prepared from recombinant DNA or RNA, or of synthetic origin, or some combination thereof, which (1) is not associated with proteins that it is normally found within nature, (2) is isolated from the cell in which it normally occurs, (3) is isolated free of other proteins from the same cellular source, (4) is expressed by a cell from a different species, or (5) does not occur in nature.

The terms “label” or “labeled” refer to incorporation or attachment, optionally covalently or non-covalently, of a detectable marker into a molecule, such as a polypeptide. Various methods of labeling polypeptides are known in the art and may be used. Examples of labels for polypeptides include, but are not limited to, the following: radioisotopes, fluorescent labels, heavy atoms, enzymatic labels or reporter genes, chemiluminescentgroups, biotinyl groups, predetermined polypeptide epitopes recognized by a secondary reporter (e.g., leucine zipper pair sequences, binding sites for secondary antibodies, metal binding domains, epitope tags). Examples and use of such labels are described in more detail below. In some embodiments, labels are attached by spacer arms of various lengths to reduce potential steric hindrance.

In some embodiments, the term “neuronal degeneration” or “neuron degeneration” includes any loss of neuronal cells in specified regions of the nervous system. The loss may be rapid, or it may be slow and progressive. Progressive loss of neural tissues includes death of neurons over a period of time. Degeneration may be the results of inability of the neurons to self-regenerate after neurodegenerative cell death or severe damage that occurs to the neural tissue. Loss of neurons in a single subject may be qualified in any number of ways, including comparison of neuronal number or density comparison to a population average based on age or other demographics. As another example, loss of neuronal number or density can be qualified as an initial measurement of neuronal number or density, and a lower number or loss of density at a second measurement in time can indicate neuronal degeneration. Alternatively, degeneration of neurons may be quantified by measurement of metabolites or striatal DA, such as 3,4-dihydroxyphenylacetic acid (DOPAC).

As used herein, “pathogenic α-synuclein” can refer to an increase in the amount of misfolded and/or mutated α-synuclein, or to an increase in the overall levels of any form, including wild type, of α-synuclein in cells or tissues to predispose or cause a pathogenic condition (e.g., a α-synucleinopathy or a cancer disclosed herein) in a subject. Pathogenic α-synuclein may also refer to a form of α-synuclein showing differential posttranslational modification. Although trace levels of phosphorylated α-synuclein are detectable in healthy brains, much of the α-synuclein accumulated within Lewy bodies in Parkinson's disease brains is phosphorylated on serine 129 (Ser-129). Therefore, increased levels of α-synuclein may refer not only to total levels of wild-type α-synuclein, but any mutated form of α-synuclein, such as α-synuclein phosphorylated on serine 129 (Ser-129).

The “tumor microenvironment” is an art-recognized term and refers to the cellular environment in which the tumor exists, and includes, for example, interstitial fluids surrounding the tumor, surrounding blood vessels, immune cells, other cells, fibroblasts, signaling molecules, and the extracellular matrix.

In some embodiments, the term “subject” refers to a mammalian subject, such as a rodent, primate, or human.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “treatment,” as used herein, is defined as the application or administration of a therapeutic agent to a patient, or application or administration of a therapeutic agent to an isolated tissue or cell line from a patient, who has a disease or disorder, a symptom of a disease or disorder or a predisposition toward a disease or disorder, with the purpose of curing, healing, alleviating, relieving, altering, remedying, ameliorating, improving or affecting the disease or disorder, the symptoms of disease or disorder or the predisposition toward a disease or disorder. A therapeutic agent includes, but is not limited to, polypeptides, small molecules, peptides, peptidomimetics, nucleic acid molecules, antibodies, ribozymes, siRNA molecules, and sense and antisense oligonucleotides described herein

As used herein, the term “FNDC5” refers to fibronectin type III domain containing 5 proteins and are intended to include fragments, variants (e.g., allelic variants) and derivatives thereof. The nucleotide and amino acid sequences of mouse FNDC5, which correspond to GenBank Accession number NM_027402.4 and NP_081678.1 respectively, are set forth in SEQ ID NOs: 4 and 5. At least three splice variants encoding distinct human FNDC5 isoforms exist (isoform 1, NM_001171941.3, NP_001165412.1; isoform 2, NM_153756.3, NP_715637.2; and isoform 3, NM_001171940.2, NP_001165411.2). Representative nucleic acid and polypeptide sequences of human FNDC5 are respectively set forth in SEQ ID NOs: 6-7. Nucleic acid and polypeptide sequences of FNDC5 orthologs in organisms other than mice and human are well-known and include, for example, chimpanzee FNDC5 (XM_001155446.4, and XP_001155446.4), monkey FNDC5 (XM_001098747.2 and XP_001098747.2), rat FNDC5 (XM_002729542.3 and XP_002729588.2), and chicken FNDC5 (XM_417814.2; XP_417814.2, respectively set forth in SEQ ID NOs: 8 and 9). In addition, numerous anti-FNDC5 antibodies having a variety of characterized specificities and suitabilities for various immunochemical assays are commercially available and well-known in the art, including antibody LS-C486450 from Lifespan Biosciences, antibodies AG-25B-0027 and -0027B from Adipogen, antibody HPA051290 from Atlas Antibodies, antibodies PAN576Hu01, Hu02, Mu01, and Mu02 from Uscn Lifesciences, antibodies OACD03594 and OACD03595 from Aviva Systems Biology, antibody or b39441 from Biorbyt, antibody ab93373 from Abcam, antibody NBP2-14024 from Novus Biologicals, antibodies 509549 and 044959 from United States Biological, antibody ABCA2332953 from Abgent, and the like.

As used herein, the term “irisin” refers to the N-terminal transmembrane domain of FNDC5 and is intended to include fragments, variants (e.g., glycosylation mutants) and derivatives thereof. The amino acid sequence of irisin is conserved 100% between mice and humans (e.g., amino acid 29-140 of mouse FNDC5, or amino acid 32-143 of humanFNDC5, as set forth in SEQ ID NO: 2).

In some embodiments, irisin glycosylation mutant and biologically active fragments thereof are described and employed. The irisin glycosylation mutant polypeptide can comprise homology or identity to the irisin portion of FNDC5 (e.g., SEQ ID NO: 2) and the glycosylated residue at position 36, a glycosylated residue at position 81, and a non-N-linked glycosylated residue at position 45 (e.g., SEQ ID NO: 1 or SEQ ID NO: 13). In some embodiments, the irisin glycosylation mutant polypeptide can comprise site-specific glycosylation mutations that exhibit increased stability and receptor binding affinity. In some embodiments, the irisin glycosylation mutant polypeptide can comprise site-specific glycosylation mutations that exhibit increased half-life. In some embodiments, the non-N-linked glycosylated residue at position 45 is a non-canonical N-linked glycosylated residue at position 45. In some embodiments, the irisin polypeptide is a non-naturally occurring form of irisin. In some embodiments, the residue at position 45 is mutated such that it cannot be N-linked glycosylated. In some embodiments, the irisin polypeptide comprises a Q residue at position 45. In some embodiments, the irisin polypeptide comprises a non-hydrophobic residue at position 45. In some embodiments, exemplary non-hydrophobic residue at position 45 may be glutamine, threonine, serine, cysteine, arginine, histidine, lysine, aspartic acid, or glutamic acid. In some embodiments, the non-hydrophobic residue at position 45 does not cause misfolding within the irisin polypeptide. The irisin polypeptide may comprise an amino acid sequence having at least 80%, 85%, 90%, 95%, or 99% sequence identity to the amino acid sequence set forth in SEQ ID NO: 1 or SEQ ID NO: 13. In some embodiments, the irisin polypeptide may comprise SEQ ID NO: 1 or SEQ ID NO: 13.

In some embodiments, the irisin glycosylation mutant polypeptide does not comprise a signal polypeptide. In some embodiments, the irisin glycosylation mutant polypeptide does not comprise a FNDC5 signal polypeptide. In some embodiments, the irisin glycosylation mutant polypeptide comprises a signal polypeptide. In some embodiments, the irisin glycosylation mutant polypeptide further comprises a FNDC5 signal polypeptide. The FNDC5 signal polypeptide may comprise the amino acid sequence of SEQ ID NO: 12 (MEWSWVFLFFLSVTTGVHS). An irisin polypeptide disclosed herein can comprise at least one fibronectin domain of irisin protein without containing the full-length irisin protein sequence. In some embodiments, irisin polypeptides or fragments thereof disclosed herein can further comprise one or more polyhistidine (His)-Tag(s), a human rhinovirus 3C protease (HRV-3C) protease tag, a GFP sequence, or at least one Strep-II tag. In some embodiments, the irisin polypeptide further comprises an amino acid modification, post-translational modification, and/or a heterologous an amino acid sequence that stabilizes the irisin polypeptide and/or increases its half-life.

TABLE 1
SEQ ID NO: 1 Irisin-N45Q Recombinant Amino Acid Sequence
DSPSAPVNVTVRHLKAQSAVVSWDVLEDEVVIGFAISQQKKDVRMLRFIQEVNTTTRSCALWDLEEDTE
YIVHVQAISIQGQSPASEPVLFKTPREAEKMASKNKDEVTMKE
SEQ ID NO: 2 Irisin-WT Amino Acid Sequence
DSPSAPVNVTVRHLKANSAVVSWDVLEDEVVIGFAISQQKKDVRMLRFIQEVNTTTRSCALWDLEEDTE
YIVHVQAISIQGQSPASEPVLFKTPREAEKMASKNKDEVTMKE
SEQ ID NO: 3 Irisin-WT Recombinant Amino Acid Sequence
DSPSAPVNVTVRHLKANSAVVSWDVLEDEVVIGFAISQQKKDVRMLRFIQEVNTTTRSCALWDLEEDTE
YIVHVQAISIQGQSPASEPVLFKTPREAEKMASKNKDEVTMKEHHHHHHHHHHRDPLEVLFQ
SEQ ID NO: 4 Mouse FNDC5 cDNA Sequence
atg ccc cca ggg ccg tgc gcc tgg ccg ccc cgc gcc gcg ctc cgc ctg
tgg cta ggc tgc gtc tgc ttc gcg ctg gtg cag gcg gac agc ccc tca
gcc cct gtg aac gtg acc gtc cgg cac ctc aag gcc aac tct gcc gtg
gtc agc tgg gat gtc ctg gag gat gaa gtg gtc att ggc ttt gcc atc
tct cag cag aag aag gat gtg cgg atg ctc cgg ttc att cag gag gtg
aac acc acc acc cgg tcc tgc gct ctc tgg gac ctg gag gag gac aca
gaa tat atc gtc cat gtg cag gcc atc tcc atc cag gga cag agc cca
gcc agt gag cct gtg ctc ttc aag acc cca cgc gag gct gaa aag atg
gcc tca aag aac aaa gat gag Gtg acc atg aag gag atg ggg agg aac
cag cag ctg cga acg (ggg) gag gtg ctg atc att gtt gtg gtc ctc ttc
atg tgg gca ggt gtt ata gct ctc ttc tgc cgc cag tat gat atc Atc
aag gac aac gag ccc aat aac aac aag gag aaa acc aag agc gca tca
gaa acc agc Aca ccg gag cat cag ggt ggg ggt ctc ctc cgc agc aag
ata tga
SEQ ID NO: 5 Mouse FNDC5 Amino Acid Sequence
M P P G P C A W P P R A A L R L W L G C V C F A L V Q A D S P S A
P V N V T V R H L K A N S A V V S W D V L E D E V V I G F A I S Q
Q K K D V R M L R F I Q E V N T T T R S C A L W D L E E D T E Y I
V H V Q A I S I Q G Q S P A S E P V L F K T P R E A E K M A S K N
K D E V T M K E M G R N Q Q L R T G E V L I I V V V L F M W A G V
I A L F C R Q Y D I I K D N E P N N N K E K T K S A S E T S T P E
H Q G G G L L R S K I
SEQ ID NO: 6 Human FNDC5cDNA Sequence

| | |
|---|---|
| 1 | aaagagaaaa gagagagaga ggtgctctgg ctccggggct cttctcccaa acgggtcgag |
| 61 | gtcccagctg aggctctccc aggacaagtc tctcagcccc agtgaacgtc accgtcaggc |
| 121 | acctcaaggc caactctgca gtggtgagct gggatgttct ggaggatgag gttgtcatcg |
| 181 | gatttgccat ctcccagcag aagaaggatg tgcggatgct gcgcttcatc caggaggtga |
| 241 | acaccaccac ccgctcatgt gccctctggg acctggagga ggatacggag tacatagtcc |
| 301 | acgtgcaggc catctccatt cagggccaga gcccagccag cgagcctgtg ctcttcaaga |
| 361 | ccccgcgtga ggctgagaag atggcctcca agaacaaaga tgaggtaacc atgaaagaga |
| 421 | tggggaggaa ccaacagctg cggacaggcg aggtgctgat catcgtcgtg gtcctgttca |
| 481 | tgtgggcagg tgtcattgcc ctcttctgcc gccagtatga catcatcaag gacaatgaac |
| 541 | ccaataacaa caaggaaaaa accaagagtg catcagaaac cagcacacca gagcaccagg |
| 601 | gcggggggct tctccgcagc aaggtgaggg caagacctgg gcctgggtgg gccaccctgt |
| 661 | gcctcatgct ctggtaatcc ctggactgca gagggggtca gctcggggac tggcatggcg |
| 721 | acagctgggc agagccagac ttggctgctg cctgtgtgac acagggacac tgcaagttga |
| 781 | ttttggatcc tctccttttg cagggtgcca ggggccagtg gtgatttgaa gaaaaaaaaa |
| 841 | aaaaaaaaa |

SEQ ID NO: 7 Human FNDC5 Amino Acid Sequence

| | |
|---|---|
| 1 | mhpgspsawp praraalrlw lgcvcfalvq adspsapvnv tvrhlkansa vvswdvlede |
| 61 | vvigfaisqq kkdvrmlrfi qevntttrsc alwdleedte yivhvqaisi qgqspasepv |
| 121 | lfktpreaek masknkdevt mkemgrnqql rtgevliivv vlfmwagvia lfcrqydiik |
| 181 | dnepnnnkek tksasetstp ehqgggllrs ki |

SEQ ID NO: 8 Chicken FNDC5 cDNA Sequence

| | |
|---|---|
| 1 | atggagaaga acagggacgg ccgcggcccc cctggtgtcc atctggggat ggagaaggaa |
| 61 | gatgatttag agcccggtga cacgccgggg ctgcgcgaag ccctggtggc gagatgtcac |
| 121 | cgctgccgcg cacccgccgg gggtctcacc gggacgggcc ccgtttgctc cttccggcga |
| 181 | tggggagcgg tccgggccga gggctcccgg tcccgcctgg gggaaactga ggcagacggc |
| 241 | ggggccgggc ggggcggggg ccgagccgcc cccgggccgg gggagggacc ggagcggggc |
| 301 | tgcccagcgc tgcagegggc ggagccgggg ctcggcgggg cegcctcccg gccgagccga |
| 361 | gccgaaccga gccgcgctgc cgagggcege cgagcccgca gccgcccccg gccgaaccgg |
| 421 | gcggccccgc cggttccggg ccccggagct ctccgcggtg ctgaacggcg ccgccgcgcc |
| 481 | cgcgggacgc cggccccgga geggctcggc cccggcgcgg cgcggcgggc cgcgggggga |
| 541 | tggagccctt cctgggctgc accggcgccg cgctcctgct ctgctttcag ctacgccggt |
| 601 | ctgcggccgg tggaggcaga cagcccttcg gctccggtca atgtcacagt caaacacctg |
| 661 | aaggccaact cagctgtagt gacttgggac gttctggagg atgaagttgt cattggattt |
| 721 | gccatttccc agcagaagaa ggacgtgcgg atgctgcgct tcatccagga ggtgaacacc |
| 781 | accacccgct cctgtgccct ctgggaccta gaggaggaca ctgagtacat tgtgcatgtc |
| 841 | caggccatca gcatccaagg ccagagccct gccagtgage cagtcctctt caagaccccc |
| 901 | agggaagctg agaaactggc ttctaaaaat aaagatgagg tgacaatgaa ggagatggcg |
| 961 | aagaaaaacc aacagctgcg cgcaggggaa atactcatca ttgtggtggt gttgtttatg |
| 1021 | tgggcagggg tgatcgccct gttctgcagg cagtacgaca tcatcaaaga caacgagccg |
| 1081 | aacaacagca aggagaaagc caagagcgcc tcagagaaca gcacccccga gcaccagggt |
| 1141 | ggggggctgc tccgcagcaa gttcccaaaa aacaaaccct cagtgaacat cattgaggca |
| 1201 | taa |

SEQ ID NO: 9 Chicken FNDC5 Amino Acid Sequence

| | |
|---|---|
| 1 | meknrdgrgp pgvhlgmeke ddlepgdtpg lrealvarch rcrapagglt gtgpvcsfrr |
| 61 | wgavraegsr srlgeteadg gagrgggraa pgpgegperg cpalqraepg lggaasrpsr |
| 121 | aepsraaegr rarsrprpnr aappvpgpga lrgaerrrra rgtpaperlg pgaarraagg |
| 181 | wspswaapap rscsafsyag lrpveadsps apvnvtvkhl kansavvtwd vledevvigf |
| 241 | aisqqkkdvr mlrfiqevnt ttrscalwdl eedteyivhv qaisiqgqsp asepvlfktp |
| 301 | reaeklaskn kdevtmkema kknqqlrage iliivvvlfm wagvialfcr qydiikdnep |
| 361 | nnskekaksa senstpehqg ggllrskfpk nkpsvniiea |

SEQ ID NO: 10 Fragment of Murine FNDC5 Nucleic Acid Sequence that encodes irisin
(amino acid residues 29-140) of murine FNDC5

| | |
|---|---|
| 104 | gacagcc cctcagcccc |
| 121 | tgtgaacgtg accgtccggc acctcaaggc caactctgcc gtggtcagct gggatgtcct |
| 181 | ggaggatgaa gtggtcattg gctttgccat ctctcagcag aagaaggatg tgcggatgct |
| 241 | ccggttcatt caggaggtga acaccaccac ccggtcctgc gctctctggg acctggagga |
| 301 | ggacacagaa tatatcgtcc atgtgcaggc catctccate cagggacaga gcccagccag |
| 361 | tgagcctgtg ctcttcaaga ccccacgcga ggctgaaaag atggcctcaa agaacaaaga |
| 421 | tgaggtgacc atgaaggag |

SEQ ID NO: 11 Irisin Fragment of Human FNDC5 Nucleic Acid Sequence

| | |
|---|---|
| 161 | gacagtccct cagccccagt |
| 181 | gaacgtcacc gtcaggcacc tcaaggccaa ctctgcagtg gtgagctggg atgttctgga |
| 241 | ggatgaggtt gtcatcggat ttgccatctc ccagcagaag aaggatgtgc ggatgctgcg |
| 301 | cttcatccag gaggtgaaca ccaccacccg ctcatgtgcc ctctgggacc tggaggagga |
| 361 | tacggagtac atagtccacg tgcaggccat ctccattcag ggccagagcc cagccagcga |
| 421 | gcctgtgctc ttcaagaccc cgcgtgaggc tgagaagatg gcctccaaga acaaagatga |
| 481 | ggtaaccatg aaagag |

SEQ ID NO: 13 Irisin-N45Q Recombinant Amino Acid Sequence
MSPSAPVNVTVRHLKAQSAVVSWDVLEDEVVIGFAISQQKKDVRMLRFIQEVNTTTRSCALWDLEE
DTEYIVHVQAISIQGQSPASEPVLFKTPREAE
SEQ ID NO: 14 Irisin Amino Acid Sequence Used for the Modeling
MSPSAPVNVTVRHLKANSAVVSWDVLEDEVVIGFAISQQKKDVRMLRFIQEVNTTTRSCALWDLEE
DTEYIVHVQAISIQGQSPASEPVLFKTPREAE
SEQ ID NO: 15 Irisin-N45Q Recombinant Amino Acid Sequence (with His Tag and
HRV3C sequence)
DSPSAPVNVTVRHLKAQSAVVSWDVLEDEVVIGFAISQQKKDVRMLRFIQEVNTTTRSCALWDLEEDTE
YIVHVQAISIQGQSPASEPVLFKTPREAEKMASKNKDEVTMKEHHHHHHHHHHRDPLEVLFQ

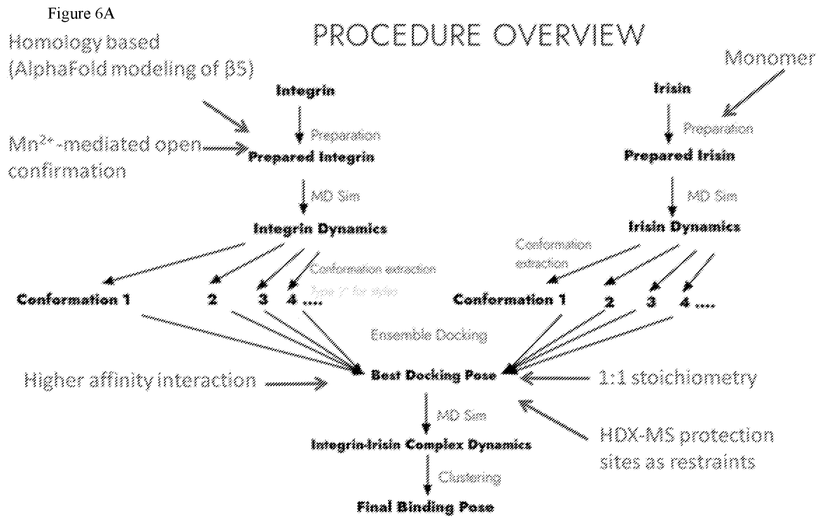

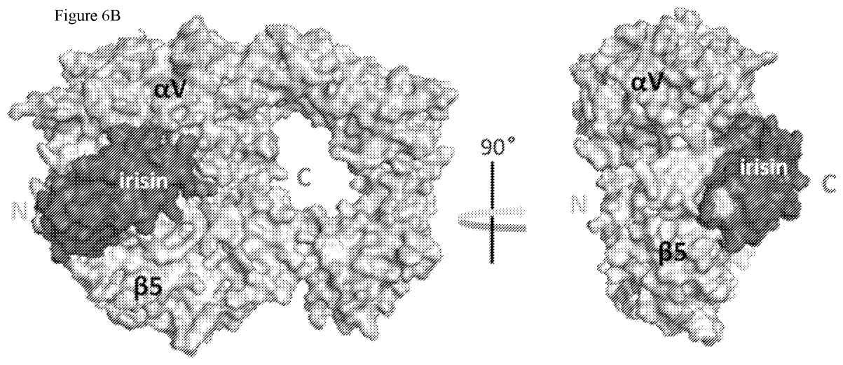

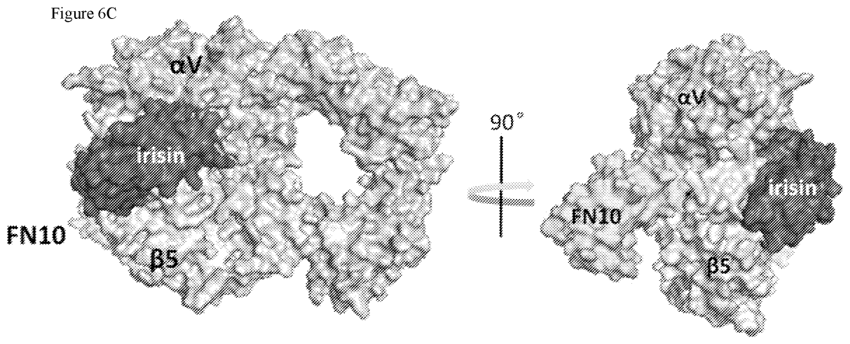

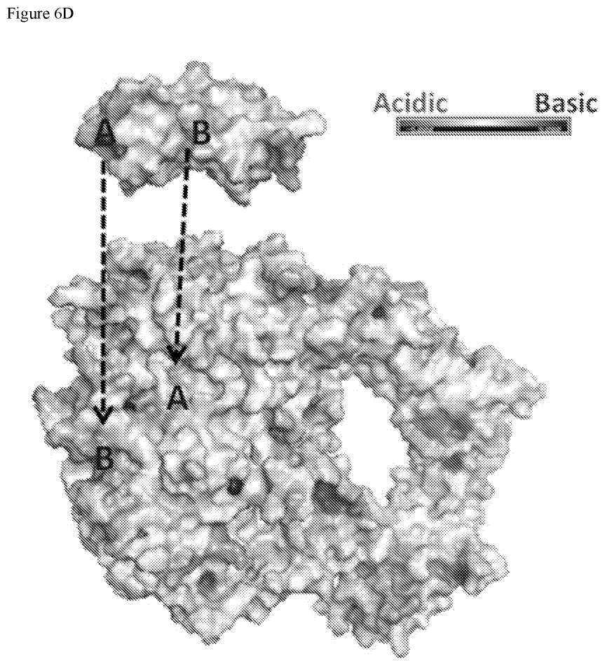

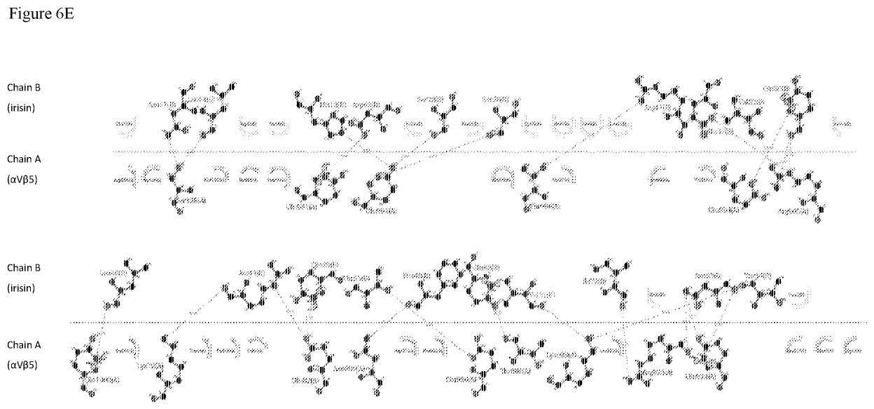

TABLE 2
Irisin residues that have interactions with αVβ5
at the irisin/αVβ5 interface (FIG. 6E)

| | List of irisin residues (numbered as |
|---|---|
| | shown in SEQ ID NO: 14 and FIG. 6E) |
| Having hydrophobic | V20, I32, F34, V43, M45, I49, T56, C59, |
| interactions with αVβ5 | A60, L61, W62, E65 |
| at the irisin αVβ5 | |
| interface | |
| Having electrostatic | H13, N17, S22, V31, Q39, R44, L46, R47, |
| interactions with αVβ5 | F48, Q50, E51, V52, N53, T54, T55, S58, |
| at the irisin αVβ5 | D63, L64, I79, Q82 |
| interface | |

In some embodiments, the irisin polypeptide comprises SEQ ID NO: 14 and a mutation to at least one residue listed in Table 2.

It will be appreciated that specific sequence identifiers (SEQ ID NOs) have been referenced throughout the specification for purposes of illustration and should therefore not be construed to be limiting. Any marker encompassed by the present invention, including, but not limited to, the markers described in the specification and markers described herein, are well-known in the art and may be used in the embodiments encompassed by the present invention.

### Polypeptides, Nucleic Acids, Antibodies, Vectors, and Host Cells Useful for Methods Described Herein

Polypeptides, nucleic acids, vectors, and host cells related to irisin, irisin glycosylation mutants, and biologically active fragments thereof are useful for carrying out the methods described herein.

In one embodiment, nucleic acid molecules encompassed by the present invention encode an irisin polypeptide disclosed herein (e.g., irisin glycosylation mutants, or biologically active fragments thereof; e.g., the irisin glycosylation mutant polypeptides comprising a glycosylated residue at position 36, a glycosylated residue at position 81, and a non-N-linked glycosylated residue at position 45). In an exemplary embodiment, the irisin polypeptide disclosed herein may include an amino acid sequence sufficiently homologous to an irisin amino acid portion of SEQ ID NO: 1 or SEQ ID NO: 13, or fragments thereof, such that the protein or portion thereof induces one or more of the following biological activities: 1) expression or activity of irisin, irisin glycosylation mutants, or biologically active fragments thereof; 2) activity-induced immediate-early gene expression of irisin, irisin glycosylation mutants, and fragments thereof, 3) preventing or reducing degeneration of dopaminergic (DA) neurons, preventing or ameliorating at least one motor deficit and/or preventing or ameliorating at least one symptom of cognitive dysfunction or dementia in a subject in need thereof; 4) reducing the level or amount of α-synuclein in the cells; 5) increasing expression of brain-derived neurotrophic factor (BDNF); 6) treating or preventing neurological diseases or disorders that would benefit from decreased neuronal cell death and/or increased neuronal survival; 7) increasing muscle physiology of a muscle tissue; 8) preventing or treating muscular atrophy or muscular dystrophy; or 9) preventing or treating neurological disease.

As used herein, the language “sufficiently homologous” refers to proteins or portions thereof that have amino acid sequences that include a minimum number of identical or equivalent (e.g., an amino acid residue that has a similar side chain as an amino acid residue in SEQ ID NO: 1 or SEQ ID NO: 13, or fragments thereof) amino acid residues to an irisin amino acid sequence in SEQ ID NO: 1 or SEQ ID NO: 13, or fragments thereof, such that the protein or portions thereof induces one or more of the following biological activities: 1) expression or activity of irisin, irisin glycosylation mutants, or biologically active fragments thereof, 2) activity-induced immediate-early gene expression of irisin, irisin glycosylation mutants, and fragments thereof, 3) preventing or reducing degeneration of dopaminergic (DA) neurons, preventing or ameliorating at least one motor deficit and/or preventing or ameliorating at least one symptom of cognitive dysfunction or dementia in a subject in need thereof, 4) reducing the level or amount of α-synuclein in the cells; 5) increasing expression of brain-derived neurotrophic factor (BDNF); 6) treating or preventing neurological diseases or disorders that would benefit from decreased neuronal cell death and/or increased neuronal survival; 7) increasing muscle physiology of a muscle tissue; 8) preventing or treating muscular atrophy or muscular dystrophy; or 9) preventing or treating neurological disease.

In some embodiments, the irisin protein disclosed herein is at least about 50%, at least about 60%, at least about 70%, 75%, 80%, 85%, 90%, 91%, 92%, 93%, 94%, 95%, 96%, 97%, 98%, 99% or more homologous to the entire irisin amino acid sequence within SEQ ID NO: 1 or SEQ ID NO: 13, or fragments thereof.

Portions of proteins encoded by irisin nucleic acid molecules are biologically active portions of irisin. As used herein, the term “biologically active portion” is intended to include a portion, e.g., a domain/motif, of irisin that has one or more of the biological activities of the full-length irisin protein, such as listed above. In some embodiments, the targeted motif of the irisin glycosylation mutant polypeptides comprises a glycosylated residue at position 36, a glycosylated residue at position 81, and a non-N-linked glycosylated residue at position 45.

Biologically active portions or biologically active fragments of the irisin polypeptide described herein (e.g., irisin glycosylation mutants) may include peptides comprising amino acid sequences derived from the amino acid sequence of the irisin polypeptide described herein (e.g., irisin glycosylation mutants), e.g., the amino acid sequence shown in SEQ ID NO: 1 or SEQ ID NO: 13, or fragments thereof, or the amino acid sequence of a protein homologous to the irisin polypeptide described herein (e.g., irisin glycosylation mutants), which include fewer amino acids than the full-length protein or the full-length protein that is homologous to the irisin polypeptide described herein (e.g., irisin glycosylation mutants), and exhibit at least one activity of the irisin polypeptide described herein (e.g., irisin glycosylation mutants), or complex thereof. In some embodiments, the irisin polypeptide may comprise a mutation to at least one residue listed in Table 2 to modulate at least one activity of the irisin polypeptide described herein (e.g., irisin glycosylation mutants), or complex thereof. In some embodiments, the irisin polypeptide comprises SEQ ID NO: 14 and a mutation to at least one residue listed in Table 2. Typically, biologically active portions (peptides, e.g., peptides that are, for example, 50, 55, 60, 65, 70, 75, 80, 85, 90, 95, 100 or more amino acids in length) comprise a domain or motif, e.g., signal peptide, extracellular domain, fibronectin domain, hydrophobic, and/or C-terminal domain). In some embodiments, the biologically active portion of the protein that includes one or more the domains/motifs described herein can decrease one of the following: 1) tremor at rest, such as a slight tremor in the hands or feet; 2) rigidity (stiffness) of limbs, neck, or shoulders; 3) difficulty balancing (postural instability); 4) slowness of movement or gradual loss of spontaneous movement (bradykinesia); 6) trouble standing after sitting; 7) stiffness in the limbs, or 8) moving more slowly. In some embodiments, the biologically active portion of the protein that includes one or more the domains/motifs described herein can decrease one of the following: confusion, poor motor coordination, loss of short-term or long-term memory, identity confusion, or impaired judgment. In some embodiments, the biologically active portion of the protein that includes one or more the domains/motifs described herein can decrease the level or amount of α-synuclein in the cells. In other embodiments, the biologically active portion of the protein that includes one or more the domains/motifs described herein can increase expression of brain-derived neurotrophic factor (BDNF). In still another embodiment, the biologically active portion of the protein that includes one or more the domains/motifs described herein can increase muscle physiology of a muscle tissue affected by muscular atrophy or muscular dystrophy.

Moreover, other biologically active portions, in which other regions of the irisin polypeptide described herein (e.g., irisin glycosylation mutants) are deleted, may be prepared by recombinant techniques and evaluated for one or more of the activities described herein. In some embodiments, the biologically active portions of the irisin polypeptide described herein (e.g., irisin glycosylation mutants) include one or more selected domains/motifs or portions thereof having biological activity. In another embodiment, a biologically active fragment of the irisin polypeptide described herein (e.g., irisin glycosylation mutants) comprises and/or consists of about amino acids 1-81, 1-82, 1-83, 1-84, 1-85, 1-90, 1-95, 1-100, 1-105, 1-110, 1-112, or any range in between residues 1 and 112 of SEQ ID NO: 1 or SEQ ID NO: 13. In some embodiments, a biologically active fragment of the irisin polypeptide described herein (e.g., irisin glycosylation mutants) comprises a portion of a full-length irisin fragment of interest that is less than 112, 110, 105, 100, 95, 90, 85, 80, 75, or 70 amino acids in length.

Standard binding assays, e.g., immunoprecipitations and yeast two-hybrid assays, as described herein, or functional assays, e.g., RNAi or overexpression experiments, may be performed to determine the ability of the irisin polypeptide disclosed herein (e.g., irisin glycosylation mutants or biologically active fragments thereof) to maintain and/or enhance a biological activity of the full-length irisin protein.

Isolated nucleic acid molecules that encode the irisin polypeptide disclosed herein (e.g., irisin glycosylation mutants or biologically active fragments thereof) are described herein. As used herein, the term “nucleic acid molecule” is intended to include DNA molecules (i.e., cDNA or genomic DNA) and RNA molecules (i.e., mRNA) and analogs of the DNA or RNA generated using nucleotide analogs. The nucleic acid molecule may be single-stranded or double-stranded. An “isolated” nucleic acid molecule is one that is separated from other nucleic acid molecules that are present in the natural source of the nucleic acid. In some embodiments, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated irisin nucleic acid molecule can contain less than about 5 kb, 4 kb, 3 kb, 2 kb, 1 kb, 0.5 kb or 0.1 kb of nucleotide sequences that naturally flank the nucleic acid molecule in genomic DNA of the cell from which the nucleic acid is derived (i.e., a brown adipocyte). Moreover, an “isolated” nucleic acid molecule, such as a cDNA molecule, may be substantially free of other cellular material, or culture medium when produced by recombinant techniques, or chemical precursors or other chemicals when chemically synthesized.

In some embodiments, the nucleic acid encoding an irisin polypeptide disclosed herein (e.g., irisin glycosylation mutants and biologically active fragments thereof) is generated by site-directed mutagenesis using the irisin DNA template described herein and a deglycosylase.

A nucleic acid molecule encompassed the irisin DNA template for the present invention, e.g., a nucleic acid molecule having the irisin portion of the nucleotide sequence of SEQ ID NO: 4, 6, or 8 (e.g., SEQ ID No: 10 or 11), or a nucleotide sequence that is at least about 50%, at least about 60%, at least about 70%, at least about 80%, at least about 90%, or at least about 95% or more (e.g., about 98%) homologous to the irisin portion of the nucleotide sequence shown in SEQ ID NOs: 4, 6, 8, 10, and 11 or a portion thereof (i.e., 100, 200, 300, 400, 450, 500, or more nucleotides), may be isolated using standard molecular biology techniques and the sequence information provided herein. For example, a human irisin cDNA may be isolated from a human cell line using all or portion of the irisin segment in SEQ ID NO: 11, or fragments thereof (e.g., nucleic acid encoding SEQ ID NO: 14), as a hybridization probe and standard hybridization techniques (i.e., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, NY, 1989). Moreover, a nucleic acid molecule encompassing all or a portion of the irisin segment in SEQ ID NOs: 4, 6, 8, 10, and 11 or a nucleotide sequence that is at least about 50%, at least about 60%, at least about 70%, at least about 80%, at least about 90%, or at least about 95% or more homologous to the nucleotide sequence shown in SEQ ID NOs: 4, 6, 8, 10, and 11, or fragments thereof (e.g., nucleic acid encoding SEQ ID NO: 14), may be isolated by the polymerase chain reaction using oligonucleotide primers designed based upon the irisin portion of the sequence of SEQ ID NOs: 4, 6, 8, 10, and 11, or fragments thereof (e.g., nucleic acid encoding SEQ ID NO: 14), or the homologous nucleotide sequence. For example, mRNA may be isolated from cells disclosed herein (i.e., by the guanidinium-thiocyanate extraction procedure of Chirgwin et al., (1979) Biochemistry 18: 5294-5299) and cDNA may be prepared using reverse transcriptase (i.e., Moloney MLV reverse transcriptase, available from Gibco/BRL, Bethesda, MD; or AMV reverse transcriptase, available from Seikagaku America, Inc., St. Petersburg, FL). Synthetic oligonucleotide primers for PCR amplification may be designed based upon the irisin portion of the nucleotide sequence shown in SEQ ID NOs: 4, 6, 8, 10, and 11, or fragments thereof (e.g., nucleic acid encoding SEQ ID NO: 14), or to the homologous nucleotide sequence. A nucleic acid encompassed by the present invention may be amplified using cDNA or, alternatively, genomic DNA, as a template and appropriate oligonucleotide primers according to standard PCR amplification techniques. The nucleic acid so amplified may be cloned into an appropriate vector and characterized by DNA sequence analysis. Furthermore, oligonucleotides corresponding to an irisin nucleotide template sequence may be prepared by standard synthetic techniques, i.e., using an automated irisin synthesizer.

For the irisin DNA template, nucleic acid molecules encoding other irisin members and thus have a nucleotide sequence that differs from the irisin portion of SEQ ID NOs: 4, 6, 8, 10, and 11, or fragments thereof (e.g., nucleic acid encoding SEQ ID NO: 14), are also contemplated. Moreover, nucleic acid molecules encoding irisin proteins from different species, and thus have a nucleotide sequence that differs from the irisin sequences in SEQ ID NOs: 4, 6, 8, 10, and 11 and a nucleotide encoding SEQ ID NO: 14 are also intended to be within the scope of the present invention. For example, rat or monkey irisin cDNA to be used for the irisin DNA template may be identified based on the nucleotide sequence of a human and/or mouse insin.

The present invention further encompasses nucleic acid molecules serving as irisin nucleic acid template that differ from the irisin portion of the nucleotide sequence shown in SEQ ID NO: 4, 6, 8, 10, or 11, or fragments thereof (e.g., a nucleic acid encoding SEQ ID NO: 14) due to degeneracy of the genetic code and thus encode the same irisin or biologically active fragments thereof as that is encoded by the irisin portion in the nucleotide sequence shown in SEQ ID NO: 4, 6, 8, 10, or 11, or fragments thereof (e.g., a nucleic acid encoding SEQ ID NO: 14). In some embodiments, an isolated nucleic acid molecule to be used as an irisin DNA template encompassed by the present invention has a nucleotide sequence encoding a protein having an irisin amino acid portion of SEQ ID NO: 1, 2, 3, 5, 7, or 9, or fragments thereof (e.g., SEQ ID NO: 14), or an irisin protein having an amino acid sequence that is at least about 70%, 75%, 80%, 85%, 90%, 91%, 92%, 93%, 94%, 95%, 96%, 97%, 98%, 99% or more homologous to the irisin amino acid portion of SEQ ID NO: 1, 2, 3, 5, 7, or 9, or fragments thereof (e.g., SEQ ID NO: 14), differs by at least 1, 2, 3, 5 or 10 amino acids but not more than 30, 20, 15 amino acids from the irisin amino acid portion of SEQ ID NO: 1, 2, 3, 5, 7, 9 or 14. In some embodiments, a nucleic acid encoding an irisin polypeptide comprises a nucleic acid sequence encoding a portion of a full-length irisin fragment of interest that is less than 195, 190, 185, 180, 175, 170, 165, 160, 155, 150, 145, 140, 135, 130, 125, 120, 115, 110, 105, 100, 95, 90, 85, 80, 75, or 70 amino acids in length.

It will be appreciated by those skilled in the art that DNA sequence polymorphisms that lead to changes in the amino acid sequences of irisin or biologically active fragments thereof may exist within a population (e.g., a mammalian population, e.g., a human population). Such genetic polymorphism in the FNDC5 gene may exist among individuals within a population due to natural allelic variation. As used herein, the terms “gene” and “recombinant gene” refer to nucleic acid molecules comprising an open reading frame encoding an FNDC5 or irisin protein, such as a mammalian, e.g., human, FNDC5 or a biologically active fragment thereof (e.g., irisin). Such natural allelic variations can typically result in 1-5% variance in the nucleotide sequence of irisin in the FNDC5 gene. Any and all such nucleotide variations and resulting amino acid polymorphisms in irisin or biologically active fragments thereof that are the result of natural allelic variation and that do not alter the functional activity of the irisin polypeptide described herein (e.g., irisin glycosylation mutants and biologically active fragments thereof) are intended to be within the scope encompassed by the present invention. Moreover, nucleic acid molecules encoding irisin or biologically active fragments thereof from other species, and thus that have a nucleotide sequence that differs from the irisin portion in the human or mouse sequences of SEQ ID NO: 4, 6, 8, 10, or 11 are intended to be within the scope encompassed by the irisin DNA template in the present invention. Nucleic acid molecules corresponding to natural allelic variants and homologues of the human or mouse cDNAs of irisin or biologically active fragments thereof encompassed by the irisin DNA template in the present invention may be isolated based on their homology to the human or mouse nucleic acid sequences of irisin or biologically active fragments thereof disclosed herein using the human or mouse cDNA, or a portion thereof, as a hybridization probe according to standard hybridization techniques under stringent hybridization conditions (as described herein).

In addition to naturally-occurring allelic variants of the sequence of irisin or biologically active fragments thereof that may exist in the population, the skilled artisan will further appreciate that changes may be introduced by mutation into the irisin DNA template (e.g., the irisin portion in the nucleotide sequence of SEQ ID NO: 4, 6, 8, 10, or 11, or fragments thereof, e.g., a nucleic acid encoding SEQ ID NO: 14), thereby leading to changes in the amino acid sequence of the encoded irisin or biologically active fragments thereof, without altering the functional ability of the irisin protein described herein (e.g., irisin glycosylation mutants). For example, nucleotide substitutions leading to amino acid substitutions at “non-essential” amino acid residues may be made in the sequence of SEQ ID NO: 4, 6, 8, 10, or 11, or fragments thereof (e.g., a nucleic acid encoding SEQ ID NO: 14). A “non-essential” amino acid residue is a residue that may be altered from the wild-type sequence of irisin or a biologically active fragments thereof (e.g., the sequence of SEQ ID NO: 2 or 14, or fragments thereof) without altering the activity of the irisin protein described herein (e.g., irisin glycosylation mutants and biologically active fragments thereof), whereas an “essential” amino acid residue is required for activity of the irisin protein described herein (e.g., irisin glycosylation mutants and biologically active fragments thereof). Other amino acid residues, however, (e.g., those that are not conserved or only semi-conserved between mouse and human) may not be essential for activity and thus are likely to be amenable to alteration without altering activity of the irisin protein described herein (e.g., irisin glycosylation mutants and biologically active fragments thereof). Furthermore, amino acid residues that are essential for functions of the irisin protein described herein (e.g., irisin glycosylation mutants) related to the methods described herein, but not essential for irisin functions related to thermogenesis, gluconeogenesis, cellular metabolism, and the like, are likely to be amenable to alteration.

“Sequence identity or homology”, as used herein, refers to the sequence similarity between two polypeptide molecules or between two nucleic acid molecules. When a position in both of the two compared sequences is occupied by the same base or amino acid monomer subunit, e.g., if a position in each of two DNA molecules is occupied by adenine, then the molecules are homologous or sequence identical at that position. The percent of homology or sequence identity between two sequences is a function of the number of matching or homologous identical positions shared by the two sequences divided by the number of positions compared×100. For example, if 6 of 10, of the positions in two sequences are the same then the two sequences are 60% homologous or have 60% sequence identity. By way of example, the DNA sequences ATTGCC and TATGGC share 50% homology or sequence identity. Generally, a comparison is made when two sequences are aligned to give maximum homology. Unless otherwise specified “loop out regions”, e.g., those arising from, from deletions or insertions in one of the sequences are counted as mismatches.

The comparison of sequences and determination of percent homology between two sequences may be accomplished using a mathematical algorithm. In one embodiment, the alignment may be performed using the Clustal Method. Multiple alignment parameters include GAP Penalty=10, Gap Length Penalty=10. For DNA alignments, the pairwise alignment parameters may be Htuple=2, Gap penalty=5, Window=4, and Diagonal saved=4. For protein alignments, the pairwise alignment parameters may be Ktuple=1, Gap penalty=3, Window=5, and Diagonals Saved=5.

In one embodiment, the percent identity between two amino acid sequences is determined using the Needleman and Wunsch (J. Mol. Biol. (48):444-453 (1970)) algorithm that has been incorporated into the GAP program in the GCG software package (available online), using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available online), using a NWSgapdna.CMP matrix and a gap weight of 40, 50, 60, 70, or 80 and a length weight of 1, 2, 3, 4, 5, or 6. In some embodiments, the percent identity between two amino acid or nucleotide sequences is determined using the algorithm of E. Meyers and W. Miller (CABIOS, 4:11-17 (1989)) that has been incorporated into the ALIGN program (version 2.0) (available online), using a PAM120 weight residue table, a gap length penalty of 12 and a gap penalty of 4.

An isolated nucleic acid molecule encoding irisin or a biologically active fragment thereof homologous to the protein of SEQ ID NO: 1 or 2, or fragments thereof, may be created by introducing one or more nucleotide substitutions, additions or deletions into the irisin portion of the nucleotide sequence of SEQ ID NO: 4, 6, 8, 10, or 11, or fragments thereof, or a homologous nucleotide sequence such that one or more amino acid substitutions, additions or deletions are introduced into the encoded protein. Mutations may be introduced into SEQ ID NO: 4, 6, 8, 10, or 11, or fragments thereof (e.g., a nucleic acid encoding SEQ ID NO: 14), or the homologous nucleotide sequence by standard techniques, such as site-directed mutagenesis and PCR-mediated mutagenesis. These mutations are in addition to the site-directed mutagenesis described herein using the irisin DNA template and a deglycosylase to generate the targeted motif of the irisin glycosylation mutant polypeptide described herein (e.g., an irisin polypeptide comprising a glycosylated residue at position 36, a glycosylated residue at position 81, and a non-N-linked glycosylated residue at position 45). In one embodiment, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), branched side chains (e.g., threonine, valine, isoleucine) and aromatic side chains (e.g., tyrosine, phenylalanine, tryptophan, histidine). Thus, a predicted nonessential amino acid residue in irisin or biologically active fragments thereof that is encoded by the irisin DNA template may be replaced with another amino acid residue from the same side chain family. Alternatively, in another embodiment, mutations may be introduced randomly along all or part of a coding sequence of the irisin DNA template, such as by saturation mutagenesis, and the resultant mutants may be screened for an activity of irisin or biologically active fragments thereof described herein to identify mutants that retain activity of irisin or biologically active fragments thereof as well as the irisin polypeptides disclosed herein (e.g., irisin glycosylation mutants). Following mutageneses of SEQ ID NO: 4, 6, 8, 10, or 11, or fragments thereof (e.g., a nucleic acid encoding SEQ ID NO: 14), the encoded protein may be expressed recombinantly (as described herein) and the activity of the protein may be determined using, for example, assays described herein.

Levels of the irisin polypeptide described herein (e.g., irisin glycosylation mutants and fragments thereof) may be assessed by any of a wide variety of well-known methods for detecting expression of a transcribed molecule or protein. Non-limiting examples of such methods include immunological methods for detection of proteins, protein purification methods, protein function or activity assays, nucleic acid hybridization methods, nucleic acid reverse transcription methods, and nucleic acid amplification methods.

In some embodiments, levels of the irisin polypeptide described herein (e.g., irisin glycosylation mutants and biologically active fragments thereof) are ascertained by measuring gene transcript (e.g., mRNA), by a measure of the quantity of translated protein, or by a measure of gene product activity. Expression levels may be monitored in a variety of ways, including by detecting mRNA levels, protein levels, or protein activity, any of which may be measured using standard techniques. Detection can involve quantification of the level of gene expression (e.g., genomic DNA, cDNA, mRNA, protein, or enzyme activity), or, alternatively, may be a qualitative assessment of the level of gene expression, in particular in comparison with a control level. The type of level being detected will be clear from the context.

In a particular embodiment, mRNA expression level of an irisin polypeptide described herein (e.g., irisin glycosylation mutants and biologically active fragments thereof) may be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA may be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well-known to those of skill in the art, such as, for example, the single-step RNA isolation process of Chomczynski (1989, U.S. Pat. No. 4,843,155).

The isolated mRNA may be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe may be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or DNA encoding an irisin polypeptide described herein (e.g., irisin glycosylation mutants and biologically active fragments thereof). Other suitable probes for use in diagnostic assays encompassed by the present invention are described herein. Hybridization of an mRNA with the probe indicates that an irisin polypeptide described herein (e.g., irisin glycosylation mutants and biologically active fragments thereof) is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in a gene chip array, e.g., an Affymetrix™ gene chip array. A skilled artisan can readily adapt known mRNA detection methods for use in detecting the level of the mRNA expression levels of an irisin polypeptide described herein (e.g., irisin glycosylation mutants and biologically active fragments thereof).

An alternative method for determining mRNA expression level of the irisin polypeptide described herein (e.g., irisin glycosylation mutants and biologically active fragments thereof) in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self-sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio Technology 6:1197), rolling circle replication (Lizardi et al., U.S. Pat. No. 5,854,033) or any other nucleic acid amplification method, followed by the detection of the amplified molecules using techniques well-known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to the mRNA of an irisin polypeptide described herein (e.g., irisin glycosylation mutants and biologically active fragments thereof).

As an alternative to making determinations based on the absolute expression level of an irisin polypeptide described herein (e.g., irisin glycosylation mutants and biologically active fragments thereof), determinations may be based on the normalized expression level of an irisin polypeptide described herein (e.g., irisin glycosylation mutants and biologically active fragments thereof). Expression levels are normalized by correcting the absolute expression level of an irisin polypeptide described herein (e.g., irisin glycosylation mutants and biologically active fragments thereof) by comparing its expression to the expression of a gene that is not an irisin polypeptide described herein (e.g., irisin glycosylation mutants and biologically active fragments thereof), e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a normal sample, or between samples from different sources.

The level or activity of an irisin polypeptide described herein (e.g., irisin glycosylation mutants and biologically active fragments thereof) may also be detected and/or quantified by detecting or quantifying the expressed polypeptide. An irisin polypeptide described herein (e.g., irisin glycosylation mutants and biologically active fragments thereof) may be detected and quantified by any of a number of means well-known to those of skill in the art. These may include analytic biochemical methods such as electrophoresis, capillary electrophoresis, high performance liquid chromatography (HPLC), thin layer chromatography (TLC), hyper-diffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, and the like. A skilled artisan can readily adapt known protein/antibody detection methods for use in determining whether cells express an irisin polypeptide described herein (e.g., irisin glycosylation mutants and biologically active fragments thereof).

Also provided are soluble, purified and/or isolated forms of an irisin polypeptide described herein (e.g., irisin glycosylation mutants and biologically active fragments thereof). Hereinafter, the irisin polypeptide described herein (e.g., irisin glycosylation mutants and biologically active fragments thereof) will be considered to be encompassed within the term “fragments of irisin.” In one aspect, a polypeptide of an irisin polypeptide described herein (e.g., irisin glycosylation mutants and biologically active fragments thereof) may comprise a full-length amino acid sequence or a full-length amino acid sequence with 1 to about 20 conservative amino acid substitutions of WT-irisin and its biologically active fragments. The amino acid sequence of an irisin polypeptide described herein may also be at least 50, 55, 60, 65, 70, 75, 80, 85, 90, 91, 92, 93, 94, 95, 96, 97, 98, 99, or 99.5% identical to an irisin sequence or biologically active fragments thereof. The amino acid sequence of any irisin polypeptide described herein may also be at least 50, 55, 60, 65, 70, 75, 80, 85, 90, 91, 92, 93, 94, 95, 96, 97, 98, 99, or 99.5% identical to an irisin polypeptide sequence or a fragment thereof. In addition, any irisin polypeptide, or fragment thereof, described herein may induce one or more of the following biological activities: 1) expression or activity of irisin, irisin glycosylation mutants, or biologically active fragments thereof; 2) activity-induced immediate-early gene expression of irisin, irisin glycosylation mutants, and fragments thereof; 3) preventing or reducing degeneration of dopaminergic (DA) neurons, preventing or ameliorating at least one motor deficit and/or preventing or ameliorating at least one symptom of cognitive dysfunction or dementia in a subject in need thereof; 4) reducing the level or amount of α-synuclein in the cells; 5) increasing expression of brain-derived neurotrophic factor (BDNF); 6) treating or preventing neurological diseases or disorders that would benefit from decreased neuronal cell death and/or increased neuronal survival; 7) increasing muscle physiology of a muscle tissue; 8) preventing or treating muscular atrophy or muscular dystrophy or 9) preventing or treating a neurological disease disclosed herein. In another aspect, the present invention contemplates a composition comprising an isolated irisin polypeptide described herein (e.g., irisin glycosylation mutants and biologically active fragments thereof) and less than about 25%, or alternatively 15%, or alternatively 5%, contaminating biological macromolecules or polypeptides.

The present invention further provides compositions related to producing, detecting, or characterizing an irisin polypeptide described herein (e.g., irisin glycosylation mutants and biologically active fragments thereof), such as nucleic acids, vectors, host cells, and the like. Such compositions may serve as compounds that modulate an expression and/or activity of an irisin polypeptide described herein (e.g., irisin glycosylation mutants and biologically active fragments thereof), such as antisense nucleic acids.

In some embodiments, an irisin polypeptide described herein (e.g., irisin glycosylation mutants and biologically active fragments thereof) comprises an amino acid modification, post-translational modification, and/or a heterologous an amino acid sequence, that stabilizes the polypeptide and/or increases its half-life. In certain embodiments, an irisin polypeptide described herein (e.g., irisin glycosylation mutants and biologically active fragments thereof) encompassed by the present invention may be a fusion protein containing a domain that increases its solubility and bioavailability and/or facilitates its purification, identification, detection, and/or structural characterization. Exemplary domains, include, for example, glutathione S-transferase (GST), protein A, protein G, calmodulin-binding peptide, thioredoxin, maltose binding protein, HA, myc, poly arginine, poly His, poly His-Asp or FLAG fusion proteins and tags. Additional exemplary domains include domains that alter protein localization in vivo, such as signal peptides, type III secretion system-targeting peptides, transcytosis domains, nuclear localization signals, etc. In various embodiments, an irisin polypeptide described herein (e.g., irisin glycosylation mutants and biologically active fragments thereof) encompassed by the present invention may comprise one or more heterologous fusions. Polypeptides may contain multiple copies of the same fusion domain or may contain fusions to two or more different domains. The fusions may occur at the N-terminus of the polypeptide, at the C-terminus of the polypeptide, or at both the N- and C-terminus of the polypeptide. It is also within the scope encompassed by the present invention to include linker sequences between a polypeptide encompassed by the present invention and the fusion domain in order to facilitate construction of the fusion protein or to optimize protein expression or structural constraints of the fusion protein. In one embodiment, the linker is a linker described herein, e.g., a linker of at least 8, 9, 10, 15, 20 amino acids. The linker may be, e.g., an unstructured recombinant polymer (URP), e.g., a URP that is 9, 10, 11, 12, 13, 14, 15, 20 amino acids in length, i.e., the linker has limited or lacks secondary structure, e.g., Chou-Fasman algorithm. In some embodiments, the polypeptide may be constructed so as to contain protease cleavage sites between the fusion polypeptide and polypeptide encompassed by the present invention in order to remove the tag after protein expression or thereafter. Examples of suitable endoproteases, include, for example, Factor Xa and TEV proteases.

In some embodiments, an irisin polypeptide described herein (e.g., irisin glycosylation mutants and biologically active fragments thereof) may be fused to an antibody (e.g., IgG 1, IgG2, IgG3, IgG4) fragment (e.g., Fc polypeptides). Techniques for preparing these fusion proteins are known, and are described, for example, in WO 99/31241 and in Cosman et. al. (2001) Immunity 14:123-133. Fusion to an Fc polypeptide offers the additional advantage of facilitating purification by affinity chromatography over Protein A or Protein G columns.

In still another embodiment, an irisin polypeptide described herein (e.g., irisin glycosylation mutants and biologically active fragments thereof) may be labeled with a fluorescent label to facilitate their detection, purification, or structural characterization. In an exemplary embodiment, an irisin polypeptide disclosed herein (e.g., irisin glycosylation mutants and biologically active fragments thereof) encompassed by the present invention may be fused to a heterologous polypeptide sequence that produces a detectable fluorescent signal, including, for example, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED).

Another aspect encompassed by the present invention pertains to the use of an irisin polypeptides described herein (e.g., irisin glycosylation mutants and biologically active fragments thereof), as well as peptide fragments suitable for use as immunogens, to raise anti-irisin antibodies. An “isolated” or “purified” protein or biologically active portion thereof is substantially free of cellular material when produced by recombinant DNA techniques, or chemical precursors or other chemicals when chemically synthesized. The language “substantially free of cellular material” includes preparations of an irisin polypeptide described herein (e.g., irisin glycosylation mutants and biologically active fragments thereof) in which the protein is separated from cellular components of the cells in which it is naturally or recombinantly produced. In one embodiment, the language “substantially free of cellular material” includes preparations of an irisin polypeptide described herein (e.g., irisin glycosylation mutants and biologically active fragments thereof) having less than about 30% (by dry weight) of non-irisin protein (also referred to herein as a “contaminating protein”), less than about 20% of non-irisin protein, less than about 10% of non-irisin protein, or less than about 5% non-irisin protein. When an irisin polypeptide described herein (e.g., irisin glycosylation mutants and biologically active fragments thereof) is recombinantly produced, it is also substantially free of culture medium, i.e., culture medium represents less than about 20%, less than about 10%, or less than about 5% of the volume of the protein preparation. The language “substantially free of chemical precursors or other chemicals” includes preparations of an irisin polypeptide described herein (e.g., irisin glycosylation mutants and biologically active fragments thereof) in which the protein is separated from chemical precursors or other chemicals that are involved in the synthesis of the protein. In one embodiment, the language “substantially free of chemical precursors or other chemicals” includes preparations of protein of an irisin polypeptide described herein (e.g., irisin glycosylation mutants and biologically active fragments thereof) having less than about 30% (by dry weight) of chemical precursors of non-irisin chemicals, less than about 20% chemical precursors of non-irisin chemicals, less than about 10% chemical precursors of non-irisin chemicals, or less than about 5% chemical precursors of non-irisin chemicals. In some embodiments, isolated proteins or biologically active portions thereof lack contaminating proteins from the same animal from which the irisin polypeptide described herein (e.g., irisin glycosylation mutants and biologically active fragments thereof) is derived. Typically, such proteins are produced by recombinant expression of, for example, an irisin polypeptide described herein (e.g., irisin glycosylation mutants and biologically active fragments thereof) in a non-human cell.

Proteins of an irisin polypeptide described herein (e.g., irisin glycosylation mutants or biologically active fragments thereof) may be produced by recombinant DNA techniques. For example, a nucleic acid molecule encoding the protein is cloned into an expression vector (as described above), the expression vector is introduced into a host cell (as described above) and an irisin polypeptide described herein (e.g., irisin glycosylation mutants or biologically active fragments thereof) is expressed in the host cell. The protein of an irisin polypeptide described herein (e.g., irisin glycosylation mutants or biologically active fragments thereof) can then be isolated from the cells by an appropriate purification scheme using standard protein purification techniques. Alternative to recombinant expression, an irisin polypeptide described herein (e.g., irisin glycosylation mutants or biologically active fragments thereof) may be synthesized chemically using standard peptide synthesis techniques. Moreover, an irisin polypeptide described herein (e.g., irisin glycosylation mutants or biologically active fragments thereof) may be isolated from body fluids like plasma or cells, for example using an anti-irisin antibody (described further below).

Also provided are chimeric or fusion proteins of an irisin polypeptide described herein (e.g., irisin glycosylation mutants or biologically active fragments thereof), as described above. As used herein, a “chimeric protein” or “fusion protein” comprises an irisin polypeptide described herein (e.g., irisin glycosylation mutants or biologically active fragments thereof) operatively linked to a non-irisin polypeptide, for example, an Fc domain, an IgG1 Fc domain, an IgG2 Fc domain, an IgG3 Fc domain, and IgG4 Fc domain, a dimerization domain, an oligomerization domain, an agent that promotes plasma solubility, albumin, a signal peptide, a peptide tag, a 6-His tag, a thioredoxin tag, a hemaglutinin tag, a GST tag, or an OmpA signal sequence tag. An “irisin polypeptide” refers to an amino acid sequence corresponding to an irisin polypeptide described herein (e.g., irisin glycosylation mutants or biologically active fragments thereof), whereas a “non-irisin polypeptide” refers to a polypeptide having an amino acid sequence corresponding to a protein that is not substantially homologous to an irisin polypeptide described herein (e.g., irisin glycosylation mutants or biologically active fragments thereof), respectively, e.g., a protein that is different from an irisin polypeptide described herein (e.g., irisin glycosylation mutants or biologically active fragments thereof) and that is derived from the same or a different organism. Within the fusion protein, the term “operatively linked” is intended to indicate that the irisin polypeptide and the non-irisin polypeptide are fused in-frame to each other. The non-irisin polypeptide may be fused to the N-terminus or C-terminus of the irisin polypeptide, respectively. For example, in one embodiment the fusion protein is an irisin-GST and/or irisin-Fc fusion protein in which the irisin sequences, respectively, are fused to the N-terminus of the GST or Fc sequences. Such fusion proteins can facilitate the purification, expression, and/or bioavailability of a recombinant protein of an irisin polypeptide described herein (e.g., irisin glycosylation mutants or biologically active fragments thereof). In some embodiments, the fusion protein is an irisin polypeptide described herein (e.g., insin glycosylation mutants or biologically active fragments thereof) containing a heterologous signal sequence at its C-terminus. In certain host cells (e.g., mammalian host cells), expression and/or secretion of an irisin polypeptide described herein (e.g., irisin glycosylation mutants or biologically active fragments thereof) may be increased through use of a heterologous signal sequence.

In some embodiments, a chimeric or fusion protein encompassed by the present invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In some embodiments, the fusion gene may be synthesized by conventional techniques including automated DNA synthesizers. Alternatively, PCR amplification of gene fragments may be carried out using anchor primers that give rise to complementary overhangs between two consecutive gene fragments that can subsequently be annealed and reamplified to generate a chimeric gene sequence (see, for example, Current Protocols in Molecular Biology, eds. Ausubel et al., John Wiley & Sons: 1992). Moreover, many expression vectors are commercially available that already encode a fusion moiety (e.g., a GST polypeptide). A nucleic acid encoding the irisin polypeptide described herein (e.g., irisin glycosylation mutants or biologically active fragments thereof) may be cloned into such an expression vector such that the fusion moiety is linked in-frame to an irisin polypeptide described herein (e.g., irisin glycosylation mutants or biologically active fragments thereof).

Also provided are homologues of an irisin polypeptide described herein (e.g., irisin glycosylation mutants or biologically active fragments thereof) that function as either an agonist (mimetic) or an antagonist of an irisin polypeptide described herein (e.g., irisin glycosylation mutants or biologically active fragments thereof). In an embodiment, the agonists and antagonists stimulate or inhibit, respectively, a subset of the biological activities of the naturally occurring form of an irisin polypeptide described herein (e.g., irisin glycosylation mutants or biologically active fragments thereof). Thus, specific biological effects may be elicited by treatment with a homologue of limited function. In one embodiment, treatment of a subject with a homologue having a subset of the biological activities of the naturally occurring form of the protein has fewer side effects in a subject relative to treatment with the naturally occurring form of an irisin polypeptide described herein (e.g., irisin glycosylation mutants or biologically active fragments thereof).

Homologues of an irisin polypeptide described herein (e.g., irisin glycosylation mutants or biologically active fragments thereof) may be generated by mutagenesis, e.g., discrete point mutation or truncation of the protein. As used herein, the term “homologue” refers to a variant form of an irisin polypeptide described herein (e.g., irisin glycosylation mutants or biologically active fragments thereof) that acts as an agonist or antagonist of the activity of an irisin polypeptide described herein (e.g., irisin glycosylation mutants or biologically active fragments thereof). An agonist can retain substantially the same, or a subset, of the biological activities of the protein. An antagonist of the protein can inhibit one or more of the activities of the naturally occurring form of the protein, by, for example, competitively binding to a downstream or upstream member of the irisin cascade, which includes an irisin polypeptide described herein (e.g., irisin glycosylation mutants or biologically active fragments thereof). Thus, the irisin polypeptide described herein (e.g., irisin glycosylation mutants or biologically active fragments thereof), and homologues thereof, encompassed by the present invention may be, for example, either positive or negative regulators of neuronal cell function.

In an alternative embodiment, homologues of an irisin polypeptide described herein (e.g., irisin glycosylation mutants or biologically active fragments thereof) may be identified by screening combinatorial libraries of mutants, e.g., truncation mutants, of irisin or biologically active fragments thereof for agonist or antagonist activity. In one embodiment, a variegated library of variants of an irisin polypeptide described herein (e.g., irisin glycosylation mutants or biologically active fragments thereof) is generated by combinatorial mutagenesis at the nucleic acid level and is encoded by a variegated gene library. A variegated library of variants of an irisin polypeptide described herein (e.g., irisin glycosylation mutants or biologically active fragments thereof) may be produced by, for example, enzymatically ligating a mixture of synthetic oligonucleotides into gene sequences such that a degenerate set of potential sequences for the irisin polypeptide described herein (e.g., irisin glycosylation mutants or biologically active fragments thereof) is expressible as individual polypeptides, or alternatively, as a set of larger fusion proteins (e.g., for phage display) containing the set of sequences therein. There are a variety of methods that may be used to produce libraries of potential homologues of the irisin polypeptide described herein (e.g., irisin glycosylation mutants or biologically active fragments thereof) from a degenerate oligonucleotide sequence. Chemical synthesis of a degenerate gene sequence may be performed in an automatic DNA synthesizer, and the synthetic gene then ligated into an appropriate expression vector. Use of a degenerate set of genes allows for the provision, in one mixture, of all of the sequences encoding the desired set of potential sequences of the irisin polypeptide disclosed herein (e.g., irisin glycosylation mutants or biologically active fragments thereof). Methods for synthesizing degenerate oligonucleotides are known in the art (see, e.g., Narang, S. A. (1983) Tetrahedron 39:3; Itakura et al., (1984) Annu. Rev. Biochem. 53:323; Itakura et al., (1984) Science 198:1056; Ike et al., (1983) Nucleic Acid Res. 11:477.

In addition, libraries of fragments of irisin may be used to generate a variegated population of irisin fragments for screening and subsequent selection of homologues of the irisin polypeptide described herein (e.g., irisin glycosylation mutants or biologically active fragments thereof). In one embodiment, a library of coding sequence fragments may be generated by treating a double stranded PCR fragment of a coding sequence of the irisin polypeptide described herein (e.g., irisin glycosylation mutants or biologically active fragments thereof) with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with Si nuclease, and ligating the resulting fragment library into an expression vector. By this method, an expression library may be derived that encodes N-terminal, C-terminal and internal fragments of various sizes of the irisin polypeptide disclosed herein (e.g., irisin glycosylation mutants or biologically active fragments thereof).

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of homologues of the irisin polypeptide described herein (e.g., irisin glycosylation mutants or biologically active fragments thereof). The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, may be used in combination with the screening assays to identify homologues of the irisin polypeptide disclosed herein (Arkin and Youvan (1992) Proc. Natl. Acad. Sci. U.S.A. 89:7811-7815; Delagrave et al., (1993) Protein Engineering 6:327-331).

In addition, useful host cells and vectors are described supra for expressing desired nucleic acids and proteins for use according to the methods described herein.

### Pharmaceutical Compositions

In some embodiments, provided herein is an agent comprising irisin glycosylation mutants (e.g., an irisin polypeptide, comprising a glycosylated residue at position 36, a glycosylated residue at position 81, and a non-N-linked glycosylated residue at position 45) or biologically active fragments thereof, either alone or in combination with other agents useful for treating or preventing an α-synucleinopathy or a cancer characterized by or caused by increased levels of α-synuclein; for increasing expression of brain-derived neurotrophic factor (BDNF), and thus treating neurological diseases or disorders; or for increasing the muscle physiology, and thus treating muscular dystrophy and/or muscular atrophy.

In some embodiments, the agent comprises an irisin glycosylation mutant (e.g., an irisin polypeptide comprising a glycosylated residue at position 36, a glycosylated residue at position 81, and a non-N-linked glycosylated residue at position 45. In such irisin polypeptide, the irisin polypeptide may comprise a Q residue at position 45). The irisin polypeptide may also comprise at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity to the amino acid sequence of SEQ ID NO: 1. In some embodiments, the irisin polypeptide does not comprise a FNDC5 signal polypeptide. The FNDC5 signal polypeptide may comprise the amino acid sequence of SEQ ID NO: 12 (MEWSWVFLFFLSVTTGVHS). In some embodiments, the irisin polypeptide further comprises one or more polyhistidine (His)-Tag(s), optionally wherein the irisin polypeptide comprises two or more, five or more, or ten or more His-Tags. In some embodiments, the irisin polypeptide further comprises a human rhinovirus 3C protease (HRV-3C) protease tag. In some embodiment, the irisin polypeptide further comprises a GFP sequence. In some embodiments, the irisin polypeptide further comprises a Strep-II tag, optionally wherein the irisin polypeptide comprises at least two, at least three, at least four, at least five, or at least six Strep-II tags. In some embodiments, the irisin polypeptide binds to an irisin receptor with at least 1-fold, at least 1.5-fold, at least 2-fold, at least 3-fold, at least 4-fold, at least 5-fold, at least 10-fold, at least 15-fold, or at least 20-fold more binding affinity to an irisin receptor compared to wild-type irisin. In some embodiments, the irisin polypeptide comprises the amino acid sequence set forth in SEQ ID NO: 15.

In some embodiments, the irisin polypeptide binds to an irisin receptor with at least 1-fold, at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold increased binding affinity to αVP5 and/or integrin compared to wild-type irisin. In some embodiments, the irisin polypeptide comprises at least 1-fold, at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold increased stability compared to wild-type irisin. In some embodiments, the irisin polypeptide comprises at least 1-fold, at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold increased half-life compared to wild-type irisin. In some embodiments, the irisin polypeptide is fused to one or more heterologous polypeptides at its N-terminus and/or C-terminus. In some embodiments, the irisin polypeptide comprises an amino acid modification, post-translational modification, and/or a heterologous an amino acid sequence that stabilizes the irisin polypeptide and/or increases its half-life.

Such agents may be formulated as pharmaceutical compositions. They may be administered in a therapeutically effective amount to a subject using pharmaceutical compositions suitable for such administration. Such compositions typically comprise the agent (e.g., nucleic acid molecule or protein) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary active compounds may also be incorporated into the compositions.

The term “effective amount” of an agent that induces expression and/or activity of the irisin glycosylation mutant or a biologically active fragment thereof is that amount necessary or sufficient to increase expression and/or activity of the irisin glycosylation mutant or a biologically active fragment thereof in the appropriate context, such as cells in vitro or ex vivo, a subject, a population of subjects, and the like. An effective amount can vary depending on such factors as the type of therapeutic agent(s) employed, the size of the subject, or the severity of the disorder.

The term “therapeutically effective amount” as used herein means that amount of an agent that increases the expression or activity of the irisin glycosylation mutant or a biologically active fragment thereof, or composition comprising an agent that increases such expression or activity, which is effective for producing some desired therapeutic effect, e.g., expression or activity of the irisin glycosylation mutants or biologically active fragments thereof in subjects, at a reasonable benefit/risk ratio.

A pharmaceutical composition used in therapeutic methods encompassed by the present invention may be formulated to be compatible with its intended route of administration. Administration may be systemic or local. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH may be adjusted with acids or bases, such as hydrochloric acid or sodium hydroxide. The parenteral preparation may be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (for example, glycerol, propylene glycol, and liquid polyetheylene glycol, and the like), and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, polyalcohols such as mannitol, sorbitol, and sodium chloride are included in the composition. Prolonged absorption of the injectable compositions may be brought about by including in the composition an agent which delays absorption, for example, aluminum monostearate and gelatin.

Sterile injectable solutions may be prepared by incorporating the irisin glycosylation mutant or a biologically active fragment thereof, or an enhancer of such a polypeptide's expression or activity, in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, some methods of preparation are vacuum drying and freeze-drying which yields a powder of the active ingredient plus any additional desired ingredient from a previously sterile-filtered solution thereof.

Oral compositions generally include an inert diluent or an edible carrier. They may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents, and/or adjuvant materials may be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration may also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, the active compounds are formulated into ointments, salves, gels, or creams as generally known in the art.

The agents, comprising an irisin glycosylation mutants described herein or biologically active fragments thereof, or nucleic acids encoding the irisin glycosylation mutants described herein or biologically active fragments thereof, may also be prepared in the form of suppositories (e.g., with conventional suppository bases such as cocoa butter and other glycerides) or retention enemas for rectal delivery.

In one embodiment, the agents that modulate irisin expression or activity are prepared with carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art. The materials may also be obtained commercially from Alza Corporation and Nova Pharmaceuticals, Inc. Liposomal suspensions (including liposomes targeted to infected cells with monoclonal antibodies to viral antigens) may also be used as pharmaceutically acceptable carriers. These may be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. In some embodiments, the specification for the dosage unit forms encompassed by the present invention are dictated by and directly dependent on the unique characteristics of the agents described herein and the particular therapeutic effect to be achieved, and the limitations inherent in the art of compounding such agents for the treatment of subjects.

Toxicity and therapeutic efficacy of such agents described herein may be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and may be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies may be used in formulating a range of dosage for use in humans. In certain embodiments, the dosage of such agents disclosed herein lies within a range of circulating concentrations that include the ED50 with little or no toxicity. The dosage may vary within this range depending upon the dosage form employed and the route of administration utilized. For any agent used in therapeutic methods encompassed by the present invention, the therapeutically effective dose may be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, from about 0.01 to 25 mg/kg body weight, from about 0.1 to 20 mg/kg body weight, or from about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or a series of treatments.

In a one example, a subject is treated with a polypeptide in the range of between about 0.1 to 20 mg/kg body weight, one time per week for between about 1 to 10 weeks, between 2 to 8 weeks, between about 3 to 7 weeks, or for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents that modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depend upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect that the practitioner desires the small molecule to have upon a nucleic acid or polypeptide encompassed by the present invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of the irisin glycosylation mutant or a biologically active fragment thereof, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated, e.g., the intended use of the agonist or antagonize.

Further, agents described herein may be conjugated to additional therapeutic moieties of interest, such as a growth factor, intracellular targeting domain, and the like, that are well-known in the art. Conjugates encompassed by the present invention may be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Nucleic acid molecules encompassed by the methods of the present invention may be inserted into vectors and used as gene therapy vectors. Gene therapy vectors may be delivered to a subject by, for example, intravenous injection, local administration (see U.S. Pat. No. 5,328,470) or by stereotactic injection (see, e.g., Chen et al., (1994) Proc. Natl. Acad. Sci. U.S.A. 91:3054-3057). A pharmaceutical preparation of the gene therapy vector may include the gene therapy vector in an acceptable diluent, or may comprise a slow release matrix in which the gene delivery vehicle is imbedded. Alternatively, where the complete gene delivery vector may be produced intact from recombinant cells, e.g., retroviral vectors, the pharmaceutical preparation may include one or more cells that produce the gene delivery system.

As defined herein, a therapeutically effective amount of vector particle (i.e., an effective dosage) ranges from at least 1×10² GC/kg particles, at least 1×10³ GC/kg particles, at least 1×10⁴ GC/kg particles, at least 1×10⁵ GC/kg particles, at least 1×10⁶ GC/kg particles, at least 1×10⁷ GC/kg particles, at least 1×10⁸ GC/kg particles, at least 1×10⁹ GC/kg particles, at least 1×10¹⁰ GC/kg particles, at least 1×10¹¹ GC/kg particles, at least 1×10¹² GC/kg particles, at least 1×10¹³ GC/kg particles, at least 1×10¹⁴ GC/kg particles, at least 1×10¹⁵ GC/kg particles, at least 1×10¹⁶ GC/kg particles, at least 1×10¹⁷ GC/kg particles, at least 1×10¹⁸ GC/kg particles, at least 1×10¹⁹ GC/kg particles, at least 1×10²⁰ GC/kg particles, at least 1×10²¹ GC/kg particles, at least 1×10²² GC/kg particles, at least 1×10²³ GC/kg particles, at least 1×10²⁴ GC/kg particles, at least 1×10²⁵ GC/kg particles, at least 1 xi10²⁶ GC/kg particles, at least 1×10²⁷ GC/kg particles, at least 1×10²⁸ GC/kg particles, at least 1×10²⁶ GC/kg particles, at least 1×10²⁷ GC/kg particles, at least 1×10²⁸ GC/kg particles, at least 1×10²⁹ GC/kg particles, or at least 1×10³⁰ GC/kg particles. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a nucleic acid vector can include a single treatment or a series of treatments.

Any means for the introduction of a polynucleotide into mammals, human or non-human, or cells thereof may be adapted to the practice of the present for the delivery of various constructs encompassed by the present invention into the intended recipient. In one embodiment encompassed by the present invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. In certain embodiments, the colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner et al., (1995) Ann. NY Acad. Sci., 126-139). Formulation of DNA, e.g. with various lipid or liposome materials, may then be effected using known methods and materials and delivered to the recipient mammal. See, e.g., Canonico et al., (1994)Am. J. Respir. Cell. Mol. Biol., 10:24-29; Tsan et al., (1994)Am. J. Physiol., 268; Alton et al., (1993) Nat. Genet., 5:135-142; and U.S. Pat. No. 5,679,647 by Carson et al.

The targeting of liposomes may be classified based on anatomical and mechanistic factors. Anatomical classification is based on the level of selectivity, for example, organ-specific, cell-specific, and organelle-specific. Mechanistic targeting may be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups may be incorporated into the lipid bilayer of the liposome in order to maintain the targeting ligand in stable association with the liposomal bilayer. Various linking groups may be used for joining the lipid chains to the targeting ligand. Naked DNA or DNA associated with a delivery vehicle, e.g., liposomes, may be administered to several sites in a subject (see below).

Nucleic acids may be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno-associated virus), HIV (human immunodeficiency virus), BIV (bovine immunodeficiency virus), and MLV (murine leukemia virus). Nucleic acids may be administered in any desired format that provides sufficiently efficient delivery levels, including in virus particles, in liposomes, in nanoparticles, and complexed to polymers.

Nucleic acids encoding a protein or nucleic acids of interest may be in a plasmid or viral vector, or other vector as is known in the art. Such vectors are well-known and any may be selected for a particular application. In one embodiment encompassed by the present invention, the gene delivery vehicle comprises a promoter and a demethylase coding sequence. In some embodiments, promoters are tissue-specific promoters and promoters, which are activated by cellular proliferation, such as the thymidine kinase and thymidylate synthase promoters. Other promoters include promoters, which are activated by infection with a virus, such as the α- and β-interferon promoters, and promoters, which are activated by a hormone, such as estrogen. Other promoters that may be used include the Moloney virus LTR, the CMV promoter, and the mouse albumin promoter. A promoter may be constitutive or inducible.

In some embodiments, naked polynucleotide molecules are used as gene delivery vehicles, as described in WO 90/11092 and U.S. Pat. No. 5,580,859. Such gene delivery vehicles may be either growth factor DNA or RNA and, in certain embodiments, are linked to killed adenovirus. Curiel et al., Hum. Gene. Ther. 3:147-154, 1992. Other vehicles that can optionally be used include DNA-ligand (Wu et al., J. Biol. Chem. 264:16985-16987, 1989), lipid-DNA combinations (Felgner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences may be selected from viruses such as astrovirus, coronavirus, orthomyxovirus, papovavirus, paramyxovirus, parvovirus, picornavirus, poxvirus, retrovirus, togavirus or adenovirus. In an embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles may be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Other viral vector systems that may be used to deliver a polynucleotide encompassed by the present invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth, Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al., (1988) Gene, 68:1-10), and several RNA viruses. In Viruses include, but are not limited to, an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al., (1990) J. Virol., 64:642-650).

In other embodiments, target DNA in the genome may be manipulated using well-known methods in the art. For example, the target DNA in the genome may be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis. In some embodiments, genome editing may be used to modulate the copy number or genetic sequence of a protein of interest, such as constitutive or induced knockout or mutation of a protein of interest, such as of the irisin polypeptide disclosed herein (e.g., irisin glycosylation mutants or biologically active fragments thereof, e.g., the irisin glycosylation mutant polypeptides comprises a glycosylated residue at position 36, a glycosylated residue at position 81, and a non-N-linked glycosylated residue at position 45) encompassed by the present invention. For example, the CRISPR-Cas system may be used for precise editing of genomic nucleic acids (e.g., for creating non-functional or null mutations). In such embodiments, the CRISPR guide RNA and/or the Cas enzyme may be expressed. For example, a vector containing only the guide RNA may be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al., (2009) Cell 139:945-956; Karginov and Hannon (2010)Mol. Cell 37:7; U.S. Pat. Publ. 2014/0087426 and 2012/0178169; Boch et al., (2011) Nat. Biotech. 29:135-136; Boch et al., (2009) Science 326:1509-1512; Moscou and Bogdanove (2009) Science 326:1501; Weber et al., (2011) PLoS One 6:e19722; Li et al., (2011) Nucl. Acids Res. 39:6315-6325; Zhang et al., (2011) Nat. Biotech. 29:149-153; Miller et al., (2011) Nat. Biotech. 29:143-148; Lin et al., (2014) Nucl. Acids Res. 42:e47). Such genetic strategies can use constitutive expression systems or inducible expression systems according to well-known methods in the art.

In other embodiments, the irisin glycosylation mutants or biologically active fragments thereof, as described herein, may be administered to subjects. In some embodiments, fusion proteins may be constructed that have enhanced biological properties (e.g., Fc fusion proteins discussed above) and administered. In addition, the irisin glycosylation mutants described herein or biologically active fragments thereof may be modified according to well-known pharmacological methods in the art (e.g., pegylation, glycosylation, oligomerization, etc.) in order to further enhance desirable biological activities, such as increased bioavailability and decreased proteolytic degradation.

### Methods of Treatment

The present invention provides for both prophylactic and therapeutic methods of preventing and/or treating a condition that would benefit from preventing or reducing degeneration of neurons, preventing or ameliorating at least one motor deficit and/or preventing or ameliorating at least one symptom of cognitive dysfunction or dementia, such as in a subject afflicted with an α-synucleinopathy; or lowering the levels of α-synuclein, such as in a subject afflicted with an α-synucleinopathy or a cancer characterized by or caused by increased levels of α-synuclein, in a subject (e.g., a human) who is at risk of (or susceptible to) the condition, by administering to the subject an agent comprising i) an irisin polypeptide disclosed herein (e.g., a novel and non-naturally occurring irisin polypeptide such as an irisin glycosylation mutant, comprising a glycosylated residue at position 36, a glycosylated residue at position 81, and a non-N-linked glycosylated residue at position 45) or biologically active fragments thereof, or ii) the nucleic acid encoding an irisin polypeptide disclosed herein or biologically active fragments thereof, such that the condition is prevented or treated. In some embodiments, which includes both prophylactic and therapeutic methods, the agent is administered in a pharmaceutically acceptable formulation.

Accordingly, one aspect of the present invention provides a method of reducing or lowering the levels of α-synuclein in cells (e.g., neuronal cells, muscular cells, or cancer cells), the method comprising contacting the cells with an agent comprising i) an irisin glycosylation mutant disclosed herein or biologically active fragments thereof, or ii) a nucleic acid encoding the irisin glycosylation mutant disclosed herein or biologically active fragments thereof, thereby lowering the levels of α-synuclein in the cells. This method may be performed in vivo, ex vivo, or in vitro. The cells or tissues may be in need of treatment if they are affected by a condition disclosed herein, such as an α-synucleinopathy or a cancer characterized by or caused by increased levels of α-synuclein.

Another aspect of the present invention discloses methods of treating or preventing Parkinson's disease, Lewy body dementia, Alzheimer's disease, multiple system atrophy (MSA), a neuroaxonal dystrophy, or Lou Gehrig's disease (ALS) in a subject, comprising administering to the subject the agent comprising: i) an irisin glycosylation mutant disclosed herein or biologically active fragments thereof, or ii) a nucleic acid encoding the irisin glycosylation mutantdisclosed herein or the biologically active fragment thereof, in a therapeutically effective amount to treat or prevent diseases or disorders disclosed herein. In some other embodiments, the invention discloses methods of increasing expression of brain-derived neurotrophic factor (BDNF) by a cell, comprising contacting the cell with an agent (e.g., in vivo, ex vivo, or in vitro), wherein the agent comprises: i) an irisin glycosylation mutant disclosed herein or biologically active fragments thereof, or ii) a nucleic acid encoding an irisin glycosylation mutant disclosed herein or biologically active fragments thereof. The cell may be neurons (e.g., hippocampal neurons, cerebellar neurons, sciatic nerve neurons, dopaminergic neurons, and substantia nigra neurons).

In another aspect, the invention discloses methods for treating or preventing a neurological disease or disorder that would benefit from decreased neuronal cell death and/or increased neuronal survival in a subject, comprising the step of administering to the subject an agent comprising: i) an irisin glycosylation mutant disclosed herein or biologically active fragment a thereof, or ii) a nucleic acid encoding the irisin glycosylation mutant disclosed herein or biologically active fragment a thereof, that increases BDNF expression or activity in central or peripheral nervous system of the subject, such that the neurological disease or disorder is treated or prevented.

Also disclosed herein are methods of increasing muscle physiology of a muscle tissue (e.g., skeletal muscle tissue, cardiac muscle tissue, and/or smooth muscle tissue), the method comprising contacting the muscle tissue with an agent (e.g., in vivo, ex vivo, or in vitro), and the agent comprises: i) an irisin glycosylation mutant disclosed herein or biologically active fragments thereof, or ii) an nucleic acid encoding an irisin glycosylation mutant disclosed herein or biologically active fragments thereof. The muscle tissue may be affected by a muscular dystrophy (MD; e.g., Becker, congenital, Duchenne, distal, Emery-Dreifuss, facioscapulohumeral, limb-girdle, myotonic, and oculopharyngeal muscular dystrophy) or comprise a muscle cell having a mutation in a gene associated with a muscular dystrophy, optionally wherein the gene is dystrophin. The muscle tissue may be affected by muscular atrophy, optionally resulting from disuse, trauma or a disease other than muscular dystrophy (e.g., Charcot-Marie-Tooth disease or spinal muscular atrophy).

In some other embodiments, the invention discloses methods for preventing or treating muscular atrophy or muscular dystrophyin a subject in need thereof, comprising the step of administering to the subject an agent comprising: i) an irisin glycosylation mutant disclosed herein or biologically active fragments thereof, or ii) a nucleic acid encoding an irisin glycosylation mutant disclosed herein or biologically active fragments thereof.

The compositions disclosed herein may be administered over any period of time effective to achieve the desired therapeutic response for a particular patient, composition, and mode of administration, without being toxic to the patient. The period of time may be at least 1 day, at least 10 days, at least 20 days, at least 30, days, at least 60 days, at least three months, at least six months, at least a year, at least three years, at least five years, or at least ten years. The dose may be administered when needed, sporadically, or at regular intervals. For example, the dose may be administered monthly, weekly, biweekly, triweekly, once a day, or twice a day. In certain embodiments, a dose of the composition is administered at regular intervals over a period of time. In some embodiments, a dose of the composition is administered at least once a week. In some embodiments, a dose of the composition is administered at least twice a week. In certain embodiments, a dose of the composition is administered at least three times a week. In some embodiments, a dose of the composition is administered at least once a day. In some embodiments, a dose of the composition is administered at least twice a day. In some embodiments, doses of the composition are administered for at least 1 week, for at least 2 weeks, for at least 3 weeks, for at least 4 weeks, for at least 1 month, for at least 2 months, for at least 3 months, for at least 4 months, for at least 5 months, for at least 6 months, for at least 1 year, for at least two years, at least three years, or at least five years.

In some embodiments, the method further comprises contacting the cell and/or tissue, and/or administering to the subject with an additional agent that increases the expression or activity of the irisin glycosylation mutant or a biologically active fragment thereof.

With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect encompassed by the present invention provides methods for tailoring a subject's prophylactic or therapeutic treatment with agents described according to that individual's drug response genotype. Pharmacogenomics allows a clinician or physician to target prophylactic or therapeutic treatments to patients who will most benefit from the treatment and to avoid treatment of patients who will experience toxic drug-related side effects.

### A. Prophylactic Methods

In one aspect, the present invention provides a method for preventing a condition in a subject that would benefit from lowering or reducing the levels of α-synuclein. As a non-limiting, representative example, a method of treating or preventing a α-synucleinopathy (e.g., Parkinsons' disease, Lewy body dementia, Alzheimer's disease, multiple system atrophy (MSA), or a neuroaxonal dystrophy) is provided involving administering to the subject with an irisin glycosylation mutant (e.g., an irisin polypeptide, comprising a glycosylated residue at position 36, a glycosylated residue at position 81, and a non-N-linked glycosylated residue at position 45) or biologically active fragments thereof or a nucleic acid that encodes the irisin glycosylation mutant or biologically active fragments thereof, and/or an enhancer of expression and/or activity of the irisin glycosylation mutant or biologically active fragment thereof or of nucleic acid encoding the irisin polypeptide or biologically active fragment thereof. Subjects at risk for a disease or disorder disclosed herein may be identified by, for example, any or a combination of the diagnostic or prognostic assays described herein. The subjects may be at risk for a disease or disorder disclosed herein. Administration of a prophylactic agent can occur prior to the manifestation of symptoms characteristic of a disease or disorder disclosed herein, such that the condition or symptom thereof, is prevented or, alternatively, delayed in its progression.

### B. Additional Therapeutic Methods

In one aspect, the present invention provides a method for treating a disease or disorder disclosed herein. As a non-limiting, representative example, a method comprises administering to a subject an irisin glycosylation mutant (e.g., an irisin polypeptide, comprising a glycosylated residue at position 36, a glycosylated residue at position 81, and a non-N-linked glycosylated residue at position 45) or biologically active fragments thereof or a polynucleotide encoding a irisin polypeptide disclosed herein or biologically active fragments thereof, and/or an enhancer of such a polypeptide/nucleic acid expression or activity.

Accordingly, another aspect encompassed by the present invention pertains to methods of modulating expression or activity of irisin or biologically active fragment thereof for therapeutic purposes and for use in treatment of a disease or disorder characterized by or caused by increased levels of α-synuclein or an α-synucleinopathy, such as Parkinsons' disease, Lewy body dementia, multiple system atrophy (MSA), Alzheimer's disease. Lou Gehrig's disease (ALS), or a neuroaxonal dystrophy. In an exemplary embodiment, modulatory methods encompassed by the present invention involve reducing the level or amount of α-synuclein in the cells of a subject in need thereof, the method comprising administering to the subject with the irisin glycosylation mutant or a biologically active fragment thereof or a polynucleotide encoding the irisin glycosylation mutant or biologically active fragment thereof, or an enhancer of such a polypeptide's or nucleic acid's expression or activity. In one embodiment, the agent simulates one or more activities of irisin or biologically active fragments thereof. These modulatory methods may be performed in vitro or ex vivo (e.g., by culturing the cell with the agent) or, alternatively, in vivo (e.g., by administering the agent to a subject). The cells can be neurons (e.g., hippocampal neurons, cerebellar neurons, sciatic nerve neurons, dopaminergic neurons, and substantia nigra neurons), glia, or cancer cells (e.g., melanoma cells). In one embodiment, the method involves administering an agent (e.g., an agent identified by a screening assay described herein), or combination of agents that modulate irisin expression or activity or are otherwise useful for treating or preventing an α-synucleinopathy or a cancer characterized by or caused by increased levels of α-synuclein.

Increasing expression or activity of the irisin glycosylation mutant or a biologically active fragment thereof leads to treatment or prevention of the condition that would benefit from preventing or reducing degeneration of dopaminergic (DA) neurons, preventing or ameliorating at least one motor deficit and/or preventing or ameliorating at least one symptom of cognitive dysfunction or dementia, such as Parkinson's disease, Lewy body dementia, multiple system atrophy (MSA), Alzheimer's disease, Lou Gehrig's disease (ALS), or a neuroaxonal dystrophy, therefore providing a method for treating, preventing, and/or assessing the condition of interest in a subject. The motor deficit is at least one is selected from the group consisting of: tremor at rest, such as a slight tremor in the hands or feet; rigidity (stiffness) of limbs, neck, or shoulders; difficulty balancing (postural instability); slowness of movement or gradual loss of spontaneous movement (bradykinesia); trouble standing after sitting; stiffness in the limbs; and moving more slowly. The symptom of cognitive dysfunction or dementia is at least one selected from the group consisting of: confusion; poor motor coordination; loss of short-term or long-term memory; identity confusion; and impaired judgment.

In some embodiments, expression brain-derived neurotrophic factor (BDNF) can be increased by contacting cells (e.g., neurons, glia, cancer cells) with an agent in vivo, ex vivo, or in vitro, where the agent comprises: an irisin glycosylation mutant (e.g., an irisin polypeptide, comprising a glycosylated residue at position 36, a glycosylated residue at position 81, and a non-N-linked glycosylated residue at position 45) or a biologically active fragment thereof, or a polynucleotide encoding a irisin polypeptide disclosed herein or biologically active fragment thereof.

In another aspect, neurological diseases or disorders in a subject can be treated or prevented by a method comprising the step of administering to the subject an agent comprising: an irisin glycosylation mutant (e.g., an irisin polypeptide, comprising a glycosylated residue at position 36, a glycosylated residue at position 81, and a non-N-linked glycosylated residue at position 45) or a biologically active fragment thereof or a polynucleotide encoding a irisin polypeptide disclosed herein or biologically active fragment thereof, and/or an enhancer of such a polypeptide/nucleic acid expression or activity, that increases BDNF expression or activity in central or peripheral nervous system of the subject, such that the neurological disease or disorder is treated or prevented. The neurological disease or disorder would benefit from decreased neuronal cell death and/or increased neuronal survival, optionally wherein the neurological disease or disorder is selected from the group consisting of: Alzheimer's disease, Parkinson's disease, Huntington's disease, Pick's disease, Kuf's disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dytonia syndrome, Leigh syndrome, Leber hereditary optic neuropathy (LHON), parkinsonism, dystonia, motor neuron disease, neuropathy-ataxia and retinitis pimentosa (NARP), maternal inherited Leigh syndrome (MILS), Friedreich ataxia, hereditary spastic paraplegia, Mohr-Tranebjaerg syndrome, Wilson disease, sporatic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, korsakoff's psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity and movement disorders.

In one aspect, the present invention provides a method for treating a condition that would benefit from increased muscle physiology, such as a muscle wasting (e.g., atrophy), lack of exercise, a muscular dystrophy, etc.). As a non-limiting, representative example, a method of contacting a muscle cell and/or tissue with an agent comprising i) the irisin glycosylation mutant disclosed herein or biologically active fragment thereof, or ii) the nucleic acid encoding a irisin glycosylation mutant disclosed herein or biologically active fragment thereof, thereby increasing muscle cell function and/or integrity, such as muscle cell survival. The muscle physiology is at least one selected from the group consisting of: Increasing the expression of at least one neuromuscular junction biomarker (e.g., PGC-1α, acetylcholine receptor cluster (Chme), acetylcholinesterase (AchE), utrophin, and GA binding protein transcription factor subunit alpha (GABPA)); decreasing a biomarker of muscle injury, optionally wherein the biomarker is creatine kinase; decreasing the proportion of injured muscle cells in a tissue, optionally wherein the injured muscle cells are detected using an Evans blue staining assay; increasing the time of muscle activity, optionally wherein the time of muscle activity is measured using a running assay; increasing muscle strength, optionally wherein the muscle strength is measured using a forelimb grip strength assay; increasing lean muscle mass; regeneration of muscle tissue; decreasing fat mass; and inhibition of muscle atrophy.

Accordingly, another aspect encompassed by the present invention pertains to methods of treating a muscular dystrophy, such as Becker, congenital, Duchenne, distal, Emery-Dreifuss, facioscapulohumeral, limb-girdle, myotonic, and oculopharyngeal muscular dystrophy, comprising the step of administering to the subject an agent comprising i) the irisin glycosylation mutant disclosed herein or biologically active fragment thereof, or ii) the nucleic acid encoding a irisin glycosylation mutant disclosed herein or biologically active fragment thereof.

Increasing expression or activity of irisin leads to treatment or prevention of the condition that would benefit from increased muscle physiology, such as a muscular dystrophy (e.g., DMD), therefore providing a method for treating, preventing, and/or assessing the condition of interest.

Another aspect encompassed by the present invention pertains to methods of treating muscular atrophy resulting from disuse, trauma or a disease other than muscular dystrophy such as Charcot-Marie-Tooth disease or spinal muscular atrophy.

A variety of techniques may be used to increase the expression, synthesis, or activity of irisin using an irisin polypeptide, comprising a glycosylated residue at position 36, a glycosylated residue at position 81, and a non-N-linked glycosylated residue at position 45, or a nucleic acid encoding such polypeptide, or an enhancer of such a polypeptide's expression or activity.

For example, the irisin glycosylation mutant or a biologically active fragment thereof or a polynucleotide encoding a irisin glycosylation mutant disclosed herein or biologically active fragment thereof, and/or an enhancer of such a polypeptide/polynucleotide expression or activity protein may be administered to a subject. Any of the techniques discussed below may be used for such administration. One of skill in the art will readily know how to determine the concentration of effective, non-toxic doses of the protein, utilizing techniques such as those described below.

Additionally, nucleic acid sequences, such as RNA sequences encoding such irisin polyeptides may be directly administered to a subject, at a concentration sufficient to produce a level of an irisin glycosylation mutant polypeptide or a biologically active fragment thereof or an enhancer of such a polypeptide's expression or activity, such that expression or activity of the irisin glycosylation mutant or a biologically active fragment thereof in cells is increased. Any of the techniques discussed below, which achieve intracellular administration of compounds, such as, for example, liposome administration, may be used for the administration of such nucleic acid molecules. RNA molecules may be produced, for example, by recombinant techniques such as those described herein. Other pharmaceutical compositions, medications, or therapeutics may be used in combination with the agents described herein. Further, subjects may be treated by gene replacement therapy. For example, one or more copies of a polynucleotide encoding an irisin glycosylation mutant polypeptide or biologically active fragment thereof, or an enhancer of such a polypeptide's expression or activity, may be inserted into cells using vectors which include, but are not limited to adenovirus, adeno-associated virus, and retrovirus vectors, in addition to other particles that introduce DNA into cells, such as liposomes. Additionally, techniques such as those described above may be used for the introduction of desired gene sequences into human cells. Furthermore, expression or activity of transcriptional activators which act upon irisin may be increased to thereby increase expression or activity of the irisin glycosylation mutant polypeptide or biologically active fragment thereof. Small molecules that enhance the expression or activity of the irisin glycosylation mutant or biologically active fragment thereof, either directly or indirectly, may also be used.

Cells, such as, autologous cells, containing irisin glycosylation mutant expressing gene sequences may then be introduced or reintroduced into the subject. Such cell replacement techniques may be well-suited for use in treating or preventing a disease, for example, when the gene product is a secreted, extracellular gene product.

### Screening Assays

Methods (also referred to herein as a “screening assay”) are provided for identifying enhancers of the expression or activity of the irisin (e.g., an irisin polypeptide, such as novel and non-naturally occurring irisin polypeptides, such as those comprising a glycosylated residue at position 36, a glycosylated residue at position 81, and a non-N-linked glycosylated residue at position 45) or a biologically active fragment thereof, which promote or enhance expression or activity of irisin or a biologically active fragment thereof. Agents identified using assays described herein may be useful for modulating irisin or a biologically active fragment thereof, e.g., increasing irisin expression or activity. Thus, these agents would be useful for treating or preventing an α-synucleinopathy as administration of irisin polypeptide disclosed herein (e.g., a glycosylation mutant disclosed herein) or a biologically active fragment thereof to a subject having an α-synucleinopathy can improve symptoms. Additionally, the agents disclosed herein would be useful for treating or preventing cancers caused by or characterized by increased α-synuclein as administration of an irisin glycosylation mutant or biologically active fragments thereof to a subject having cancers caused by or characterized by increased α-synuclein can improve symptoms. Thus, administration of these agents disclosed herein may be effective to prevent or treat diseases such as Parkinson's disease, Lewy body dementia, Alzheimer's disease, multiple system atrophy (MSA), a neuroaxonal dystrophy, or Lou Gehrig's disease (ALS). These agents may be also administered to a subject to increase expression of brain-derived neurotrophic factor (BDNF) or activity in central or peripheral nervous system of the subject, resulting in decreased neuronal cell death and/or increased neuronal survival, and thus treating or preventing a neurological disease or disorder in the subject. Such agents disclosed herein would be also useful to treat or prevent muscular dystrophy (e.g., Becker, congenital, Duchenne, distal, Emery-Dreifuss, facioscapulohumeral, limb-girdle, myotonic, and oculopharyngeal muscular dystrophy) and muscular atrophy (e.g., Charcot-Marie-Tooth disease or spinal muscular atrophy) because administration of such agents to the muscle tissue affected by muscular dystrophy and muscular atrophy can increase muscle physiology (e.g., muscle function and strength) of such affected muscle tissue.

These assays are designed to identify agents that replicate the function of irisin or a biologically active fragment thereof, bind to or interact with such a protein, or bind to or interact with other intracellular or extracellular proteins that interact with such a protein. Such agents may include, but are not limited to peptides, antibodies, nucleic acid molecules, siRNA molecules, or small organic or inorganic compounds. Such compounds may also include other cellular proteins.

Agents identified via assays such as those described herein may be useful, for example, for increasing expression or activity of an irisin polypeptide disclosed herein or biologically active fragment thereof, or activity-induced gene expression and/or physiology in neurons and/or cancerous tissues, such as tissues from cancers that are characterized by or caused by an increased level of α-synuclein, or, for example, maintaining integrity or decreasing degradation of neuronal cells. Thus, these agents would be useful for treating or preventing an α-synucleinopathy. In some embodiments, increased activity or expression of irisin or a biologically active fragment thereof is sufficiently effective to treat or prevent an α-synucleinopathy. For example, a partial agonist or an agonist administered in a dosage or for a length of time to increase expression or activity of irisin or a biologically active fragment thereof would act to lower levels of pathological α-synuclein in tissues.

In some embodiments, agents identified via assays such as those described herein may be useful, for example, for increasing expression or activity of an irisin polypeptide disclosed herein or biologically active fragment thereof, or activity-induced gene expression and/or physiology in muscle cells and/or tissues, such as skeletal muscle, and, for example, maintaining integrity of muscle cells and/or neuromuscular junctions. Thus, these compounds would be useful for treating or preventing a muscular dystrophy (e.g., DMD) and/or a muscular atrophy. In some embodiments, increased activity or expression of irisin or a biologically active fragment thereof is sufficiently effective to treat or prevent a muscular dystrophy (e.g. DMD) and/or a muscular atrophy. For example, a partial agonist or an agonist administered in a dosage or for a length of time to increase expression or activity of irisin or a biologically active fragment thereof would act to increase muscle cell integrity or maintain proper neuromuscular junction morphology, and treat or prevent a muscular dystrophy (e.g., DMD) and/or a muscular atrophy.

In one embodiment, the present invention provides assays for screening candidate or test agents which interact with substrates of irisin or a biologically active fragment thereof. In some embodiments, the present invention provides assays for screening candidate or test compounds which modulate the activity of irisin or a biologically active fragment thereof. In still another embodiment, the present invention provides assays for screening candidate irisin glycosylation mutant or a biologically active fragment thereof having desired functional characteristics described herein (e.g., reduce a level or amount of α-synuclein, increase expression of brain-derived neurotrophic factor (BDNF), or increase muscle physiology). The test agents encompassed by the present invention may be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide or peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries may be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J Mol. Biol. 222:301-310); (Ladner supra.).

In one embodiment, an assay is a cell-based assay in which a cell is contacted with a test agent, such as an irisin glycosylation mutant or biologically active fragments thereof, and the ability of the test agent to perform the intended functions, is determined. Determining the ability of the test agent to perform the functions discussed herein may be accomplished by monitoring biomarkers described herein, for example, biopsy, biomarker expression, physical assays, and the like. In some embodiments, the cell may be a neuronal cell or a cancer cell; and the intended functions may be the ability of the test agent to prevent or reduce degeneration of neurons, prevent or ameliorate at least one motor deficit and/or prevent or ameliorate at least one symptom of cognitive dysfunction or dementia to treat or prevent an α-synucleinopathy, or reduce the level or amount of α-synuclein in the cells of a subject.

In some other embodiments, the cell may be of mammalian origin, e.g., a neuron; the intended functions may be the ability of the test compound to modulate BDNF expression or activity; and determining the ability of the test agent to modulate BDNF expression or activity, and thus preventing or treating neurological diseases or disorders, can be accomplished by monitoring, for example, neuronal survival, BDNF expression levels, the level of transcription of genes downstream of BDNF, and the like. In some embodiments, the cell is a muscle cell, such as a skeletal muscle cell, cardiac muscle cell, and/or smooth muscle cell; the intended functions may be the ability of the test compound to modulate muscle cell or tissue physiology, such as integrity and/or neuromuscular junction morphology; and determining the ability of the test agent to modulate muscle cell or tissue physiology may be accomplished by monitoring biomarkers described herein, for example, plasma levels of creatine kinase, biopsy, neuromuscular biomarker expression, physical assays, and the like.

The ability of the test agent to modulate the binding of an irisin polypeptide disclosed herein (e.g., irisin glycosylation mutants or biologically active fragments thereof) to a substrate may also be determined. Determining the ability of the test agent to modulate such binding may be accomplished, for example, by coupling the substrate with a radioisotope or enzymatic label such that binding of the substrate to an irisin polypeptide disclosed herein (e.g., irisin glycosylation mutants or biologically active fragments thereof) may be determined by detecting the labeled substrate in a complex. The irisin polypeptide disclosed herein (e.g., irisin glycosylation mutants or biologically active fragments thereof) may also be coupled with a radioisotope or enzymatic label to monitor the ability of a test agent to modulate irisin binding to the substrate in a complex. For example, such agents described herein may be labeled with ¹²⁵I, ³⁵S, ¹⁴C, or ³H, either directly or indirectly, and the radioisotope detected by direct counting of radio emmission or by scintillation counting. Agents can further be enzymatically labeled with, for example, horseradish peroxidase, alkaline phosphatase, or luciferase, and the enzymatic label detected by determination of conversion of an appropriate substrate to product.

It is also within the scope of the present invention to determine the ability of an agent to interact with substrates of irisin or a biologically active fragment thereof (e.g., integrins, modulators of BDNF expression or activity), with or without the labeling of any of the interactants. For example, a microphysiometer may be used to detect the interaction without labeling any component (McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS).

In some embodiments, a candidate agent, such as an irisin glycosylation mutant described herein or a biologically active fragment thereof, as a modulator of expression or activity of irisin or a biologically active fragment thereof, may be identified in a method wherein a cell is contacted with a candidate agent and the expression of mRNA or protein of irisin or a biologically active fragment thereof in the cell is determined. The level of expression of mRNA or protein of irisin or a biologically active fragment thereof in the presence of the candidate agent is compared to that in the absence of the candidate agent. When expression of mRNA or protein of irisin or a biologically active fragment thereof is greater (statistically significantly greater) in the presence of the candidate agent than in its absence, the candidate agent is identified as a stimulator of mRNA or protein expression of irisin or a biologically active fragment thereof. The level of mRNA or protein expression of irisin or a biologically active fragment thereof in the cells may be determined by methods described herein for detecting mRNA or protein of irisin or a biologically active fragment thereof.

For example, in some embodiments, modulators of BDNF expression are identified in a method wherein a cell is contacted with a candidate agent, such as irisin glycosylation mutants described herein, or fragments thereof, and the expression of BDNF mRNA or protein in the cell is determined. The level of expression of BDNF mRNA or protein in the presence of the candidate agent is compared to the level of expression of BDNF mRNA or protein in the absence of the candidate agent. When expression of BDNF mRNA or protein is greater (statistically significantly greater) in the presence of the candidate agent than in its absence, the candidate agent is identified as a stimulator of BDNF mRNA or protein expression. The level of BDNF mRNA or protein expression in the cells can be determined by methods described herein for detecting BDNF mRNA or protein.

In some embodiments, assays described herein may be conducted in cell-free formats using known components of gene expression or activity of irisin or a biologically active fragment thereof. In some embodiments, the assays can be conducted in cell-free formats using known components such as α-synuclein, known components of BDNF gene expression (e.g., Npas4), or known components of muscular physiology (e.g., PGC-1α). It may be desirable to immobilize certain components of the assay, such as irisin or a biologically active fragment thereof and such embodiments may benefit from the use of well-known adaptations for biomolecule immobilization, such as the use of microtitre plates, beads, test tubes, micro-centrifuge tubes in combination with derivatizable moieties, such as fusion protein domains, biotinylation, antibodies, and the like. Gene or nucleic acid expression patterns may also be utilized to assess the levels of the known component(s) described herein in a cell. The present invention further pertains to novel agents identified by the above-described screening assays. Accordingly, it is within the scope of this invention to further use an agent identified as described herein in an appropriate animal model.

Any of the agents, including, but not limited to, compounds, such as those identified in the foregoing assay systems, may be tested for an agent capable of ameliorating a condition disclosed herein, comprising the ability of the agent to modulate nucleic acid expression or polypeptide expression and/or activity of irisin or a biologically active fragment thereof, thereby identifying a agent capable of ameliorating the condition. Cell-based and animal model-based assays for the identification of agents/compounds exhibiting such an ability to ameliorate the condition(s) (e.g., prevent or reduce degeneration of dopaminergic (DA) neurons, motor deficit, or at least one symptoms of cognitive dysfunction or dementia; decrease or reduce a level or amount of α-synuclein in cells; treat or prevent Parkinson's disease, Lewy body dementia, Alzheimer's disease, multiple system atrophy (MSA), a neuroaxonal dystrophy, or Lou Gehrig's disease (ALS); increase expression of brain-derived neurotrophic factor (BDNF); treat or prevent neurological diseases or disorders; increase muscle physiology of a muscle tissue; treat or prevent a muscular dystrophy or muscular atrophy) described herein.

In one aspect, cell-based systems, as described herein, may be used to identify agents such as a nucleic acid encoding an irisin glycosylation mutant or biologically active fragment a thereof that modulate the irisin polypeptide expression or irisin polypeptide activity or treat a cancer that are characterized by or caused by an increased level of α-synuclein or an α-synucleinopathy. In other embodiments, cell-based systems, as described herein, may be also used to identify agents such as irisin glycosylation mutant polypeptide, or fragments thereof, that modulate BDNF nucleic acid expression or BDNF polypeptide activity or treat neurological diseases or disorders. Such cell-based systems disclosed herein may also be useful to determine agents for increasing muscle physiology, treating, preventing, or assessing a muscular dystrophy (e.g., DMD) or muscular atrophy. For example, such cell systems may be exposed to an agent at a sufficient concentration and for a time sufficient to elicit such an amelioration of disease symptoms in the exposed cells. After exposure, the cells may be examined to determine whether one or more of the disease phenotypes has been altered to resemble a more normal or more wild type phenotype.

In addition, animals or animal-based disease systems, such as those described herein, may be used to identify such agents. Such animal models may be used as test substrates for the identification of drugs, pharmaceuticals, therapies, and interventions which may be effective in modulating irisin or a biologically active fragment thereof, such as to treat or prevent a cancer that are characterized by or caused by an increased level of α-synuclein or an α-synucleinopathy; or to modulate PGC-1α and α-synuclein, treating or preventing neurological diseases or disorders; or to treat or prevent a muscular dystrophy (e.g., DMD) or a muscular atrophy. Such animal models may also be used as test substrates for the identification of drugs, pharmaceuticals, therapies, and interventions which may be effective in modulating PGC-1α, treating or preventing neurological diseases or disorders. In some embodiments, the parameters of the assay are defined to allow for systemic or serum expression of the agent to cross the blood-brain barrier.

Additionally, gene expression patterns may be utilized to assess the ability of an agent to modulate expression or activity of irisin or a biologically active fragment thereof. Thus, these agents would be useful for intended purposes such as treating, preventing, or assessing a cancer that are characterized by or caused by an increased level of α-synuclein or an α-synucleinopathy; treating, preventing, or assessing a neurological disease or disorder; or for treating, preventing, or assessing a muscular dystrophy (e.g., DMD) or a muscular atrophy. For example, the expression pattern of one or more genes may form part of a “gene expression profile” or “transcriptional profile” which may be then be used in such an assessment. “Gene expression profile” or “transcriptional profile,” as used herein, includes the pattern of mRNA expression obtained for a given tissue or cell type under a given set of conditions. Gene expression profiles may be generated, for example, by utilizing a differential display procedure, Northern analysis and/or RT-PCR. Gene expression profiles may be characterized for known states within the cell- and/or animal-based model systems. Subsequently, these known gene expression profiles may be compared to ascertain the effect a test compound has to modify such gene expression profiles, and to cause the profile to more closely resemble that of a more desirable profile. For example, to assess the ability of an agent to modulate BDNF expression or activity, thus treating, preventing, or assessing a neurological disease or disorder, useful markers are described herein and include, without limitation, markers of mitochondrial function such as LDH2, Ndujb5, COX6α1, and ATP5j, markers of neuronal activity, such as immediate early genes, NF-H, NF-M, MOBP, ATPa1, and ATP1a2, upstream and downstream regulators of BDNF gene expression, and the like.

Additionally, also provided herein are methods of screening subjects by measuring or calculating the amount or level of irisin's expression and activity component disclosed herein (e.g., irisin, α-synuclein, BDNF) in the cells of a subject to determine if the subject would benefit from a treatment method described herein. In some embodiments, provided herein are method of administrating of an agent comprising: i) an irisin glycosylation mutant or biologically active fragments thereof, or ii) a nucleic acid encoding the irisin glycosylation mutant or biologically active fragments thereof if cells within the subject exhibit increased levels of α-synuclein.

The tested components described herein for irisin's expression and activity (e.g., α-synuclein, BDNF, and irisin) may be measured by any method known in the art. For example, a biological sample may be taken from a patient. Samples may be obtained by any means known in the art. Samples may also be taken directly from the nervous system, the tumor, or tumor microenvironment.

A detection method encompassed by the present invention may be used to detect mRNA, protein, or genomic DNA of the tested component described herein or a pathogenic form of the tested component described herein in a biological sample in vitro, as well as in vivo. For example, in vitro techniques for detection of mRNA include Northern hybridizations and in situ hybridizations. In vitro techniques for detection of protein include enzyme linked immunosorbent assays (ELISAs), Western blots, immunoprecipitations and immunofluorescence. In vitro techniques for detection of genomic DNA include Southern hybridizations. Furthermore, in vivo techniques for detection of protein include introducing into a subject a labeled antibody against the desired protein to be detected. For example, the antibody may be labeled with a radioactive marker whose presence and location in a subject may be detected by standard imaging techniques.

Antibodies directed against the tested component or a pathogenic or form thereof may also be used in disease diagnostics and prognostics. Such antibodies are well-known in the art (see, for example, antibody ab138501 or ab212184 from Abcam, antibody Cat #32-8100 or Cat #MA 1-90346 from ThermoFisher. In addition, such diagnostic methods, may be used to detect abnormalities in the level of such polypeptide expression, or abnormalities in the structure and/or tissue, cellular, or subcellular location of such polypeptides. Structural differences may include, for example, differences in the size, electronegativity, or antigenicity of the mutant polypeptide relative to the normal polypeptide. Protein from the tissue or cell type to be analyzed may easily be detected or isolated using techniques that are well-known to one of skill in the art, including but not limited to Western blot analysis. For a detailed explanation of methods for carrying out Western blot analysis, see Sambrook et al, 1989, supra, at Chapter 18. The protein detection and isolation methods employed herein may also be such as those described in Harlow and Lane, for example (Harlow, E. and Lane, D., 1988, “Antibodies: A Laboratory Manual”, Cold Spring Harbor Laboratory Press, Cold Spring Harbor, New York), which is incorporated herein by reference in its entirety.

This may be accomplished, for example, by immunofluorescence techniques employing a fluorescently labeled antibody (see below) coupled with light microscopic, flow cytometric, or fluorimetric detection. The antibodies (or fragments thereof) useful according to the present invention may, additionally, be employed histologically, as in immunofluorescence or immunoelectron microscopy, for in situ detection of irisin or a biologically active fragment thereof. In situ detection may be accomplished by removing a histological specimen from a subject, and applying thereto a labeled antibody of the present invention. The antibody (or fragment) is may be applied by overlaying the labeled antibody (or fragment) onto a biological sample. Through the use of such a procedure, it is possible to determine not only the presence of irisin or a biologically active fragment thereof, but also its distribution in the examined tissue. Using the present invention, one of ordinary skill will readily perceive that any of a wide variety of histological methods (such as staining procedures) may be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier may be either soluble to some extent or insoluble for the purposes encompassed by the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Supports include, but are not limited to, polystyrene beads. Those skilled in the art will know many other suitable carriers for binding antibody or antigen, or will be able to ascertain the same by use of routine experimentation.

One means for labeling an antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, MD; Voller, et al., J Clin. Pathol. 31:507-520 (1978); Butler, Meth. Enzymol. 73:482-523 (1981); Maggio, (ed.) Enzyme Immunoassay, CRC Press, Boca Raton, FL, 1980; Ishikawa, et al., (eds.) Enzyme Immunoassay, Kgaku Shoin, Tokyo, 1981). The enzyme, which is bound to the antibody, will react with an appropriate substrate, such as a chromogenic substrate, in such a manner as to produce a chemical moiety that may be detected, for example, by spectrophotometric, fluorimetric or by visual means. Enzymes that may be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection may be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope may be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody may also be detectably labeled using fluorescence emitting metals such as ¹⁵²Eu, or others of the lanthanide series. These metals may be attached to the antibody using such metal chelating groups as diethylenetriaminepentacetic acid (DTPA) or ethylenediaminetetraacetic acid (EDTA).

The antibody also may be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody encompassed by the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Diagnostic procedures may also be performed in situ directly upon tissue sections (fixed and/or frozen) of subject tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

Additionally, also provided herein are methods of screening subjects by detecting duplication of the α-synuclein locus in the genome of cells within a subject to determine if the subject would benefit from a treatment method described herein. Duplication, triplication and of the α-synuclein locus can cause an α-synucleinopathy. Polymorphisms in the α-synuclein gene can increase or decrease one's rise of developing α-synucleinopathy, based on the expression of α-synuclein.

The present invention further provides methods for detecting single nucleotide polymorphisms in a gene encoding α-synuclein or duplication of the α-synuclein locus, or in an irisin-encoding gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each subject. Several methods have been developed to facilitate the analysis of such single nucleotide polymorphisms.

In one embodiment, the single base polymorphism may be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide presents in the polymorphic site of the target molecule was complementary to that of the nucleotide derivative used in the reaction. This method has the advantage that it does not require the determination of large amounts of extraneous sequence data.

In some embodiments encompassed by the present invention, a solution-based method is used for determining the identity of the nucleotide of a polymorphic site (Cohen, D. et al. (French Patent 2, 650,840; PCT Application No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Application No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2, 650,840; PCT Appln. No. WO91/02087) the method of Goelet, P. et al. is, in some embodiments, a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A. -C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J Hum. Genet. 52:46-59 (1993)).

Another method to detect the level of tested components described herein for irisin's expression and activity (e.g., α-synuclein, BDNF, and irisin) in the subject's cells includes an in vitro amplification technology, designated “real-time quaking-induced conversion (RT-QUIC),” for detection of a form of protein. This technique differs from other amplification techniques by “quaking” the sample. The “quaking” in the name of the technique refers to the fact that samples in the RT-QuIC assay are literally subjected to shaking. This action breaks apart aggregates of pathogenic protein that are then further incubated, and amplified to detectable levels. In some embodiments, patients are stratified and chosen for a method disclosed herein following RT-QUIC analysis of α-synuclein in the subject's cells. Further details regarding this technique include Bargar, C., et al (2021). Streamlined α-synuclein RT-QuIC assay for various biospecimens in Parkinson's disease and dementia with Lewy bodies. Acta neuropathologica communications, 9(1), 62; Poggiolini, I., et al. (2021). Diagnostic value of cerebrospinal fluid α-synuclein seed quantification in synucleinopathies. Brain: a journal of neurology, awab43L Advance online publication; Zerr I (2021). RT-QuIC for detection of prodromal α-synucleinopathies. The Lancet. Neurology, 20(3), 165-166).

For determining the identity of the allelic variant of a polymorphic region located in the coding region of a gene encoding irisin, or a gene encoding α-synuclein or a duplication of the α-synuclein locus, yet other methods than those described above may be used. For example, identification of an allelic variant that encodes a mutated protein may be performed by using an antibody specifically recognizing the mutant protein in, e.g., immunohistochemistry or immunoprecipitation. Antibodies to wild-type irisin or a biologically active fragment thereof, or mutated forms of such proteins may be prepared according to methods known in the art.

### Predictive Medicine

The present invention also pertains to the field of predictive medicine in which diagnostic assays, prognostic assays, and monitoring of clinical trials are used for prognostic (predictive) purposes to thereby treat an individual prophylactically. Accordingly, one aspect of the present invention relates to diagnostic assays for determining the levels of protein and/or nucleic acid expression or activity of, in the context of a biological sample (e.g., blood, serum, fluid, cells, or tissue, e.g., cancer cells, neuronal cells, neural tissues, or muscle tissues) to thereby determine whether an individual is afflicted with a condition that would benefit from reducing or lowing the level or amount of α-synuclein, increasing expression of brain-derived neurotrophic factor (BDNF), decreasing neuronal cell death and/or increased neuronal survival, increasing muscle physiology of a muscle tissue, or has a risk of developing a condition disclosed herein. The present invention also provides for prognostic (or predictive) assays for determining whether an individual is at risk of developing the condition (e.g., α-synucleinopathy, neurological diseases or disorders, muscular dystrophy or muscular atrophy). Such individual described herein may benefit from administration of the agents disclosed herein.

One particular embodiment includes a method for assessing whether a subject is afflicted with an α-synucleinopathy or a cancer characterized by or caused by an increase in α-synuclein or is at risk of developing an α-synucleinopathy or a cancer characterized by or caused by an increase in α-synuclein comprising detecting the expression or activity of FNDC5 or a biologically active fragment thereof (e.g., irisin) in cell (e.g., a cancer cell or a neuronal cell), such as from a sample from a subject, wherein a decrease in the expression or activity thereof indicates the presence of a α-synucleinopathy or a cancer characterized by or caused by an increase in α-synuclein or the risk of developing a α-synucleinopathy or a cancer characterized by or caused by an increase in α-synuclein in the subject. Subject samples tested may comprise, for example, cancer cells or neuronal cells.

One particular embodiment includes a method for assessing whether a subject is afflicted with a neurological disease or disorder or is at risk of developing a neurological disease or disorder comprising detecting the expression or activity of the FNDC5 or irisin polypeptide, or fragments thereof in a cell or tissue sample of a subject, wherein a decrease in the expression or activity thereof indicates the presence of a neurological disease or disorder or the risk of developing a neurological disease or disorder in the subject. In this embodiment, subject samples tested are, for example, cerebrospinal fluid, spinal fluid, and neural tissue.

Additionally, one aspect of the present invention relates to a method for assessing whether a subject is afflicted with a muscular dystrophy (e.g., DMD) or is at risk of developing a muscular dystrophy (e.g., DMD) comprising detecting the expression or activity of FNDC5 or a biologically active fragment thereof (e.g., irisin) in a muscle cell or muscle tissue, such as from a sample from a subject, wherein a decrease in the expression or activity thereof indicates the presence of a muscular dystrophy (e.g., DMD) or the risk of developing a muscular dystrophy (e.g., DMD) in the subject. Subject samples tested may comprise, for example, skeletal muscle, cardiac muscle, and/or smooth muscle.

Another aspect encompassed by the present invention pertains to monitoring the influence of agents comprising the irisin polypeptides disclosed herein (the irisin glycosylation mutants or biologically active fragments thereof; irisin polypeptide, e.g., the irisin glycosylation mutants comprising a glycosylated residue at position 36, a glycosylated residue at position 81, and a non-N-linked glycosylated residue at position 45) in preventing and treating in clinical trials.

### A. Prognostic and Diagnostic Assays

To determine whether a subject is afflicted with a condition disclosed herein, or has a risk of developing such a condition, a biological sample may be obtained from a subject and the biological sample may be contacted with a compound or an agent capable of detecting an irisin polypeptide or a biologically active fragment thereof or a polynucleotide (e.g., mRNA or genomic DNA) encoding the irisin polypeptide or biologically active fragment thereof, in the biological sample. An agent for detecting the mRNA or genomic DNA may comprise a labeled nucleic acid probe capable of hybridizing to the mRNA or genomic DNA. The nucleic acid probe may be, for example, a sequence that is complementary to an Fndc5 or irisin nucleic acid set forth in Table 1, or a portion thereof, such as an oligonucleotide of at least 15, 20, 25, 30, 25, 40, 45, 50, 100, 250 or 500 nucleotides in length and sufficient to specifically hybridize under stringent conditions to the desired mRNA or genomic DNA. Other suitable probes for use in diagnostic assays encompassed by the present invention are described herein.

The term “biological sample” is intended to include tissues, cells, and biological fluids isolated from a subject, as well as tissues, cells, and fluids present within a subject. That is, a detection method encompassed by the present invention may be used to detect mRNA, protein, or genomic DNA of Fndc5 or a biologically active fragment thereof (e.g., irisin) in a biological sample in vitro, as well as in vivo. For example, in vitro techniques for detection of mRNA include Northern hybridizations and in situ hybridizations. In vitro techniques for detection of protein include enzyme linked immunosorbent assays (ELISAs), Western blots, immunoprecipitations and immunofluorescence. In vitro techniques for detection of genomic DNA include Southern hybridizations. Furthermore, in vivo techniques for detection of protein include introducing into a subject a labeled antibody against the desired protein to be detected. For example, the antibody may be labeled with a radioactive marker whose presence and location in a subject may be detected by standard imaging techniques.

In another embodiment, the methods further involve obtaining a control biological sample from a control subject, contacting the control sample with a compound or agent capable of detecting protein, mRNA, or genomic DNA, such that the presence of the desired protein, mRNA or genomic DNA is detected in the biological sample, and comparing the presence of the protein, mRNA or genomic DNA in the control sample with the presence of the protein, mRNA or genomic DNA in the test sample.

Analysis of one or more polymorphic regions of nucleic acids of irisin or a biologically active fragment thereof in a subject may be useful for predicting whether a subject has or is likely to develop a condition that would benefit from a decrease in the level or amount of α-synuclein in cells of the subject. In some embodiments, methods encompassed by the present invention may be characterized as comprising detecting, in a sample of cells from the subject, the presence or absence of a specific allelic variant of one or more polymorphic regions of the gene, such as a premature truncation that does not encode a biologically active protein or a mutation in the stop codon. The allelic differences may be: (i) a difference in the identity of at least one nucleotide or (ii) a difference in the number of nucleotides, which difference may be a single nucleotide or several nucleotides. The present invention also provides methods for detecting differences in a gene encoding Fndc5 or a biologically active fragment thereof (e.g., irisin), such as chromosomal rearrangements, e.g., chromosomal dislocation. The present invention may also be used in prenatal diagnostics.

A detection method may be allele-specific hybridization using probes overlapping the polymorphic site and having about 5, 10, 20, 25, or 30 nucleotides around the polymorphic region. In one embodiment encompassed by the present invention, several probes capable of hybridizing specifically to allelic variants are attached to a solid phase support, e.g., a “chip.” Oligonucleotides may be bound to a solid support by a variety of processes, including lithography. For example, a chip may hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes may be identified in a simple hybridization experiment. For example, the identity of the allelic variant of the nucleotide polymorphism in the 5′ upstream regulatory element may be determined in a single hybridization experiment.

In other detection methods, it is necessary to first amplify at least a portion of nucleic acid prior to identifying the allelic variant. Amplification may be performed, e.g., by PCR and/or LCR (see Wu and Wallace, (1989) Genomics 4:560), according to methods known in the art. In one embodiment, genomic DNA of a cell is exposed to two PCR primers and amplification for a number of cycles sufficient to produce the required amount of amplified DNA. In some embodiments, the primers are located between 150 and 350 base pairs apart.

Alternative amplification methods include: self-sustained sequence replication (Guatelli, J. C. et al., 1990, Proc. Natd. Acad. Sci. USA 87:1874-1878), transcriptional amplification system (Kwoh, D. Y. et al., 1989, Proc. Natd. Acad. Sci. USA 86:1173-1177), Q-Beta Replicase (Lizardi, P. M. et al., 1988, Bio Technology 6:1197), and self-sustained sequence replication (Guatelli et al., (1989) Proc. Nat. Acad. Sci. 87:1874), and nucleic acid based sequence amplification (NABSA), or any other nucleic acid amplification method, followed by the detection of the amplified molecules using techniques well-known to those of skill in the art. These detection schemes are especially useful for the detection of nucleic acid molecules if such molecules are present in very low numbers.

In one embodiment, any of a variety of sequencing reactions known in the art may be used to directly sequence at least a portion of an Fndc5- or irisin-encoding gene, or portion thereof, and detect allelic variants, e.g., mutations, by comparing the sequence of the sample sequence with the corresponding reference (control) sequence. Exemplary sequencing reactions include those based on techniques developed by Maxam and Gilbert (Proc. Natl Acad Sci USA (1977) 74:560) or Sanger (Sanger et al. (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled “DNA Sequencing by Mass Spectrometry Via Exonuclease Degradation” by H. Koster), and U.S. Pat. No. 5,605,798 and International Patent Application No. PCT/US96/03651 entitled DNA Diagnostics Based on Mass Spectrometry by H. Köster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, may be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”.

In some cases, the presence of a specific allele of an Fndc5- or irisin-encoding gene in DNA from a subject may be shown by restriction enzyme analysis. For example, a specific nucleotide polymorphism can result in a nucleotide sequence comprising a restriction site that is absent from the nucleotide sequence of another allelic variant.

In a further embodiment, protection from cleavage agents (such as a nuclease, hydroxylamine or osmium tetroxide and with piperidine) may be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of an Fndc5 allelic variant with a sample nucleic acid, e.g., RNA or DNA, obtained from a tissue sample. The double-stranded duplexes are treated with an agent that cleaves single-stranded regions of the duplex such as duplexes formed based on base pair mismatches between the control and sample strands. For instance, RNA/DNA duplexes may be treated with RNase and DNA/DNA hybrids treated with Si nuclease to enzymatically digest the mismatched regions. In other embodiments, either DNA/DNA or RNA/DNA duplexes may be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al. (1988) Proc. Natl Acad Sci USA 85:4397; Saleeba et al. (1992) Methods Enzymol. 217:286-295. In one embodiment, the control or sample nucleic acid is labeled for detection.

In another embodiment, an allelic variant may be identified by denaturing high-performance liquid chromatography (DHPLC) (Oefner and Underhill (1995) Am. J Human Gen. 57:Suppl. A266). DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility is used to identify the type of desired allelic variant. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between two nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions that permit hybridization only if a perfect match is found (Saiki et al. (1986) Nature 324:163); Saiki et al (1989) Proc. Natl Acad. Sci USA 86:6230; and Wallace et al. (1979) Nucl. Acids Res. 6:3543). Such allele specific oligonucleotide hybridization techniques may be used for the simultaneous detection of several nucleotide changes in different polymorphic regions of Fndc5- or irisin-encoding genes. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology that depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition, it may be desirable to introduce a novel restriction site in the region of the mutation to create cleavage-based detection (Gasparini et al. (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren, U. et al., (1988) Science 241:1077-1080. The OLA protocol uses two oligonucleotides that are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., (1990) Proc. Natl. Acad. Sci. (U.S.A.) 87:8923-8927. In this method, PCR is used to achieve the exponential amplification of target DNA, which is then detected using OLA.

Several techniques based on this OLA method have been developed and may be used to detect specific allelic variants of a polymorphic region of an Fndc5 gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction may be detected by using hapten specific antibodies that are labeled with different enzyme reporters, alkaline phosphatase or horseradish peroxidase. This system permits the detection of the two alleles using a high throughput format that leads to the production of two different colors.

If a polymorphic region is located in an exon, either in a coding or non-coding portion of the gene, the identity of the allelic variant may be determined by determining the molecular structure of the mRNA, pre-mRNA, or cDNA. The molecular structure may be determined using any of the above described methods for determining the molecular structure of the genomic DNA.

The methods described herein may be performed, for example, by utilizing pre-packaged diagnostic kits, such as those described above, comprising at least one probe or primer nucleic acid described herein, which may be conveniently used, e.g., to determine whether a subject has or is at risk of developing a disease associated with a specific allelic variant of interest. Sample nucleic acid to be analyzed by any of the above-described diagnostic and prognostic methods may be obtained from any cell type or tissue of a subject. For example, a subject's bodily fluid (e.g., blood) may be obtained by known techniques (e.g., venipuncture). Alternatively, nucleic acid tests may be performed on dry samples (e.g., hair or skin). Fetal nucleic acid samples may be obtained from maternal blood as described in International Patent Application No. WO91/07660 to Bianchi. Alternatively, amniocytes or chorionic villi may be obtained for performing prenatal testing.

In addition to methods that focus primarily on the detection of one nucleic acid sequence, profiles may also be assessed in such detection schemes. Fingerprint profiles may be generated, for example, by utilizing a differential display procedure, Northern analysis and/or RT-PCR.

B. Monitoring of Effects during Clinical Trials

The present invention further provides methods for determining the effectiveness of the irisin glycosylation mutants described herein (e.g., an irisin polypeptide, comprising a glycosylated residue at position 36, a glycosylated residue at position 81, and a non-N-linked glycosylated residue at position 45; or further comprising a mutation to at least one residue listed in Table 2 to modulate at least one activity of the irisin polypeptide described herein (e.g., irisin glycosylation mutants), or complex thereof) or a biologically active fragment thereof, or enhancer of expression or activity thereof, in treating or preventing a condition that would benefit from reducing or lowering the levels or amount of α-synuclein, increasing the levels or amount of brain-derived neurotrophic factor (BDNF), increasing the muscle physiology of muscle tissue, and the like, or assessing risk of developing such a condition (e.g., a condition disclosed herein). For example, the effectiveness of such an enhancer (e.g., the agents comprising the irisin glycosylation mutants or biologically active fragments thereof, as described herein; e.g., the irisin glycosylation mutant is an irisin polypeptide, comprising a glycosylated residue at position 36, a glycosylated residue at position 81, and a non-N-linked glycosylated residue at position 45) on irisin expression or activity may be monitored in clinical trials of subjects. In such clinical trials, the expression or activity of the irisin glycosylation mutants disclosed herein or biologically active fragments thereof that have been implicated in, for example, an expression pathway of irisin or biologically active fragments thereof may be used as a “read out” or marker of the phenotype of a particular cell.

For example, and not by way of limitation, genes, including irisin or a biologically active fragment thereof, that are modulated in cells by treatment with an agent that increases expression or activity of irisin, e.g., due to increased expression or activity of the irisin polypeptide described herein (e.g., irisin glycosylation mutants, or biologically active fragments thereof; e.g., the irisin glycosylation mutant polypeptides comprises a glycosylated residue at position 36, a glycosylated residue at position 81, and a non-N-linked glycosylated residue at position 45) may be identified. Thus, to study the effect of agents that increases irisin expression or activity in subjects suffering from or at risk of developing the condition, or agents to be used as a prophylactic, for example, a clinical trial, cells may be isolated and RNA prepared and analyzed for the levels of expression or activity of irisin or a biologically active fragment thereof (e.g., the irisin glycosylation mutants or biologically active fragments thereof, as described herein), and other genes implicated in the pathway of irisin or a biologically active fragment thereof. The levels of gene expression (e.g., a gene expression pattern) may be quantified by Northern blot analysis or RT-PCR, as described herein, or alternatively by measuring the amount of protein produced, by one of the methods described herein, or by measuring the levels of activity of irisin or biologically active fragments thereof, or the irisin glycosylation mutants described herein or biologically active fragments thereof. In this way, the gene expression pattern may serve as a marker, indicative of the physiological response of the cells to the agent that increases expression or activity of irisin or a biologically active fragment thereof (e.g., via increased expression and activity of the irisin glycosylation mutants described herein or biologically active fragments thereof). This response state may be determined before, and at various points during treatment of the individual with the agent that increases expression or activity of irisin or a biologically active fragment thereof (e.g., irisin or irisin glycosylation mutant).

In one embodiment, the present invention provides a method for monitoring the effectiveness of treatment of a subject with an agent that increases expression or activity of irisin or a biologically active fragment thereof (e.g., agent comprising the irisin glycosylation mutants described herein or biologically active fragments thereof) (e.g., an agonist, antagonist, peptidomimetic, protein, peptide, nucleic acid, siRNA, or small molecule identified by the screening assays described herein) including the steps of (i) obtaining a pre-administration sample from a subject prior to administration of the agent (comprising the irisin polypeptide disclosed herein; e.g., irisin glycosylation mutants, or biologically active fragments thereof; e.g., the irisin glycosylation mutant polypeptides comprises a glycosylated residue at position 36, a glycosylated residue at position 81, and a non-N-linked glycosylated residue at position 45), such as a sample comprising cancer cells or neuronal cells; (ii) detecting the level of expression of an irisin protein, mRNA, or genomic DNA in the pre-administration sample; (iii) obtaining one or more post-administration samples from the subject; (iv) detecting the level of expression or activity of irisin or a biologically active fragment thereof (e.g., the irisin glycosylation mutants or biologically active fragments thereof, as described herein) in the post-administration samples; (v) comparing the level of expression or activity of irisin or a biologically active fragment thereof (e.g., the irisin glycosylation mutants or biologically active fragments thereof, as described herein) in the pre-administration sample with irisin or a biologically active fragment thereof (e.g., the irisin glycosylation mutants or biologically active fragments thereof, as described herein) in the post administration sample or samples; and (vi) altering the administration of the agent to the subject accordingly. For example, increased administration of the agent may be desirable to increase the expression or activity of irisin or a biologically active fragment thereof (e.g., the irisin glycosylation mutants or biologically active fragments thereof, as described herein) to higher levels than detected, i.e., to increase the effectiveness of the agent. According to such an embodiment, expression or activity of irisin or a biologically active fragment thereof (e.g., the irisin glycosylation mutants or biologically active fragments thereof, as described herein) may be used as an indicator of the effectiveness of an agent, even in the absence of an observable phenotypic response.

### EXEMPLIFICATION

This invention is further illustrated by the following examples, which should not be construed as limiting.

### Example 1: Materials and Methods for Example 2

### Animals

Mice used for this study were housed at 22° C., unless stated differently. They were housed with a 12 h light/dark cycle and had unlimited access to food and water. Wild-type, 8 week old, male mice for exercise experiments were obtained from The Jackson Laboratory (C57BL/6J, #000664). All procedures were performed according to the NIH Guide for the Care and Use of Experimental Animal and approved Institutional Animal Care and Use Committee of Beth Israel Deaconess Medical Center.

### Acute Exercise Protocol

Mice were trained on a motorized treadmill (Columbus Instruments) for three consecutive days. The exercise protocol was adapted from Reddy et al. with minor modifications (Reddy, A., et al., pH-Gated Succinate Secretion Regulates Muscle Remodeling in Response to Exercise. Cell, 2020. 183(1): p. 62-75 e17). In brief, mice were trained 5 min at 12 m/min followed by a 1 min rest. Subsequently, mice run another 5 min at 12 m/min and 5 min at 14 m/min. On the third day of training, sedentary mice were removed from the treadmill and exercise mice were kept running for a total of 45 min with ramped up speed of 2 m/min every 5 mins and maximum speed of 26 m/min. Mice were sacrificed either 0 h, 30 min, 60 min, 2 h, or 4 h after the run, as indicated. Blood samples were taken, the gastrocnemius muscle was dissected and IF was isolated. Blood was allowed to clot for 15 min at room temperature, and centrifuged 10 min at 10,000×g to remove the clot. Tissue, IF, and serum samples were used for western blot analysis. IF isolated 60 min post exercise was used for proteomic analysis.

### Interstitial Fluid Isolation

Rapid IF isolation was modified from previous procedures (Spinelli, J. B., et al., Metabolic recycling of ammonia via glutamate dehydrogenase supports breast cancer biomass. Science, 2017. 358(6365): p. 941-946; Wiig, H., K. Aukland, and O. Tenstad, Isolation of interstitial fluid from rat mammary tumors by a centrifugation method. Am J Physiol Heart Circ Physiol, 2003. 284(1): p. H416-24; Sullivan, M. R., et al., Quantification of microenvironmental metabolites in murine cancers reveals determinants of tumor nutrient availability. Elife, 2019. 8; Reddy, A., et al., pH-Gated Succinate Secretion Regulates Muscle Remodeling in Response to Exercise. Cell, 2020. 183(1): p. 62-75 e17). Briefly, gastrocnemius muscle was dissected, placed into a 20 μm nylon mesh (Millipore Sigma NY2004700), and fixed in a 1.5 ml tube. Subsequently, tissue was centrifuged at low speed (600-800×g) for 10 min at 4° C. IF was snap frozen and kept at −80° C. for further processing and analysis.

### Immunodepletion of IF and Serum

Serum and IF samples were immunodepleted using R&D Systems™ Proteome Purify 2 Mouse Serum Protein Immunodepletion Resin (R&D Systems MIDR002020). The protocol was performed as described by the manufacturer. Briefly, 10 μl IF or serum was mixed with 1 ml of the immunodepletion resin and incubated on a rotator shaker at room temperature for 45 min. Subsequently 1 ml of resin was equally split into two SpinX filter tubes (R&D Systems SPINX8160036) and centrifuged at 1,500 g for 2 min. Flowthrough was collected, protein concentration was analyzed using Micro BCA™ Protein Assay Kit (Thermo Scientific 23235), and samples were snap frozen and kept at −80° C. for further analysis.

### Protein Digest, Peptide Isobaric Labeling, Mass Spectrometry Analysis of Interstitial Fluids

50-100 μg of immunodepleted IF samples were mixed 1:1 with protein lysis buffer (2% SDS, 150 mM NaCl, 50 mM Hepes pH 8.8, 5 mM dithiothreitol (DTT), Phosphatase Inhibitor (Sigma Aldrich 04906837001), Protease Inhibitor (Sigma Aldrich 11836170001)) and vortexed for 2 min. Samples were placed at 60° C. for 30 min and subsequently cooled down to room temperature (RT) for 10 min. To reduce disulfide bonds and alkylate cysteine residues, 14 mM iodoacetamide was added and incubated for 45 min at RT in the dark. DTT was added to a final concentration of 5 mM and incubated 15 min in the dark. Next, proteins were precipitated. Therefore, 1 volume of 100% TCA stock (Sigma Aldrich T0699) was added to 4 volumes of protein sample, mixed thoroughly, and placed on ice to precipitate overnight. Subsequently, samples were centrifuged at 17,000 g for 10 min at 4° C. Protein pellets were washed 4× with 1 ml of ice cold HPLC grade methanol (Fisher Scientific A4544). Protein pellets were dried and resuspended in 200 mM EPPS buffer (Fisher Scientific J61476). For protein digestion LysC (1/100 enzyme/protein ratio) and trypsin (1/200 enzyme/protein ratio) were added and incubated overnight at 37° C. Next, samples were acidified with formic acid (FA) to a pH ˜2. Peptides were labeled using 16-plex tandem mass tag (TMT) reagents (Thermo Fisher Scientific Rockford, IL). 5.0 mg of reagents were dissolved in 252 μl acetonitrile (ACN) (Honeywell) and 1/10 of the solution was added to 100 μg of peptides dissolved in 100 μl of 200 mM EPPS. After 1 hour (RT), the reaction was quenched by adding 3 μl of 5% hydroxylamine. Labeled peptides were combined and acidified prior to C18 SPE on Sep-Pak cartridges (Waters WAT054955). Peptides were eluted in 70% acetonitrile, 1% formic acid and dried by vacuum centrifugation. The peptides were resuspended in 10 mM ammonium bicarbonate pH 8, 5% acetonitrile and fractionated by basic pH reverse phase HPLC. In total 24 fractions were collected. The fractions were dried in a vacuum centrifuge, resuspended in 5% acetonitrile, 1% formic acid and desalted by stage-tip. Final peptides were eluted in, 70% acetonitrile, 1% formic acid, dried, and finally resuspended in 5% acetonitrile, 5% formic acid. In the end, 12 of 24 fractions were analyzed by LC-MS/MS.

All data were collected on an Orbitrap Eclipse mass spectrometer (ThermoFisher Scientific) coupled to a Proxeon EASY-nLC 1000 LC pump (ThermoFisher Scientific). Peptides were separated using a 90-min gradient at 500 nL/min on a 30 cm column (i.d. 100 μm, Accucore, 2.6 μm, 150 Å) packed inhouse. MS1 data were collected using the Orbitrap (60,000 resolution; maximum injection time 50 ms; AGC 10×10⁵). Determined charge states between 2 and 6 were required for sequencing, and a 60 s dynamic exclusion window was used. Data dependent mode was set as cycle time (3 s). MS2 scans were performed in the Orbitrap with HCD fragmentation (isolation window 0.5 Da; 50,000 resolution; NCE 37.5%; maximum injection time 300 ms; AGC 1×10⁵).

Raw files were first converted to mzXML, and monoisotopic peaks were re-assigned using Monocle (Rad, R., et al., Improved Monoisotopic Mass Estimation for Deeper Proteome Coverage. J Proteome Res, 2021. 20(1): p. 591-598). Database searching included all mouse entries from Uniprot (downloaded in July, 2014). The database was concatenated with one composed of all protein sequences in the reversed order. Sequences of common contaminant proteins (e.g., trypsin, keratins, etc.) were appended as well. Searches were performed using the comet search algorithm. Searches were performed using a 50-ppm precursor ion tolerance and 0.02 Da product ion tolerance. TMTpro on lysine residues and peptide N termini (+304.2071 Da) and carbamidomethylation of cysteine residues (+57.0215 Da) were set as static modifications, while oxidation of methionine residues (+15.9949 Da) was set as a variable modification.

Peptide-spectrum matches (PSMs) were adjusted to a 1% false discovery rate (FDR) (Elias, J. E. and S. P. Gygi, Target-decoy search strategy for increased confidence in large-scale protein identifications by mass spectrometry. Nat Methods, 2007. 4(3): p. 207-14). PSM filtering was performed using linear discriminant analysis (LDA) as described previously (Huttlin, E. L., et al., A tissue-specific atlas of mouse protein phosphorylation and expression. Cell, 2010. 143(7): p. 1174-89), while considering the following parameters: comet log expect, different sequence delta comet log expect (percent difference between the first hit and the next hit with a different peptide sequence), missed cleavages, peptide length, charge state, precursor mass accuracy, and fraction of ions matched. Each run was filtered separately. Protein-level FDR was subsequently estimated at a data set level. For each protein across all samples, the posterior probabilities reported by the LDA model for each peptide were multiplied to give a protein-level probability estimate. Using the Picked FDR method (Savitski, M. M., et al., A Scalable Approach for Protein False Discovery Rate Estimation in Large Proteomic Data Sets. Mol Cell Proteomics, 2015. 14(9): p. 2394-404), proteins were filtered to the target 1% FDR level.

For reporter ion quantification, a 0.003 Da window around the theoretical m z of each reporter ion was scanned, and the most intense m z was used. Reporter ion intensities were adjusted to correct for the isotopic impurities of the different TMTpro reagents according to manufacturer specifications. Peptides were filtered to include only those with a summed signal-to-noise (S/N) of 160 or greater across all channels. For each protein, the filtered peptide TMTpro S/N values were summed to generate protein quantification.

Protein in-Gel Digestion, Peptide Isobaric Labeling, LC-MS/MS Analysis and Mass Spectrometry Data Processing

Silver-stained gel bands were excised, destained with acetonitrile, and digested in 100 mM EPPS, pH 8.5 containing 1 μg of trypsin (Promega) (overnight at 37° C.). Digests buffer was removed and 5 μL of TMTpro reagents (Thermo Fisher) was added to each solution for 1 hr at room temperature (25° C.). After incubating, the reaction was quenched by adding 1 μL of 5% (w/v) hydroxylamine. Labelled peptides were combined and subsequently desalted by C18 StageTips (Empore 3M).

Data were collected using an Orbitrap Fusion Lumos mass spectrometer (Thermo Fisher Scientific, San Jose, CA) coupled with a Proxeon EASY-nLC 1200 LC pump (Thermo Fisher Scientific). Peptides were separated on a 100 μm inner diameter microcapillary column packed with 35 cm of Accucore C18 resin (2.6 μm, 100 Å, Thermo Fisher Scientific). Peptides were separated using a 3 hr gradient of 6-22% acetonitrile in 0.125% formic acid with a flow rate of ˜400 nL/min. Each analysis used an MS3-based TMT method. The data were acquired using a mass range of m z 400-1400, resolution at 120,000, AGC target of 1×10⁶, a maximum injection time 100 ms, dynamic exclusion of 180 seconds for the peptide measurements in the Orbitrap.

Data dependent MS2 spectra were acquired in the ion trap with a normalized collision energy (NCE) set at 35%, AGC target set to 2.0×10⁵ and a maximum injection time of 120 ms. MS3 scans were acquired in the Orbitrap with a HCD collision energy set to 45%, AGC target set to 1.5×10⁵, maximum injection time of 200 ms, resolution at 50,000 and with a maximum synchronous precursor selection (SPS) precursors set to 10.

In-house developed software was used to convert acquired mass spectrometric data from the .RAW file to the mzXML format. Erroneous assignments of peptide ion charge state and monoisotopic m/z were also corrected by the internal software. SEQUEST algorithm was used to assign MS2 spectra by searching the data against a protein sequence database including Mouse Uniprot Database (downloaded June 2017) and known contaminants such as mouse albumin and human keratins. A forward (target) database component was followed by a decoy component including all listed protein sequences. Searches were performed using a 20 ppm precursor ion tolerance and requiring both peptide termini to be consistent with trypsin specificity. 16-plex TMT labels on lysine residues and peptide N termini (+304.2071 Da) were set as static modifications and oxidation of methionine residues (+15.99492 Da) as a variable modification. An MS2 spectra assignment false discovery rate (FDR) of less than 1% was implemented by applying the target-decoy database search strategy. Filtering was performed using a linear discrimination analysis method to create one combined filter parameter from the following peptide ion and MS2 spectra properties: XCorr and ΔCn, peptide ion mass accuracy, and peptide length. Linear discrimination scores were used to assign probabilities to each MS2 spectrum for being assigned correctly and these probabilities were further used to filter the data set with an MS2 spectra assignment FDR to obtain a protein identification FDR of less than 1%.

For reporter ion quantification, a 0.003 m/z window centred on the theoretical m/z value of each reporter ion was monitored for ions, and the maximum intensity of the signal to the theoretical m/z value was recorded. Reporter ion intensities were normalized by multiplication with the ion accumulation time for each MS2 or MS3 spectrum and adjusted based on the overlap of isotopic envelopes of all reporter ions. Following extraction of the reporter ion signal, the isotopic impurities of the TMT reagent were corrected using the values specified by the manufacturer's specification. Total signal-to-noise values for all peptides were summed for each TMT channel and all values were adjusted to account for variance and a total minimum signal-to-noise value of 200 was implemented.

### Plasmids:

### Irisin Plasmids:

To generate the wild-type irisin plasmid (irisin-WT) for recombinant protein production, the irisin portion of human/mouse FNDC5 (amino acids 32-143) was fused with the an N-terminal mouse immunoglobulin heavy chain leader sequence (MEWSWVFLFFLSVTTGVHS (accession number: AON1R4_MOUSE) as set forth in SEQ ID NO: 12) and a C-terminal 10×His, and the codon optimized DNA sequence of the fusion irisin protein was synthesized and cloned into a modified pEGFP-N1 vector (Smith O et al, A domestication history of dynamic adaptation and genomic deterioration in Sorghum. Nat Plants 5(4), 369-79 (2019)) with a HRV3C protease recognition site in between irisin-His and GFP, and a twin Strep-II tag at the C-terminus of GFP, allowing the mammalian irisin expression, secretion and affinity purification. N45Q mutant plasmid (irisin-N45Q) was further generated by site-directed mutagenesis using the above mentioned irisin WT plasmid as the DNA template and deglycosylase mixII (NEB #P6044).

Irisin-mam and irisin-bac constructs used for recombinant protein production were generated as previously described (Schumacher, M. A., et al., The structure of irisin reveals a novel intersubunit beta-sheet fibronectin type III (FNIII) dimer: implications for receptor activation. J Biol Chem, 2013. 288(47): p. 33738-33744; Kim, H., et al., Irisin Mediates Effects on Bone and Fat via alphaV Integrin Receptors. Cell, 2018. 175(7): p. 1756-1768 e17).

### Integrin αVβ5 Plasmids:

Integrin αVβ5 ectodomain constructs were generated for recombinant protein production in mammalian cells as previously described (Nishida, N., et al., Activation of leukocyte beta2 integrins by conversion from bent to extended conformations. Immunity, 2006. 25(4): p. 583-94; Takagi, J., H. P. Erickson, and T. A. Springer, C-terminal opening mimics ‘inside-out’ activation of integrin alpha5beta1. Nat Struct Biol, 2001. 8(5): p. 412-6; Xie, C., et al., Structure of an integrin with an alpha1 domain, complement receptor type 4. EMBO J, 2010. 29(3): p. 666-79). In brief, soluble, heterodimeric αVβ5 construct was prepared from wild-type human αV and β5 cDNAs by PCR and standard molecular cloning techniques. αV subunit cDNA encoding the signal sequence and the ectodomain residues 32-991 was fused to a C-terminal peptide encoding an acidic α-helical coiled-coil region flanked by an N-terminal HRV3C protease recognition site and a C-terminal twin Strep-II tag and inserted into EcoRI and BamH1 sites of the pcDNA3.1 vector. Similarly, a cDNA encoding the signal sequence and the ectodomain residues 24-717 of β5 was fused to a C-terminal sequence encoding a basic α-helical coiled-coil region flanked by an N-terminal HRV3C protease recognition site and a C-terminal 8×His tag and inserted into EcoRI and BamH1 sites of the pcDNA3.1 vector. Cys residues were introduced into both of the above constructs right in front of the HRV3C sites for formation of a disulfide bond potentially. For cellular assays, αVβ5 full-length constructs were purchased from Sino Biologicals (αV: HG11269-CY; β5: HG10779-CM).

### Human Hsp90α Plasmids:

Human Hsp90α full-length cDNA was amplified by PCR from the plasmid purchased from Sino Biologicals (HG11445-CF), and cloned into a C7 vector containing an HRV3C protease site and a 10×His tag at the C-terminus using FX cloning system (Addgene 1000000039).

### Plasmids of Human Fibronectin 10^{th} FNIII Domain:

Human fibronectin 10^{th} FNIII domain cDNA was synthesized by IDT and cloned into C7 vector containing a HRV3C protease site and a 10×His tag at the C-terminus using FX cloning system (Addgene 1000000039).

### Recombinant Protein Expression and Purification

For protein expression in mammalian cells, 2.8×10⁶ Expi293F cells (Life Technologies A14527) grown in 1 L Expi293F expression medium (Life Technologies A1435101) were transfected with 1 mg DNA mixture containing 0.6 mg αV plasmid and 0.4 mg β5 plasmid, and 3 mg sterile 25 kDa linear PEI mix in Opti-PlexComplexation Buffer (Life Technologies A4096801). Proteins were expressed at 37° C., 8% CO₂, >80% humidity with shaking at 125 rpm for 4 days. Enhancers (Life Technologies A14524) were added 22 hrs post transfection to boost protein expression.

For protein expression in E. coli, T7-express (NEB C2566) E. coli was transformed with the corresponding plasmid (Hsp90α, irisin-bac and FN10) and was grown in Terrific Broth until OD₆₀₀ reached 2.5. 0.2 mM IPTG was added to induce protein expression at 22° C. for 10 hrs.

For αVβ5 ectodomain protein purification from Expi293F cells, all of the following steps were performed at 4° C. or on ice. Cells were pelleted at 600×g for 20 min, and the medium was subjected to an additional 2 hrs of centrifugation at 1000×g. The supernatant was filtered through 0.22 μm filter unit and was concentrated 10-fold using Tangential Filtration System with 50,000 kDa MWCO (Paul) before being applied to Ni-Excel affinity column (Cytiva 17371201) equilibrated in PB (10 mM Hepes pH7.4, 150 mM NaCl, 5 mM CaCl₂)). After thorough washes with PB, the column was eluted with PB supplemented with 500 mM imidazole. The eluted protein was subsequently applied to Strep-Tactin column (IBA 2-1208) equilibrated in PB. After thorough washes with PB, the column was either: 1) treated with HRV3C protease in PB (at 1:100 enzyme to substrate molar ratio) for 16 hrs at 4° C. followed by 3 column PB washes to obtain unclasped and untagged αVβ5; or 2) eluted with strep elution buffer (PB+ 5 mM dethiobiotin) to obtain clasped and tagged αVβ5. For the highly purified material, protein was then loaded onto MonoQ ion exchange column (Cytiva 17516701) and eluted with a salt gradient (50 mM to 500 mM NaCl with 10 mM Tris at pH7.4). The peak fractions containing αVβ5 ectodomain were concentrated with 50,000 MWCO Amicon Ultra-15 filter unit (Millipore) before further purification through Superdex 200 10/300 GL gel-filtration column (Cytiva 17-5175-01) equilibrated with Mg/Ca buffer (10 mM Hepes pH7.4, 150 mM NaCl, 1 mM MgCl₂, 1 mM CaCl₂)). Protein was further concentrated to >5 μM before being aliquoted, frozen in liquid nitrogen, and stored at −80° C. In all cases, the Hepes pH given is at 23° C.

For purification of Hsp90α, FN10 and irisin-bac, all following steps were performed at 4° C. or on ice. Cells were pelleted by centrifugation at 600×g for 20 min at 4° C. Supernatant was removed and the cell pellet was washed once with PBS and resuspended in EB (100 mM Tris pH 8, 500 mM NaCl, 20 mM imidazole, 0.5 mM TCEP, 10% glycerol, 1× Halt Protease Inhibitors (Thermo Fisher Scientific 78439)). Resuspended cells were then sonicated for 45 min (5 s on and 10 s off pulse) at 50% amplitude. Cell debris was removed by ultracentrifugation at 185,000×g for 1 h. For Hsp90α and FN10, the supernatant was passed through Ni-NTA (Thermo Scientific 25214) column, followed by thorough washes with WB (10 mM Tris pH 8, 250 mM NaCl, 40 mM imidazole, 0.5 mM TCEP). For Hsp90α, the column was then thoroughly washed with Mg²⁺/ATP buffer (100 mM Tris pH7.4, 50 mM KCl, 5 mM ATP, 25 mM MgCl₂, 500 mM sucrose and 25% glycerol), followed by a minimum wash step with WB. The column was then treated with HRV3C protease in WB (at 1:100 enzyme to substrate molar ratio) for 16 hrs at 4° C., followed by 3 column volume WB washes to obtain the untagged Hsp90α and FN10. For irisin-bac, the supernatant was passed through Pierce GlutathioneArgarose (Thermo Fisher Scientific 16100) column, followed by thorough washes with WBG (10 mM Tris pH 8, 250 mM NaCl, 0.5 mM TCEP). The column was then treated with HRV3C protease in WBG (at 1:100 enzyme to substrate molar ratio) for 16 hrs at 4° C. followed by 3 column volume WBG wash to obtain untagged irisin-bac. The eluted proteins were concentrated with 30,000 MWCO (for Hsp90α) or 3,000 MWCO (for FN10 and irisin-bac) Amicon Ultra-15 filter unit (Millipore) before further purification through Superdex 200 10/300 GL gel-filtration column (Cytiva 17-5175-01) equilibrated with FPLC buffer (10 mM Hepes pH7.4, 150 mM NaCl, 10% glycerol). Proteins were further concentrated to >50 μM before being aliquoted, frozen in liquid nitrogen, and stored at −80° C. In all cases, Hepes pH given is at 23° C. A portion of the prepared Hsp90α planned for mammalian cell culture treatment was further purified using Toxin eraser Endotoxin Removal Kit (GeneScript L00338), and the level of endotoxin was measured using ToxinSensor Chromogenic LAL Endotoxin Assay Kit (GeneScript L00350). The endotoxin-free Hsp90α was dialyzed into PBS and aliquoted, frozen in liquid nitrogen, and stored at −80° C.

For biochemical reconstitution of the αVβ5/Hsp90α complex in stoichiometry, 1 μM clasped and tagged αVβ5 was incubated with 20 μM untagged Hsp90α in Mg/Ca buffer with end-over-end mixing at 4° C. for 5 hrs. Protein mix was purified through TALON Metal Affinity column (Takara 635503) equilibrated in Mg/Ca buffer with extensive Mg/Ca buffer washing. The eluted protein complex was concentrated with100,000 MWCO Amicon Ultra-15 filter unit (Millipore) before further purification through Superdex 200 10/300 GL gel-filtration column (Cytiva 17-5175-01) equilibrated with Mg/Ca buffer. Protein complex was further concentrated to >5 μM before being aliquoted, frozen in liquid nitrogen, and stored at −80° C.

Proteins were dialyzed into the corresponding assaying buffers overnight at 4° C. before the individual experiments were conducted.

### Protein Binding Assays

Biolayer Inferometry (BLI) measurements were conducted by Octet RED384. 5 nM clasped and tagged αVβ5 was immobilized on Streptavidin biosensors (FortéBio) and binding kinetics was measured with varying amounts of irisin in the Mg/Ca buffer. Data analysis HT software was used for the fitting and K_{d} calculations.

For TALON pull-down of purified Hsp90α, 1 μM clasped and tagged αVβ5 or control peptide dimer (HRV3C-acidic stretch-2×strepII/HRV3C-basic stretch-8×His) was incubated with 2 μM untagged Hsp90α in Mg/Ca buffer with end-over-end mixing at 4° C. for 1 hr. Separately, 0.2 volumes of 50% TALON Metal Affinity bead slurry (Takara 635503) was washed in the Mg/Ca buffer before adding to protein mix. After 1 hr incubation at 4° C., beads were washed with the Mg/Ca buffer. The bead pellet was probed by either Coomassie staining or by western blot with anti-Hsp90α antibody (Invitrogen PA3-013). For TALON pull-down of purified Hsp90α recharged with different nucleotides, 10 μM Hsp90α was incubated with 1 mM EDTA on ice for 5 min and dialyzed into DB (10 mM Hepes pH7.4, 150 mM NaCl, 5 mM MgCl₂) to obtain Hsp90α-apo. 1 mM ATP, AMP-PNP or ADP was then added into the αVβ5/Hsp90α mix for the pulldown assay.

For immuno-precipitation from cell extracts, SK-Mel2 cells were chilled on ice and incubated with 5 μg/mL anti-Hsp90α (Invitrogen PA3-013) for 1 hr at 4° C. with shaking (7 see-saw movements per minute). The cells were than washed with cold PBS and lysed with prechilled IP buffer (50 mM Hepes pH7.4, 150 mM NaCl, 1% Triton X-100, 0.1% Na-deoxycholate, protease inhibitor cocktail (Roche 11873580001), PhosStop (Roche 49068450001), Pierce Universal Nuclease (ThermoFisher Scientific 88700)). Cell debris was removed by centrifugation at 17,000×g for 20 min at 4° C. The supernatant was then incubated with Protein A beads (Thermo Scientific 20333) equilibrated in IP buffer for 3 hrs. Beads were washed with the IP buffer, and the bound proteins were eluted with the SDS sample buffer. They were then resolved by SDS-PAGE, and probed with anti-Hsp90α (Invitrogen PA3-013), anti-αV (Cell Signaling 60896) or anti-β5 (Abcam ab184312).

Fluorescence polarization anisotropy measurements were conducted using irisin or FN10 labeled with Alexa Fluor 488 (A488), as previously described (Ramabhadran, V., A. L. Hatch, and H. N. Higgs, Actin monomers activate inverted formin 2 by competing with its autoinhibitory interaction. J Biol Chem, 2013. 288(37): p. 26847-55). For the anisotropy binding assays, 50 nM A488 labeled ligand proteins were mixed with varying concentrations of unlabeled receptor proteins or protein complexes dialyzed into the indicated buffers. For the anisotropy competition binding assay, 50 nM A488 labeled ligand proteins and fixed concentrations of the indicated receptor proteins or protein complexes dialyzed into the indicated buffers were mixed with varying concentrations of unlabeled ligand competitor proteins dialyzed into the indicated assaying buffers. Fluorescence anisotropy was measured using the Clariostar plate reader. Binding curves were fit using standard hyperbolic saturation fitting (Hulme, E. C. and M. A. Trevethick, Ligand binding assays at equilibrium: validation and interpretation. Br J Pharmacol, 2010. 161(6): p. 1219-37). The K_{d}^{app} values were calculated based on Benedetti, M. S., et al., Intestinal metabolism of tyramine by both forms of monoamine oxidase in the rat. Biochem Pharmacol, 1983. 32(1): p. 47-52; Marechal, J. D., et al., In silico and in vitro screening for inhibition of cytochrome P450 CYP3A4 by comedications commonly used by patients with cancer. Drug Metab Dispos, 2006. 34(4): p. 534-8; and Burnett, J. C., et al., A refined pharmacophore identifies potent 4-amino-7-chloroquinoline-based inhibitors of the botulinum neurotoxin serotype A metalloprotease. J Med Chem, 2007. 50(9): p. 2127-36.

### Detection of Integrin Signaling in HEK293T Cells

HEK293T cells were seeded onto the 10 cm dishes (4×10⁶ cells/dish) and incubated at 37° C., 5% CO₂ overnight. Cells were then transfected with either 10 μg of control plasmid, or 10 μg of DNA mixture containing 0.6 μg of full-length αV, 0.4 μg of full-length β5 and 9 μg of control plasmid, as well as 24 μL Lipofectamine 2000 (Invitrogen 52932), followed by 6 hrs of incubation. The transfected cells were then split into 6-well dishes (400,000 cells/well) and incubated overnight. Prior to irisin and/or Hsp90α treatment, cells were switched into FreeStyle293 expression medium (Life Technologies 12338-018) followed by 4-5 hrs of incubation. For cells that received Hsp90α, 1 nM Hsp90α was added directly into the cultures 1 hr before the irisin treatment, and then irsin was applied to the cells to the indicated final doses with minimum mechanical disturbance. Medium was removed after 5 minutes and cells were lysed with Pierce RIPA buffer (Thermo Scientific 89900), supplemented with Protease inhibitor cocktail (Roche 11836170001), PhosStop (Roche 04906837001) and Pierce Universal Nuclease (Thermo Fisher Scientific 88700). Cell lysates were scraped off the dishes and centrifuged at 17,000×g for 10 min at 4° C. The protein concentration of the supernatant was measured by Pierce Detergent Compatible Bradford Assay Reagent (Thermo Scientific 1863028), and 10 μg of total protein from each sample was loaded and resolved by SDS-PAGE. Total FAK and the FAKphosphorylated at Y397 site (pFAK Y397) were probed with anti-FAK (Cell Signaling 3285) and anti-pFAK (Cell Signaling 3283).

### Fixed Cell Imaging and Quantification

For irisin binding in HEK293T cells, cells were transfected as described above. The transfected cells were then split into the MatTek dishes (MatTek Life Sciences P35G-1.5-14-C) (200,000 cells/dish) and incubated overnight in the medium without phenol red. The next day, the cells were switched into FreeStyle293 expression medium (Life Technologies 12338-018) for 4-5 hrs. For the 1 hr Hsp90α pretreatment, 2 nM recombinant Hsp90α was added directly into the medium. The cells were then switched into prewarmed FreeStyle293 expression medium containing 2 nM A488-irisin for 5 min. After exactly 5 min, medium was removed and cells were immediately fixed with 4% paraformaldehyde (Electron Microscopy Sciences 15710) at room temperature for 20 min. Cells were then washed with PBS and stained with DAPI (Calbiochem 268298) without permeabilization for 30 min at room temperature in the dark, followed by PBS wash. For irisin binding in SK-Mel2 cells, cells were chilled on ice and then were given control antibody (R&D MAB002) or anti-Hsp90α (Enzo ADI-SPA-830-F) (1:50 dilution) in cold medium for 1 hr at 4° C. Cells were washed with PBS and then switched into A647-irisin-containing warm FreeStyle293 medium for irisin binding following the same procedure described above. For immunostaining of cell surface Hsp90α, SK-Mel2 cells were seeded onto the MatTek dishes (400,000 cells/dish). After overnight incubation, cells were chilled on ice and then were given control antibody (R&D MAB002) or Hsp90α antibody (Enzo ADI-SPA-830-F) (1:50 dilution) in cold PBS for 1 hr on ice. Cells were washed with cold PBS and fixed with 4% paraformaldehyde at room temperature for 20 min. After PBS wash, cells were incubated with A647-anti-mouse IgG (Invitrogen A21235) for 1 hr at room temperature followed by DAPI (Calbiochem 268298) staining in dark for 30 min. Cells were finally washed into PBS. Cells were imaged in PBS using Yokogawa spinning disc confocal on an inverted Nikon Ti fluorescence microscope and Hamamatsu ORCA-R₂ cooled CCD camera. Images were taken on the middle z-plane of the cells.

The number of A647-positive cells was quantified in confocal 640 nm channel based on the mean intensity values of the whole cell area (I_{c}, referring to the bright field) and the nucleus region (Iₙ, referring to the DAPI staining). Control dish with only DAPI staining was used to set up the baseline (I_{b}). I_{b}=(I_{c}−Iₙ)/N; N is the total number of the cells quantified. Cells with their (I_{c}−Iₙ) value above I_{b} were counted as positive. For the Hsp90α IF experiment, one Region of Interest (ROI) from each one biological replicate (total four biological repeats) was taken for quantification. For the A647-irisin binding experiment, three ROIs from three biological replicates were taken for quantification.

### Cell Viability Assay

Crystal violet assay was used for quantification of cell viability. SK-Mel2 cells were seeded onto 24-well dishes (50,000 cells/well) and incubated overnight. Indicated amounts of irisin were added directly into the culture and cells were incubated for 24 hrs. Cells were then washed with PBS and stained with crystal violet solution for 15 min at room temperature followed by thorough washes with milliQ water. The stained cells were incubated in methanol supplemented with 10% acetic acid for 20 min, and the optical density at OD₅₇₀ was measured using the Clariostar plate reader, and final OD₅₇₀ values of the wells with cells were obtained by subtracting the average OD₅₇₀ of the wells without cells.

### Negative-Stain Electron Microscopy

Hsp90α was diluted to a concentration of 0.02 mg/mL. To improve structural intactness of Hsp90α on the grids, 0.10% glutaraldehyde was added to the sample and incubated for 30 min on ice. Crosslinked and non-crosslinked Hsp90α samples were then applied onto glow-discharged continuous carbon grids (Electron Microscopy Sciences, Inc.), respectively. After 1 min of adsorption, the grids were blotted with filter paper to remove excess sample, and immediately washed twice with 4 μL of 1.5% uranyl formate solution followed by an incubation with 4 μL of 1.5% uranyl formate solution for additional 90 s. The grids were then further blotted with filter paper to remove the uranyl formate solution, air dried at room temperature, and examined with a Tecnai T12 electron microscope (Thermo Fisher Scientific) equipped with an LaB6 filament and operated at 120-kV acceleration voltage, using a nominal magnification of 69,000× at a pixel size of 1.68 Å.

### Cryo-EM and Image Processing

Cryo-EM grids of αVβ5 or αVβ5/Hsp90α were prepared using a Vitrobot Mark IV (Thermo Fisher Scientific). 3 μL aliquots of purified complex at concentrations between 0.5 and 0.8 mg/mL were applied onto glow-discharged C-flat holey carbon grids (R1.2/1.3, 400 mesh copper, Electron Microscopy Sciences). The grids were blotted for 6 s with a blot force of 15 and 100% humidity before being plunged into liquid ethane cooled by liquid nitrogen.

Images were acquired on a Titan Krios microscope equipped with a BioQuantum K3 Imaging Filter (slit width 25 eV) and a K3 direct electron detector (Gatan) and operating at an acceleration voltage of 200 kV. Images were recorded at a defocus range of −1.8 μm to −2.5 μm with a nominal magnification of 36,000×, resulting in a pixel size of 1.1 Å. Each image was dose-fractionated into 50 movie frames with a total exposure time of 4.5 s, resulting in a total dose of ˜58.4 electrons per Å². SerialEM was used for data collection (Schorb, M., et al., Software tools for automated transmission electron microscopy. Nat Methods, 2019. 16(6): p. 471-477).

Images were processed using cryoSPARC (Punjani, A., et al., cryoSPARC: algorithms for rapid unsupervised cryo-EM structure determination. Nat Methods, 2017. 14(3): p. 290-296). For method 1, using template-free blob picker in cryoSPARC, 294,676 particles of αVβ5 were picked from 1,100 motion-corrected micrographs and extracted from the micrographs to run 2D classification. 148,637 particles were retained following 2D classification after selecting classes with well-resolved features. 367,732 particles of αVβ5/Hsp90α were picked from 1,171 motion-corrected micrographs and 193,293 particles were retained following 2D classification after selecting classes. Then, for both selected particles of αVβ5 and αVβ5/Hsp90α, we randomly split them at the same number of 140,000. For each dataset with 140,000 particles, we performed a 2D classification with 100 classes, respectively. Based on the 2D classes, we analyzed and calculated the “open”, “likely open”, “extended closed” and “closed” states of particles. For method 2, 6 2D classes generated from αVβ5 using blob picking, which contained “open”, “extended closed” and “closed” states, were used as templates to pick particles from both αVβ5 and αVβ5/Hsp90α micrographs. After selecting classes from 2D classification, total number of 314,469 αVβ5 particles and 313,849 αVβ5/Hsp90α particles were analyzed into “open”, “likely open”, “extended closed” and “closed” states, respectively. Method 3 followed a similar scheme to that described above for method 2, but the templates were generated from αVβ5/Hsp90α using blob picking. 330,301 particles of αVβ5 and 324,872 particles of αVβ5/Hsp90α were analyzed into “open”, “likely open”, “extended closed” and “closed” states, respectively.

### Size-Exclusion Chromatography and Multiangle Light Scattering (SEC-MALS)

Absolute molecular weights of irisin-mam and its conjugated glycan were determined using MALS coupled in-line with size-exclusion chromatography. 100 μg albumin (Thermo Scientific 23209) or irisin-mam was loaded onto Superdex 200 column equilibrated in 10 mM Hepes pH7.4, 150 mM NaCl. Light scattering from the column eluent was recorded at 16 different angles using a DAWN-HELEOS MALS detector (Wyatt Technology Corp.) operating at 658 nm. The detectors at different angles were calibrated using albumin (Thermo Scientific 23209). Protein concentration of the eluent was determined using an in-line Optilab DSP Interferometic Refractometer (Wyatt Technology Corp.). The weight-averaged molecular weight of species within defined chromatographic peaks was calculated using the ASTRA7 software (Wyatt Technology Corp.), by construction of Debye plots (KC/Rθ versus sin²[θ/2]) at 1^{-s} data intervals. The weight-averaged molecular weight was then calculated at each point of the chromatographic trace from the Debye plot intercept and an overall average molecular weight was calculated by averaging across the peak. Two concentration detectors (RI and UV) were used simultaneously during the MALS data collection and Protein Conjugate Analysis (Strop, P. and A. T. Brunger, Refractive index-based determination of detergent concentration and its application to the study of membrane proteins. Protein Sci, 2005. 14(8): p. 2207-11; Pasternack, S. G., A. Veis, and M. Breen, Solvent-dependent changes in proteoglycan subunit conformation in aqueous guanidine hydrochloride solutions. J Biol Chem, 1974. 249(7): p. 2206-11) was applied to SEC-MALS data using glycan dn/dc value of 0.145 mg/L.

### Hydrogen/Deuterium Exchange (HDX) Mass Spectrometry

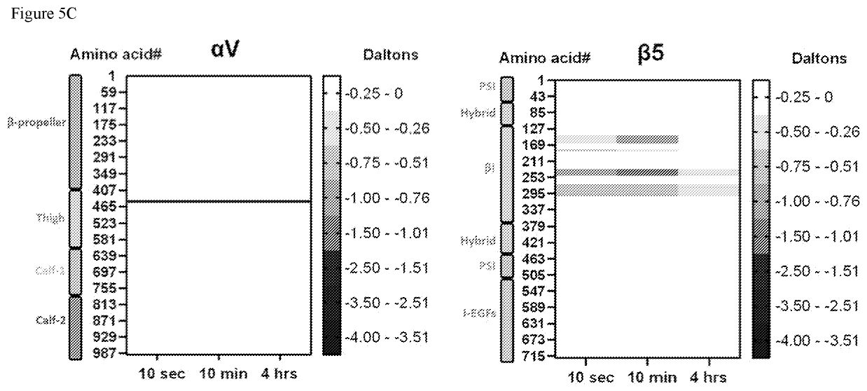

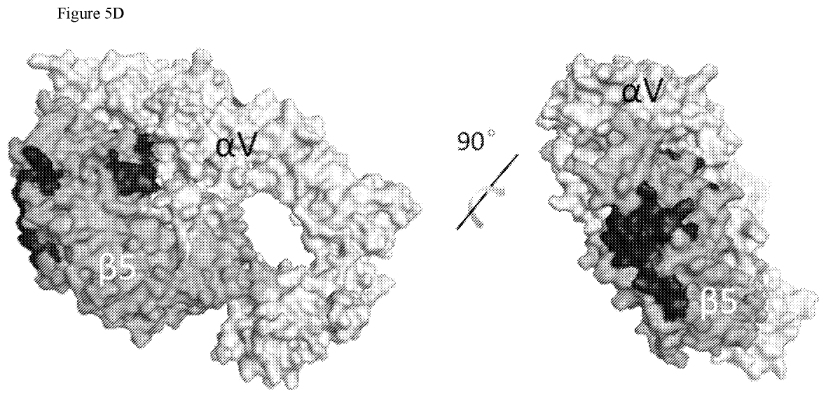

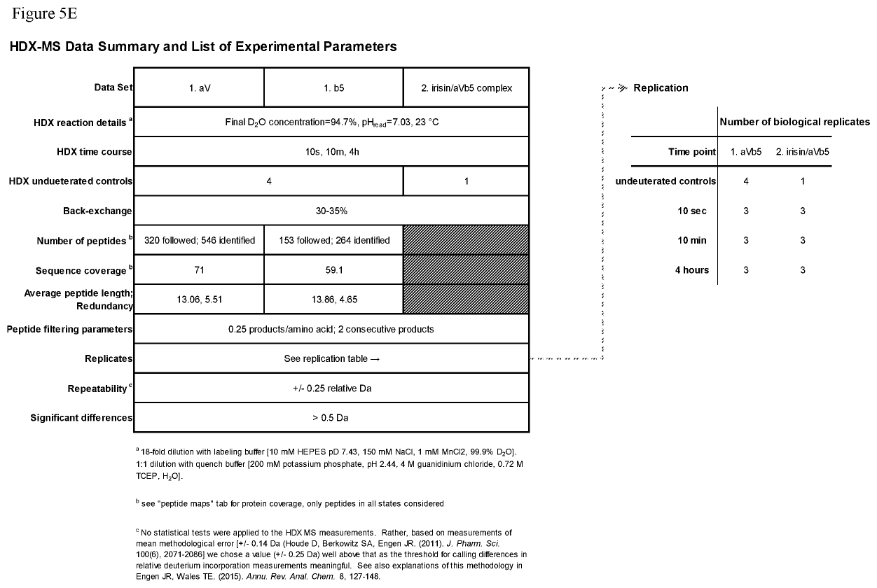

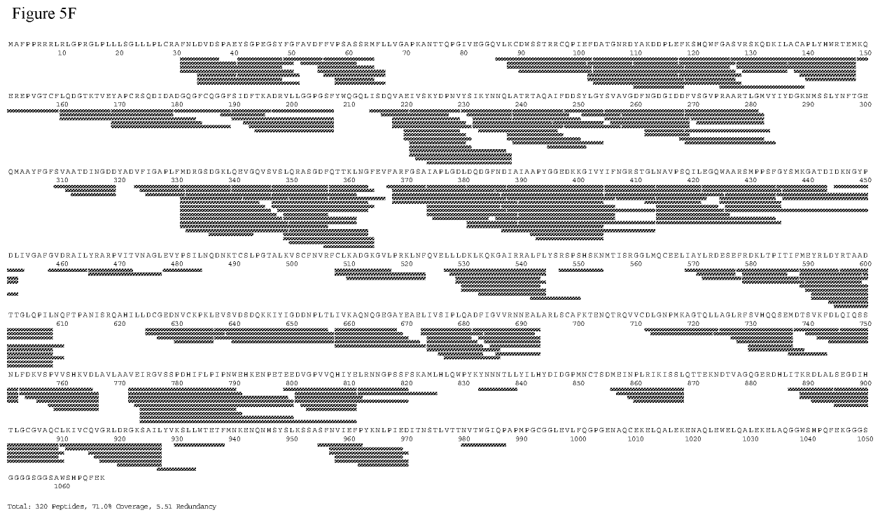

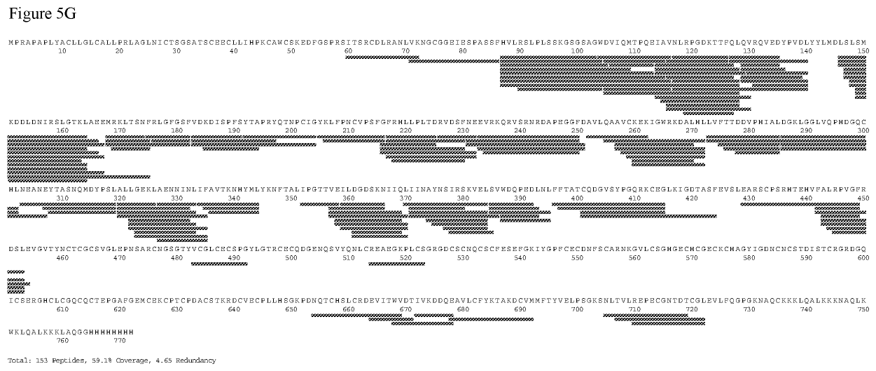

In addition to the following descriptions, comprehensive experimental details and parameters are provided in FIG. 5E in the recommended (PMID 34723319) tabular format. All HDX-MS data have been deposited to the ProteomeXchange Consortium via the PRIDE (PMID 34723319) partner repository with the dataset identifier PXD035397.

Deuterium labeling: for integrin protection, 20 μM irisin-His (from mammalian cells) was mixed with 4 μM αVβ5 ectodomain protein in Mn-buffer (10 mM Hepes pH7.4, 150 mM NaCl, 1 mM MnCl₂, H₂O); for irisin protection, 20 μM αVβ5 ectodomain protein was mixed with 4 μM irisin-His (from mammalian cells) in Mn-buffer. 20 μM irisin alone or αVβ5 alone were used as the “Apo” conditions. Deuterium labeling was initiated with an 18-fold dilution into D₂O buffer (18 μL, 10 mM Hepes pD 7.43, 150 mM NaCl, 1 mM MnCl₂, 99.9% D₂O) at 23° C. After each labeling time (10 seconds, 10 minutes, and 4 hours) at 23° C., the labeling reaction was quenched with the addition of 19 μL of ice-cold quenching buffer (200 mM potassium phosphate, pH 2.44, 4 M guanidinium chloride, 0.72 M TCEP, H₂O) and held on ice for 1 minute prior to LC/MS analysis.

LC/MS: Deuterated and control samples were digested online at 15° C. using an in-house-packed pepsin column. The cooling chamber of the UPLC system, which housed all the chromatographic elements, was held at 0.0±0.1° C. for the entire time of the measurements. Peptides were trapped and desalted on a VanGuard Pre-Column trap [2.1 mm×5 mm, ACQUITY UPLC BEH C18, 1.7 μm (Waters, 186002346)] for 3 minutes at 100 μL/min, eluted from the trap using a 5%-35% gradient of acetonitrile over 10 minutes at a flow rate of 100 μL/min, and separated using an ACQUITY UPLC HSS T3, 1.8 μm, 1.0 mm×50 mm column (Waters, 186003535). Mass spectra were acquired using a Waters Synapt G2-Si HDMS^{E} mass spectrometer in ion mobility mode. A conventional electrospray source was used, and the instrument was scanned over the range 50 to 2000 m/z. The error of determining the deuterium levels was ±0.25 Da in this experimental setup. Deuterium levels were not corrected for back exchange and thus reported as relative (PMID 16208684)

HDX-MS data processing. Peptides were identified from replicate HDMS^{E} analyses (as detailed in the FIGS. 5E-5G) of undeuterated control samples using PLGS 3.0.1 (Waters Corporation). The peptides identified in PLGS were filtered and processed with DynamX 3.0 software (Waters Corporation). All spectra were inspected manually. The relative amount of deuterium in each peptide was determined with the software by subtracting the centroid mass of the undeuterated form of each peptide from the deuterated form, at each time point, for each condition. These deuterium uptake values were used to generate all uptake graphs and difference maps.

### Modeling and Molecular Dynamics Simulations

The best ranked AlphaFold model of the integrin αVβ5 heterodimer and the crystal structure of the irisin protein (PDB 4LSD) were subjected to individual molecular dynamics runs. Each protein was prepared using CHARMM-GUI's Solution Builder for the integrin αVβ5 model, as an initial step, Mn²⁺ ions were added to the model based on their approximate positions taken from the integrin αVβ3 crystal structure (PDB 1M1X). Adequate protonation was assigned using the ProteinPrepare webserver, at a pH of 7.4. The protein was placed in a rectangular TIP3P waterbox and neutralized, with an additional 150 mM NaCl added in order to properly replicate cellular conditions. The prepared system was minimized and equilibrated for 1 ns (NVT ensemble) and further subjected to a 360 ns production run (NPT ensemble, 298.15 K). From the resulting trajectory, 35 snapshots were extracted at equal intervals, using VMD. In the case of irisin (PDB 4LSD), we first removed all crystallographic water molecules and only kept chain A from the initial structure. The resulting structure was prepared using a similar protocol to integrin αVβ5, with a production run of 500 ns. The first 150 ns of the production run trajectory were discarded and then 35 snapshots were extracted at equal intervals from the remaining 350 ns. All MD simulations and further clustering were conducted using the AMBER 2018 with the FF14SB forcefield and AmberTools.

Chain B: Irisin amino acid sequence used for the
modeling (SEQ ID NO: 14):
MSPSAPVNVTVRHLKANSAVVSWDVLEDEVVIGFAISQQKKDVRMLRFIQ
EVNTTTRSCALWDLEEDTEYIVHVQAISIQGQSPASEPVLFKTPREAE
Chain A: Integrin aVB5 amino acid sequence used
for the modeling (B5 is in italic)(SEQ ID NO: 16):
MAFPPRRRLRLGPRGLPLLLSGLLLPLCRAFNLDVDSPAEYSGPEGSYFG
FAVDFFVPSASSRMFLLVGAPKANTTQPGIVEGGQVLKCDWSSTRRCQPI
EFDATGNRDYAKDDPLEFKSHQWFGASVRSKQDKILACAPLYHWRTEMKQ
EREPVGTCFLQDGTKTVEYAPCRSQDIDADGQGFCQGGFSIDFTKADRVL
LGGPGSFYWQGQLISDQVAEIVSKYDPNVYSIKYNNQLATRTAQAIFDDS
YLGYSVAVGDFNGDGIDDFVSGVPRAARTLGMVYIYDGKNMSSLYNFTGE
QMAAYFGFSVAATDINGDDYADVFIGAPLFMDRGSDGKLQEVGQVSVSLQ
RASGDFQTTKLNGFEVFARFGSAIAPLGDLDQDGFNDIAIAAPYGGEDKK
GIVYIFNGRSTGLNAVPSQILEGQWAARSMPPSFGYSMKGATDIDKNGYP
DLIVGAFGVDRAILYRARPVITVNAGLEVYPSILNQDNKTCSLPGTALKV
SCFNVRFCLKADGKGVLPRKLNFQVELLLDKLKQKGAIRRALFLYSRSPS
HSKNMTISRGGLMQCEELIAYLRDESEFRDKLTPITIFMEYRLDYRTAAD
TTGLQPILNQFTPANISRQAHILLDCGEDNVCKPKLEVSVDSDQKKIYIG
DDNPLTLIVKAQNQGEGAYEAELIVSIPLQADFIGVVRNNEALARLSCAF
KTENQTRQVVCDLGNPMKAGTQLLAGLRFSVHQQSEMDTSVKFDLQIQSS
NLFDKVSPVVSHKVDLAVSPASSFHVLRSLPLSSKGSGSAGWDVIQMTPQ
EIAVNLRPGDKTTFQLQVRQVEDYPVDLYYLMDLSLSMKDDLDNIRSLGT
KLAEEMRKLTSNFRLGFGSFVDKDISPFSYTAPRYQTNPCIGYKLFPNCV
PSFGFRHLLPLTDRVDSFNEEVRKQRVSRNRDAPEGGFDAVLQAAVCKEK
IGWRKDALHLLVFTTDDVPHIALDGKLGGLVQPHDGQCHLNEANEYTASN
QMDYPSLALLGEKLAENNINLIFAVTKNHYMLYKNFTALIPGTTVEILDG
DSKNIIQLIINAYNSIRSKVELSVWDQPEDLNLFFTATCQDGVSYPGQRK
CEGLKIGDTASFEVSLEARSCPSRHTEHVFALRPVGFRDSLEVGVTYNCT
C

### Docking

The resulting snapshots (35 for each protein) were further prepared using PDBTools in order to generate proper ensembles and submitted for docking to the HADDOCK webserver. Docking was performed on the 2 resulting ensembles using the default parameters provided by the Guru interface: in the case of integrin αVβ5, active residues were defined based on the provided HDX-MS data. Passive residues were selected automatically by HADDOCK, based on the proximity to active residues. In the case of irisin, all residues in the structure were defined as active.

HADDOCK clustered 44 structures in 9 cluster(s), which represents 22.0% of the water-refined models HADDOCK generated. Currently the maximum number of models considered for clustering is 200. The statistics of the top 10 clusters are shown in Table 3 below. The top cluster is the most reliable according to HADDOCK. Its Z-score indicates how many standard deviations from the average this cluster is located in terms of score (the more negative the better).

TABLE 3
Statistics of the top 10 HADDOCK clusters

| CLUSTER 2 | |
|---|---|
| HADDOCK score | 141.0 +/− 26.1 |
| Cluster size | 7 |
| RMSD from the overall lowest-energy structure | 13.3 +/− 0.7 |
| Van der Waals energy | −93.6 +/− 4.8 |
| Electrostatic energy | −337.1 +/− 20.2 |
| Desolvation energy | 30.2 +/− 12.3 |
| Restraints violation energy | 2719.3 +/− 168.76 |
| Buried Surface Area | 3039.1 +/− 133.1 |
| Z-Score | −1.5 |
| CLUSTER 9 | |
| HADDOCK score | 150.0 +/− 21.1 |
| Cluster size | 4 |
| RMSD from the overall lowest-energy structure | 11.6 +/− 0.1 |
| Van der Waals energy | −109.0 +/− 16.4 |
| Electrostatic energy | −252.4 +/− 120.7 |
| Desolvation energy | 25.2 +/− 12.3 |
| Restraints violation energy | 2843.0 +/− 227.96 |
| Buried Surface Area | 3023.5 +/− 193.2 |
| Z-Score | −0.9 |
| CLUSTER 7 | |
| HADDOCK score | 156.2 +/− 18.2 |
| Cluster size | 4 |
| RMSD from the overall lowest-energy structure | 12.7 +/− 0.2 |
| Van der Waals energy | −100.0 +/− 9.4 |
| Electrostatic energy | −309.9 +/− 51.5 |
| Desolvation energy | 38.0 +/− 7.4 |
| Restraints violation energy | 2802.0 +/− 82.23 |
| Buried Surface Area | 3053.5 +/− 248.4 |
| Z-Score | −0.5 |
| CLUSTER 6 | |
| HADDOCK score | 156.6 +/− 26.4 |
| Cluster size | 4 |
| RMSD from the overall lowest-energy structure | 12.5 +/− 0.6 |
| Van der Waals energy | −87.6 +/− 11.2 |
| Electrostatic energy | −337.9 +/− 59.4 |
| Desolvation energy | 28.0 +/− 12.9 |
| Restraints violation energy | 2838.2 +/− 165.88 |
| Buried Surface Area | 2827.1 +/− 213.0 |
| Z-Score | −0.4 |
| CLUSTER 8 | |
| HADDOCK score | 159.7 +/− 28.1 |
| Cluster size | 4 |
| RMSD from the overall lowest-energy structure | 7.9 +/− 0.2 |
| Van der Waals energy | −78.8 +/− 3.1 |
| Electrostatic energy | −282.6 +/− 60.6 |
| Desolvation energy | 22.2 +/− 17.3 |
| Restraints violation energy | 2728.0 +/− 235.56 |
| Buried Surface Area | 2550.6 +/− 166.5 |
| Z-Score | −0.2 |
| CLUSTER 3 | |
| HADDOCK score | 166.9 +/− 32.0 |
| Cluster size | 5 |
| RMSD from the overall lowest-energy structure | 12.2 +/− 0.2 |
| Van der Waals energy | −107.0 +/− 9.2 |
| Electrostatic energy | −157.3 +/− 38.9 |
| Desolvation energy | 24.2 +/− 4.8 |
| Restraints violation energy | 2811.2 +/− 277.79 |
| Buried Surface Area | 2989.5 +/− 162.7 |
| Z-Score | 0.3 |
| CLUSTER 1 | |
| HADDOCK score | 167.0 +/− 27.1 |
| Cluster size | 8 |
| RMSD from the overall lowest-energy structure | 13.4 +/− 0.9 |
| Van der Waals energy | −89.6 +/− 9.3 |
| Electrostatic energy | −399.9 +/− 97.1 |
| Desolvation energy | 50.4 +/− 5.1 |
| Restraints violation energy | 2861.5 +/− 100.80 |
| Buried Surface Area | 2838.6 +/− 271.0 |
| Z-Score | 0.3 |
| CLUSTER 4 | |
| HADDOCK score | 174.3 +/− 23.3 |
| Cluster size | 4 |
| RMSD from the overall lowest-energy structure | 12.5 +/− 0.3 |
| Van der Waals energy | −93.6 +/− 3.5 |
| Electrostatic energy | −222.7 +/− 62.2 |
| Desolvation energy | 25.6 +/− 9.5 |
| Restraints violation energy | 2868.7 +/− 231.87 |
| Buried Surface Area | 2698.5 +/− 106.0 |
| Z-Score | 0.8 |
| CLUSTER 5 | |
| HADDOCK score | 195.4 +/− 5.1 |
| Cluster size | 4 |
| RMSD from the overall lowest-energy structure | 12.9 +/− 0.9 |
| Van der Waals energy | −97.5 +/− 10.2 |
| Electrostatic energy | −227.8 +/− 11.7 |
| Desolvation energy | 40.8 +/− 11.5 |
| Restraints violation energy | 2976.4 +/− 85.20 |
| Buried Surface Area | 2700.5 +/− 161.1 |
| Z-Score | 2.2 |

### Final MD Refinement Stage

The best docking pose from the top ranked cluster (pose 1, cluster 2) was further subjected to a short MD simulation for an additional 100 ns to further optimize the αVβ5-irisin interaction. The resulting trajectory was clustered using AmberTools with the k-means clustering algorithm. The structures from the most populated clusters were visually inspected to select the final model of the αVβ5-irisin interaction.

### Statistical Analysis

All data are represented as mean±s.e.m. with at least 3 independent experiments. Fluorescence anisotropy binding curves were fit using KaleidaGraph 3.51. Statistical analysis was performed using GraphPad Prism 7. Differences between 2 means and among multiple means were assessed by unpaired two-tailed student t test and ANOVA, respectively. Assessments with P<0.05 were considered significant. For proteomic data, Statistical analysis was performed using Perseus (Tyanova, S., et al., The Perseus computational platform for comprehensive analysis of (prote)omics data. Nat Methods, 2016. 13(9): p. 731-40). Total quantified proteins were filtered to remove unreviewed TrREMBL sequences and proteins quantified using a single peptide. Significant changes were determined using a permutation-based FDR with the following settings—FDR—0.05, S0-0.1, and number of randomizations—250.

### Data Availability

The mass spectrometry proteomics data have been deposited to the ProteomeXchange Consortium via the PRIDE (PMID 34723319) partner repository with the dataset identifier PXD031982 for the secretome dataset and PXD035397 for HDX-MS dataset.
Reviewer account details:
For the secretome dataset
Username: reviewer_pxd031982@ebi.ac.uk
Password: zvStEV5L

### Example 2: Characterization of Irisin/Receptor Complex and the Role of Glycosylation on Irisin-Induced Biological Effects

In bone and fat, the effects of irisin are mediated via αv integrins, which use αVβ5 as their major receptor (Kim et al., Cell. 2018; 175(7):1756-68 e17). Irisin is glycosylated, and contains a fibronectin type III domain, which is relatively common and is shared by fibronectin plus many other protein (Bork et al., Proc Natl Acad Sci USA. 1992; 89(19):8990-4). However, irisin does not contain the RGD motif that has been identified in most integrin ligands as the key integrin-binding motif (Van Agthoven et al., Nat Struct Mol Biol. 2014; 21(4):383-8), indicating a non-canonical way of ligand binding for integrins. Recombinant irisin from mammalian cells exhibits a short half-life in vivo, and thus is not ideally suited as a direct treatment for gaining exercise-induced beneficial effects. Understanding how irisin interacts with and how it signals through integrin may reveal a novel mechanism of hormone-induced integrin signaling, and therapeutically, it will assist drug and antibody development to treat patients with obesity, aged-related diseases and neuro- or muscular degenerative disorders.

The protein PGC1α was initially identified as a transcriptional coregulator of PPARγ and other nuclear receptors which regulates mitochondrial biogenesis and adipose thermogenesis (Fernandez-Marcos, P. J. and J. Auwerx, Regulation of PGC-1alpha, a nodal regulator of mitochondrial biogenesis. Am J Clin Nutr, 2011. 93(4): p. 884S-90; Puigserver, P., et al., A cold-inducible coactivator of nuclear receptors linked to adaptive thermogenesis. Cell, 1998. 92(6): p. 829-39; Reutzel, M., et al., Cerebral Mitochondrial Function and Cognitive Performance during Aging: A Longitudinal Study in NMRI Mice. Oxid Med Cell Longev, 2020. 2020: p. 4060769; Uldry, M., et al., Complementary action of the PGC-1 coactivators in mitochondrial biogenesis and brown fat differentiation. Cell Metab, 2006. 3(5): p. 333-41). PGC1α is induced in the skeletal muscle of humans and rodents with exercise and it stimulates many important adaptations of muscle to exercise (Baar, K., et al., Adaptations of skeletal muscle to exercise: rapid increase in the transcriptional coactivator PGC-1. FASEB J, 2002. 16(14): p. 1879-86; Norrbom, J., et al., PGC-1alpha mRNA expression is influenced by metabolic perturbation in exercising human skeletal muscle. J Appl Physiol (1985), 2004. 96(1): p. 189-94; Correia, J. C., D. M. Ferreira, and J. L. Ruas, Intercellular: local and systemic actions of skeletal muscle PGC-1s. Trends Endocrinol Metab, 2015. 26(6): p. 305-14; Handschin, C., et al., Skeletal muscle fiber-type switching, exercise intolerance, and myopathy in PGC-1alpha muscle-specific knock-out animals. J Biol Chem, 2007. 282(41): p. 30014-21). PGC1a transgenic mice were then used as a platform for the discovery of muscle-secreted proteins regulated by PGC1α and exercise. These studies identified irisin, a cleaved and secreted product from a type-1 membrane protein FNDC5 (fibronectin-domain III (FNIII) containing 5), whose mRNA is increased upon forced PGC1α expression (Bostrom, P., et al., A PGC1-alpha-dependent myokine that drives brown-fat-like development of white fat and thermogenesis. Nature, 2012. 481(7382): p. 463-8).

The amino acid sequence of irisin is 100% conserved between mouse and human and both are heavily glycosylated. Irisin circulates in the blood as a 12 kDa (112 aa) polypeptide containing the entire N-terminal FNIII domain of FNDC5 (Bostrom, P., et al., A PGC1-alpha-dependent myokine that drives brown-fat-like development of white fat and thermogenesis. Nature, 2012. 481(7382): p. 463-8). Structural studies showed that the folding of irisin represents a typical FNIII domain (Schumacher, M. A., et al., The structure of irisin reveals a novel intersubunit beta-sheet fibronectin type III (FNIII) dimer: implications for receptor activation. J Biol Chem, 2013. 288(47): p. 33738-33744). The plasma levels of irisin, using absolute quantification by mass spectrometry, reveal that irisin circulates at typical polypeptide hormonal levels (3-5 ng/ml range). Exercise elevates irisin circulating concentrations in both humans and mice (Jedrychowski, M. P., et al., Detection and Quantitation of Circulating Human Irisin by Tandem Mass Spectrometry. Cell Metab, 2015. 22(4): p. 734-740; Kim, H., et al., Irisin Mediates Effects on Bone and Fat via alphaV Integrin Receptors. Cell, 2018. 175(7): p. 1756-1768 e17; Lee, P., et al., Irisin and FGF21 are cold-induced endocrine activators of brown fat function in humans. Cell Metab, 2014. 19(2): p. 302-9). Previous studies revealed a variety of biological effects mediated by irisin. Peripherally, irisin causes “browning” of subcutaneous adipose tissue (beige fat) and promotes bone resorption and bone remodeling (Bostrom, P., et al. Nature, 2012. 481(7382): p. 463-8; Kim, H., et al. Cell, 2018. 175(7): p. 1756-1768 e17). Most importantly, peripherally administrated irisin crosses the blood brain barrier (BBB) where it improves cognition and resistance to neurodegeneration in several animal models (Islam, M. R., et al., Exercise hormone irisin is a critical regulator of cognitive function. Nat Metab, 2021. 3(8): p. 1058-1070). In bone, fat and hippocampus, irisin appears to function primarily via αV integrin receptors (Kim, H., et al. Cell, 2018. 175(7): p. 1756-1768 e17; Islam, M. R., et al., Nat Metab, 2021. 3(8): p. 1058-1070). Since interactions between integrins and their typical large ligands, such as ECM protein, are usually rather complex, how a small protein like irisin can interact with and function through an integrin receptor is not clear. Additionally, irisin has certain bioactivities, such as effects on cognition, not typically associated with integrins.

Integrins are heterodimeric membrane receptors composed of noncovalently associated α and β subunits that bind extracellular matrix proteins, such as fibronectin, or counter receptors, such as I-CAM. In doing so, they mediate cell-matrix or cell-cell adhesion, respectively (Hynes, R. O., Integrins: bidirectional, allosteric signaling machines. Cell, 2002. 110(6): p. 673-87). 18 α and 8 β subunits have been identified in vertebrates that assemble at least 24 integrin known heterodimers with different ligand specificities and signaling properties (Hynes, R. O. Cell, 2002. 110(6): p. 673-87; Kadry, Y. A. and D. A. Calderwood, Chapter 22: Structural and signaling functions of integrins. Biochim Biophys Acta Biomembr, 2020. 1862(5): p. 183206). Integrins are composed of a large ectodomain responsible for ligand binding, a single-pass transmembrane domain and a short cytoplasmic tail (Campbell, I. D. and M. J. Humphries, Integrin structure, activation, and interactions. Cold Spring Harb Perspect Biol, 2011. 3(3)). Integrins populate an ensemble of at least three conformational states with different affinity for their ligands: low affinity-closed state, extended closed state, and a high-affinity open state (Springer, T. A. and M. L. Dustin, Integrin inside-out signaling and the immunological synapse. Curr Opin Cell Biol, 2012. 24(1): p. 107-15).

On the surface of the cells under resting conditions, the energy landscape favors the closed state (>99%) and maintains only a very small fraction (˜0.1%) of the integrins in the open confirmation (Li, J., et al., Conformational equilibria and intrinsic affinities define integrin activation. EMBO J, 2017. 36(5): p. 629-645; Su, Y., et al., Relating conformation to function in integrin alpha5beta1. Proc Natl Acad Sci USA, 2016. 113(27): p. E3872-81; Li, J. and T. A. Springer, Energy landscape differences among integrins establish the framework for understanding activation. J Cell Biol, 2018. 217(1): p. 397-412; Li, J. and T. A. Springer, Integrin extension enables ultrasensitive regulation by cytoskeletal force. Proc Natl Acad Sci USA, 2017. 114(18): p. 4685-4690). Therefore, integrins need to be activated for high-affinity extracellular ligand binding. The mechanism of integrin activation is complex, and the prevailing view of this indicates that the interplay between the intrinsic integrin thermodynamic equilibrium and the intracellular adaptors that target the integrin cytoplasmic tail is crucial (Sun, Z., M. Costell, and R. Fassler, Integrin activation by talin, kindlin and mechanical forces. Nat Cell Biol, 2019. 21(1): p. 25-31). However, extracellular factors that could mediate integrin activation, to our knowledge, have not been identified. The conformation and ligand-binding affinity of integrin can be affected by metal ions which bind integrin at the metal-ion dependent adhesion site (MIDAS) (Xiong, J. P., et al., Crystal structure of the extracellular segment of integrin alpha Vbeta3. Science, 2001. 294(5541): p. 339-45; Xiao, T., et al., Structural basis for allostery in integrins and binding to fibrinogen-mimetic therapeutics. Nature, 2004. 432(7013): p. 59-67; Anderson, J. M., J. Li, and T. A. Springer, Regulation of integrin alpha5beta1 conformational states and intrinsic affinities by metal ions and the ADMIDAS. Mol Biol Cell, 2022. 33(6): p. ar56). Of note, Mn2+, through a rather unclear mechanism, shifts integrins to their open states and maintains integrins in their fully activated, high-affinity confirmations (Anderson, J. M., J. Li, and T. A. Springer. Mol Biol Cell, 2022. 33(6): p. ar56).

αV integrins are one of the integrin families that lack the inserted αI domain and recognize the RGD motif—a conserved recognition sequence shared by many integrin ligands (Huhtala, M., et al., Integrin evolution: insights from ascidian and teleost fish genomes. Matrix Biol, 2005. 24(2): p. 83-95). For the FNIII domain-containing integrin ligands, the RGD motif typically resides in a flexible loop that forms a small interface with both α and β integrin heads at the RGD-binding site (Xiao, T., et al. Nature, 2004. 432(7013): p. 59-67; Xiong, J. P., et al., Crystal structure of the extracellular segment of integrin alpha Vbeta3 in complex with an Arg-Gly-Asp ligand. Science, 2002. 296(5565): p. 151-5). However, irisin is composed of a single FNIII domain without an RGD motif (Schumacher, M. A., et al. J Biol Chem, 2013. 288(47): p. 33738-33744); furthermore, irisin lacks additional domains that often help ligands increase their affinities for integrins. Since irisin circulates at fairly low concentrations (single digit ng/ml), so an additional question arises as to how the interaction between irisin and its integrin receptor(s) could be robust enough to drive the conformational changes required for signal activation.

We have now identified a new component of the irisin mediated interaction with the integrin receptor. We show that extracellular Hsp90α (eHsp90α) is an exercise-induced factor that mediates integrin activation via direct binding to the ectodomain of αVβ5. The αVβ5 receptor, activated by eHsp90a, has a very high affinity (Kdapp of ˜30 nM) for irisin. Through biophysical and biochemical experiments, refined by multiple steps of MD simulations, we were able to generate and refine a docking model with 2.98 Å RMSD of the irisin/αVβ5 complex. This structure has novel implications for integrin-small ligand dynamics, and suggests how irisin mediates its effects.

To show that Hsp90α enhances irisin-induced myogenesis of skeletal muscle cells and the “beiging” of inguinal white adipocytes, the role of Hsp90α in irisin-induced biological effects was uncovered by (a) testing the role of Hsp90α in irisin-mediated effects on cultured muscle and fat cells; and (b) testing how Hsp90α influences irisin-mediated “beiging” of white adipose tissue (WAT) in vivo in mice.

To show that glycosylation influences the interaction between irisin and integrin and/or irisin and Hsp90α and thereby regulates irisin-induced integrin signaling, the effects of glycosylation on irisin were established by (a) generating irisin glycosylation (N to Q) mutant recombinant proteins and evaluating their stability and their affinity for the integrin/Hsp90α complex; (b) assessing how glycosylation affects irisin-mediated integrin signaling; (c) measuring the half-lives of recombinant WT irisin and of its glycosylation mutants in mice.

eHsp90α is Required for Irisin Binding to Integrin αVP5

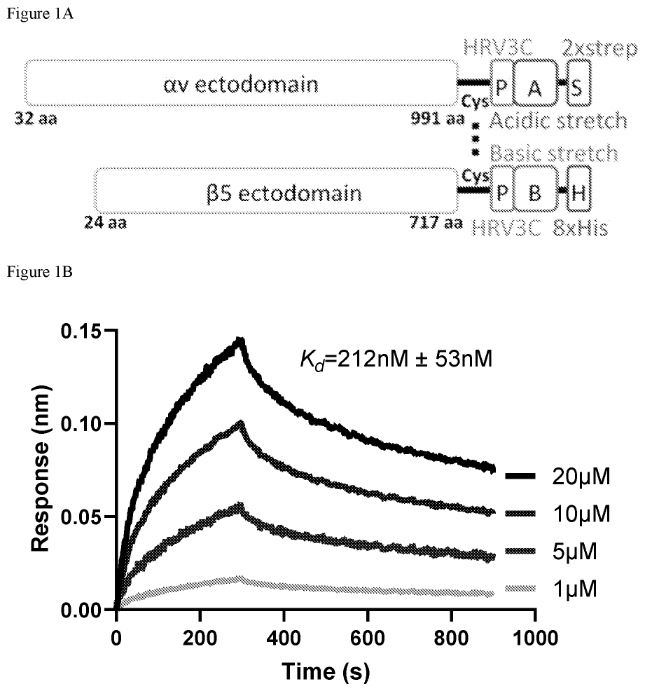

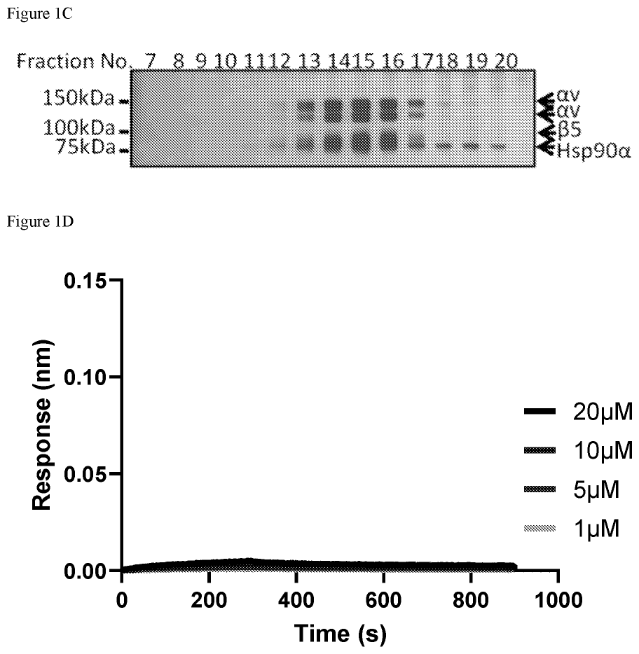

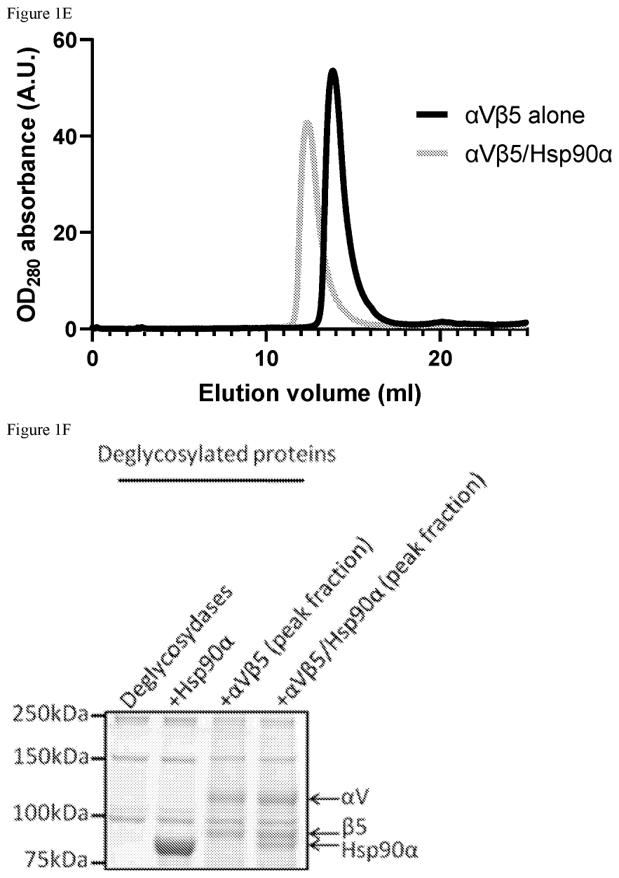

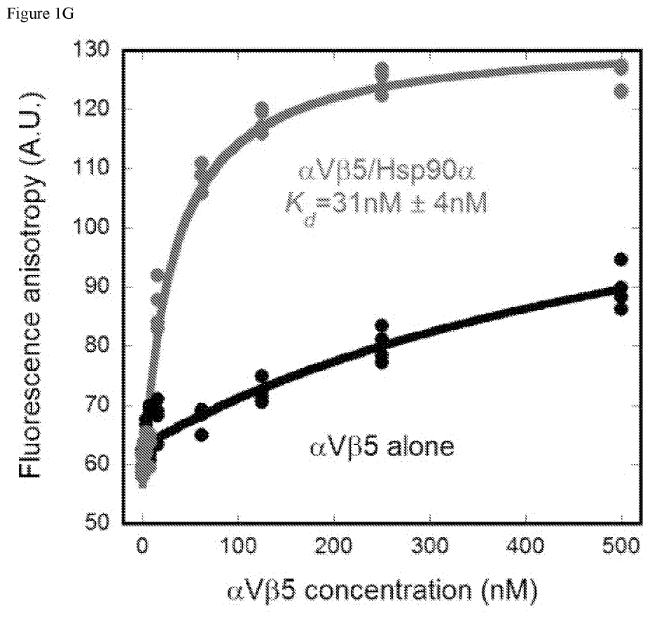

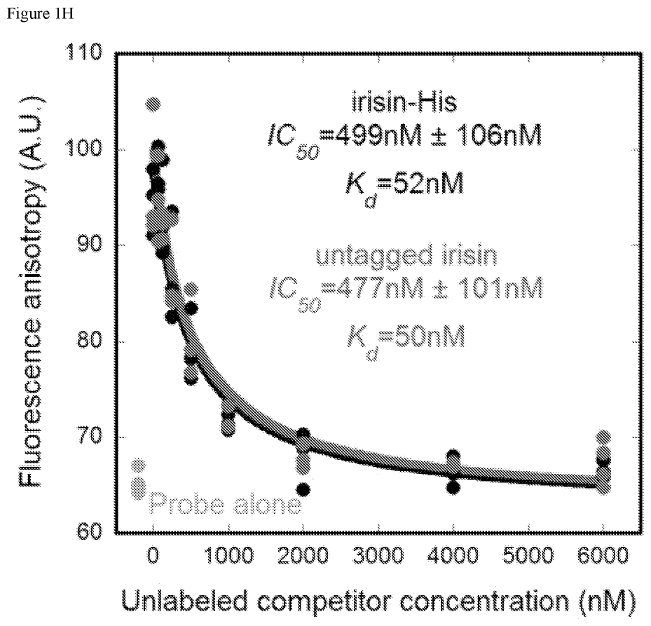

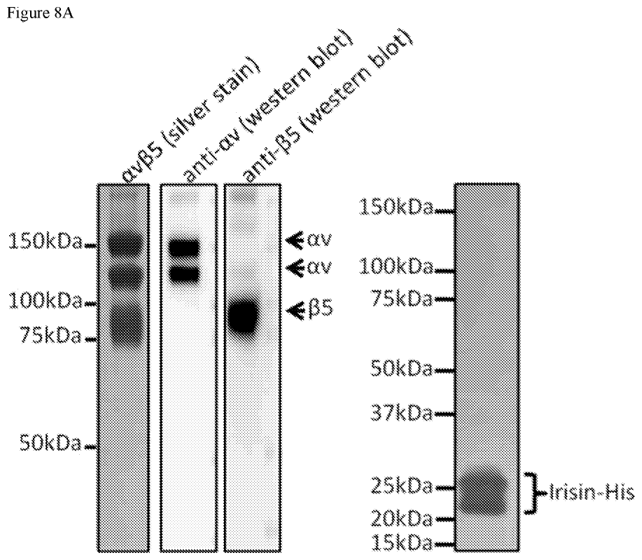

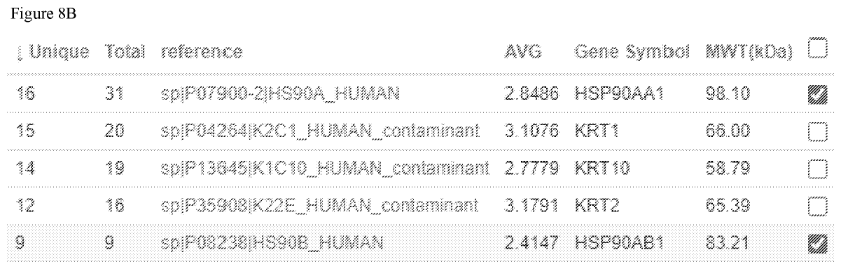

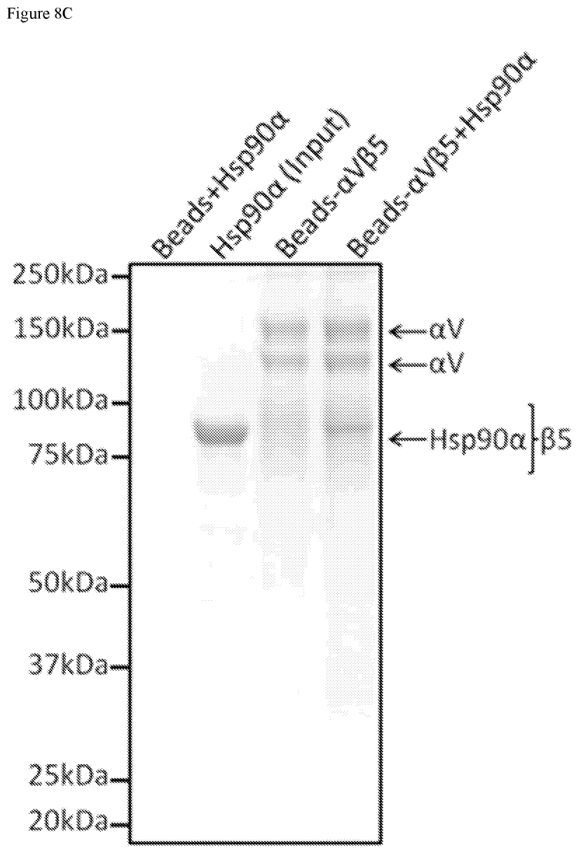

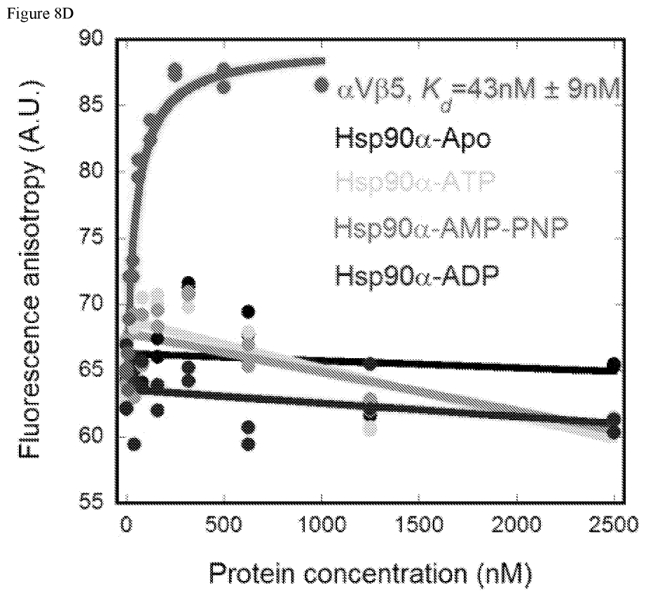

To biochemically and biophysically characterize the interaction between irisin and integrin αVβ5, we expressed the ectodomain of this integrin and adapted the previously developed constructs for producing recombinant αVβ5 ectodomain. This integrin heterodimer was prepared as a C-terminally clasped, affinity tagged fusion protein from mammalian HEK293 cells (FIG. 1A) (Nishida, N., et al., Activation of leukocyte beta2 integrins by conversion from bent to extended conformations. Immunity, 2006. 25(4): p. 583-94). The affinity-purified clasped αVβ5 was subsequently subjected to HRV3C protease cleavage, to generate the unclasped and untagged form (FIG. 1A). Both subunits of the recombinant αVβ5 ectodomain are glycosylated with heterogeneous glycans, which can be removed by glycosidase treatment (FIG. 8A and FIG. 1F). Wild-type, glycosylated recombinant irisin (His tagged or untagged) was produced from mammalian HEK293 cells as previously described (see FIG. 8A and Kim, H., et al. Cell, 2018. 175(7): p. 1756-1768 e17).

Next, Bio-Layer Interferometry (BLI) was used to analyze the binding of irisin to αVβ5, and yielded an apparent dissociation constant (K_{d}^{app}) of 212 nM±53 nM (FIG. 1B). To improve the purity of the recombinant αVβ5 ectodomain for structural analysis, we applied additional chromatographic steps and separated a “contaminating” protein which had a similar molecular mass (˜90 kDa) to the β5 subunit (FIG. 1C). Notably, the binding affinity of irisin for this more highly purified αVβ5 was almost eliminated (FIG. 1D). This result may be explained if one (or more) proteins that initially co-purified with the αVβ5 were necessary for high-affinity binding between irisin and αVβ5. Using mass spectrometry, we identified a major protein associated with the initial αVβ5 preparations as Hsp90α (FIG. 1C and FIG. 8B).

To determine whether Hsp90α alone binds αVβ5 directly, recombinant human Hsp90α was purified from E. coli and was used in a biochemical pull-down experiment. Clasped αVβ5 or control peptide containing only the tagged dimerization motifs was immobilized and incubated with an equal molar concentration of Hsp90a. The Hsp90α co-precipitated with αVβ5, but not with the control peptide (FIG. 8C). We then forced the maximal stoichiometry of the αVβ5/Hsp90α complex by adding 20-fold molar excess of Hsp90α, followed by extensive washing steps to remove the unbound Hsp90α. The αVβ5 and its bound Hsp90α were eluted off the beads by HRV3C protease cleavage. The reconstituted αVβ5/Hsp90α eluted as a stable high-mass particle by gel filtration chromatography, compared to αVβ5 alone (FIG. 1E). The peak fractions of αVβ5/Hsp90α and αVβ5 alone were deglycosylated, and quantification of the protein bands in the complex peak fraction revealed an Hsp90α:αV:β5 ratio of ˜1:1:1 (FIG. 1F).

These data suggest that Hsp90α might facilitate the interaction between irisin and αVβ5. To measure the binding affinity in solution, we used the polarization of fluorescently labeled irisin (A488-irisin) to examine its binding to either αVβ5 or the αVβ5/Hsp90α complex. In contrast to αVβ5 alone, which showed a very low affinity for irisin, the αVβ5/Hsp90α complex binds irisin with much higher affinity (K_{d}^{app} of 31 nM±4 nM) (FIG. 1G). To determine whether the A488 label or the His tag (fused to irisin) contributed to the binding, we used competition assays in which fixed concentrations of A488-irisin and αVβ5/Hsp90α were mixed with varying concentrations of unlabeled His-tagged irisin (irisin-His), or unlabeled and untagged irisin. Irisin-His and untagged irisin showed similar affinity for the αVβ5/Hsp90α complex, with K_{d}^{app} of 52 nM and 50 nM, respectively (FIG. 1H). Taken together, Hsp90α was identified as an extracellular factor from cultured mammalian cells that mediates irisin/αVβ5 interaction by associating directly with αVβ5.

eHsp90a Level is Increased with Exercise in Muscle Extracellular Fluid and in Plasma

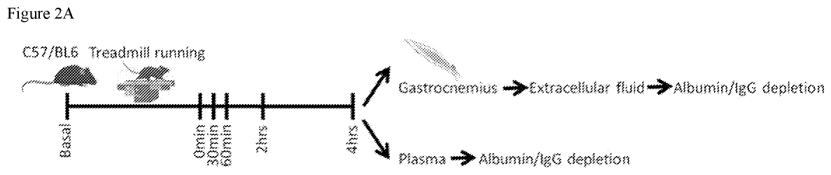

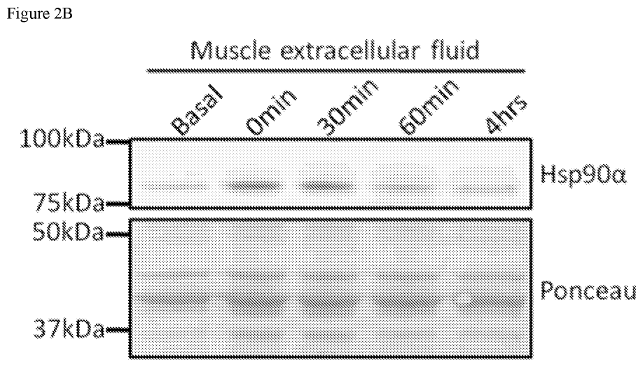

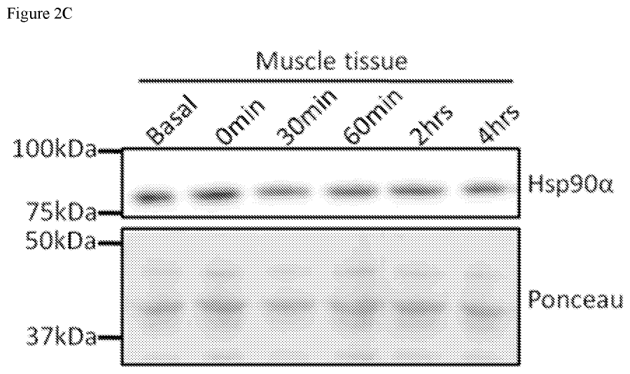

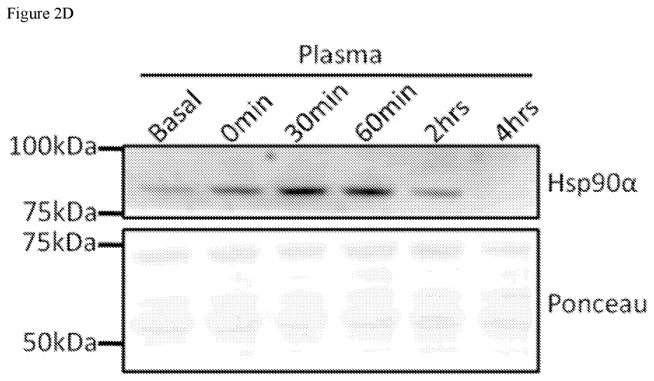

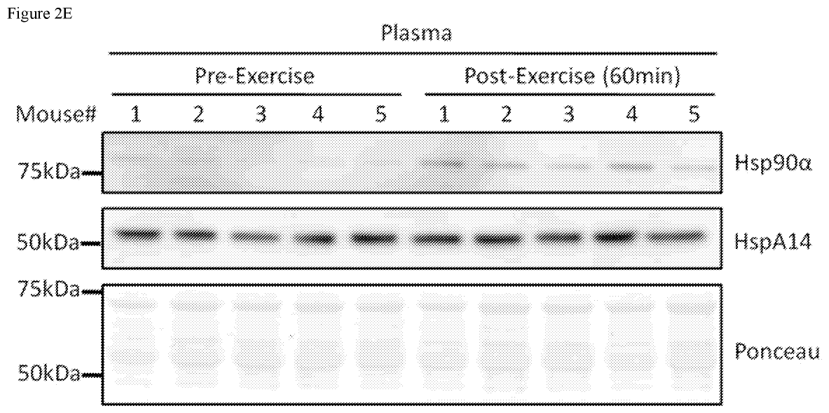

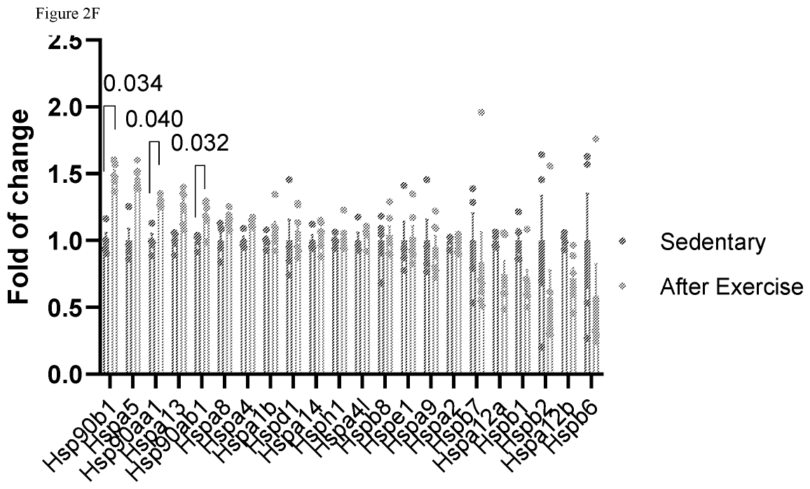

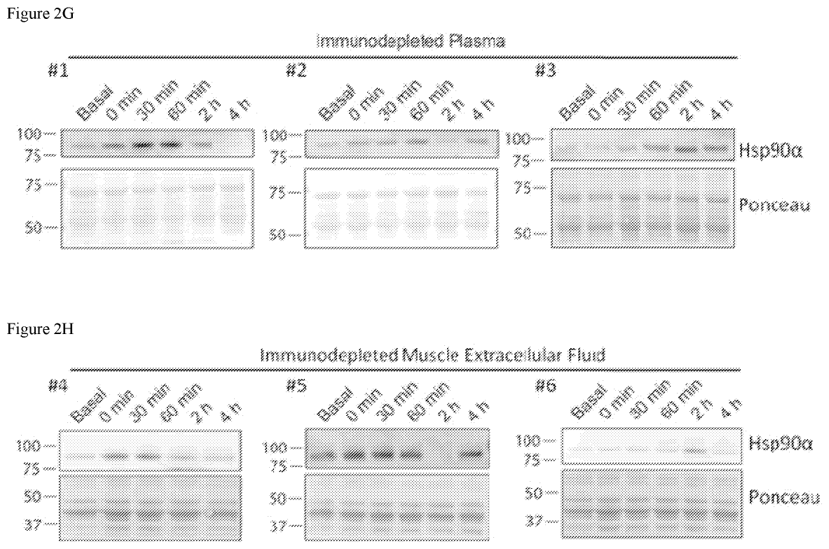

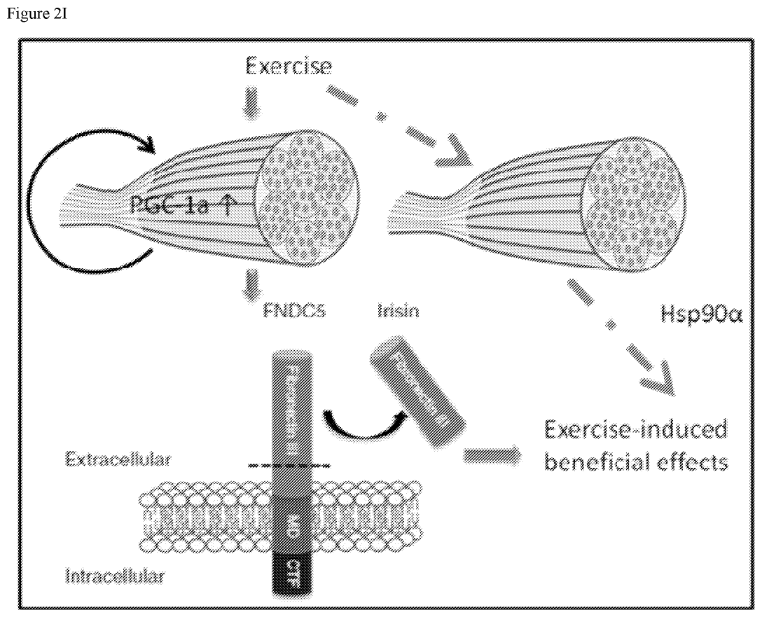

Hsp90α does not contain an N-terminal signal sequence, and is released from cells through an unconventional secretion mechanism (Wang, X., et al., The regulatory mechanism ofHsp90alpha secretion and its function in tumor malignancy. Proc Natl Acad Sci USA, 2009. 106(50): p. 21288-93; McCready, J., et al., Secretion of extracellular hsp90alpha via exosomes increases cancer cell motility: a role for plasminogen activation. BMC Cancer, 2010. 10: p. 294). eHsp90α binds a number of cell surface receptors, and modulates their downstream signaling pathways through as yet unidentified mechanisms (Gopal, U., et al., A novel extracellular Hsp90 mediated co-receptor function for LRPJ regulates EphA2 dependent glioblastoma cell invasion. PLoS One, 2011. 6(3): p. e17649; Murshid, A., J. Gong, and S. K. Calderwood, Heat shock protein 90 mediates efficient antigen cross presentation through the scavenger receptor expressed by endothelial cells-I. J Immunol, 2010. 185(5): p. 2903-17; Sidera, K., et al., A critical role for HSP90 in cancer cell invasion involves interaction with the extracellular domain of HER-2. J Biol Chem, 2008. 283(4): p. 2031-41). Since irisin levels increase with exercise (Jedrychowski, M. P., et al. Cell Metab, 2015. 22(4): p. 734-740; Kim, H., et al., Cell, 2018. 175(7): p. 1756-1768 e17; Lee, P., et al. Cell Metab, 2014. 19(2): p. 302-9), we evaluated eHsp90α levels in response to a single, intense bout of exercise in mice. To harvest the interstitial fluids (IF) of muscle, we adapted a technique that was used previously for analyzing metabolites of the gastrocnemius muscle (FIG. 2A and Spinelli, J. B., et al., Metabolic recycling of ammonia via glutamate dehydrogenase supports breast cancer biomass. Science, 2017. 358(6365): p. 941-946; Wiig, H., K. Aukland, and O. Tenstad, Isolation of interstitial fluid from rat mammary tumors by a centrifugation method. Am J Physiol Heart Circ Physiol, 2003. 284(1): p. H416-24; Sullivan, M R., et al., Quantification of microenvironmental metabolites in murine cancers reveals determinants of tumor nutrient availability. Elife, 2019. 8; Reddy, A., et al., pH-Gated Succinate Secretion Regulates Muscle Remodeling in Response to Exercise. Cell, 2020. 183(1): p. 62-75 e17). We found that levels of eHsp90α protein in the IF samples were elevated with exercise, while the total levels of Hsp90α protein within muscle tissue remained unchanged (FIGS. 2B and 2C). Interestingly, eHsp90α protein levels were also upregulated in plasma taken from mice that had rested for different amounts of time post-exercise (FIG. 2D). This is in contrast to another extracellular heat shock chaperone protein, HspA14, whose levels remain constant before and after exercise (FIG. 2E).

To ensure that the elevation of eHsp90α in IF is a specific, exercise-mediated regulatory process, rather than a nonspecific release of cellular content from damaged cells, we studied the acute exercise-induced muscle-specific secretome dataset (by FDR analysis) and found that only 3 out of 22 identified chaperone proteins, including Hsp90α (encoded by Hspaa1), were significantly upregulated in the IF of the exercised mice compared to the sedentary group (FIG. 2F).

eHsp90a is Required for Optimal Cellular Actions of Irisin

We then tested the role of Hsp90α in the binding of irisin to αVβ5 in live cells, along with certain irisin-mediated cellular effects. Cultured HEK293T cells, with or without forced expression of integrin αV and β5 subunits, were used in gain-of-function experiments, as previously described (Kim, H., et al., Cell, 2018. 175(7): p. 1756-1768 e17), and to assess irisin binding to the transfected cells using A488-irisin

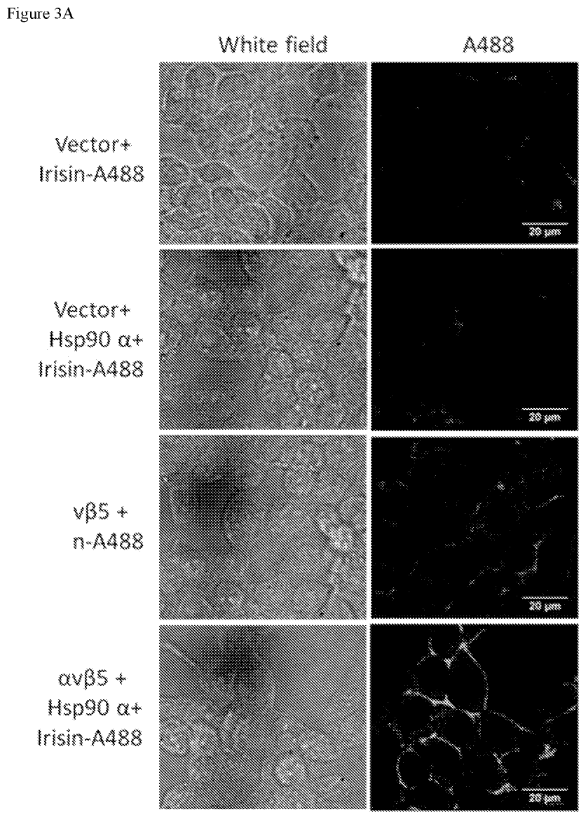

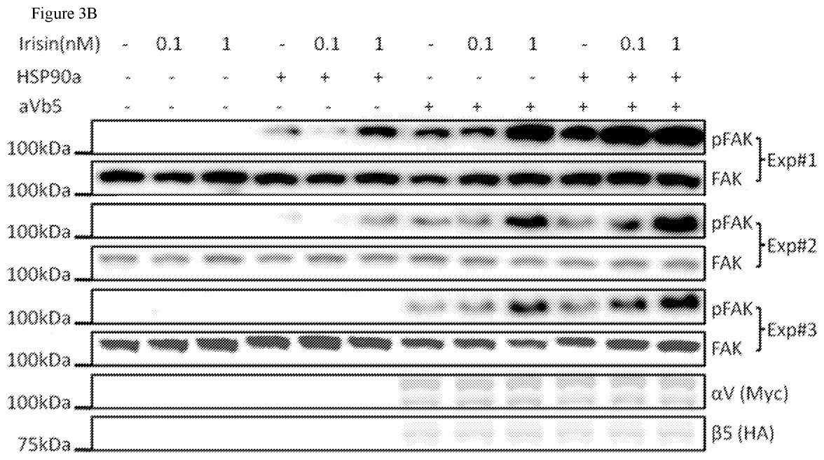

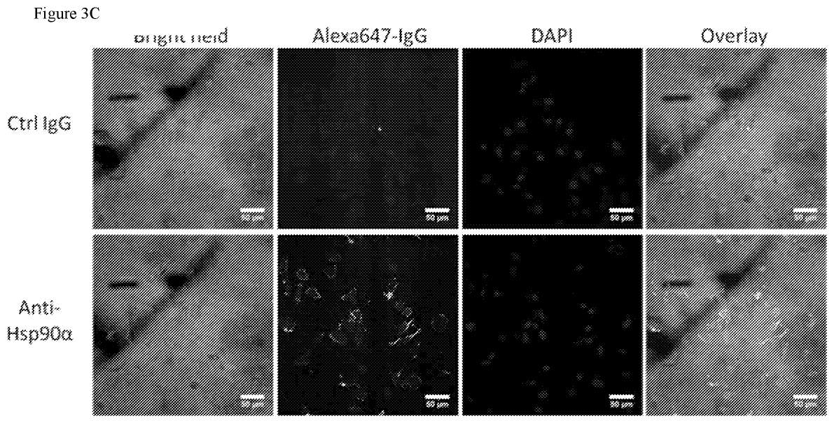

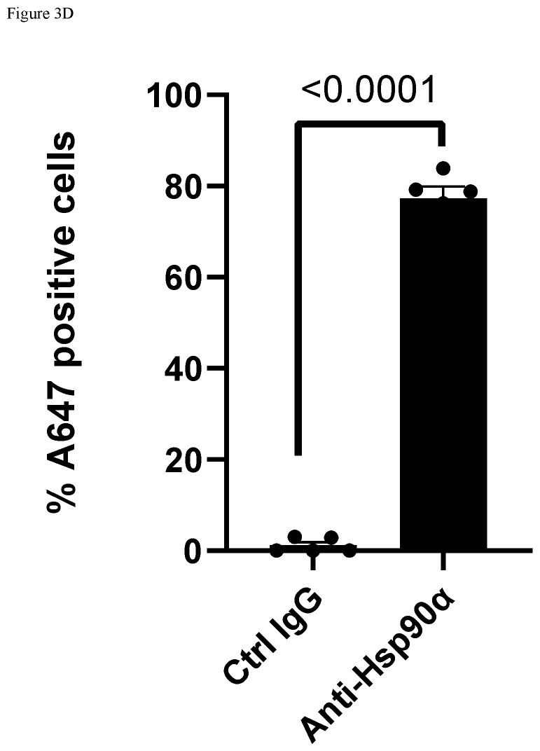

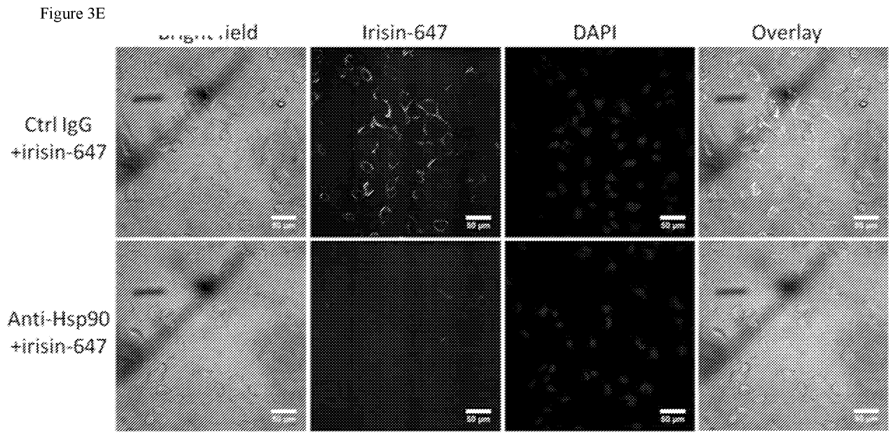

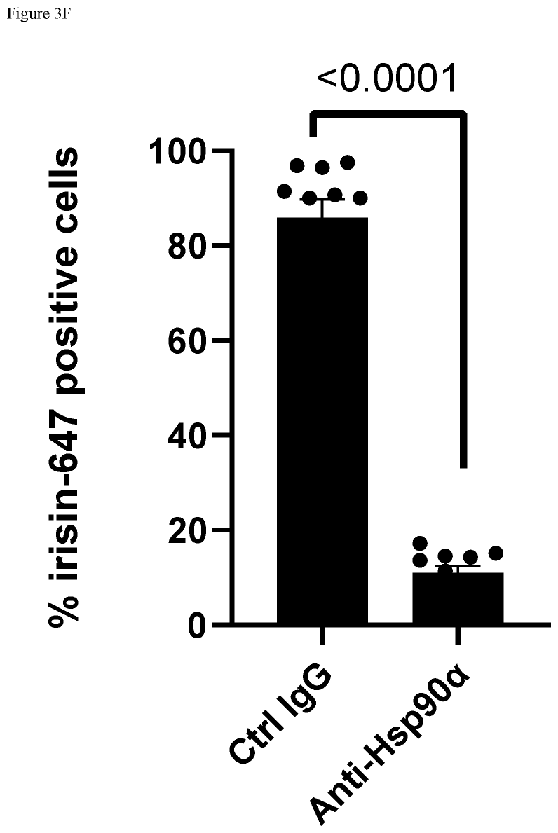

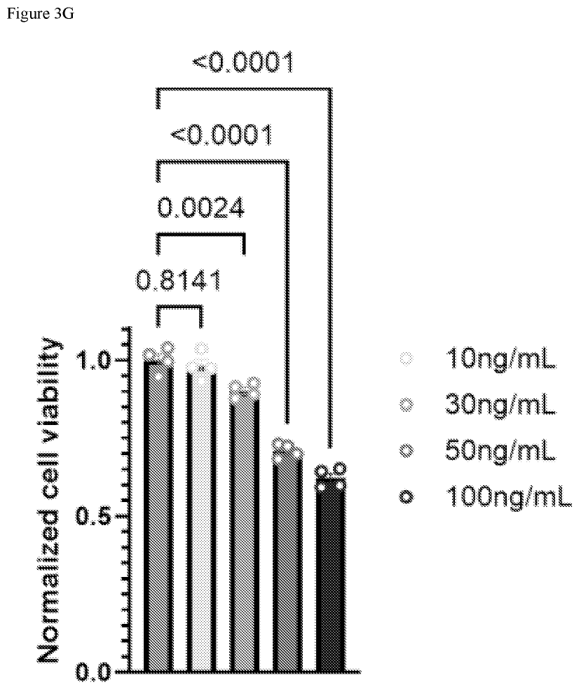

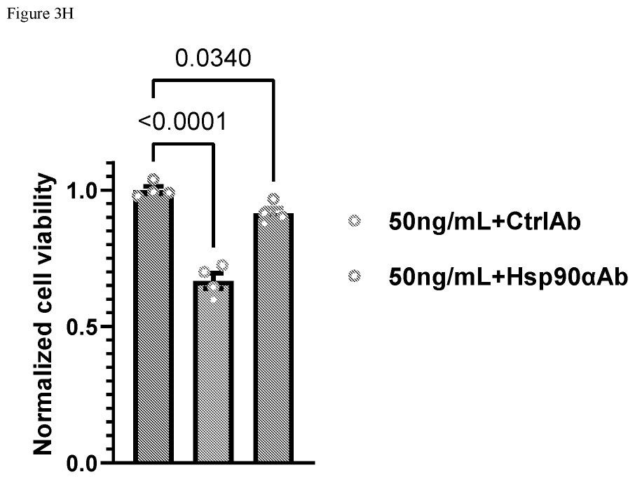

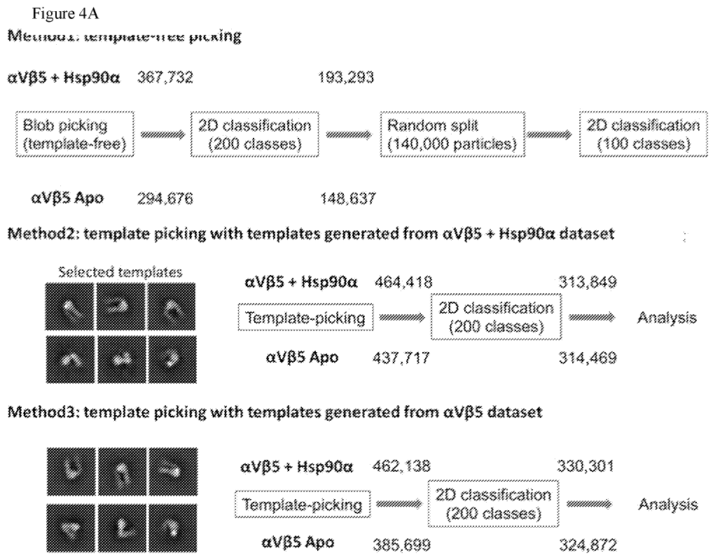

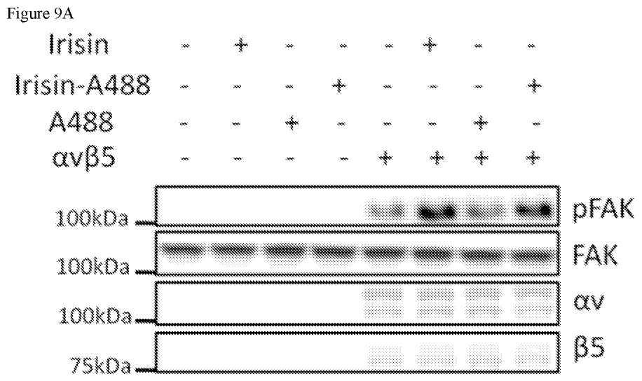

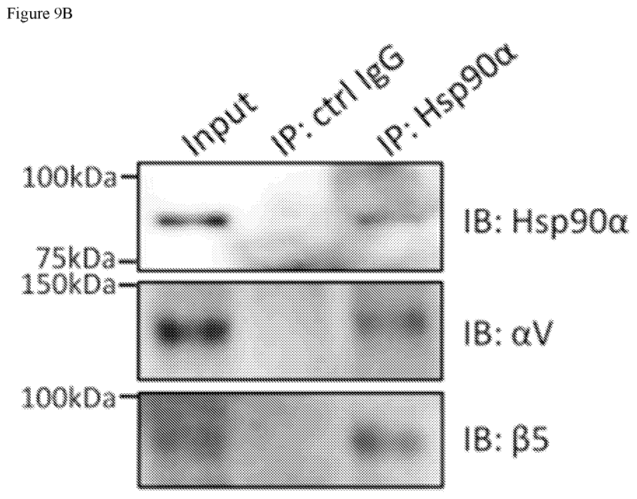

Cellular bioactivity of the fluorescently labeled irisin was validated by treating HEK293T cells transfected with control plasmid or full-length αV and β5 plasmids with unlabeled irisin, fluorophore A488 alone, or A488-irisin. Irisin-induced integrin signaling was tested by probing the canonical FAK phosphorylation, as shown previously (Kim, H., et al., Cell, 2018. 175(7): p. 1756-1768 e17). A488-irisin plus unlabeled irisin (but not A488 alone) induced similar integrin signaling in cells that ectopically expressed αV and β5 subunits (FIG. 9A). Irisin binding was also examined in live cells by tracing A488 fluorescence using confocal microscopy; A488-irisin binding was detected in cells that expressed αVβ5 ectopically, but not in the cells transfected with control plasmid. Importantly, this binding was significantly enhanced in cells that were pretreated with recombinant Hsp90α (FIG. 3A). This same cellular system was then used to investigate integrin signaling. These data confirmed that irisin treatment induced little integrin signaling in control HEK293T cells, but cells that ectopically expressed αVβ5 showed irisin-induced phosphorylation of FAK in a dose-dependent manner (FIG. 3B). Of note, pretreatment with recombinant Hsp90α shifted the dose response leftwards. A maximum pFAK signal was induced by 1.0 nM irisin in cells not receiving Hsp90α, but 0.1 nM was sufficient to stimulate a maximum signal in cells with added external HSP90α (FIG. 3B).

eHsp90α is present on the surface of many cell types, and mediates specific cellular functions, such as melanoma migration (Li, W., et al., Extracellular heat shock protein-90alpha: linking hypoxia to skin cell motility and wound healing. EMBO J, 2007. 26(5): p. 1221-33; Sidera, K., et al., Involvement of cell surface HSP90 in cell migration reveals a novel role in the developing nervous system. J Biol Chem, 2004. 279(44): p. 45379-88; Stellas, D., A. Karameris, and E. Patsavoudi, Monoclonal antibody 4C5 immunostains human melanomas and inhibits melanoma cell invasion and metastasis. Clin Cancer Res, 2007. 13(6): p. 1831-8). αV integrins are expressed in many melanoma cell lines, and αVβ5 is on the surface of a variety of melanoma cells that are highly metastatic (Danen, E. H., et al., Alpha v-integrins in human melanoma: gain of alpha v beta 3 and loss of alpha v beta 5 are related to tumor progression in situ but not to metastatic capacity of cell lines in nude mice. Int J Cancer, 1995. 61(4): p. 491-6). However, the relation between eHsp90α and αVβ5, as well as their molecular functions in melanoma cells, have not been addressed yet. We chose human melanoma cells (SK-Mel2) to explore the role of the endogenous eHsp90α in irisin binding and irisin-mediated cellular effects. Immunofluorescent staining was used to confirm the expression of cell surface eHsp90α on SK-Mel2 cells. Live cells were chilled on ice and incubated with Hsp90α antibody to visualize the Hsp90α on the plasma membrane. Cell surface eHsp90α was detected on more than 70% of the cells (FIGS. 3C and 3D). Then we tested the cellular interaction between eHsp90α and integrin αVβ5 on the cell surface using co-immunoprecipitation. Both αV and β5 subunits co-immunoprecipitated with cell surface eHsp90α (FIG. 9B). Next, to examine the binding of irisin to SK-Mel2 cells, A647-irisin, instead of A488-irisin, was used to avoid the high background signal generated by melanoma cellular autofluorescence. More than 80% of the SK-Mel2 cells pretreated with a control antibody showed irisin binding, and pre-treatment with anti-Hsp90α antibody significantly reduced the number of A647-irisin-positive cells to ˜10% (FIGS. 3E and 3F). Finally, we assessed the viability of SK-Mel2 cells in response to irisin treatment. Crystal violet staining revealed a dose-dependent irisin-mediated reduction in cell viability; treatment with 30˜100 ng/mL irisin led to 10˜40% reduction of the cell viability, respectively. Importantly, this reduction of viability could be inhibited by pre-treatment with an Hsp90α antibody but not with a control antibody (FIGS. 3G and 3H).

Taken together, these data indicate a role for exogenous and endogenous Hsp90α in irisin actions in cultured cells.

### Hsp90a Activates αVP5 for Irisin Binding

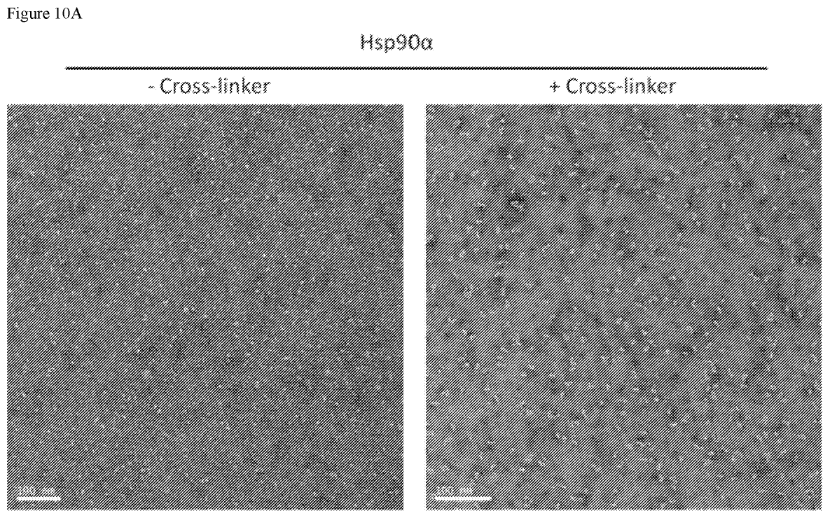

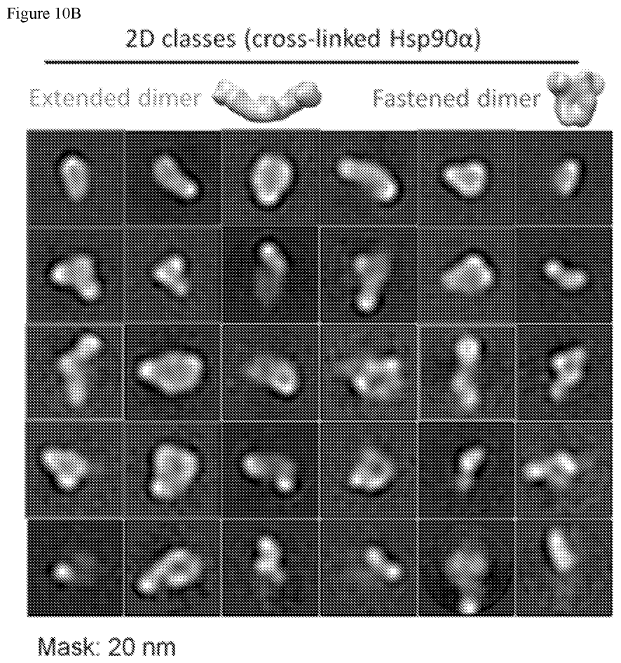

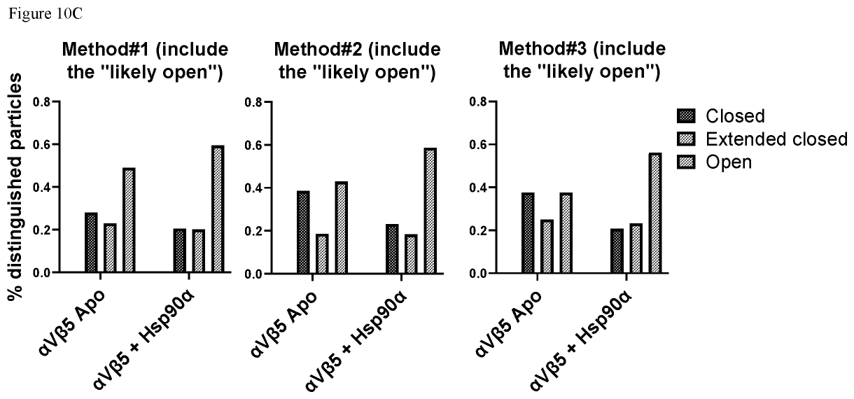

αV integrins often exist in a “closed” state, a state that is not permissive for ligand binding (Arnaout, M. A., Integrin structure: new twists and turns in dynamic cell adhesion. Immunol Rev, 2002. 186: p. 125-40). Most well-studied integrin ligands are very large proteins, like fibronectin, that contain separate domains providing additional integrin binding sites (the so called “synergy sites”) to increase the ligand binding affinity. The improved affinity is important for shifting the integrin thermodynamic equilibrium to favor the open state, allowing efficient RGD-mediated integrin signaling (Sechler, J. L., S. A. Corbett, and J. E. Schwarzbauer, Modulatory roles for integrin activation and the synergy site of fibronectin during matrix assembly. Mol Biol Cell, 1997. 8(12): p. 2563-73). In theory, Hsp90α could facilitate the interaction between irisin and αVβ5 either by “opening” αVβ5 into its high-affinity state, or by associating with αVβ5 to provide additional sites for irisin binding, or both. To investigate the molecular mechanism underlying the role of Hsp90α in mediating the irisin/αVβ5 interaction, cryogenic electronic microscopy (cryo-EM) was used to study the conformation of the individual αVβ5 particles in the presence or absence of Hsp90a. We first looked at Hsp90α alone using negative staining EM, and observed a large population of particles which are significantly smaller and more heterogeneous than the previously revealed Hsp90α dimer particles under EM (Southworth, D. R. and D. A. Agard, Species-dependent ensembles of conserved conformational states define the Hsp90 chaperone ATPase cycle. Mol Cell, 2008. 32(5): p. 631-40). The observed structural changes were most likely introduced during EM grid preparation. To avoid this apparently non-native heterogeneity, we first treated the samples with chemical cross-linking. This succeeded and we saw a preponderance of two states: an “extended” (Apo or ADP-bound state) and a “fastened” (ATP bound) states of the Hsp90α (FIGS. 10A and 10B) (Southworth, D. R. and D. A. Agard. Mol Cell, 2008. 32(5): p. 631-40).

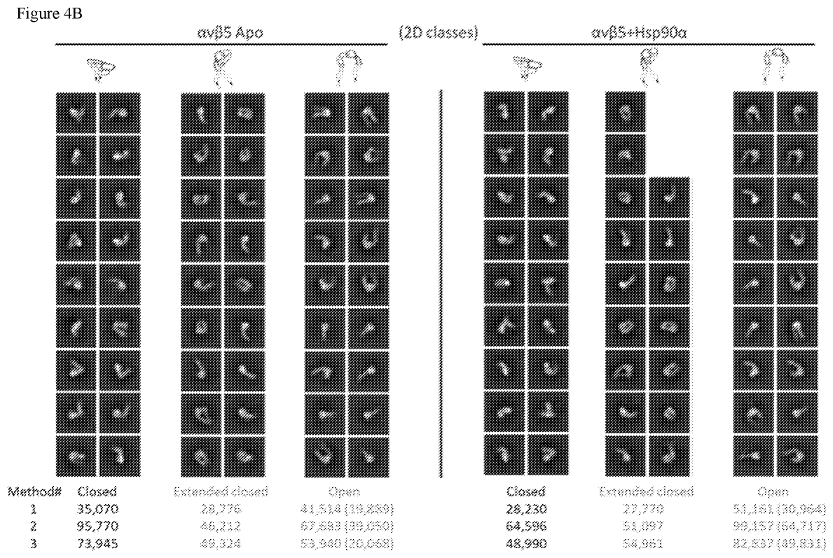

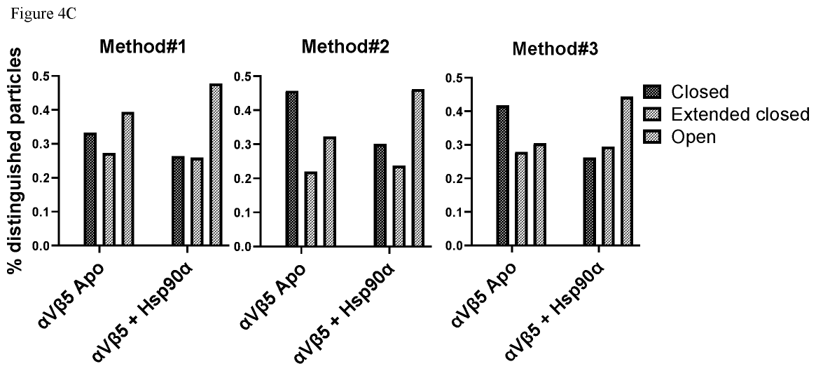

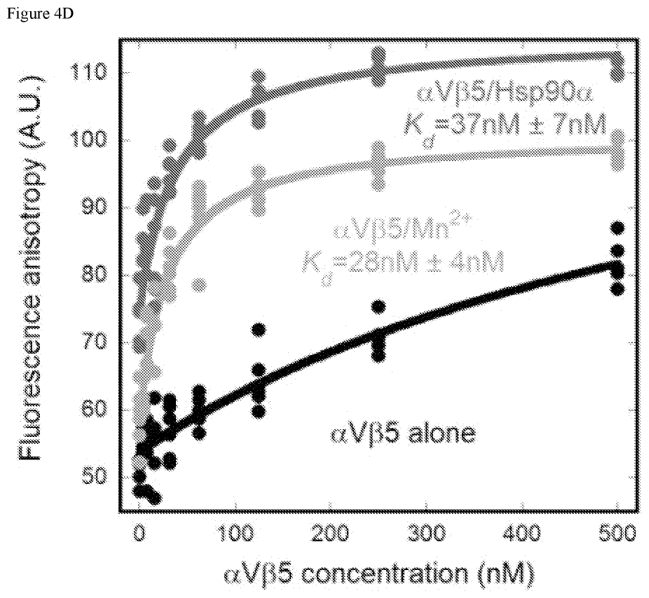

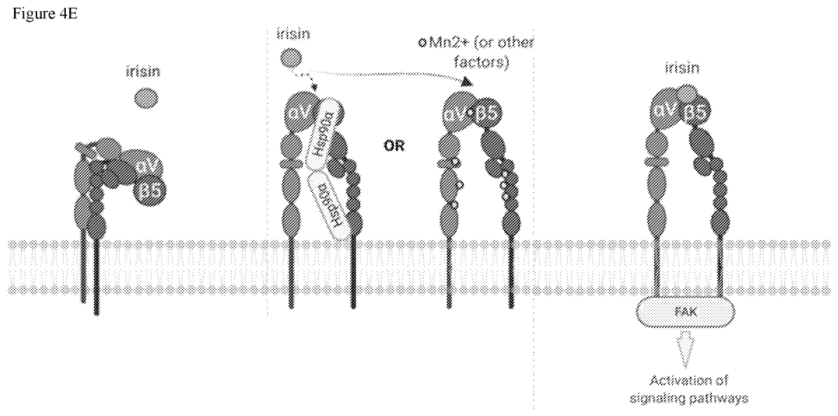

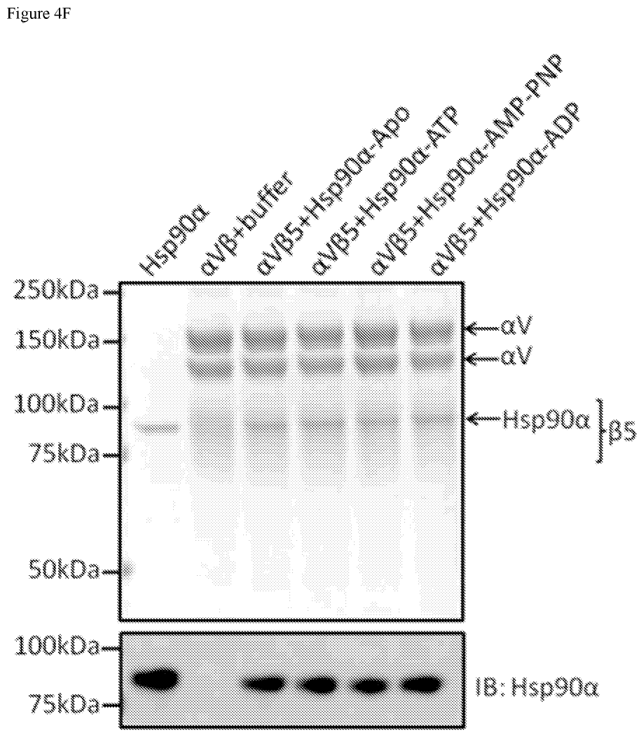

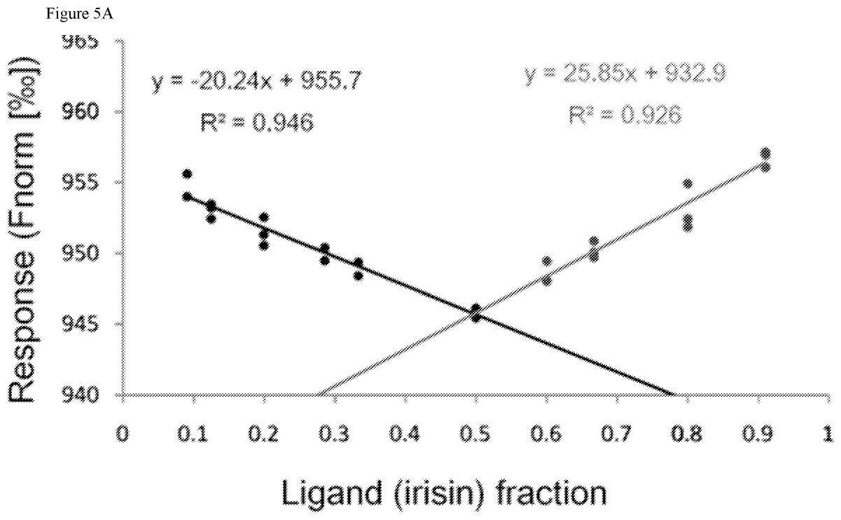

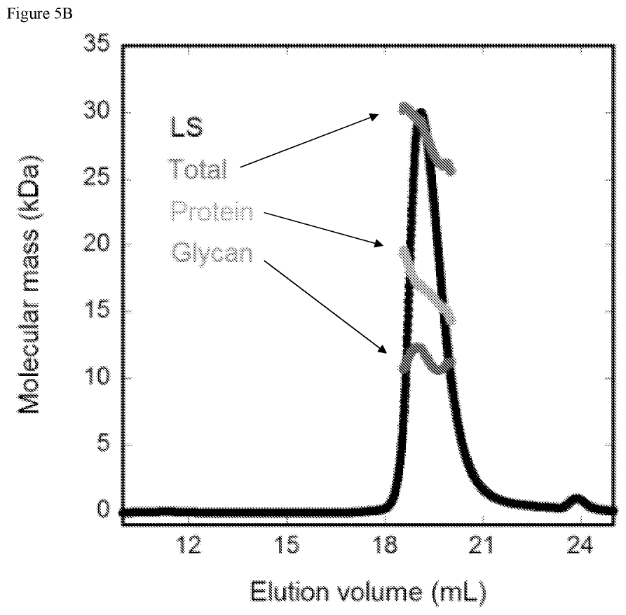

Next, we sought to determine how Hsp90α affects the conformational state of αVβ5. The αVβ5 and αVβ5/Hsp90α cryo-EM samples were prepared purposely without using cross-linker so that Hsp90α particles could be filtered out by size during particle selection. We adopted both “template-free” (method 1) and “template-directed” (method 2 and 3) methods to pick particles for analysis (see methods). Briefly, for unbiased “template-free” method, we used random blob picker to select particles for 2D classification, and the generated 2D classes were further analyzed based on the previously described ensembles of three distinct confirmations: closed (low affinity state), extended closed and open (high affinity state) (Springer, T. A. and M. L. Dustin. Curr Opin Cell Biol, 2012. 24(1): p. 107-15); for “template-directed” methods, six 2D classes containing “closed”, “extended closed” and “open” states were first generated from either αVβ5 or αVβ5/Hsp90α samples using blob picking, and the generated 2D classes were subsequently used as the templates to pick particles from the αVβ5 and αVβ5/Hsp90α micrographs (FIGS. 4A and 4B). The number and percentage of particles generated in each state were quantified for both αVβ5 and αVβ5/Hsp90α samples, with or without particles classified into a “likely open” group included. Analyzed by all three different methods, we saw more “open” and fewer “closed” αVβ5 particles in the αVβ5/Hsp90α compared to the αVβ5 alone sample, suggesting that Hsp90α functions to “open” αVβ5 for tight irisin binding (FIGS. 4B-4C and FIG. 10C). This result is similar to the measured effects of certain exogenous antibodies which bind integrin extracellular domains and activate the receptors from a low- to a high-affinity state for ligand binding (Frelinger, A. L., 3rd, et al., Monoclonal antibodies to ligand-occupied conformers of integrin alpha IIb beta 3 (glycoprotein IIb-IIIa) alter receptor affinity, specificity, and function. J Biol Chem, 1991. 266(26): p. 17106-11; Arroyo, A. G., et al., Regulation of the VLA integrin-ligand interactions through the beta 1 subunit. J Cell Biol, 1992. 117(3): p. 659-70; Kovach, N. L., et al., A monoclonal antibody to beta 1 integrin (CD29) stimulates VLA-dependent adherence of leukocytes to human umbilical vein endothelial cells and matrix components. J Cell Biol, 1992. 116(2): p. 499-509).

We next examined whether Hsp90α affects the affinity of irisin for αVβ5. To do this, we took advantage of another well-known method to chemically “open” a variety of integrins, that is Mn²⁺ ion supplementation (Anderson, J. M., J. Li, and T. A. Springer, Regulation of integrin alpha5beta1 conformational states and intrinsic affinities by metal ions and the ADMIDAS. Mol Biol Cell, 2022. 33(6): p. ar56; Elices, M. J., L. A. Urry, and M. E. Hemler, Receptor functions for the integrin VLA-3: fibronectin, collagen, and laminin binding are differentially influenced by Arg-Gly-Asp peptide and by divalent cations. J Cell Biol, 1991. 112(1): p. 169-81; Dransfield, I., et al., Divalent cation regulation of the function of the leukocyte integrin LFA-1. J Cell Biol, 1992. 116(1): p. 219-26). αVβ5 was activated either by adding Mn²⁺ ion, or by forming a complex with Hsp90α (see FIGS. 1F and 1G), and the affinity of αVβ5/Mn²⁺ and αVβ5/Hsp90α for A488-irisin was compared using fluorescence anisotropy. In contrast to αVβ5 alone in Mg²⁺/Ca²⁺ buffers, the binding affinities of αVβ5/Mn²⁺ and αVβ5/Hsp90α for irisin were very similar, with the K_{d}^{app} of 28 nM±4 nM and 37 nM±7 nM, respectively (FIG. 4D). These results strongly suggest that the main function of Hsp90α is to “open” αVβ5. Once this integrin structure is “opened”, Hsp90α does not appear to further improve the affinity of the receptor for irisin (FIG. 4E).

Intracellular Hsp90α is an ATP-dependent chaperone. We therefore investigated whether the ATPase activity of Hsp90α is essential for mediating the irisin-αVβ5 interaction. After stripping off the originally bound nucleotide on Hsp90α, we recharged it with various nucleotides (see methods). Subsequently, the binding between αVβ5 and recombinant Hsp90α was investigated in different nucleotide states: Apo, ATP, nonhydrolyzable ATP (AMP-PNP) or ADP. Using a pull-down assay, we detected that, regardless of the nucleotide state, a similar amount of Hsp90α co-precipitated with αVβ5. This suggests that the classic chaperone activity of Hsp90α, depending on ATP, is not required for the Hsp90α-αVβ5 interaction (FIG. 4F).

### Biophysical Characterization of the Irisin/αVβ5 Complex Suggests an Unconventional Ligand-Integrin Interaction

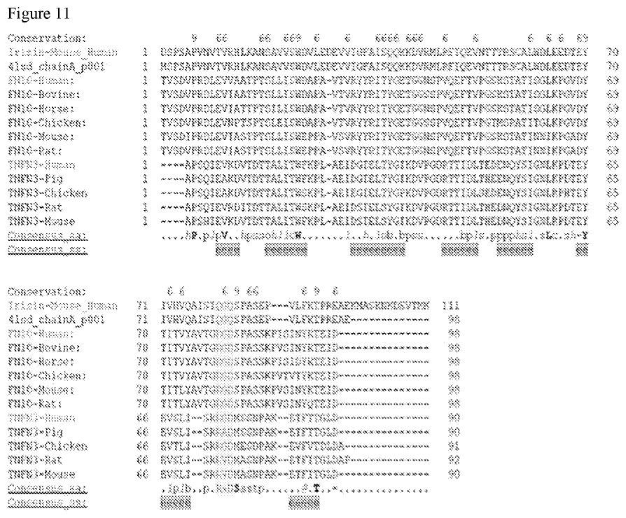

Irisin differs in several ways from other integrin ligands: first, as a small polypeptide hormone ligand, irisin contains only one distinguished functional domain that shares limited sequence homology (15˜20% identity), but great structural homology, to fibronectin III domains (FIG. 11) (Schumacher, M. A., et al. J Biol Chem, 2013. 288(47): p. 33738-33744). Furthermore, it lacks additional domains that contain “synergy sites” mediating high-affinity integrin-binding. Moreover, irisin also does not contain an RGD motif that is considered to be the hallmark of integrin ligands (FIG. 11). While these results argue that eHsp90α serves as a novel physiological pathway to activate integrin, mediating both irisin binding and signaling, the exact mode of activation of integrins by irisin would require understanding at the atomic level.

To generate a structural model of the irisin-αVβ5 complex, we first estimated the binding stoichiometry between irisin and αVβ5, using a form of the classic Job plot where the concentration is held constant and mole fraction is varied. In this case, we detected the binding by MircroScale Thermophoresis (MST) (Moran Jerabek-Willemsen, T. A., Randy Wanner, Heide Marie Roth, Stefan Duhr, Philipp Baaske, Dennis Breitsprecher, MicroScale Thermophoresis: Interaction analysis and beyond. Journal of Molecular Structure, 2014. 1077: p. 101-113). Briefly, the MST response is plotted against the mole fraction of the ligand, and the maximum amount of complex formation (minimum response in the MST measurement) occurs at the mole fraction that corresponds to the complex composition. Two linear fitting curves intersected at ˜50% mole fraction of irisin (see FIG. 5A), suggesting that one irisin molecule binds to one αVβ5 heterodimer. The binding stoichiometry suggested that the irisin used here, expressed in mammalian cells (irisin-mam), is a monomer. This is quite distinct from the previously characterized dimeric form of irisin that was produced in bacteria (irisin-bac) (Schumacher, M. A., et al. J Biol Chem, 2013. 288(47): p. 33738-33744). Insin-mam is heavily glycosylated, and appears to be much larger than its protein molecular mass (˜14 kDa) in silver-stained SDS-PAGE (FIG. 8A). To determine the oligomeric state of irisin-mam, we used SEC-MALS to measure the absolute molar mass of irisin protein and its conjugated glycans. Irisin eluted as monodispersed particles by size exclusion chromatography with an average molecular mass of 27.3 kDa, as detected by light scattering. We further performed conjugate analysis and determined the irisin protein mass of 16.7 kDa plus the glycan mass of 10.8 kDa, indicating that irisin-mam is a monomer (FIG. 5B).

To characterize the effects of binding between irisin and αVβ5, we used hydrogen/deuterium exchange mass spectrometry (HDX-MS). We did this with large amounts of highly purified recombinant αVβ5 ectodomain in the presence of Mn²⁺ which permitted much more robust HDX than our previous work. Regions of αVβ5 were found protected in the complex (FIG. 5C and FIGS. 5E-5G). Of note, the sites that are protected by irisin are mostly on the β5 subunit (FIG. 5D), and most of the sites are distinct from the RGD binding sites mapped in other αV integrins by HDX (Wang, J., et al., General structural features that regulate integrin affinity revealed by atypical alphaVbeta8. Nat Commun, 2019. 10(1): p. 5481). The loop of the β propeller domain of the αV subunit containing the Mn²⁺ binding site close to the thigh domain was strongly protected from HDX in the complex, indicating that irisin binding further stabilized αVβ5 in the extended open confirmation (Xiong, J. P., et al., Science, 2002. 296(5565): p. 151-5). Little change in HDX signal was observed within irisin itself, indicating that conformational rearrangements or changes to conformational dynamics did not occur in irisin as a result of binding to this integrin.

### Atomic Resolution Model of the Irisin/αVP5 Complex

Finally, we generated a docking model of the irisin/αVβ5 complex, refined by multiple steps of MD simulation. The experimental results of binding stoichiometry, as well as the HDX-MS results were incorporated as restraints during the docking process (FIG. 6A). Notably, the model revealed an extensive binding interface, burying 1586 Å² of irisin that involves five of its six inter-strand loops and four of its seven β-sheets. On the integrin side, most of the binding interface is on the βI domain in the β5 head, and only three loops of the β propeller domain in the αV head right at the α/β subunit interface are involved. Complete exposure of the irisin binding site requires αVβ5 to be in the “open” state (FIG. 6B). This is in striking contrast to the binding of FN10 to its integrin receptors in which their contact area is only a few hundreds of Å² and is solely contributed by the RGD loop of FN10 (FIG. 6C). The binding of irisin to integrin is dominated by electrostatic interactions, where one acidic patch and one basic patch on irisin well fit into the corresponding basic and acidic grooves on αVβ5 (FIG. 6D). Several residues at the irisin/αVβ5 interface stabilize the intermolecular interactions by bonding with more than one residue. For example, E991 in the β5 subunit and E82 in the αV subunit within the acidic groove binds H13, S22, S58, and R44, Q39 in irisin, respectively; R905 and K923 sitting in the basic groove of β5 subunit interact with Q50, E51, V52, and V31, T54 in irisin, respectively (FIG. 6E). The irisin residues having interactions (electrostatic interactions or hydrophobic interactions) with αVβ5 at the irisin/αVβ5 interface include: H13, N17, V20, S22, V31, 132, F34, Q39, V43, R44, M45, L46, R47, F48, 149, Q50, E51, V52, N53, T54, T55, T56, S58, C59, A60, L61, W62, D63, L64, E65, 179, Q82. The irisin residues having hydrophobic interactions with αVβ5 at the irisin/αVβ5 interface include V20, I32, F34, V43, M45, I49, T56, C59, A60, L61, W62, E65. The irisin residues having electrostatic interactions with αVβ5 at the irisin/αVβ5 interface include H13, N17, S22, V31, Q39, R44, L46, R47, F48, Q50, E51, V52, N53, T54, T55, S58, D63, L64, I79, Q82. Given that the N- and C-termini of irisin align with those of integrin in the same direction, it is unlikely that FNDC5, as a membrane protein, can serve as a “counter receptor” of integrin. Although irisin is a small polypeptide integrin ligand, this unusually large interface likely gives rise to the high-affinity integrin binding.

Besides the difference in the size of the binding interface which likely accounts for the different binding affinity, we also compared the integrin binding sites between irisin and FN10. FN10-αVβ3 complex structure (PDB 4MMX) was used for fibronectin alignment to dock FN10 onto αVβ5. Distinct from the canonical RGD-mediated integrin ligand binding site, irisin binds to the opposite side of αVβ5 (FIG. 6C). Lastly, the irisin-αVβ5 interface in the model is largely overlapping with the irisin dimer interface, which predicts a significantly lower affinity of the bacterial irisin as dimer compared to the higher affinity of mammalian irisin monomer for αVβ5 (Schumacher, M. A., et al. J Biol Chem, 2013. 288(47): p. 33738-33744).

### Validation of the Irisin/αVβ5 Complex Model

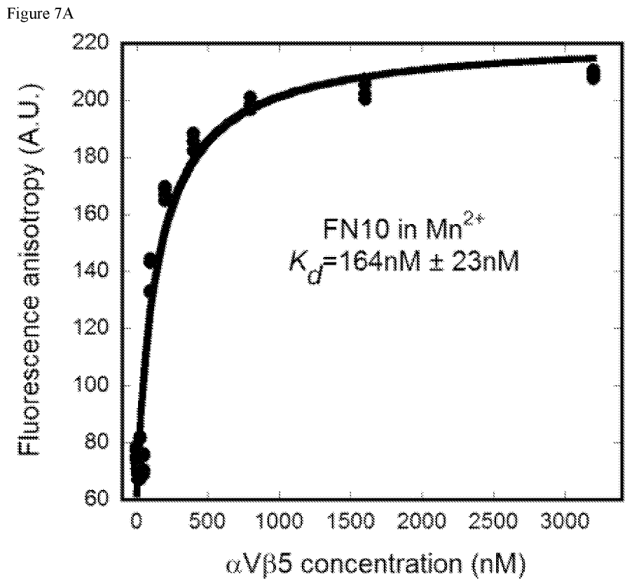

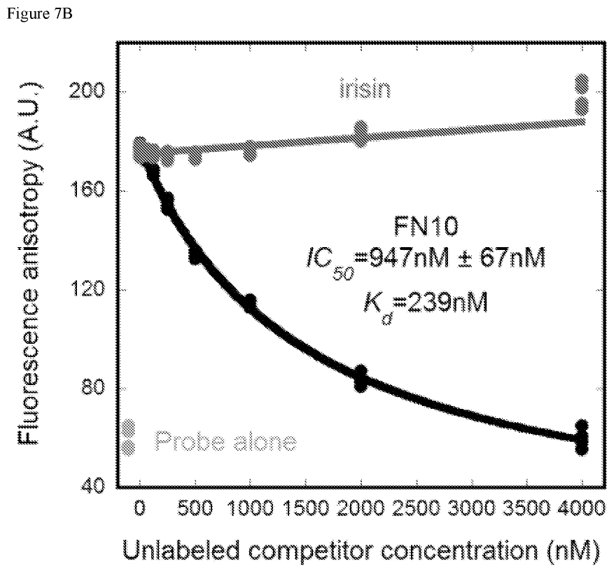

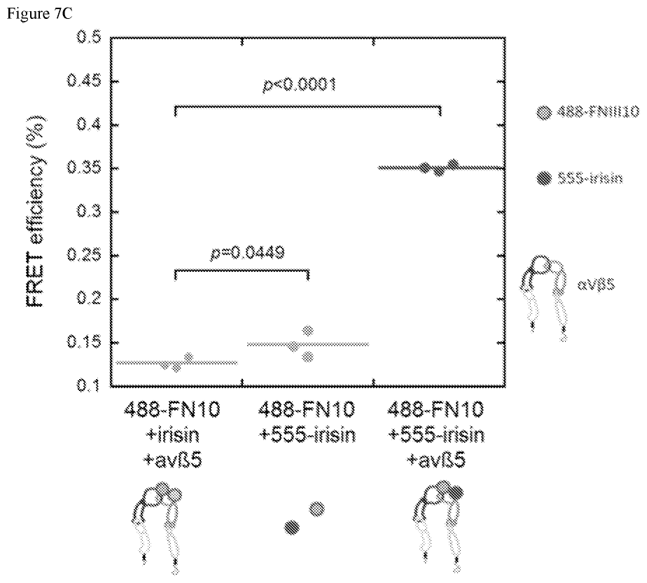

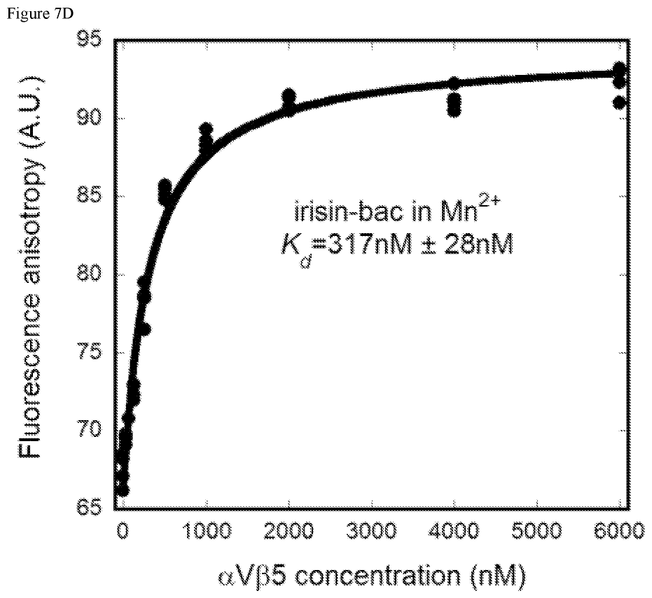

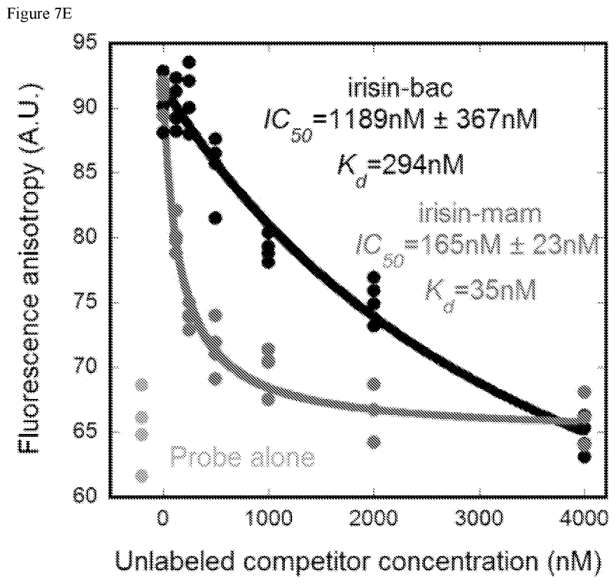

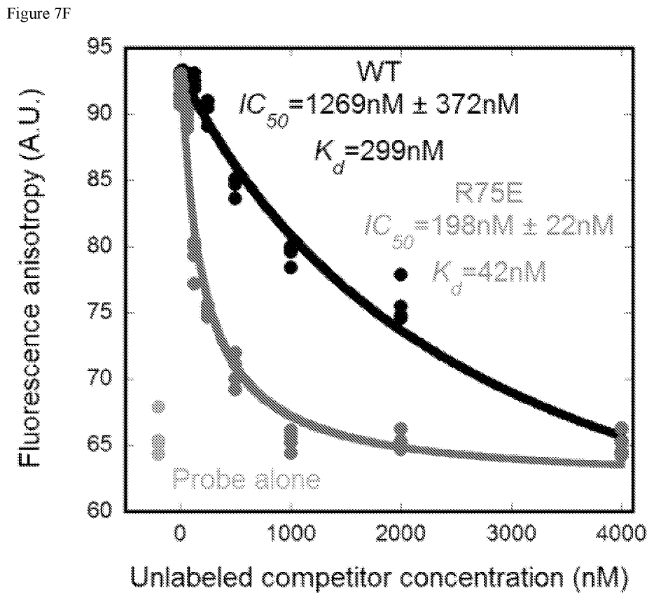

This model of the irisin/αVβ5 complex is surprising for at least two reasons. First, the irisin binding site on αVβ5 is completely distinct from the binding sites for other FNIII domain-containing ligands. Hence, they should not be competitive with irisin in binding to αVβ5. Second, the large irisin-αVβ5 interface in this model likely accounts for the high affinity binding of the irisin monomer, rather than the dimer, to αVβ5. This is because the irisin-αVβ5 interface largely overlaps with the irisin dimer interface, so that the αVβ5 binding site on irisin could be masked by the other irisin subunit in the irisin dimer. Therefore, αVβ5 would sterically compete with one irisin subunit in the dimer for binding to the other irisin monomer, resulting in apparently low affinity of irisin dimer for αVβ5. To examine these predictions, we first compared the binding of irisin to αVβ5 with that of a classical FNIII-domain integrin ligand. Irisin shares structural homology with the fibronectin FNIII 10^{th} domain (FN10) (Schumacher, M. A., et al. J Biol Chem, 2013. 288(47): p. 33738-33744), but lacks an RGD that is present in this and many other FNIII domain-containing αVβ5 ligands (FIG. 11). We prepared a 10 kDa protein containing just the FN10 domain of fibronectin and tested the affinity between FN10 and αVβ5 using the fluorescence anisotropy binding assay with A488-labeled FN10. This yielded a K_{d}^{app} of 146 nM±23 nM for the FN10 protein, almost 5-fold weaker than the affinity between irisin and αVβ5 (FIG. 7A). To determine whether irisin binds αVβ5 at a different site from that occupied by the canonical RGD-mediated integrin binding adopted by FN10, a fluorescence anisotropy competition assay was used. This employed fixed concentrations of A488-FN10 and αVβ5, and varying amounts of either unlabeled irisin or FN10. While unlabeled FN10 binds αVβ5 competitively with a K_{d}^{app} of 239 nM, the unlabeled irisin did not show any competition with FN10. This indicates that these two FNIII domain containing proteins, irisin and FN10, do not compete for binding at the same integrin site (FIG. 7B). To further emphasize this point, we asked whether a single αVβ5 could bind irisin and the FN10 protein simultaneously. A Fluorescence Resonance Energy Transfer (FRET) assay was performed using A488-FN10 and A555-irisin, and significant FRET was detected only in the presence of αVβ5. This data indicates that FN10 and irisin can bind αVβ5 simultaneously and in close proximity (FIG. 7C).

Next, we examined whether irisin monomer has higher affinity for αVβ5 compared to irisin dimer. As shown in FIG. 5B and Schumacher, M. A., et al. J Biol Chem, 2013. 288(47): p. 33738-33744, irisin-mam is a glycosylated monomer and irisin-bac is a non-glycosylated dimer. We compared the affinities of αVβ5 for irisin-mam and irisin-bac. The latter binds αVβ5 with a K_{d}^{app} of 256 nM±14 nM, about 8-fold weaker than that for irisin-mam, when measured by the fluorescence anisotropy binding assay with A488-irisin-bac (FIG. 7D). To examine whether αVβ5 sterically competes with one of the irisin subunits in the dimer to bind the other irisin monomer, we then investigated whether irisin-mam competes with irisin-bac for the same binding site on αVβ5. We used fluorescence anisotropy in a competition assay with fixed concentrations of A488-irisin-bac and αVβ5, and varying amounts of unlabeled irisin-bac or irisin-mam, which yielded a K_{d}^{app} of 294 nM and 35 nM, respectively. It is noteworthy that both unlabeled irisin-bac and irisin-mam achieved 100% inhibition at higher concentrations, indicating that they both bind αVβ5 at the same site (FIG. 7E). Lastly, we generated the non-glycosylated irisin monomer mutant R75E from bacteria as previously described (Schumacher, M. A., et al. J Biol Chem, 2013. 288(47): p. 33738-33744), and performed the same anisotropy competition assay as FIG. 7E. The monomeric mutant binds αVα5 with similarly high affinity (K_{d}^{app} of 42 nM) as irisin-mam, compared to the wild type irisin bacterial dimer (K_{d}^{app} of 299 nM), and 100% inhibition was achieved at the micromolar concentration range of both forms of irisin (FIG. 7F). Collectively, these data provide validation of key aspects of this model of irisin-αVβ5 binding.

### The Role of Hsp90a in Irisin-Induced Biological Effects

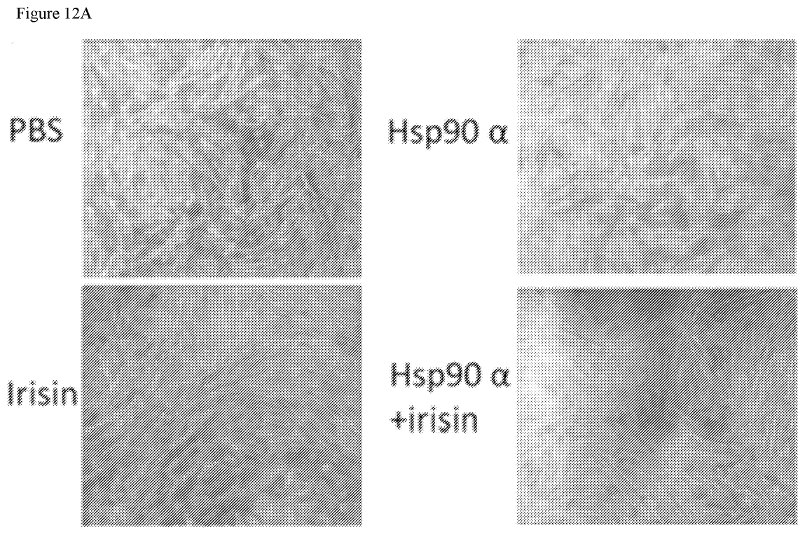

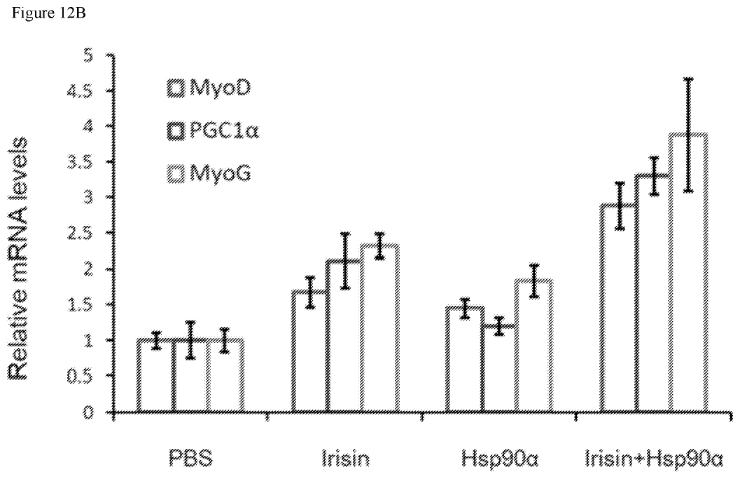

Previous work suggested that irisin induces the “beiging” of inguinal subcutaneous white adipose tissues (iWAT) (Bostrom et al., A PGC1-alpha-dependent myokine that drives brown-fat-like development of white fat and thermogenesis. Nature. 2012; 481(7382):463-8), promotes myogenesis, and prevents skeletal muscle atrophy in hind limb suspended mice (Reza et al., Nat Commun. 2017; 8(1):1104, Colaianni et al., Irisin prevents and restores bone loss and muscle atrophy in hind-limb suspended mice. Sci Rep. 2017; 7(1):2811). While αVβ5 is known to mediate irisin-induced effects in white fat tissues (Kim et al., Cell. 2018; 175(7):1756-68 e17), the integrin that functions as the major receptor for irisin in skeletal muscle has not yet been identified. Therefore, it is necessary to uncover the role of Hsp90α in primary iWAT (where αVβ5 is known to be the major irisin receptor), and in the C2C12 cell line (where αVβ5 may or may not be used for irisin-mediated effects). The goal was to test Hsp90α in cell types with known irisin biology, and evaluate whether Hsp90α is a general cofactor for irisin-mediated effects, or whether it functions in a tissue- or cell type-specific manner. Data suggested that treating C2C12 cells with irisin and Hsp90α increases myogenesis relative to treatments with irisin alone or Hsp90α alone (FIGS. 12A and 12B). These findings could be further verified in the following experiments.

### Testing the Role of Hsp90α in Irisin-Mediated Effects on Cultured Muscle and Fat Cells

For muscle cells, experiments were done to validate whether irisin induces integrin signaling in C2C12 cells, and if so, which irisin concentrations maximally stimulate integrin signaling. Recombinant irisin (0.1-100 nM) was added to C2C12 cells on day 3 or 4 of differentiation, as described in (Reza et al., Nat Commun. 2017; 8(1):1104), and levels of pFAK and FAK were probed to establish the intensity of integrin signaling. Further tests were done to determine whether the influence of irisin is mediated via αVβ5. The anti-integrin αVβ5 neutralizing antibody (Abcam) effectively blocks irisin-induced signaling in bones (Kim et al., Cell. 2018; 175(7):1756-68 e17, Estell et al., Elife. 2020; 9): this antibody was used to test if αVβ5 is the major integrin receptor for irisin in C2C12. Control IgG (Mouse IgG1 Isotype Control, R&D Systems) served as a control. Finally, undifferentiated C2C12 cells were seeded on coverslips and experiments in FIG. 12A were repeated. On day 4, 6 and 10, cells were fixed and stained with Alexa568-phalloidin and DAPI, and fluorescence microscopy was used to visualize and quantify the number, diameter, and length of the muscle fibers, as well as the number of nucleus in each cell to assess the degree of fusion. Additionally, cell extracts were be used to detect levels of MyoD, Myf5, Desmin, IGF-1, Akirin-1 and atrogin-1 protein. In the fat cell experiments, preadipocytes were extracted from inguinal fat pads, and treated with PBS, 1 nM irisin alone, 1 nM Hsp90α alone, or 1 nM irisin plus 1 nM Hsp90α on a daily basis from Day 1 of differentiation. Levels of expression of genes involved in adipogenesis and thermogenesis were measured, using Q-PCR to evaluate how Hsp90α influences irisin-induced effects in iWAT.

### Testing how Hsp90a Influences Irisin-Mediated “Beiging” of White Adipose Tissue In Vivo in Mice.

Animal protocols and mouse models for overexpression and loss-of-function of irisin were established. Mice injected in the tail vein with AAV8-irisin-flag showed elevated plasman irisin level; and FNDC5 KO mice were validated in multiple ways (Kim et al., Cell. 2018; 175(7):1756-68 e17, 31. Wrann et al., Exercise induces hippocampal BDNF through a PGC-1alpha/FNDC5 pathway. Cell Metab. 2013; 18(5):649-59) & (Islam et al., 2021 Nature Metabolism). To investigate how Hsp90α influences irisin-mediated metabolic effects in mice, Hsp90α was administrated intraperitoneally into WT C57/BL6 (littermates of FNDC KO mice) injected with either AAV8-GFP or AAV8-irisin-flag, and FNDC KO mice injected with either AAV8-GFP or AAV8-irisin-flag. As above mentioned, AAV8-irisin-flag (AAV8-GFP) was delivered by tail-vein injection when mice are four-week old, and Hsp90α was administrated at the beginning of week 7, daily for two weeks. The level of plasma Hsp90α was monitored by ELISA. Inguinal WATs were collected at the end of week 9 for 1) detection of thermogenic gene expression via Q-PCR, 2) quantification of UCP1 protein level by western blot, and 3) Immunohistochemistry against UCP1, as previously described (Bostrom et al., Nature. 2012; 481(7382):463-8).

### Establishing how Glycosylation Affects Irisin Activity

Three glycosylation sites were identified in irisin: N36, N45 and N81. In recombinant irisin, all three sites are 100% glycosylated, but N45 of irisin from human plasma is only 2% glycosylated (Table 4).

TABLE 4
Characterization of glycosylation sites N36, N45 and N8 in irisin

| Glycosylation | | | | | # |
|---|---|---|---|---|---|
| Site | Peptide | Intensity | Xcorr | ACorr | Ions |
| N36 | A.DSPSAPVN#VTVR.H | 3700370000.0 | 2.625 | 0.827 | 12/22 |
| N45 | K.AN#SAVVSWDVLEDEVVIGFAISQQK. | 22804800.0 | 5.422 | 0.783 | 23/96 |
| | K | | | | |
| N81 | R.FIQEVN#TTTR.S | 4015870000.0 | 2.522 | 0.616 | 11/18 |

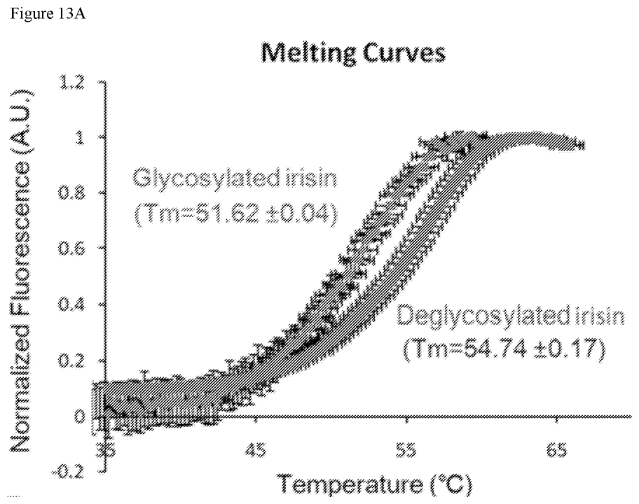

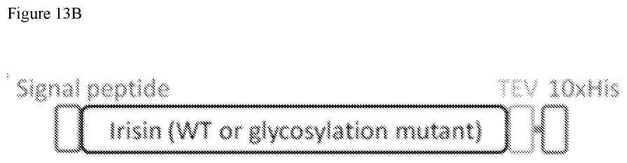

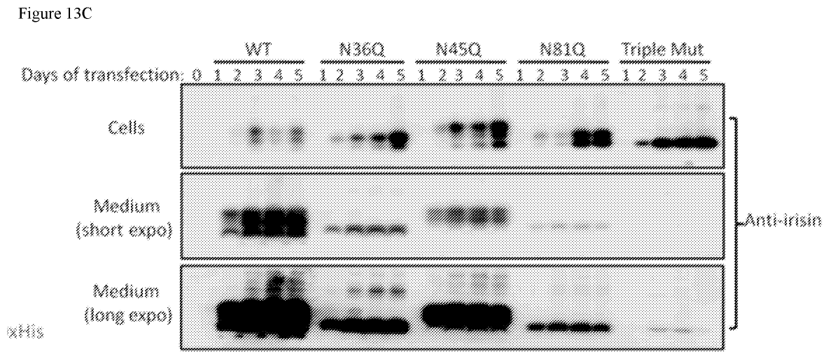

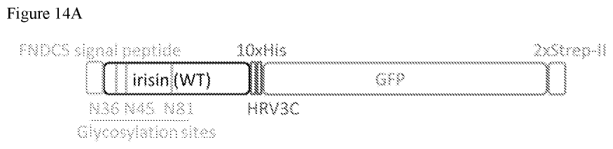

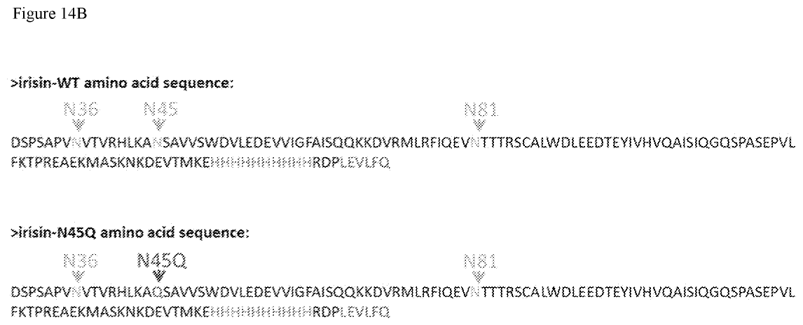

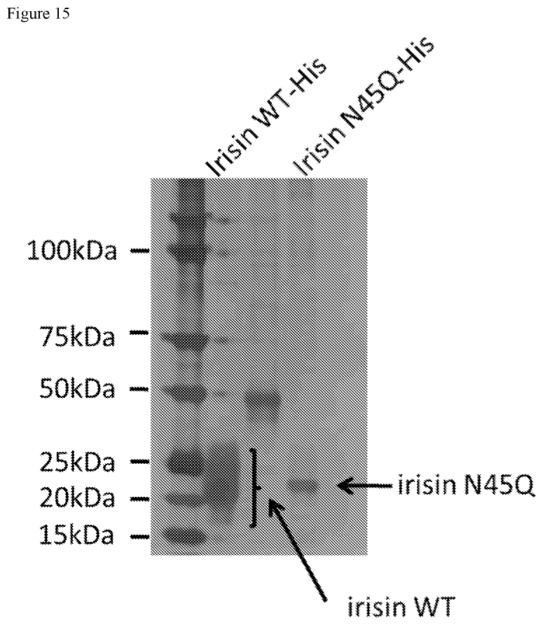

Given that recombinant irisin has an extremely short half-life when tested in vivo in mice, it was hypothesized that the differential glycosylation patterns of different forms of irisin are related to the differences in their bioactivities and half-lives. To test whether glycosylation affects the stability of irisin protein, recombinant irisin was deglycosylated with deglycosylase mixII (NEB #P6044), and compared its thermal stability to that of the native irisin. Notably, irisin without glycosylation was more stable (FIG. 13A). Combining the results shown in FIG. 13C, further experimental data showed that glycosylation mediates the affinity of irisin for integrin and Hsp90a, and thereby alters the bioactivity of irisin; also that the biological activity of irisin is positively related to the speed at which the irisin/integrin complex is internalized, which terminates irisin-mediated signaling. To identify the function of each glycosylation site, single and triple irisin N-to-Q mutants were generated, and the mutant expression in and secretion from mammalian Expi293 cells were assessed. It was found that 1) while changes in irisin glycosylation do not affect intracellular irisin protein levels, their levels of secreted protein are dramatically different; 2) some sites are cooperatively glycosylated: for instance, N36Q and N8TQ mutants share a similar glycosylation pattern; 3) the triple mutant has no other detectable glycosylation species, indicating that the three sites identified above are, at least, the major glycosylation sites (FIG. 13C). Secreted WT and irisin glycosylation mutant were purified from the cell culture medium using Ni-NTA IMAC chromatography. FIG. 14A shows the recombinant irisin production construct. FIG. 14B shows the sequences and modifications of the recombinant irisin and the irisin N45Q mutant. FIG. 15 shows the silver staining of recombinant irisin and irisin N45Q mutant purified from mammalian Expi293 cells. The results show that compared to WT irisin, its glycosylation mutant (e.g., irisin N45Q mutant) runs in SDS gels as a much more homogeneous band.

Irisin Glycosylation (N to Q) Mutant Recombinant Proteins and their Stability and their Affinity for the Integrin/Hsp90α Complex.

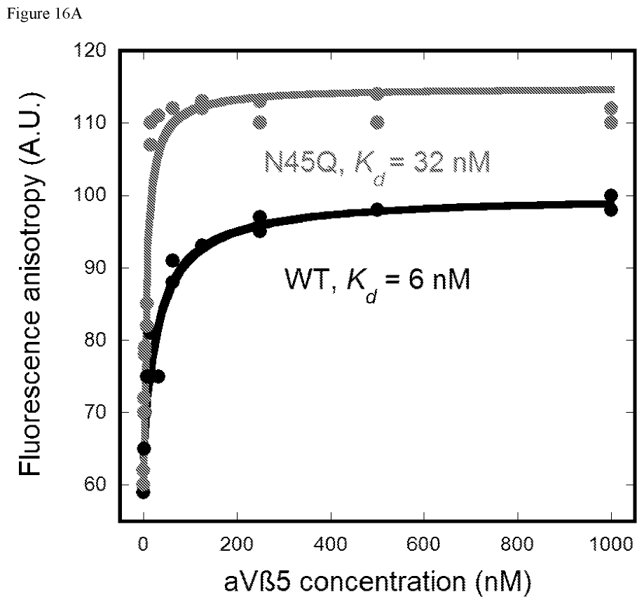

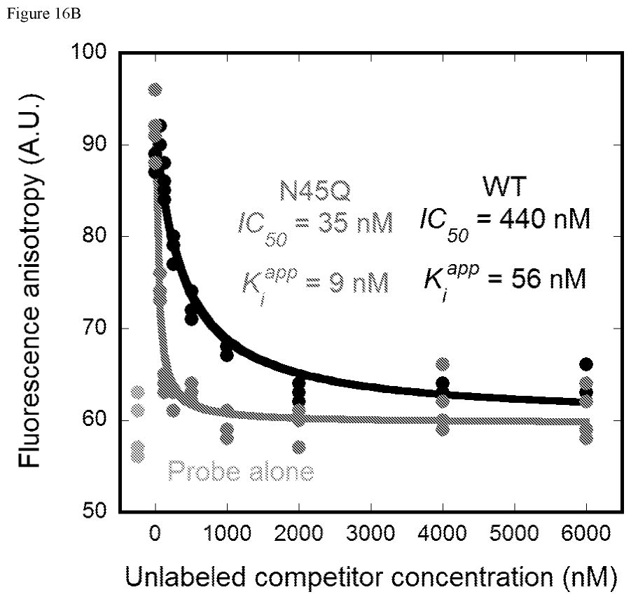

FIG. 13B shows a schematic of the construct designed for recombinant (WT and mutants) irisin production. His-tagged and non-tagged irisin proteins were both produced using Expi293 cells. Thermal stability of all recombinant irisin proteins was evaluated in parallel. A competition fluorescence anisotropy assay was used to compare the affinity of WT and irisin glycosylation mutant for the αVD5/Hsp90 complex (FIG. 16B). 5 nM irisin-Atto488 protein (same as the experiment described above), and a concentration (two times of the K_{d}) of the αVD5/Hsp90 complex based on the K_{d} derived above, to enable enough starting anisotropy signal for the competition assay. A wide range of concentrations (10 nM˜10 μM) of non-tagged and unlabeled WT and irisin glycosylation mutants were tested to ensure close to compete competition with WT irisin (control). FIGS. 16A-16B show that compared to the WT irisin, the irisin glycosylation mutant (e.g., N45Q) binds stronger to its receptor by about 5-10 folds.

### Assessment of how Glycosylation Affects Irisin-Mediated Integrin Signaling.

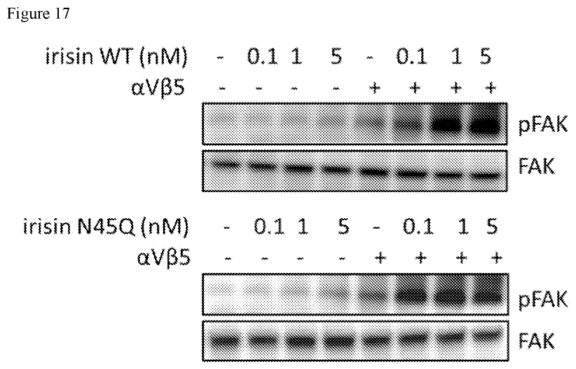

Experimental procedures described above were used to compare cellular activity of WT irisin protein and its glycosylation mutants. Briefly, HEK293T cells that ectopically expressed αVβ5 and were pretreated with Hsp90α were further treated with WT irisin or its glycosylation mutants. FAK and Phosphorylated FAK were used as activity readouts. FIG. 17 shows the western blot illustrating the irisin-induced integrin signaling in HEK293 cells with or without ectopically expressed αVβ5 receptor. FIG. 17 shows that the irisin N45Q mutant reaches its maximal signaling at 0.1 nM while WT irisin has not even at 5 nM. This result indicated that compared to the WT irisin, the irisin glycosylation mutant (e.g., N45Q) induces integrin signaling more potently, by about 5-10 folds.

### Measurement of the Half-Lives of Recombinant WT Irisin and of its Glycosylation Mutants.

As described in (Colaianni et al., Sci Rep. 2017; 7(1):2811), recombinant irisin proteins (WT and glycosylation mutants) were administrated to mice i.v. Plasma samples were taken at different time points post administration for measurement of the percentage of initial dose, by ELISA (R&D kit, (Weimer et al., Prolonged in-vivo half-life of factor VIIa by fusion to albumin. Thromb Haemost. 2008; 99(4):659-67)); the half-lives were derived from the generated curves. The N45Q mutant was found to have intermediate thermal stability, bioactivity and in vivo half-life, compared to WT (low stability, high bioactivity and short half-life) and triple mutant (high stability, low bioactivity and longer half-life).

### INCORPORATION BY REFERENCE

The contents of all references, patent applications, patents, and published patent applications, as well as the Figures and the Sequence Listing, cited throughout this application are hereby incorporated by reference.

### EQUIVALENTS

Those skilled in the art will recognize, or be able to ascertain using no more than routine experimentation, many equivalents to the specific embodiments encompassed by the present invention described herein. Such equivalents are intended to be encompassed by the following claims.

1. An irisin polypeptide, comprising a glycosylated residue at position 36, a glycosylated residue at position 81, and a non-N-linked glycosylated residue at position 45.

2. The irisin polypeptide of claim 1, wherein the non-N-linked glycosylated residue at position 45 is a non-canonical N-linked glycosylated residue at position 45.

3. The irisin polypeptide of claim 1, wherein the irisin polypeptide comprises a non-hydrophobic residue at position 45.

4. The irisin polypeptide of claim 3, wherein the non-hydrophobic residue at position 45 is selected from the group consisting of glutamine, threonine, serine, cysteine, arginine, histidine, lysine, aspartic acid, and glutamic acid.

5. The irisin polypeptide of claim 3, wherein the non-hydrophobic residue at position 45 does not cause misfolding within the irisin polypeptide.

6. The irisin polypeptide of claim 1, wherein the irisin polypeptide comprises a Q residue at position 45.

7. The irisin polypeptide of any one of claims 1 to 6, wherein the irisin polypeptide comprises an amino acid sequence having at least 80% sequence identity to the amino acid sequence set forth in SEQ ID NO: 1 or SEQ ID NO: 13.

8. The irisin polypeptide of any one of claims 1 to 7, wherein the irisin polypeptide comprises at least 85% identity to SEQ ID NO: 1 or SEQ ID NO: 13.

9. The irisin polypeptide of any one of claims 1 to 8, wherein the irisin polypeptide comprises at least 90% identity to SEQ ID NO: 1 or SEQ ID NO: 13.

10. The irisin polypeptide of any one of claims 1 to 9, wherein the irisin polypeptide comprises at least 95% identity to SEQ ID NO: 1 or SEQ ID NO: 13.

11. The irisin polypeptide of any one of claims 1 to 10, wherein the irisin polypeptide comprises at least 99% identity to SEQ ID NO: 1 or SEQ ID NO: 13.

12. The irisin polypeptide of any one of claims 1 to 11, wherein the irisin polypeptide comprises SEQ ID NO: 1 or SEQ ID NO: 13.

13. The irisin polypeptide of any one of claims 1 to 12, wherein the irisin polypeptide does not comprise a signal polypeptide.

14. The irisin polypeptide of any one of claims 1 to 13, wherein the irisin polypeptide does not comprise a FNDC5 signal polypeptide.

15. The irisin polypeptide of claim 14, wherein the FNDC5 signal polypeptide comprises the amino acid sequence of SEQ ID NO: 12.

16. The irisin polypeptide of any one of claims 1 to 12, wherein the irisin polypeptide further comprises a signal polypeptide.

17. The irisin polypeptide of any one of claims 1 to 16, wherein the irisin polypeptide further comprises one or more polyhistidine (His)-Tag(s), optionally wherein the irisin polypeptide comprises two or more, five or more, or ten or more His-Tags.

18. The irisin polypeptide of any one of claims 1 to 17, wherein the irisin polypeptide further comprises a human rhinovirus 3C protease (HRV-3C) protease tag.

19. The irisin polypeptide of any one of claims 1 to 18, wherein the irisin polypeptide further comprises a GFP sequence.

20. The irisin polypeptide of any one of claims 1 to 19, wherein the irisin polypeptide further comprises a Strep-II tag, optionally wherein the irisin polypeptide comprises at least two Strep-II tags.

21. The irisin polypeptide of any one of claims 1 to 20, wherein the irisin polypeptide binds to an irisin receptor with at least 1-fold, at least 5-fold, or at least 10-fold more binding affinity to an irisin receptor compared to wild-type irisin.

22. The irisin polypeptide of any one of claims 1 to 21, wherein the irisin polypeptide binds to an irisin receptor with at least 1-fold, at least-5 fold, or at least-10 fold increased binding affinity to αVβ5 and/or integrin compared to wild-type irisin.

23. The irisin polypeptide of any one of claims 1 to 22, wherein the irisin polypeptide comprises at least 1-fold, at least 5-fold, or at least 10-fold increased stability compared to wild-type irisin.

24. The irisin polypeptide of any one of claims 1 to 23, wherein the irisin polypeptide comprises at least 1-fold, at least 5-fold, or at least 10-fold increased half-life compared to wild-type irisin.

25. The irisin polypeptide of any one of claims 1 to 24, wherein the irisin polypeptide is fused to one or more heterologous polypeptides at its N-terminus and/or C-terminus, and/or within any interstrand loop regions.

26. The irisin polypeptide of any one of claims 1 to 24, wherein the irisin polypeptide comprises an amino acid modification, post-translational modification, and/or a heterologous an amino acid sequence that stabilizes the irisin polypeptide and/or increases its half-life.

27. An irisin polypeptide comprising a mutation to at least one residue listed in Table 2.

28. A nucleic acid encoding the irisin polypeptide of any one of claims 1 to 27.

29. The nucleic acid of claim 28, wherein the nucleic acid is comprised within an expression vector, optionally wherein the expression vector is a viral expression vector, optionally wherein the viral expression vector is an adeno-associated viral (AAV) vector.

30. A cell comprising the irisin polypeptide of any one of claims 1 to 27 or the nucleic acid of claim 28 or 29.

31. A method of preventing or reducing degeneration of dopaminergic (DA) neurons, preventing or ameliorating at least one motor deficit and/or at least one symptom of cognitive dysfunction or dementia in a subject in need thereof, the method comprising administering to the subject an agent comprising: i) the irisin polypeptide of any one of claims 1 to 27 or a biologically active fragment thereof, or ii) the nucleic acid of claim 28 or 29 or a biologically active fragment thereof.

32. The method of claim 31, wherein the subject is afflicted with an α-synucleinopathy.

33. The method of claim 31 or 32, wherein the subject is afflicted with Parkinson's disease, Lewy body dementia, Alzheimer's disease, multiple system atrophy (SA), a neuroaxonal dystrophy, or Lou Gehrig's disease (ALS).

34. The method of any one of claims 31 to 33, wherein the at least one motor deficit is selected from the group consisting of:
a) tremor at rest, such as a slight tremor in the hands or feet;
b) rigidity (stiffness) of limbs, neck, or shoulders;
c) difficulty balancing (postural instability);
d) slowness of movement or gradual loss of spontaneous movement (bradykinesia);
e) trouble standing after sitting;
f) stiffness in the limbs; and
g) moving more slowly.

35. The method of any one of claims 31 to 34, wherein the at least one symptom of cognitive dysfunction or dementia is selected from the group consisting of:
a) confusion;
b) poor motor coordination;
c) loss of short-term or long-term memory;
d) identity confusion; and
e) impaired judgment.

36. A method of decreasing or reducing a level or amount of α-synuclein in cells of a subject in need thereof, the method comprising administering to the subject an agent comprising: i) the irisin polypeptide of any one of claims 1 to 27 or a biologically active fragment thereof, or ii) the nucleic acid of claim 28 or 29 or a biologically active fragment thereof.

37. The method of claim 36, wherein the subject is afflicted with an α-synucleinopathy.

38. The method of claim 36 or 37, wherein the cells are neurons or glia.

39. The method of any one of claims 36 to 38, wherein the subject is afflicted with Parkinson's disease, Lewy body dementia, Alzheimer's disease, multiple system atrophy (MSA), a neuroaxonal dystrophy, or Lou Gehrig's disease (ALS).

40. The method of claim 36 or 37, wherein the cells are cancer cells, optionally wherein the cells are melanoma cells.

41. The method of claim 40, wherein the subject is afflicted with a cancer characterized by or caused by an increase of the α-synuclein.

42. The method of any one of claims 36 to 41, wherein the α-synuclein is pathogenic α-synuclein.

43. A method of treating or preventing Parkinson's disease, Lewy body dementia, Alzheimer's disease, multiple system atrophy (MSA), a neuroaxonal dystrophy, or Lou Gehrig's disease (ALS) in a subject, the method comprising administering to the subject an agent comprising: i) the irisin polypeptide of any one of claims 1 to 27 or a biologically active fragment thereof, or ii) the nucleic acid of claim 28 or 29 or a biologically active fragment thereof.

44. The method of claim 43, wherein the agent is administered in a therapeutically effective amount to treat Parkinson's disease, Lewy body dementia, Alzheimer's disease, multiple system atrophy (MSA), a neuroaxonal dystrophy, or Lou Gehrig's disease (ALS).

45. A method of increasing expression of brain-derived neurotrophic factor (BDNF) by a cell, comprising contacting the cell with an agent, wherein the agent comprising: i) the irisin polypeptide of any one of claims 1 to 27 or a biologically active fragment thereof, or ii) the nucleic acid of claim 28 or 29 or a biologically active fragment thereof.

46. The method of claim 45, wherein the step of contacting occurs in vivo, ex vivo, or in vitro.

47. The method of claim 45, wherein the cells are neurons.

48. The method of claim 47, wherein the neurons are selected from the group consisting of hippocampal neurons, cerebellar neurons, sciatic nerve neurons, dopaminergic neurons, and substantia nigra neurons.

49. The method of claim 45, further comprising contacting the cell with an additional agent that increases the expression of the BDNF.

50. A method for treating or preventing a neurological disease or disorder in a subject, comprising the step of administering to the subject an agent comprising: i) the irisin polypeptide of any one of claims 1 to 27 or a biologically active fragment thereof, or ii) the nucleic acid of claim 28 or 29 or a biologically active fragment thereof, that increases BDNF expression or activity in the central or peripheral nervous system of the subject, such that the neurological disease or disorder is treated or prevented.

51. The method of claim 50, wherein the neurological disease or disorder would benefit from decreased neuronal cell death and/or increased neuronal survival, optionally wherein the neurological disease or disorder is selected from the group consisting of: Alzheimer's disease, Parkinson's disease, Huntington's disease, Pick's disease, Kuf's disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dytonia syndrome, Leigh syndrome, Leber hereditary optic neuropathy (LHON), parkinsonism, dystonia, motor neuron disease, neuropathy-ataxia and retinitis pimentosa (NARP), maternal inherited Leigh syndrome (MILS), Friedreich ataxia, hereditary spastic paraplegia, Mohr-Tranebjaerg syndrome, Wilson disease, sporatic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, korsakoff's psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity and movement disorders.

52. A method of increasing muscle physiology of a muscle tissue, the method comprising contacting the muscle tissue with an agent comprising: i) the irisin polypeptide of any one of claims 1 to 27 or a biologically active fragment thereof, or ii) the nucleic acid of claim 28 or 29 or a biologically active fragment thereof.

53. The method of claim 52, wherein the step of contacting occurs in vivo, ex vivo, or in vitro.

54. The method of claim 52 or 53, wherein the muscle tissue is skeletal muscle tissue, cardiac muscle tissue, and/or smooth muscle tissue.

55. The method of any one of claims 52 to 54, wherein the muscle tissue is affected by a muscular dystrophy and/or comprises a muscle cell having a mutation in a gene associated with a muscular dystrophy, optionally wherein the gene is dystrophin.

56. The method of claim 55, wherein the muscular dystrophy is selected from the group consisting of Becker, congenital, Duchenne, distal, Emery-Dreifuss, facioscapulohumeral, limb-girdle, myotonic, and oculopharyngeal muscular dystrophy.

57. The method of claim 55 or 56, wherein the muscular dystrophy is Duchenne muscular dystrophy (DMD).

58. The method of any one of claims 52 to 57, wherein the muscle tissue is affected by muscular atrophy.

59. The method of any one of claims 52 to 54, wherein the muscle tissue is affected by muscular atrophy resulting from disuse, trauma or a disease other than muscular dystrophy.

60. The method of claim 59, wherein the disease is Charcot-Marie-Tooth disease or spinal muscular atrophy.

61. The method of any one of claims 52 to 60, wherein the muscle physiology is selected from the group consisting of:
a) increasing the expression of at least one neuromuscular junction biomarker;
b) decreasing a biomarker of muscle injury, optionally wherein the biomarker is creatine kinase;
c) decreasing the proportion of injured muscle cells in a tissue, optionally wherein the injured muscle cells are detected using an Evans blue staining assay;
d) increasing the time of muscle activity, optionally wherein the time of muscle activity is measured using a running assay;
e) increasing muscle strength, optionally wherein the muscle strength is measured using a forelimb grip strength assay;
f) increasing lean muscle mass;
g) regeneration of muscle tissue;
h) decreasing fat mass; and
i) inhibition of muscle atrophy.

62. The method of claim 61, wherein the at least one neuromuscular junction biomarker is selected from the group consisting of PGC-1α, acetylcholine receptor cluster (Chrne), acetylcholinesterase (AchE), utrophin, and GA binding protein transcription factor subunit alpha (GABPA).

63. A method for preventing or treating muscular atrophy in a subject, comprising the step of administering to the subject an agent comprising: i) the irisin polypeptide of any one of claims 1 to 27 or a biologically active fragment thereof, or ii) the nucleic acid of claim 28 or 29 or a biologically active fragment thereof.

64. The method of claim 63, wherein the muscular atrophy is caused by disuse, trauma, or disease.

65. The method of claim 64, wherein the disease is Charcot-Marie-Tooth disease or spinal muscular atrophy.

66. A method for preventing or treating a muscular dystrophy in a subject, comprising the step of administering to the subject an agent comprising: i) the irisin polypeptide of any one of claims 1 to 27 or a biologically active fragment thereof, or ii) the nucleic acid of claim 28 or 29 or a biologically active fragment thereof.

67. The method of claim 66, wherein muscle cells of the subject comprise a mutation in a gene associated with a muscular dystrophy, optionally wherein the gene is dystrophin.

68. The method of any one of claims 66 to 67, wherein the muscular dystrophy is selected from the group consisting of Becker, congenital, Duchenne, distal, Emery-Dreifuss, facioscapulohumeral, limb-girdle, myotonic, and oculopharyngeal muscular dystrophy.

69. The method of claim 68, wherein the muscular dystrophy is Duchenne muscular dystrophy (DMD).

70. The method of any one of claims 31 to 69, further comprising administering conjointly to the subject an additional agent that increases an expression or activity of the irisin polypeptide.

71. The method of any one of claims 31 to 70, wherein the agent is administered systemically, optionally wherein systemic administration is intravenous or subcutaneous.

72. The method of any one of claims 31 to 71, wherein the agent is administered in a pharmaceutically acceptable formulation.

73. The method of any one of claims 31 to 72, wherein the agent is administered at least once a day, at least one a week, or at least once a month.

74. The method of any one of claims 31 to 73, wherein the agent is administered to the subject for greater than a number of months equal to 3, 4, 5, 6, 7, 8, 9, 10, 11, 12, 13, 14, 15, 16, 17, 18, 19, 20, 21, 22, 23, or 24, optionally the agent is administered to the subject for the duration of the subject's life.

75. The method of any one of claims 31 to 74, wherein the subject is a mammal, optionally wherein the mammal is a rodent, a primate, or a human.


SEQUENCE LISTING (36 sequences)

SEQ ID NO: 1 - protein, 112 aa, synthetic construct
DSPSAPVNVTVRHLKAQSAVVSWDVLEDEVVIGFAISQQKKDVRMLRFIQEVNTTTRSCALWDLEEDTEYIVHVQAISIQGQSPASEPVLFKTPREAEKMASKNKDEVTMKE

SEQ ID NO: 2 - protein, 112 aa, synthetic construct
DSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKE

SEQ ID NO: 3 - protein, 131 aa, synthetic construct
DSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKEHHHHHHHHHHRDPLEVLFQ

SEQ ID NO: 4 - DNA, 630 bp, Mus musculus
  source 1..630: mol_type = genomic DNA
atgcccccagggccgtgcgcctggccgccccgcgccgcgctccgcctgtggctaggctgcgtctgcttcgcgctggtgcaggcggacagcccctcagcccctgtgaacgtgaccgtccggcacctcaaggccaactctgccgtggtcagctgggatgtcctggaggatgaagtggtcattggctttgccatctctcagcagaagaaggatgtgcggatgctccggttcattcaggaggtgaacaccaccacccggtcctgcgctctctgggacctggaggaggacacagaatatatcgtccatgtgcaggccatctccatccagggacagagcccagccagtgagcctgtgctcttcaagaccccacgcgaggctgaaaagatggcctcaaagaacaaagatgaggtgaccatgaaggagatggggaggaaccagcagctgcgaacgggggaggtgctgatcattgttgtggtcctcttcatgtgggcaggtgttatagctctcttctgccgccagtatgatatcatcaaggacaacgagcccaataacaacaaggagaaaaccaagagcgcatcagaaaccagcacaccggagcatcagggtgggggtctcctccgcagcaagatatga

SEQ ID NO: 5 - protein, 209 aa, Mus musculus
MPPGPCAWPPRAALRLWLGCVCFALVQADSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKEMGRNQQLRTGEVLIIVVVLFMWAGVIALFCRQYDIIKDNEPNNNKEKTKSASETSTPEHQGGGLLRSKI

SEQ ID NO: 6 - DNA, 849 bp, Homo sapiens
  source 1..849: mol_type = genomic DNA
aaagagaaaagagagagagaggtgctctggctccggggctcttctcccaaacgggtcgaggtcccagctgaggctctcccaggacaagtctctcagccccagtgaacgtcaccgtcaggcacctcaaggccaactctgcagtggtgagctgggatgttctggaggatgaggttgtcatcggatttgccatctcccagcagaagaaggatgtgcggatgctgcgcttcatccaggaggtgaacaccaccacccgctcatgtgccctctgggacctggaggaggatacggagtacatagtccacgtgcaggccatctccattcagggccagagcccagccagcgagcctgtgctcttcaagaccccgcgtgaggctgagaagatggcctccaagaacaaagatgaggtaaccatgaaagagatggggaggaaccaacagctgcggacaggcgaggtgctgatcatcgtcgtggtcctgttcatgtgggcaggtgtcattgccctcttctgccgccagtatgacatcatcaaggacaatgaacccaataacaacaaggaaaaaaccaagagtgcatcagaaaccagcacaccagagcaccagggcggggggcttctccgcagcaaggtgagggcaagacctgggcctgggtgggccaccctgtgcctcatgctctggtaatccctggactgcagagggggtcagctcggggactggcatggcgacagctgggcagagccagacttggctgctgcctgtgtgacacagggacactgcaagttgattttggatcctctccttttgcagggtgccaggggccagtggtgatttgaagaaaaaaaaaaaaaaaaaa

SEQ ID NO: 7 - protein, 212 aa, Homo sapiens
MHPGSPSAWPPRARAALRLWLGCVCFALVQADSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKEMGRNQQLRTGEVLIIVVVLFMWAGVIALFCRQYDIIKDNEPNNNKEKTKSASETSTPEHQGGGLLRSKI

SEQ ID NO: 8 - DNA, 1203 bp, Gallus gallus
  source 1..1203: mol_type = genomic DNA
atggagaagaacagggacggccgcggcccccctggtgtccatctggggatggagaaggaagatgatttagagcccggtgacacgccggggctgcgcgaagccctggtggcgagatgtcaccgctgccgcgcacccgccgggggtctcaccgggacgggccccgtttgctccttccggcgatggggagcggtccgggccgagggctcccggtcccgcctgggggaaactgaggcagacggcggggccgggcggggcgggggccgagccgcccccgggccgggggagggaccggagcggggctgcccagcgctgcagcgggcggagccggggctcggcggggccgcctcccggccgagccgagccgaaccgagccgcgctgccgagggccgccgagcccgcagccgcccccggccgaaccgggcggccccgccggttccgggccccggagctctccgcggtgctgaacggcgccgccgcgcccgcgggacgccggccccggagcggctcggccccggcgcggcgcggcgggccgcggggggatggagcccttcctgggctgcaccggcgccgcgctcctgctctgctttcagctacgccggtctgcggccggtggaggcagacagcccttcggctccggtcaatgtcacagtcaaacacctgaaggccaactcagctgtagtgacttgggacgttctggaggatgaagttgtcattggatttgccatttcccagcagaagaaggacgtgcggatgctgcgcttcatccaggaggtgaacaccaccacccgctcctgtgccctctgggacctagaggaggacactgagtacattgtgcatgtccaggccatcagcatccaaggccagagccctgccagtgagccagtcctcttcaagacccccagggaagctgagaaactggcttctaaaaataaagatgaggtgacaatgaaggagatggcgaagaaaaaccaacagctgcgcgcaggggaaatactcatcattgtggtggtgttgtttatgtgggcaggggtgatcgccctgttctgcaggcagtacgacatcatcaaagacaacgagccgaacaacagcaaggagaaagccaagagcgcctcagagaacagcacccccgagcaccagggtggggggctgctccgcagcaagttcccaaaaaacaaaccctcagtgaacatcattgaggcataa

SEQ ID NO: 9 - protein, 400 aa, Gallus gallus
MEKNRDGRGPPGVHLGMEKEDDLEPGDTPGLREALVARCHRCRAPAGGLTGTGPVCSFRRWGAVRAEGSRSRLGETEADGGAGRGGGRAAPGPGEGPERGCPALQRAEPGLGGAASRPSRAEPSRAAEGRRARSRPRPNRAAPPVPGPGALRGAERRRRARGTPAPERLGPGAARRAAGGWSPSWAAPAPRSCSAFSYAGLRPVEADSPSAPVNVTVKHLKANSAVVTWDVLEDEVVIGFAISQQKKDVRMLRFIQEVNTTTRSCALWDLEEDTEYIVHVQAISIQGQSPASEPVLFKTPREAEKLASKNKDEVTMKEMAKKNQQLRAGEILIIVVVLFMWAGVIALFCRQYDIIKDNEPNNSKEKAKSASENSTPEHQGGGLLRSKFPKNKPSVNIIEA

SEQ ID NO: 10 - DNA, 336 bp, Mus sp.
  source 1..336: mol_type = genomic DNA
gacagcccctcagcccctgtgaacgtgaccgtccggcacctcaaggccaactctgccgtggtcagctgggatgtcctggaggatgaagtggtcattggctttgccatctctcagcagaagaaggatgtgcggatgctccggttcattcaggaggtgaacaccaccacccggtcctgcgctctctgggacctggaggaggacacagaatatatcgtccatgtgcaggccatctccatccagggacagagcccagccagtgagcctgtgctcttcaagaccccacgcgaggctgaaaagatggcctcaaagaacaaagatgaggtgaccatgaaggag

SEQ ID NO: 11 - DNA, 336 bp, Homo sapiens
  source 1..336: mol_type = genomic DNA
gacagtccctcagccccagtgaacgtcaccgtcaggcacctcaaggccaactctgcagtggtgagctgggatgttctggaggatgaggttgtcatcggatttgccatctcccagcagaagaaggatgtgcggatgctgcgcttcatccaggaggtgaacaccaccacccgctcatgtgccctctgggacctggaggaggatacggagtacatagtccacgtgcaggccatctccattcagggccagagcccagccagcgagcctgtgctcttcaagaccccgcgtgaggctgagaagatggcctccaagaacaaagatgaggtaaccatgaaagag

SEQ ID NO: 12 - protein, 19 aa, Mus sp.
MEWSWVFLFFLSVTTGVHS

SEQ ID NO: 13 - protein, 98 aa, synthetic construct
MSPSAPVNVTVRHLKAQSAVVSWDVLEDEVVIGFAISQQKKDVRMLRFIQEVNTTTRSCALWDLEEDTEYIVHVQAISIQGQSPASEPVLFKTPREAE

SEQ ID NO: 14 - protein, 98 aa, synthetic construct
MSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAE

SEQ ID NO: 15 - protein, 131 aa, synthetic construct
DSPSAPVNVTVRHLKAQSAVVSWDVLEDEVVIGFAISQQKKDVRMLRFIQEVNTTTRSCALWDLEEDTEYIVHVQAISIQGQSPASEPVLFKTPREAEKMASKNKDEVTMKEHHHHHHHHHHRDPLEVLFQ

SEQ ID NO: 16 - protein, 1151 aa, synthetic construct
MAFPPRRRLRLGPRGLPLLLSGLLLPLCRAFNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVSPASSFHVLRSLPLSSKGSGSAGWDVIQMTPQEIAVNLRPGDKTTFQLQVRQVEDYPVDLYYLMDLSLSMKDDLDNIRSLGTKLAEEMRKLTSNFRLGFGSFVDKDISPFSYTAPRYQTNPCIGYKLFPNCVPSFGFRHLLPLTDRVDSFNEEVRKQRVSRNRDAPEGGFDAVLQAAVCKEKIGWRKDALHLLVFTTDDVPHIALDGKLGGLVQPHDGQCHLNEANEYTASNQMDYPSLALLGEKLAENNINLIFAVTKNHYMLYKNFTALIPGTTVEILDGDSKNIIQLIINAYNSIRSKVELSVWDQPEDLNLFFTATCQDGVSYPGQRKCEGLKIGDTASFEVSLEARSCPSRHTEHVFALRPVGFRDSLEVGVTYNCTC

SEQ ID NO: 17 - protein, 8 aa, synthetic construct
HHHHHHHH

SEQ ID NO: 18 - protein, 6 aa, synthetic construct
HHHHHH

SEQ ID NO: 19 - protein, 10 aa, synthetic construct
HHHHHHHHHH

SEQ ID NO: 20 - protein, 14 aa, synthetic construct
  SITE 9: note = glycosylated-Asparagine
ADSPSAPVNVTVRH

SEQ ID NO: 21 - protein, 27 aa, synthetic construct
  SITE 3: note = glycosylated-Asparagine
KANSAVVSWDVLEDEVVIGFAISQQKK

SEQ ID NO: 22 - protein, 12 aa, synthetic construct
  SITE 7: note = glycosylated-Asparagine
RFIQEVNTTTRS

SEQ ID NO: 23 - protein, 1066 aa, Homo sapiens
MAFPPRRRLRLGPRGLPLLLSGLLLPLCRAFNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPGCGGLEVLFQGPGENAQCEKELQALEKENAQLEWELQALEKELAQGGWSHPQFEKGGGSGGGGSGGSAWSHPQFEK

SEQ ID NO: 24 - protein, 854 aa, Homo sapiens
MPRAPAPLYACLLGLCALLPRLAGLNICTSGSATSCEECLLIHPKCAWCSKEDFGSPRSITSRCDLRANLVKNGCGGEIESPASSFHVLRSLPLSSKGSGSAGWDVIQMTPQEIAVNLRPGDKTTFQLQVRQVEDYPVDLYYLMDLSLSMKDDLDNIRSLGTKLAEEMRKLTSNFRLGFGSFVDKDISPFSYTAPRYQTNPCIGYKLFPNCVPSFGFRHLLPLTDRVDSFNEEVRKQRVSRNRDAPEGGFDAVLQAAVCKEKIGWRKDALHLLVFTTDDVPHIALDGKLGGLVQPHDGQCHLNEANEYTASNQMDYPSLALLGEKLAENNINLIFAVTKNHYMLYKNFTALIPGTTVEILDGDSKNIIQLIINAYNSIRSKVELSVWDQPEDLNLFFTATCQDGVSYPGQRKCEGLKIGDTASFEVSLEARSCPSRHTEHVFALRPVGFRDSLEVGVTYNCTCGCSVGLEPNSARCNGSGTYVCGLCECSPGYLGTRCECQDGENQSVYQNLCREAEGKPLCSGRGDCSCNQCSCFESEFGKIYGPFCECDNFSCARNKGVLCSGHGECHCGECKCHAGYIGDNCNCSTDISTCRGRDGQICSERGHCLCGQCQCTEPGAFGEMCEKCPTCPDACSTKRDCVECPLLHSGKPDNQTCHSLCRDEVITWVDTIVKDDQEAVLCFYKTAKDCVMMFTYVELPSGKSNLTVLREPECGNTPNAMTILLAVVGSILLVGLALLAIWKLLVTIHDRREFAKFQSERSRARYEMASNPLYRKPISTHTVDFTFNKFNKSYNGTVDDTCGLEVLFQGPGKNAQCKKKLQALKKKNAQLKWKLQALKKKLAQGGHHHHHHHH

SEQ ID NO: 25 - protein, 111 aa, synthetic construct
DSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMK

SEQ ID NO: 26 - protein, 98 aa, Homo sapiens
TVSDVPRDLEVVAATPTSLLISWDAPAVTVRYYRITYGETGGNSPVQEFTVPGSKSTATISGLKPGVDYTITVYAVTGRGDSPASSKPISINYRTEID

SEQ ID NO: 27 - protein, 98 aa, Bos sp.
TVSDVPRDLEVIAATPTSLLISWDAPAVTVRYYRITYGETGGSSPVQEFTVPGSKSTATISGLKPGVDYTITVYAVTGRGDSPASSKPVSINYRTEID

SEQ ID NO: 28 - protein, 98 aa, Equus caballus
TVSDVPRDLEVIATTPTSILISWDAPAVTVRYYRITYGETGGNSPVQEFTVPGSKSTATISGLKPGVDYTITVYAVTGRGDSPASSKPISIDYRTEID

SEQ ID NO: 29 - protein, 98 aa, Gallus gallus
TVSDVPRDLEVNPTSPTSLEISWDAPAVTVRYYRITYGETGGSSPVQEFTVPGTMSRATITGLKPGVDYTITVYAVTGRGDSPASSKPVTVTYKTEID

SEQ ID NO: 30 - protein, 98 aa, Mus sp.
TVSDIPRDLEVIASTPTSLLISWEPPAVSVRYYRITYGETGGNSPVQEFTVPGSKSTATINNIKPGADYTITLYAVTGRGDSPASSKPVSINYKTEID

SEQ ID NO: 31 - protein, 98 aa, Rattus sp.
TVSDVPRDLEVIASTPTSLLISWEPPAVSVRYYRITYGETGGNSPVQEFTVPGSKSTATINNIKPGADYTITVYAVTGRGDSPASSKPVSINYQTEID

SEQ ID NO: 32 - protein, 90 aa, Homo sapiens
APSQIEVKDVTDTTALITWFKPLAEIDGIELTYGIKDVPGDRTTIDLTEDENQYSIGNLKPDTEYEVSLISRRGDMSSNPAKETFTTGLD

SEQ ID NO: 33 - protein, 90 aa, Sus sp.
APSQIEAKDVTDTTALITWFKPLAEIDGIELTYGIKDVPGDRTTIDLTHEENQYSIGNLKPDTEYEVSLISRRADMSSNPAKETFTTGLD

SEQ ID NO: 34 - protein, 91 aa, Gallus gallus
APSQIEAKDVTDTTALITWSKPLAEIEGIELTYGPKDVPGDRTTIDLSEDENQYSIGNLRPHTEYEVTLISRRGDMESDPAKEVFVTDLDA

SEQ ID NO: 35 - protein, 92 aa, Rattus sp.
APSQIEVKDITDTTALITWSKPLAEIDSIELSYGIKDVPGDRTTIDLTHEDNQYSIGNLKPDTEYEVSLISRRVDMSSNPAKETFITGLDAP

SEQ ID NO: 36 - protein, 90 aa, Mus sp.
APSHIEVKDVTDTTALITWFKPLAEIDSIELSYGIKDVPGDRTTIDLTHEDNQYSIGNLRPDTEYEVSLISRRVDMASNPAKETFITGLD